(19) United States
(12) Patent Application Publication
(10) Pub. No.: US 2014/0243335 A1
(43) Pub. Date: Aug. 28, 2014

(54) SUBSTITUTED HYDOXAMIC ACIDS AND USES THEREOF

(71) Applicants: Christopher Blackburn, Natick, MA (US); Emily F. Calderwood, Framingham, MA (US); Kenneth M. Gigstad, Westford, MA (US); Alexandra E. Gould, Cambridge, MA (US); Sean J. Harrison, Belmont, MA (US); He Xu, Needham, MA (US)

(72) Inventors: Christopher Blackburn, Natick, MA (US); Emily F. Calderwood, Framingham, MA (US); Kenneth M. Gigstad, Westford, MA (US); Alexandra E. Gould, Cambridge, MA (US); Sean J. Harrison, Belmont, MA (US); He Xu, Needham, MA (US)

(73) Assignee: Millennium Pharmaceuticals, Inc., Cambridge, MA (US)

(21) Appl. No.: 14/270,028

(22) Filed: May 5, 2014

Related U.S. Application Data
(63) Continuation of application No. 13/184,600, filed on Jul. 18, 2011.
(60) Provisional application No. 61/426,293, filed on Dec. 22, 2010.
(60) Provisional application No. 61/365,500, filed on Jul. 19, 2010.

(51) Int. Cl. C07D 239/26; C07D 211/64; C07C 259/18; C07D 213/75; C07D 213/56; C07D 231/12; C07D 207/34; C07D 213/68

(52) U.S. Cl. CPC C07D 239/26; C07D 207/34; C07D 211/64; C07D 213/68; C07D 213/75; C07D 213/56; C07D 231/12; C07C 259/18
USPC 514/235.5; 514/253.01; 514/256; 514/319; 514/351; 514/352; 514/357; 514/406; 514/563; 544/141; 544/329; 544/360; 546/205; 546/300; 546/309; 546/337; 548/377.1; 560/315; 564/173

(57) ABSTRACT

This invention provides compounds of formula (I):

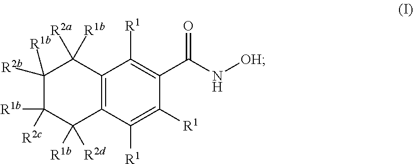

wherein R¹, R^{1b}, R^{2a}, R^{2b}, R^{2c}, and R^{2d} have values as described in the specification, useful as inhibitors of HDAC6. The invention also provides pharmaceutical compositions comprising the compounds of the invention and methods of using the compositions in the treatment of proliferative, inflammatory, infectious, neurological or cardiovascular diseases or disorders.

### PRIORITY CLAIM

This application is a continuation of U.S. patent application Ser. No. 13/184,600, filed Jul. 18, 2011 (now pending), which claims the benefit under 35 U.S.C. §119(e) of U.S. Provisional Patent Application Ser. No. 61/365,500, filed Jul. 19, 2010, incorporated by reference in its entirety, and U.S. Provisional Patent Application Ser. No. 61/426,293, filed Dec. 22, 2010, incorporated by reference in its entirety.

### FIELD OF THE INVENTION

The invention relates to compounds and methods for the selective inhibition of HDAC6. The present invention relates to compounds useful as HDAC6 inhibitors. The invention also provides pharmaceutical compositions comprising the compounds of the invention and methods of using the compositions in the treatment of various diseases.

### BACKGROUND OF THE INVENTION

Histone deacetylase 6 (HDAC6) is a member of a family of amidohydrolases commonly referred as histone or lysine deacetylases (HDACs or KDACs) as they catalyze the removal of acetyl groups from the ε-amino group of lysine residues from proteins. The family includes 18 enzymes which can be divided in 3 main classes based on their sequence homology to yeast enzymes Rpd3 (Class I), Hda1 (Class II) and Sir2 (Class III). A fourth class was defined with the finding of a distinct mammalian enzyme—HDAC11 (reviewed in Yang, et al., Nature Rev. Mol. Cell. Biol. 2008, 9:206-218 and in Saunders and Verdin, Oncogene 2007, 26(37):5489-5504). Biochemically, Class I (HDAC1, 2, 3, 8) and Class II (HDAC4, 5, 6, 7, 9, 10) and Class IV (HDAC11) are Zn²⁺-dependent enzymes, while Class III (SIRT1-7) are dependent on nicotinamide adenine dinucleotide (NAD⁺) for activity. Unlike all other HDACs, HDAC6 resides primarily in the cytosol, it has 2 functional catalytic domains and a carboxy-terminal Zn²⁺-finger ubiquitin binding domain. HDAC6 has been shown to bind ubiquitinated misfolded proteins (Kawaguchi et al., Cell 2003, 115(6):727-738), ubiquitin (Boyaullt et al., EMBO J. 2006, 25(14): 3357-3366), as well as the ubiquitin-like modifier, FAT10 (Kalveram et al., J. Cell Sci. 2008, 121(24):4079-4088). Known substrates of HDAC6 include cytoskeletal proteins α-tubulin and cortactin; β-catenin which forms part of adherens junctions and anchors the actin cytoskeleton; the chaperone Hsp90; and the redox regulatory proteins peroxiredoxin (Prx) I and Prx II (reviewed in Boyault et al., Oncogene 2007, 26(37):5468-5476; Matthias et al., Cell Cycle 2008, 7(1):7-10; Li et al., J Biol. Chem. 2008, 283(19):12686-12690; Parmigiani et al., Proc. Natl. Acad. Sci. USA 2009, 105(28):9633-9638). Thus, HDAC6 mediates a wide range of cellular functions including microtubule-dependent trafficking and signaling, membrane remodeling and chemotactic motility, involvement in control of cellular adhesion, ubiquitin level sensing, regulation of chaperone levels and activity, and responses to oxidative stress. All of these functions may be important in tumorigenesis, tumor growth and survival as well as metastasis (Simms-Waldrip et al., Mol. Genet. Metabolism 2008, 94(3):283-286; Rodriguez-Gonzalez et al., Cancer Res. 2008, 68(8):2557-2560; Kapoor, Int. J. Cancer 2009, 124:509; Lee et al., Cancer Res. 2008, 68(18):7561-7569). Recent studies have shown HDAC6 to be important in autophagy, an alternative pathway for protein degradation that compensates for deficiencies in the activity of the ubiquitin-proteasome system or the expression of proteins prone to form aggregates and can be activated following treatment with a proteasome inhibitor (Kawaguchi et al., Cell 2003, 115(6):727-738; Iwata et al., J. Biol. Chem. 2005, 280(48): 40282-40292; Ding et al., Am. J. Pathol. 2007, 171:513-524, Pandey et al., Nature 2007, 447(7146):860-864). Although the molecular mechanistic details are not completely understood, HDAC6 binds ubiquitinated or ubiquitin-like conjugated misfolded proteins which would otherwise induce proteotoxic stress and then serves as an adaptor protein to traffic the ubiquitinated cargo to the microtubule organizing center using the microtubule network via its known association with dynein motor protein. The resulting perinuclear aggregates, known as aggresomes, are then degraded by fusion with lysosomes in an HDAC6- and cortactin-dependent process which induces remodeling of the actin cytoskeleton proximal to aggresomes (Lee et al., EMBO J. 2010, 29:969-980). In addition, HDAC6 regulates a variety of biological processes dependent on its association with the microtubular network including cellular adhesion (Tran et al., J. Cell Sci. 2007, 120(8):1469-1479) and migration (Zhang et al., Mol. Cell. 2007, 27(2):197-213; reviewed in Valenzuela-Fernandez et al., Trends Cell. Biol. 2008, 18(6):291-297), epithelial to mesenchymal transition (Shan et al., J. Biol. Chem. 2008, 283(30):21065-21073), resistance to anoikis (Lee et al., Cancer Res. 2008, 68(18):7561-7569), epithelial growth factor-mediated Wnt signaling via β-catenin deacetylation (Li et al., J. Biol. Chem. 2008, 283(19):12686-12690) and epithelial growth factor receptor stabilization by endocytic trafficking (Lissanu Deribe et al., Sci. Signal. 2009, 2(102): ra84; Gao et al., J. Biol. Chem. 2010, 285:11219-11226); all events that promote oncogenesis and metastasis (Lee et al., Cancer Res. 2008, 68(18):7561-7569). HDAC6 activity is known to be upregulated by Aurora A kinase in cilia formation (Pugacheva et al., Cell 2007, 129(7):1351-1363) and indirectly by farnesyl transferase with which HDAC6 forms a complex with microtubules (Zhou et al., J. Biol. Chem. 2009, 284(15): 9648-9655). Also, HDAC6 is negatively regulated by tau protein (Perez et al., J. Neurochem. 2009, 109(6):1756-1766).

Diseases in which selective HDAC6 inhibition could have a potential benefit include cancer (reviewed in Simms-Waldrip et al., Mol. Genet. Metabolism 2008, 94(3):283-286 and Rodriguez-Gonzalez et al., Cancer Res. 2008, 68(8):2557-2560), specifically: multiple myeloma (Hideshima et al., Proc. Natl. Acad. Sci. USA 2005, I02(24):8567-8572); lung cancer (Kamemura et al., Biochem. Biophys. Res. Commun. 2008, 374(1):84-89); ovarian cancer (Bazzaro et al., Clin. Cancer Res. 2008, 14(22):7340-7347); breast cancer (Lee et al., Cancer Res. 2008, 68(18):7561-7569); prostate cancer (Mellado et al., Clin. Trans. Onco. 2009, 11(1):5-10); pancreatic cancer (Nawrocki et al., Cancer Res. 2006, 66(7):3773-3781); renal cancer (Cha et al., Clin. Cancer Res. 2009, 15(3):840-850); and leukemias such as acute myeloid leukemia (AML) (Fiskus et al., Blood 2008, 112(7):2896-2905) and acute lymphoblastic leukemia (ALL) (Rodriguez-Gonzalez et al., Blood 2008, 112(11): Abstract 1923).

Inhibition of HDAC6 may also have a role in cardiovascular disease, i.e. cardiovascular stress, including pressure overload, chronic ischemia, and infarction-reperfusion injury (Tannous et al., Circulation 2008, 117(24):3070-3078); bacterial infection, including those caused by uropathogenic Escherichia coli (Dhakal and Mulve, J. Biol. Chem. 2008, 284(1):446-454); neurological diseases caused by accumulation of intracellular protein aggregates such as Huntington's disease (reviewed in Kazantsev et al., Nat. Rev. Drug Disc. 2008, 7(10):854-868; see also Dompierre et al., J. Neurosci. 2007, 27(13):3571-3583; Kozikowski et al., J. Med. Chem. 2007, 50:3054-3061) or central nervous system trauma caused by tissue injury, oxidative-stress induced neuronal or axomal degeneration (Rivieccio et al., Proc. Natl. Acad. Sci. USA 2009, 106(46):19599-195604); and inflammation, including reduction of pro-inflammatory cytokine IL-1β (Carta et al., Blood 2006, 108(5):1618-1626), increased expression of the FOXP3 transcription factor, which induces immunosuppressive function of regulatory T-cells resulting in benefits in chronic diseases such as rheumatoid arthritis, psoriasis, multiple sclerosis, lupus and organ transplant rejection (reviewed in Wang et al., Nat. Rev. Drug Disc. 2009 8(12):969-981).

Given the complex function of HDAC6, selective inhibitors could have potential utility when used alone or in combination with other chemotherapeutics such as microtubule destabilizing agents (Zhou et al., J. Biol. Chem. 2009, 284(15): 9648-9655); Hsp90 inhibitors (Rao et al., Blood 2008, 112(5)1886-1893); inhibitors of Hsp90 client proteins, including receptor tyrosine kinases such as Her-2 or VEGFR (Bhalla et al., J. Clin. Oncol. 2006, 24(18S): Abstract 1923; Park et al., Biochem. Biophys. Res. Commun. 2008, 368(2):318-322), and signaling kinases such as Bcr-Abl, Akt, mutant FLT-3, c-Raf, and MEK (Bhalla et al., J. Clin. Oncol. 2006, 24(18S): Abstract 1923; Kamemura et al., Biochem. Biophys. Res. Commun. 2008, 374(1):84-89); inhibitors of cell cycle kinases Aurora A and Aurora B (Pugacheva et al., Cell 2007, 129(7):1351-1363; Park et al., J. Mol. Med. 2008, 86(1):117-128; Cha et al., Clin. Cancer Res. 2009, 15(3):840-850); EGFR inhibitors (Lissanu Deribe et al., Sci. Signal. 2009, 2(102): ra84; Gao et al., J. Biol. Chem. E-pub Feb. 4, 2010) and proteasome inhibitors (Hideshima et al., Proc. Natl. Acad. Sci. USA 2005, 102(24):8567-8572) or other inhibitors of the ubiquitin proteasome system such as ubiquitin and ubiqutin-like activating (E1), conjugation (E2), ligase enzymes (E3, E4) and deubiquitinase enzymes (DUBs) as well as modulators of autophagy and protein homeostasis pathways. In addition, HDAC6 inhibitors could be combined with radiation therapy (Kim et al., Radiother. Oncol. 2009, 92(1):125-132.

Clearly, it would be beneficial to provide novel HDAC6 inhibitors that possess good therapeutic properties, especially for the treatment of proliferative diseases or disorders.

### DETAILED DESCRIPTION OF THE INVENTION

### 1. General Description of Compounds of the Invention

The present invention provides compounds that are effective inhibitors of HDAC6. These compounds are useful for inhibiting HDAC6 activity in vitro and in vivo, and are especially useful for the treatment of various cell proliferative diseases or disorders. The compounds of the invention are represented by formula (I):

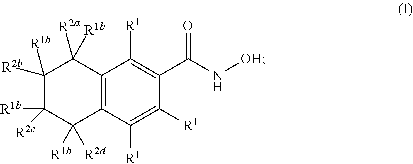

or a pharmaceutically acceptable salt thereof;

wherein:

each occurrence of R¹ is independently hydrogen, chloro, fluoro, —O—C₁₋₄ alkyl, cyano, hydroxy, C₁₋₄ alkyl, C₁₋₄ fluoroalkyl, —N(C₁₋₄ alkyl)₂, —NH(C₁₋₄ alkyl), —NH₂, or O—C₁₋₄ fluoroalkyl;

R^{2a} is G or R^{1a};

R^{2b} is G or R^{1a};

R^{2c} is G or R^{1a};

R^{2d} is G or R^{1a};

provided that one and only one of R^{2a}, R^{2b}, R^{2c}, and R^{2d} is G;

each occurrence of R^{1a} is independently hydrogen, fluoro, C₁₋₄ alkyl, or C₁₋₄ fluoroalkyl;

each occurrence of R^{1b} is independently hydrogen, fluoro, or C₁₋₄ alkyl;

or one occurrence of R^{1a} and one occurrence of R^{1b} on the same carbon atom can be taken together to form ═O or a 3-6 membered cycloaliphatic;

G is hydrogen, —R³, -V₁-R³, -V₁-L₁-R³, -L₁-V₁-R³, or -L₁-R³;

L₁ is an unsubstituted or substituted C₁₋₃ alkylene chain;

V₁ is —C(O)—, —C(S)—, —C(O)—N(R^{4a})—, —C(O)—O—, —N(R^{4a})—, —N(R^{4a})—C(O)—, —N(R^{4a})—SO₂—, —O—, —N(R^{4a})—C(O)—N(R^{4a})—, —N(R^{4a})—C(O)—O—, —O—C(O)—N(R^{4a})—, or —N(R^{4a})—SO₂—N(R^{4a})—;

R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; and

each occurrence of R^{4a} is independently hydrogen, or unsubstituted or substituted C₁₋₄ aliphatic; or when V₁ is —N(R^{4a})—C(O)—, —N(R^{4a})—SO₂—, or —N(R^{4a})—C(O)—N(R^{4a})—, one occurrence of R^{4a} can be taken together with an R^{1a} attached to a ring carbon atom that is not adjacent to the ring carbon atom to which G is attached to form a substituted or unsubstituted 5-7 membered bridged heterocyclyl;

provided that the compound is other than 8-(2-amino-8-bromo-1,6-dihydro-6-oxo-9H-purin-9-yl)-5,6,7,8-tetrahydro-N-hydroxy-2-naphthalenecarboxamide.

### 2. Compounds and Definitions

Compounds of this invention include those described generally for formula (I) above, and are further illustrated by the classes, subclasses, and species disclosed herein. As used herein, the following definitions shall apply unless otherwise indicated.

As described herein, compounds of the invention may be optionally substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which hydrogen atom can be replaced with the radical of a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

A stable compound or chemically feasible compound is one in which the chemical structure is not substantially altered when kept at a temperature from about 80° C. to about +40° C., in the absence of moisture or other chemically reactive conditions, for at least a week, or a compound which maintains its integrity long enough to be useful for therapeutic or prophylactic administration to a patient.

The phrase “one or more substituents”, as used herein, refers to a number of substituents that equals from one to the maximum number of substituents possible based on the number of available bonding sites, provided that the above conditions of stability and chemical feasibility are met.

As used herein, the term “independently selected” means that the same or different values may be selected for multiple instances of a given variable in a single compound.

As used herein, the term “aromatic” includes aryl and heteroaryl groups as described generally below and herein.

The term “aliphatic” or “aliphatic group”, as used herein, means an optionally substituted straight-chain or branched C₁₋₁₂ hydrocarbon. For example, suitable aliphatic groups include optionally substituted linear, or branched alkyl, alkenyl, alkynyl groups and hybrids thereof. Unless otherwise specified, in various embodiments, aliphatic groups have 1-12, 1-10, 1-8, 1-6, 1-4, 1-3, or 1-2 carbon atoms.

The term “alkyl”, used alone or as part of a larger moiety, refers to an optionally substituted straight or branched chain hydrocarbon group having 1-12, 1-10, 1-8, 1-6, 1-4, 1-3, or 1-2 carbon atoms.

The term “alkenyl”, used alone or as part of a larger moiety, refers to an optionally substituted straight or branched chain hydrocarbon group having at least one double bond and having 2-12, 2-10, 2-8, 2-6, 2-4, or 2-3 carbon atoms.

The term “alkynyl”, used alone or as part of a larger moiety, refers to an optionally substituted straight or branched chain hydrocarbon group having at least one triple bond and having 2-12, 2-10, 2-8, 2-6, 2-4, or 2-3 carbon atoms.

The terms “cycloaliphatic”, “carbocycle”, “carbocyclyl”, “carbocyclo”, or “carbocyclic”, used alone or as part of a larger moiety, refer to an optionally substituted saturated or partially unsaturated cyclic aliphatic ring system having from 3 to about 14 ring carbon atoms. In some embodiments, the cycloaliphatic group is an optionally substituted monocyclic hydrocarbon having 3-10, 3-8 or 3-6 ring carbon atoms. Cycloaliphatic groups include, without limitation, optionally substituted cyclopropyl, cyclobutyl, cyclopentyl, cyclopentenyl, cyclohexyl, cyclohexenyl, cycloheptyl, cycloheptenyl, cyclooctyl, cyclooctenyl, or cyclooctadienyl. The terms “cycloaliphatic”, “carbocycle”, “carbocyclyl”, “carbocyclo”, or “carbocyclic” also include optionally substituted bridged or fused bicyclic rings having 6-12, 6-10, or 6-8 ring carbon atoms, wherein any individual ring in the bicyclic system has 3-8 ring carbon atoms.

The term “cycloalkyl” refers to an optionally substituted saturated ring system of about 3 to about 10 ring carbon atoms. Exemplary monocyclic cycloalkyl rings include cyclopropyl, cyclobutyl, cyclopentyl, cyclohexyl, and cycloheptyl.

The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms. As used herein, the term “halogen” or “halo” means F, Cl, Br, or I. The term “fluoroaliphatic” refers to a haloaliphatic wherein the halogen is fluoro, including perfluorinated aliphatic groups. Examples of fluoroaliphatic groups include, without limitation, fluoromethyl, difluoromethyl, trifluoromethyl, 2-fluoroethyl, 2,2,2-trifluoroethyl, 1,1,2-trifluoroethyl, 1,2,2-trifluoroethyl, and pentafluoroethyl.

The term “heteroatom” refers to one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

The terms “aryl” and “ar-”, used alone or as part of a larger moiety, e.g., “aralkyl”, “aralkoxy”, or “aryloxyalkyl”, refer to an optionally substituted C₆₋₁₄aromatic hydrocarbon moiety comprising one to three aromatic rings. Preferably, the aryl group is a C₆₋₁₀aryl group. Aryl groups include, without limitation, optionally substituted phenyl, naphthyl, or anthracenyl. The terms “aryl” and “ar-”, as used herein, also include groups in which an aryl ring is fused to one or more cycloaliphatic rings to form an optionally substituted cyclic structure such as a tetrahydronaphthyl, indenyl, or indanyl ring. The term “aryl” may be used interchangeably with the terms “aryl group”, “aryl ring”, and “aromatic ring”.

An “aralkyl” or “arylalkyl” group comprises an aryl group covalently attached to an alkyl group, either of which independently is optionally substituted. Preferably, the aralkyl group is C₆₋₁₀arylC₁₋₆alkyl, including, without limitation, benzyl, phenethyl, and naphthylmethyl.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, 9, or 10 ring atoms; having 6, 10, or 14 π electrons shared in a cyclic array; and having, in addition to carbon atoms, from one to five heteroatoms. In some embodiments, the heteroaryl group has 5-10 ring atoms, having, in addition to carbon atoms, from one to five heteroatoms. A heteroaryl group may be mono-, or polycyclic, preferably mono-, bi-, or tricyclic, more preferably mono- or bicyclic. The term “heteroatom” refers to nitrogen, oxygen, or sulfur, and includes any oxidized form of nitrogen or sulfur, and any quaternized form of a basic nitrogen. For example, a nitrogen atom of a heteroaryl may be a basic nitrogen atom and may also be optionally oxidized to the corresponding N-oxide. When a heteroaryl is substituted by a hydroxy group, it also includes its corresponding tautomer. The terms “heteroaryl” and “heteroar-”, as used herein, also include groups in which a heteroaromatic ring is fused to one or more aryl, cycloaliphatic, or heterocycloaliphatic rings. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 4-10 membered ring, preferably a 3- to 8-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR+ (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiomorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. Additionally, a heterocyclic ring also includes groups in which the heterocyclic ring is fused to one or more aryl rings.

As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

The term “alkylene” refers to a bivalent alkyl group. An “alkylene chain” is a polymethylene group, i.e., —(CH₂)_{n′}—, wherein n′ is a positive integer, preferably from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3-7-membered ring. The substituents can be on the same or different atoms.

An alkylene chain also can be optionally interrupted by a functional group. An alkylene chain is “interrupted” by a functional group when an internal methylene unit is interrupted by the functional group. Examples of suitable “interrupting functional groups” are described in the specification and claims herein.

For purposes of clarity, all bivalent groups described herein, including, e.g., the alkylene chain linkers described above, are intended to be read from left to right, with a corresponding left-to-right reading of the formula or structure in which the variable appears.

An aryl (including aralkyl, aralkoxy, aryloxyalkyl and the like) or heteroaryl (including heteroaralkyl and heteroarylalkoxy and the like) group may contain one or more substituents and thus may be “optionally substituted”. In addition to the substituents defined above and herein, suitable substituents on the unsaturated carbon atom of an aryl or heteroaryl group also include and are generally selected from -halo, —NO₂, —CN, —R⁺, —C(R⁺)═C(R⁺)₂, —C≡C—R⁺, —OR⁺, —SR^{o}, —S(O)R^{o}, —SO₂R^{o}, —SO₃R⁺, —SO₂N(R⁺)₂, —N(R⁺)₂, —NR⁺C(O)R⁺, —NR⁺C(S)R⁺, —NR⁺C(O)N(R⁺)₂, —NR⁺C(S)N(R⁺)₂, —N(R⁺)C(═NR⁺)—N(R⁺)₂, —N(R⁺)C(═NR⁺)—R^{o}, —NR⁺CO₂R⁺, —NR⁺SO₂R^{o}, —NR⁺SO₂N(R⁺)², —O—C(O)R⁺, —O—CO₂R⁺, —OC(O)N(R⁺)₂, —C(O)R⁺, —C(S)R^{o}, —CO₂R⁺, —C(O)—C(O)R⁺, —C(O)N(R⁺)₂, —C(S)N(R⁺)₂, —C(O)N(R⁺)—OR⁺, —C(O)N(R⁺)C(═NR⁺)—N(R⁺)₂, —N(R⁺)C(═NR⁺)—N(R⁺)—C(O)R⁺, —C(═NR⁺)—N(R⁺)₂, —C(═NR⁺)—OR⁺, —N(R⁺)—N(R⁺)₂, —C(═NR⁺)—N(R⁺)—OR⁺, —C(R^{o})═N—OR⁺, —P(O)(R⁺)₂, —P(O)(OR⁺)₂, —O—P(O)—OR⁺, and —P(O)(NR⁺)—N(R⁺)₂, wherein R⁺, independently, is hydrogen or an optionally substituted aliphatic, aryl, heteroaryl, cycloaliphatic, or heterocyclyl group, or two independent occurrences of R⁺ are taken together with their intervening atom(s) to form an optionally substituted 5-7-membered aryl, heteroaryl, cycloaliphatic, or heterocyclyl ring. Each R^{o} is an optionally substituted aliphatic, aryl, heteroaryl, cycloaliphatic, or heterocyclyl group.

An aliphatic or heteroaliphatic group, or a non-aromatic carbycyclic or heterocyclic ring may contain one or more substituents and thus may be “optionally substituted”. Unless otherwise defined above and herein, suitable substituents on the saturated carbon of an aliphatic or heteroaliphatic group, or of a non-aromatic carbocyclic or heterocyclic ring are selected from those listed above for the unsaturated carbon of an aryl or heteroaryl group and additionally include the following: ═O, ═S, ═C(R*)₂, ═N—N(R*)₂, ═N—OR*, ═N—NHC(O)R*, ═N—NHCO₂R^{o}═N—NHSO₂R^{o} or ═N—R* where R^{o} is defined above, and each R* is independently selected from hydrogen or an optionally substituted C₁₋₆ aliphatic group.

In addition to the substituents defined above and herein, optional substituents on the nitrogen of a non-aromatic heterocyclic ring also include and are generally selected from R⁺, —N(R⁺)₂, —C(O)R⁺, —C(O)OR⁺, —C(O)C(O)R⁺, —C(O)CH₂C(O)R⁺, —S(O)₂R⁺, —S(O)₂N(R⁺)₂, —C(S)N(R⁺)₂, —C(═NH)—N(R⁺)₂, or —N(R⁺)S(O)₂R⁺; wherein each R⁺ is defined above. A ring nitrogen atom of a heteroaryl or non-aromatic heterocyclic ring also may be oxidized to form the corresponding N-hydroxy or N-oxide compound. A nonlimiting example of such a heteroaryl having an oxidized ring nitrogen atom is N-oxidopyridyl.

As detailed above, in some embodiments, two independent occurrences of R⁺ (or any other variable similarly defined in the specification and claims herein), are taken together with their intervening atom(s) to form a monocyclic or bicyclic ring selected from 3-13-membered cycloaliphatic, 3-12-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

Exemplary rings that are formed when two independent occurrences of R⁺ (or any other variable similarly defined in the specification and claims herein), are taken together with their intervening atom(s) include, but are not limited to the following: a) two independent occurrences of R⁺ (or any other variable similarly defined in the specification or claims herein) that are bound to the same atom and are taken together with that atom to form a ring, for example, N(R⁺)₂, where both occurrences of R⁺ are taken together with the nitrogen atom to form a piperidin-1-yl, piperazin-1-yl, or morpholin-4-yl group; and b) two independent occurrences of R⁺ (or any other variable similarly defined in the specification or claims herein) that are bound to different atoms and are taken together with both of those atoms to form a ring, for example where a phenyl group is substituted with two occurrences of OR⁺

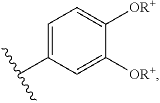

these two occurrences of R⁺ are taken together with the oxygen atoms to which they are bound to form a fused 6-membered oxygen containing ring:

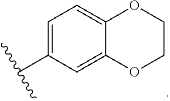 [SMILES: *c1ccc2c(c1)OCCO2]

It will be appreciated that a variety of other rings (e.g., spiro and bridged rings) can be formed when two independent occurrences of R+ (or any other variable similarly defined in the specification and claims herein) are taken together with their intervening atom(s) and that the examples detailed above are not intended to be limiting.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as analytical tools or probes in biological assays.

The terms “stereoisomer”, “enantiomer”, “diastereomer”, “epimer”, and “chiral center”, are used herein in accordance with the meaning each is given in ordinary usage by those of ordinary skill in the art. Thus, stereoisomers are compounds that have the same atomic connectivity, but differ in the spatial arrangement of the atoms. Enantiomers are stereoisomers that have a mirror image relationship, that is, the stereochemical configuration at all corresponding chiral centers is opposite. Diastereomers are stereoisomers having more than one chiral center, which differ from one another in that the stereochemical configuration of at least one, but not all, of the corresponding chiral centers is opposite. Epimers are diastereomers that differ in stereochemical configuration at only one chiral center.

It is to be understood that, when a disclosed compound has at least one chiral center, the present invention encompasses one enantiomer of the compound, substantially free from the corresponding optical isomer, a racemic mixture of both optical isomers of the compound, and mixtures enriched in one enantiomer relative to its corresponding optical isomer. When a mixture is enriched in one enantiomer relative to its optical isomer, the mixture contains, for example, an enantiomeric excess of at least 50%, 75%, 90%, 95%, 99%, or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed compound has at least two chiral centers, the present invention encompasses a diastereomer substantially free of other diastereomers, an enantiomeric pair of diastereomers substantially free of other stereoisomers, mixtures of diastereomers, mixtures of enantiomeric pairs of diastereomers, mixtures of diastereomers in which one diastereomer is enriched relative to the other diastereomer(s), and mixtures of enantiomeric pairs of diastereomers in which one enantiomeric pair of diastereomers is enriched relative to the other stereoisomers. When a mixture is enriched in one diastereomer or enantiomeric pair of diastereomers pairs relative to the other stereoisomers, the mixture is enriched with the depicted or referenced diastereomer or enantiomeric pair of diastereomers relative to other stereoisomers for the compound, for example, by a molar excess of at least 50%, 75%, 90%, 95%, 99%, or 99.5%.

As used herein, the term “diastereomeric ratio” refers to the ratio between diastereomers which differ in the stereochemical configuration at one chiral center, relative to a second chiral center in the same molecule. By way of example, a chemical structure with two chiral centers provides four possible stereoisomers: R*R, R*S, S*R, and S*S, wherein the asterisk denotes the corresponding chiral center in each stereoisomer. The diastereomeric ratio for such a mixture of stereoisomers is the ratio of one diastereomer and its enantiomer to the other diastereomer and its enantiomer ═(R*R+S*S): (R*S+S*R).

One of ordinary skill in the art will recognize that additional stereoisomers are possible when the molecule has more than two chiral centers. For purposes of the present invention, the term “diastereomeric ratio” has identical meaning in reference to compounds with multiple chiral centers as it does in reference to compounds having two chiral centers. Thus, the term “diastereomeric ratio” refers to the ratio of all compounds having R*R or S*S configuration at the specified chiral centers to all compounds having R*S or S*R configuration at the specified chiral centers. For convenience, this ratio is referred to herein as the diastereomeric ratio at the asterisked carbon, relative to the second specified chiral center.

The diastereomeric ratio can be measured by any analytical method suitable for distinguishing between diastereomeric compounds having different relative stereochemical configurations at the specified chiral centers. Such methods include, without limitation, nuclear magnetic resonance (NMR), gas chromatography (GC), and high performance liquid chromatography (HPLC) methods.

The diastereoisomeric pairs may be separated by methods known to those skilled in the art, for example chromatography or crystallization and the individual enantiomers within each pair may be separated as described above. Specific procedures for chromatographically separating diastereomeric pairs of precursors used in the preparation of compounds disclosed herein are provided the examples herein.

### 3. Description of Exemplary Compounds

In some embodiments, the compound of formula (I) is represented by:

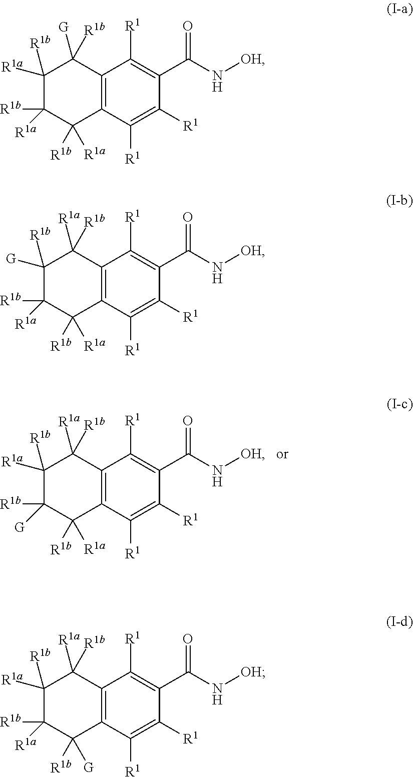

wherein R^{1a}, R^{1b}, R¹, and G have the values described herein. In certain embodiments, the compound of formula (I) is represented by formula (I-b), wherein R^{1a}, R^{1b}, R¹, and G have the values described herein. In certain embodiments, the compound of formula (I) is represented by formula (I-c), wherein R^{1a}, R^{1b}, R¹, G have the values described herein. In certain embodiments, the compound of formula (I) is represented by formula (I-d), wherein R^{1a}, R^{1b}, R¹, and G have the values described herein.

In some embodiments, the compound of formula (I) is represented by formula (II):

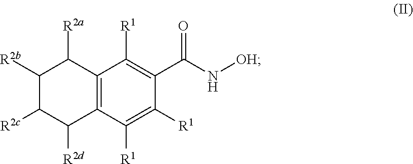

wherein R^{2a}, R^{2b}, R^{2c}, R^{2d}, and R¹ have the values described herein.

In some embodiments, the compound of formula (I) is represented by formula (II-a)-(II-d):

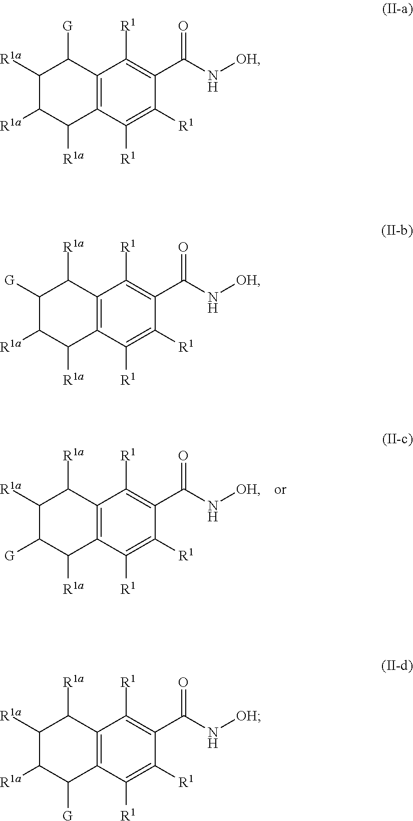

wherein R^{1a}, R¹, and G have the values described herein. In certain embodiments, the compound of formula (I) is represented by formula (II-b), wherein R^{1a}, R¹, and G have the values described herein. In certain embodiments, the compound of formula (I) is represented by formula (II-c), wherein R^{1a}, and G have the values described herein. In certain embodiments, the compound of formula (I) is represented by formula (II-d), wherein R^{1a}, R¹, and G have the values described herein.

In some embodiments, the compound of formula (I) is represented by formula (III):

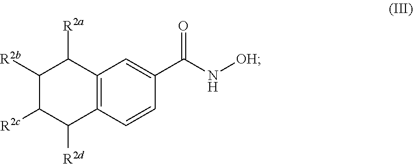

wherein R^{2a}, R^{2b}, R^{2c}, and R^{2d} have the values described herein.

In some embodiments, the compound of formula (I) is represented by formula (III-a)-(III-d):

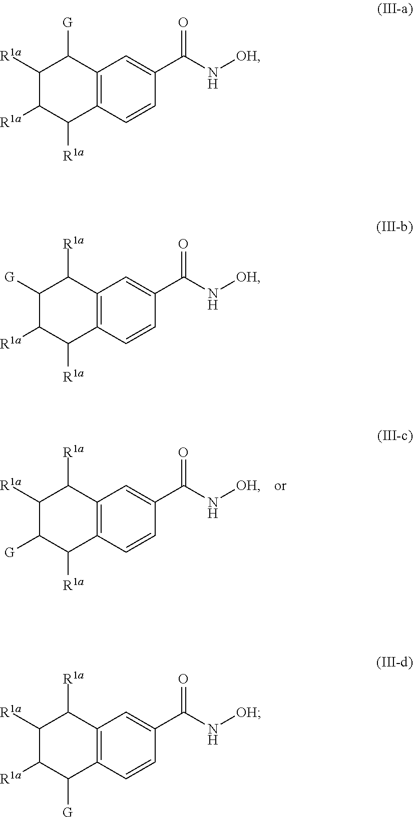

wherein R^{1a} and G have the values described herein. In certain embodiments, the compound of formula (I) is represented by formula (III-b), wherein R^{1a} and G have the values described herein. In certain embodiments, the compound of formula (I) is represented by formula (III-c), wherein R^{1a} and G have the values described herein. In certain embodiments, the compound of formula (I) is represented by formula (III-d), wherein R^{1a} and G have the values described herein.

In some embodiments, the compound of formula (I) is represented by formula (IV-a)-(IV-d):

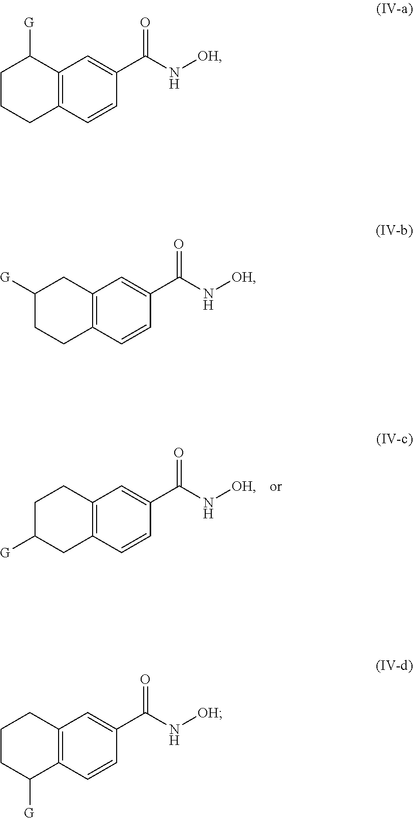

wherein G has the values described herein. In certain embodiments, the compound of formula (I) is represented by formula (IV-b), wherein G has the values described herein. In certain embodiments, the compound of formula (I) is represented by formula (IV-c), wherein G has the values described herein. In certain embodiments, the compound of formula (I) is represented by formula (IV-d), wherein G has the values described herein. In certain embodiments, the compound of formula (I) is represented by formula (IV-d), wherein G has the values described herein.

The values described below for each variable are with respect to any of formulas (I), (II), (III), (IV), or their sub-formulas as described above.

Each occurrence of the variable R^{1a} is independently hydrogen, fluoro, C₁₋₄ alkyl, or C₁₋₄ fluoroalkyl. In some embodiments, each occurrence of R^{1a} is independently hydrogen, fluoro, methyl, or trifluoromethyl. In certain embodiments, each occurrence of R^{1a} is independently hydrogen, fluoro, or methyl. In certain embodiments, each occurrence of R^{1a} is hydrogen.

Each occurrence of the variable R^{1b} is independently hydrogen, fluoro, or C₁₋₄ alkyl. In some embodiments, each occurrence of R^{1b} is independently hydrogen, fluoro, or methyl. In certain embodiments, each occurrence of R^{1b} is hydrogen.

In some embodiments, one occurrence of R^{1a} and one occurrence of R^{1b} on the same carbon atom can be taken together to form ═O or a 3-6 membered cycloaliphatic. In some embodiments, one occurrence of R^{1a} and one occurrence of R^{1b} on the same carbon atom can be taken together to form cyclopropyl, cyclobutyl, cyclopentyl, or cyclohexyl. In certain embodiments, one occurrence of R^{1a} and one occurrence of R^{1b} on the same carbon atom can be taken together to form ═O. In certain embodiments, one occurrence of R^{1a} and one occurrence of R^{1b} on the same carbon atom can be taken together to form cyclopropyl.

Each occurrence of the variable R¹ is independently hydrogen, chloro, fluoro, —O—C₁₋₄ alkyl, cyano, hydroxy, C₁₋₄ alkyl, C₁₋₄ fluoroalkyl, —N(C₁₋₄ alkyl)₂, —NH(C₁₋₄ alkyl), —NH₂, or O—C₁₋₄ fluoroalkyl. In some embodiments, each occurrence of R¹ is independently hydrogen, chloro, fluoro, cyano, hydroxy, methoxy, ethoxy, trifluoromethoxy, trifluoromethyl, methyl, —NH₂, —N(CH₃)₂, —NHCH₃, or ethyl. In certain embodiments, each occurrence of R¹ is independently hydrogen, chloro, fluoro, cyano, hydroxy, methoxy, ethoxy, trifluoromethoxy, trifluoromethyl, methyl, or ethyl. In certain embodiments, each occurrence of R¹ is independently hydrogen, fluoro, or methyl. In certain embodiments, each occurrence of R¹ is hydrogen.

One and only one of the variables R^{2a}, R^{2b}, R^{2c} and R^{2d} is G and the others are lea, wherein R^{1a} and G have the values described herein. In certain embodiments, R^{2b} is G and R^{2a}, R^{2c} and R^{2d} are R^{1a}, wherein R^{1a} and G have the values described herein. In certain embodiments, R^{2c} is G and R^{2a}, R^{2b} and R^{2d} are R^{1a}, wherein R^{1a} and G have the values described herein. In certain embodiments, R^{2d} is G and R^{2b} and R^{2c} are R^{1a}, wherein R^{1a} and G have the values described herein.

The variable G is hydrogen, —R³, -V₁-R³, -V₁-L₁-R³, -L₁-V₁-R³, or -L₁-R³, wherein L₁, V₁, and R³ have the values described herein. In some embodiments, G is —R³, -V₁-R³, -V₁-L₁-R³, -L₁-V₁-R³, or -L₁-R³, wherein L₁, V₁, and R³ have the values described herein. In some embodiments, G is -V₁-R³, -L₁-R³, or —R³, wherein L₁, V₁, and R³ have the values described herein. In certain embodiments, G is -V₁-R³, wherein V₁ and R³ have the values described herein. In certain embodiments, G is -L₁-R³, wherein L₁ and R³ have the values described herein. In certain embodiments, G is —R³, wherein R³ has the values described herein.

The variable L₁ is an unsubstituted or substituted C₁₋₃ alkylene chain. In some embodiments, L₁ is —CH₂—, —CH₂CH₂—, —CH₂CH₂CH₂—, —CR^{A}═CR^{A}, or —C≡C—. In some embodiments, L₁ is —CH₂—, —CH₂CH₂—, or —CH₂CH₂CH₂—. In certain embodiments, L₁ is —CH₂—. In certain embodiments, L₁ is —CH₂CH₂—.

Each occurrence of the variable R^{A} is independently hydrogen, fluoro, or unsubstituted or substituted C₁₋₄ aliphatic. In some embodiments, each occurrence of R^{A} is independently hydrogen, fluoro or methyl. In certain embodiments, each occurrence of R^{A} is hydrogen.

The variable V₁ is —C(O)—, —C(S)—, —C(O)—N(R^{4a})—, —C(O)—O—, —N(R^{4a})—, —N(R^{4a})—C(O)—, —N(R^{4a})—SO₂—, —O—, —N(R^{4a})—C(O)—N(R^{4a})—, —N(R^{4a})—C(O)—O—, —O—C(O)—N(R^{4a})—, or —N(R^{4a})—SO₂—N(R^{4a})—; wherein R^{4a} has the values described herein. In some embodiments, V₁ is —N(R^{4a})—, —N(R^{4a})—C(O)—, —C(O)—N)—N(R^{4a})—SO₂—, —O—, —N(R^{4a})—C(O)—O—, or —N(R^{4a})—C(O)—N(R^{4a})—, wherein R^{4a} has the values described herein. In certain embodiments, V₁ is —N(R^{4a})—, —N(R^{4a})—C(O)—, —C(O)—N(R^{4a})—, or —O—, wherein R^{4a} has the values described herein. In certain embodiments, V₁ is —NH—, —NH—C(O)—, —C(O)—NH—, —NH—SO₂—, —O—, —NH—C(O)—O—, or —NH—C(O)—NH—. In certain embodiments, V₁ is —NH—, —NH—C(O)—, —C(O)—NH—, or —O—.

Each occurrence of R^{4a} is independently hydrogen, or unsubstituted or substituted C₁₋₄ aliphatic; or when V₁ is —N(R^{4a})—C(O)—, —N(R^{4a})—SO₂—, or —N(R^{4a})—C(O)—N(R^{4a})—, R^{4a} can be taken together with any one of R^{1a} to form a substituted or unsubstituted 5-7 membered fused heterocyclyl. In some embodiments, each occurrence of R^{4a} is independently hydrogen, or unsubstituted or substituted C₁₋₄ aliphatic. In some embodiments, when V₁ is —N(R^{4a})—C(O)—, —N(R^{4a})—SO₂—, or —N(R^{4a})—C(O)—N(R^{4a})—, one occurrence of R^{4a} can be taken together with an R^{1a} attached to a ring carbon atom that is not adjacent to the ring carbon atom to which G is attached to form a substituted or unsubstituted 5-7 membered bridged heterocyclyl. In certain embodiments, each occurrence of R^{4a} is hydrogen.

The variable R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

In some embodiments, R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; wherein:

each substitutable carbon chain atom in R³ is unsubstituted or substituted with 1-2 occurrences of —R^{5dd};

each substitutable saturated ring carbon atom in R³ is unsubstituted or substituted with ═O, ═C(R⁵)₂, or R^{5aa};

each substitutable unsaturated ring carbon atom in R³ is unsubstituted or is substituted with —R^{5a};

each substitutable ring nitrogen atom in R³ is unsubstituted or substituted with —R^{9b};

wherein R^{5dd}, R⁵, R^{5a}, R^{5aa}, and R^{9b} have the values described herein.

In some embodiments, R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; wherein:

each substitutable carbon chain atom in R³ is unsubstituted or substituted with 1-2 occurrences of —R^{5dd};

each substitutable saturated ring carbon atom in R³ is unsubstituted or substituted with R^{5aa};

each substitutable unsaturated ring carbon atom in R³ is unsubstituted or is substituted with —R^{5a};

the total number of R^{5a} and R^{5aa} substituents is p; and

each substitutable ring nitrogen atom in R³ is unsubstituted or substituted with —R^{9b};

wherein R^{5dd}, R^{5a}, R^{9b} and p have the values described herein.

Each occurrence of the variable R^{5dd} is independently fluoro, hydroxy, —O(C₁₋₆ alkyl), cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, or —NHS(O)₂C₁₋₆ alkyl, wherein R⁴ has the values described herein. In some embodiments, each occurrence of R^{5dd} is independently fluoro, hydroxy, methoxy, ethoxy, —NH(C₁₋₆ alkyl), —N(C₁₋₆ alkyl)₂, or —C(O)NHCH₃.

Each occurrence of the variable R^{9b} is independently —C(O)R⁶, —C(O)N(R⁴)₂, —CO₂R⁶, —SO₂R⁶, —SO₂N(R⁴)₂, unsubstituted C₃₋₁₀ cycloaliphatic, C₃₋₁₀ cycloaliphatic substituted with 1-2 independent occurrences of R⁷ or R⁸, unsubstituted C₁₋₆ aliphatic, or C₁₋₆ aliphatic substituted with 1-2 independent occurrences of R⁷ or R⁸, wherein R⁷ and R⁸ have the values described herein. In some embodiments, each occurrence of R^{9b} is independently unsubstituted —C(O)—C₁₋₆ aliphatic, unsubstituted —C(O)—C₃₋₁₀ cycloaliphatic, or unsubstituted C₁₋₆ aliphatic. In some embodiments, each occurrence of R^{9b} is unsubstituted C₁₋₆ aliphatic. In certain embodiments, each occurrence of R^{9b} is independently methyl, ethyl, isopropyl, isobutyl, n-propyl, n-butyl, tert-butyl, —C(O)-methyl, —C(O)-ethyl, —C(O)-cyclopropyl, —C(O)-tert-butyl, —C(O)-isopropyl, or —C(O)-cyclobutyl. In certain embodiments, each occurrence of R^{9b} is independently methyl, ethyl, isopropyl, isobutyl, n-propyl, n-butyl, or tert-butyl.

Each occurrence of the variable R⁴ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two R⁴ on the same nitrogen atom, taken together with the nitrogen atom, form an unsubstituted or substituted 5- to 6-membered heteroaryl or an unsubstituted or substituted 4- to 8-membered heterocyclyl having, in addition to the nitrogen atom, 0-2 ring heteroatoms selected from nitrogen, oxygen, and sulfur.

Each occurrence of the variable R⁵ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

Each occurrence of the variable R⁶ is independently unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

Each occurrence of the variable R⁷ is independently unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

Each occurrence of the variable R⁸ is independently chloro, fluoro, —OH, —O(C₁₋₆ alkyl), —CN, —N(R⁴)₂, —C(O)(C₁₋₆ alkyl), —CO₂H, —CO₂(C₁₋₆ alkyl), —C(O)NH₂, —C(O)NH(C₁₋₆ alkyl), or —C(O)N(C₁₋₆ alkyl)₂, wherein R⁴ has the values described herein.

Each occurrence of the variable R^{5a} is independently halogen, —NO₂, —CN, —C(R⁵)═C(R⁵)₂, —C≡C—R⁵, —SR⁶, —S(O)R⁶, —SO₂R⁶, —SO₂N(R⁴)₂, —N(R⁴)₂, —NR⁴C(O)R⁶, —NR⁴C(O)N(R⁴)₂, —NR⁴CO₂R⁶, —OC(O)N(R⁴)₂, —C(O)R⁶, —C(O)N(R⁴)₂, —N(R⁴)SO₂R⁶, —N(R⁴)SO₂N(R⁴)₂, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two adjacent R^{5a}, taken together with the intervening ring atoms, form an unsubstituted or substituted fused 5-10 membered aromatic ring or an unsubstituted or substituted 4-10 membered non-aromatic ring having 0-3 heteroatoms independently selected from nitrogen, oxygen, and sulfur, wherein R⁵, R⁶, and R⁴ have the values described herein.

In some embodiments, each occurrence of R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆ alkyl)₂, 3-10-membered cycloaliphatic substituted with 0-2 occurrences of —R^{7a}, 4-10-membered heterocyclyl having 1-4 heteroatoms independently selected from nitrogen, oxygen, and sulfur substituted with 0-2 occurrences of lea, 6-10-membered aryl substituted with 0-2 occurrences of lea, or 5-10-membered heteroaryl having 1-5 heteroatoms independently selected from nitrogen, oxygen, and sulfur substituted with 0-2 occurrences of —R^{7a}, wherein R⁴, R⁵, R^{7a}, R⁷, and R⁸ have the values described herein.

In certain embodiments, each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, CH₂—N(R⁴)₂, or —NHSO₂CH₃, wherein R¹⁰ has the values described herein.

Each occurrence of the variable R¹⁰ is unsubstituted or substituted 4-10-membered heterocyclyl having 1-4 heteroatoms independently selected from nitrogen, oxygen, and sulfur. In some embodiments, each occurrence of R¹⁰ is unsubstituted or substituted 4-10-membered heterocyclyl having 1-4 heteroatoms independently selected from nitrogen, oxygen, and sulfur, wherein if substituted R¹⁰ is substituted with 0-2 occurrences of —R^{7aa}, wherein R^{7aa} has the values described herein. In some embodiments, each occurrence of R¹⁰ is pyrrolidinyl, piperidinyl, pyrrolinyl, piperazinyl, or morpholinyl, wherein each of the foregoing groups is unsubstituted or substituted with 0-1 occurrence of R^{7aa}, wherein R^{7aa} has the values described herein.

Each occurrence of the variable R^{5aa} is independently chloro, fluoro, hydroxy, unsubstituted or substituted C₁₋₆ aliphatic, —O(C₁₋₆ alkyl), —C₁₋₆ fluoroalkyl, —O—C₁₋₆ fluoroalkyl, cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl. In some embodiments, each occurrence of R^{5aa} is independently chloro, fluoro, hydroxy, methyl, ethyl, methoxy, ethoxy, trifluoromethyl, trifluoromethoxy, —C(O)NH₂, —N(C₁₋₆ alkyl)₂, —NHC₁₋₆ alkyl, or CO₂H.

Each occurrence of the variable R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆ alkyl)₂, —NHC(O)NHC₁₋₆ alkyl, —NHC(O)N(C₁₋₆ alkyl)₂, or —NHS(O)₂C₁₋₆ alkyl.

Each occurrence of the variable R^{7aa} is independently chloro, fluoro, hydroxy, unsubstituted or substituted C₁₋₆ aliphatic, —O(C₁₋₆ alkyl), —C₁₋₆ fluoroalkyl, —O—C₁₋₆ fluoroalkyl, cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl. In some embodiments, each occurrence of R^{7aa} is independently fluoro, hydroxy, methyl, ethyl, methoxy, trifluoromethyl, —C(O)NH₂, or CO₂H.

The variable p is 1-4. In some embodiments, p is 1-3. In certain embodiments, p is 1-2. In certain embodiments, p is 1.

In some embodiments, R³ is unsubstituted or substituted C₁₋₆ aliphatic. In some embodiments, each substitutable carbon chain atom in R³ is unsubstituted or substituted with 1-2 occurrences of —R^{5dd}, wherein R^{5dd} has the values described herein. In certain embodiments, R³ is methyl, ethyl, n-propyl, isopropyl, tert-butyl, n-butyl, iso-butyl, pentyl, hexyl, butenyl, propenyl, pentenyl, or hexenyl, wherein each of the forementioned groups is unsubstituted or substituted. In certain embodiments, R³ is methyl, ethyl, n-propyl, isopropyl, tert-butyl, n-butyl, iso-butyl, pentyl, hexyl, butenyl, propenyl, pentenyl, or hexenyl, wherein each substitutable carbon chain atom in R³ is unsubstituted or substituted with 1-2 occurrences of —R^{5dd}, wherein R^{5dd} has the values described herein.

In some embodiments, R³ is unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

In certain embodiments, R³ is unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur, wherein:

each substitutable saturated ring carbon atom in R³ is unsubstituted or substituted with ═O, ═C(R⁵)₂, or R^{5aa};

each substitutable unsaturated ring carbon atom in R³ is unsubstituted or is substituted with —R^{5a}; and

each substitutable ring nitrogen atom in R³ is unsubstituted or substituted with —R^{9b};

wherein R⁵, R^{5a}, R^{5aa}, and R^{9b} have the values described herein.

In certain embodiments, R³ is unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur, wherein:

each substitutable saturated ring carbon atom in R³ is unsubstituted or substituted with —R^{5aa};

each substitutable unsaturated ring carbon atom in R³ is unsubstituted or is substituted with —R^{5a};

the total number of R^{5a} and R^{5aa} substituents is p; and

each substitutable ring nitrogen atom in R³ is unsubstituted or substituted with —R^{9b};

wherein R^{5a}, R^{5aa}, R^{9b} and p have the values described herein.

In certain embodiments, R³ is furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, naphthyl, pyranyl, pyridyl, pyridazinyl, pyrimidinyl, pyrazinyl, triazinyl, indolizinyl, imidazopyridyl, indolyl, isoindolyl, indazolyl, benzimidazolyl, benzthiazolyl, benzothienyl, benzofuranyl, benzoxazolyl, benzodioxolyl, benzthiadiazolyl, 2,3-dihydrobenzofuranyl, 4H-furo[3,2-b]pyrrolyl, pyrazolopyrimidinyl, purinyl, quinolyl, isoquinolyl, tetrahydroquinolinyl, tetrahydronaphthyridinyl, tetrahydroisoquinolinyl, cinnolinyl, phthalazinyl, quinazolinyl, quinoxalinyl, naphthyridinyl, pteridinyl, tetrahydrofuranyl, tetrahydropyranyl, tetrahydrothienyl, indanyl, tetrahydroindazolyl, pyrrolidinyl, pyrrolidonyl, piperidinyl, pyrrolinyl, decahydroquinolinyl, oxazolidinyl, piperazinyl, dioxanyl, diazepinyl, oxazepinyl, thiazepinyl, morpholinyl, thiomorpholinyl, quinuclidinyl, phenanthridinyl, tetrahydronaphthyl, indolinyl, benzodioxanyl, chromanyl, cyclopropyl, cyclobutyl, cyclopentyl, cyclohexyl, cycloheptyl, cyclooctyl, cyclopentenyl, cyclohexenyl, cycloheptenyl, cyclooctenyl, bicycloheptanyl, bicyclooctanyl, or adamantyl; wherein:

each substitutable saturated ring carbon atom in R³ is unsubstituted or substituted with ═O, ═C(R⁵)₂, or R^{5aa};

each substitutable unsaturated ring carbon atom in R³ is unsubstituted or is substituted with —R^{5a}; and

each substitutable ring nitrogen atom in R³ is unsubstituted or substituted with —R^{9b};

wherein R⁵, R^{5a}, R^{5aa}, and R^{9b} have the values described herein.

In certain embodiments, R³ is furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, naphthyl, pyranyl, pyridyl, pyridazinyl, pyrimidinyl, pyrazinyl, triazinyl, indolizinyl, imidazopyridyl, indolyl, isoindolyl, indazolyl, benzimidazolyl, benzthiazolyl, benzothienyl, benzofuranyl, benzoxazolyl, benzodioxolyl, benzthiadiazolyl, 2,3-dihydrobenzofuranyl, 4H-furo[3,2-b]pyrrolyl, pyrazolopyrimidinyl, purinyl, quinolyl, isoquinolyl, tetrahydroquinolinyl, tetrahydronaphthyridinyl, tetrahydroisoquinolinyl, cinnolinyl, phthalazinyl, quinazolinyl, quinoxalinyl, naphthyridinyl, pteridinyl, tetrahydrofuranyl, tetrahydropyranyl, tetrahydrothienyl, indanyl, tetrahydroindazolyl, pyrrolidinyl, pyrrolidonyl, piperidinyl, pyrrolinyl, decahydroquinolinyl; oxazolidinyl, piperazinyl, dioxanyl, diazepinyl, oxazepinyl, thiazepinyl, morpholinyl, thiomorpholinyl, quinuclidinyl, phenanthridinyl, tetrahydronaphthyl, indolinyl, benzodioxanyl, chromanyl, cyclopropyl, cyclobutyl, cyclopentyl, cyclohexyl, cycloheptyl, cyclooctyl, cyclopentenyl, cyclohexenyl, cycloheptenyl, cyclooctenyl, bicycloheptanyl, bicyclooctanyl, or adamantyl; wherein:

each substitutable saturated ring carbon atom in R³ is unsubstituted or substituted with —R^{5aa};

each substitutable unsaturated ring carbon atom in R³ is unsubstituted or is substituted with —R^{5a};

the total number of R^{5a} and R^{5aa} substituents is p; and

each substitutable ring nitrogen atom in R³ is unsubstituted or substituted with —R^{9b};

wherein R^{5a}, R^{5aa}, R^{9b}, and p have the values described herein.

In certain embodiments, R³ is furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, pyranyl, pyridyl, pyridazinyl, pyrimidinyl, pyrazinyl, or triazinyl; wherein:

each substitutable unsaturated ring carbon atom in R³ is unsubstituted or substituted with —R^{5a};

each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;

the total number of R^{5a} substituents is p;

each substitutable ring nitrogen atom in R³ is unsubstituted or substituted with —R^{9b}; and

each occurrence of R^{9b} is independently methyl, ethyl, isopropyl, isobutyl, n-propyl, n-butyl, or tert-butyl;

wherein p and R¹⁰ have the values described herein.

In certain embodiments, R³ is indolizinyl, imidazopyridyl, indolyl, indazolyl, benzimidazolyl, benzthiazolyl, benzothienyl, benzofuranyl, benzoxazolyl, benzthiadiazolyl, pyrazolopyrimidinyl, purinyl, quinolyl, isoquinolyl, cinnolinyl, phthalazinyl, quinazolinyl, quinoxalinyl, naphthyridinyl, naphthyl, or pteridinyl; wherein:

each substitutable unsaturated ring carbon atom in R³ is unsubstituted or substituted with —R^{5a};

each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;

the total number of R^{5a} substituents is p;

each substitutable ring nitrogen atom in R³ is unsubstituted or substituted with —R^{9b}; and

each R^{9b} is independently methyl, ethyl, isopropyl, isobutyl, n-propyl, n-butyl, or tert-butyl;

wherein p and R¹⁰ have the values described herein.

In certain embodiments, R³ is tetrahydrofuranyl, tetrahydropyranyl, tetrahydrothienyl, pyrrolidinyl, pyrrolidonyl, piperidinyl, pyrrolinyl, oxazolidinyl, piperazinyl, dioxanyl, diazepinyl, oxazepinyl, thiazepinyl, morpholinyl, thiomorpholinyl, cyclopropyl, cyclobutyl, cyclopentyl, cyclohexyl, cycloheptyl, cyclooctyl, cyclopentenyl, cyclohexenyl, cycloheptenyl, or cyclooctenyl; wherein:

each substitutable saturated ring carbon atom in R³ is unsubstituted or substituted with —R^{5aa};

each substitutable unsaturated ring carbon atom in R³ is unsubstituted or is substituted with —R^{5a};

the total number of R^{5a} and R^{5aa} substituents is p;

each substitutable ring nitrogen atom in R³ is unsubstituted or substituted with —R^{9b};

each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;

each occurrence of R^{5aa} is independently chloro, fluoro, hydroxy, methyl, ethyl, methoxy, ethoxy, trifluoromethyl, trifluoromethoxy, —C(O)NH₂, —N(C₁₋₆ alkyl)₂, —NHC₁₋₆ alkyl, or CO₂H; and

each R^{9b} is independently methyl, ethyl, isopropyl, isobutyl, n-propyl, n-butyl, tert-butyl, —C(O)-methyl, —C(O)-ethyl, —C(O)-cyclopropyl, —C(O)-tert-butyl, —C(O)-isopropyl, or —C(O)-cyclobutyl;

wherein R¹⁰ and p have the values described herein.

In certain embodiments, R³ is tetrahydroindazolyl, bicycloheptanyl, bicyclooctanyl, adamantyl, isoindolyl, benzodioxolyl, 2,3-dihydrobenzofuranyl, 4H-furo[3,2-b]pyrrolyl, quinuclidinyl, tetrahydroquinolinyl, tetrahydronaphthyridinyl, tetrahydroisoquinolinyl, decahydroquinolinyl, tetrahydronaphthyl, indolinyl, benzodioxanyl, chromanyl, tetrahydroindazolyl, or indanyl; wherein:

each substitutable saturated ring carbon atom in R³ is unsubstituted or substituted with —R^{5aa};

each substitutable unsaturated ring carbon atom in R³ is unsubstituted or is substituted with —R^{5a};

the total number of R^{5a} and R^{5aa} substituents is p;

each substitutable ring nitrogen atom in R³ is unsubstituted or substituted with —R^{9b};

each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;

each occurrence of R^{5aa} is independently chloro, fluoro, hydroxy, methyl, ethyl, methoxy, ethoxy, trifluoromethyl, trifluoromethoxy, —C(O)NH₂, alkyl)₂, —NHC₁₋₆ alkyl, or CO₂H; and

each R^{9b} is independently methyl, ethyl, isopropyl, isobutyl, n-propyl, n-butyl, tert-butyl, —C(O)-methyl, —C(O)-ethyl, —C(O)-cyclopropyl, —C(O)-tert-butyl, —C(O)-isopropyl, or —C(O)-cyclobutyl;

wherein R¹⁰ and p have the values described herein.

In some embodiments, G is:

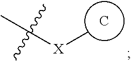

wherein X and Ring C have the values described herein.

The variable X is a bond, NH—C(O)—, —C(O)—NH—, or V₂-L₂-R^{3aa}-V₃-, wherein L₂, R^{3aa}, V₂, and V₃ have the values described herein. In some embodiments, X is a bond. In some embodiments, X is NH—C(O)—. In some embodiments, X is —C(O)—NH—. In some embodiments, X is V₂-L₂-R^{3aa}-V₃-, wherein L₂, R^{3aa}, V₂, and V₃ have the values described herein. In some embodiments, X is a bond, NH—C(O)—, —C(O)—NH—,

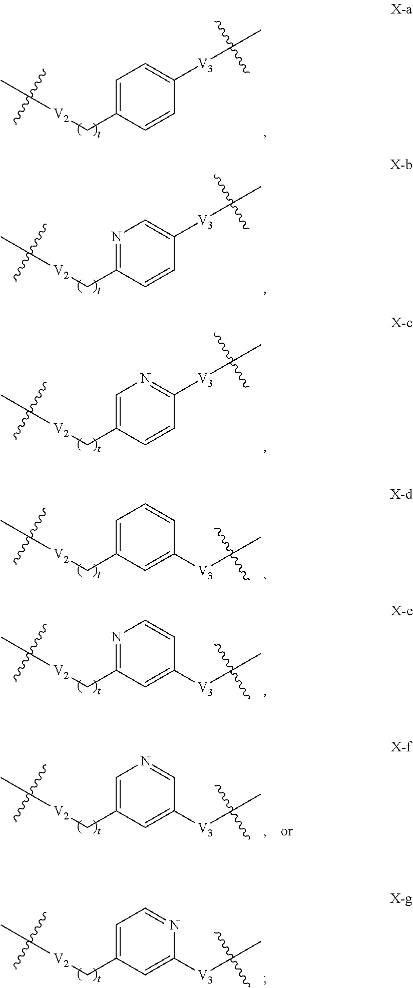

wherein V₂, V₃, and t have the values described herein.

In certain embodiments, X is a bond, NH—C(O)—, —C(O)—NH—,

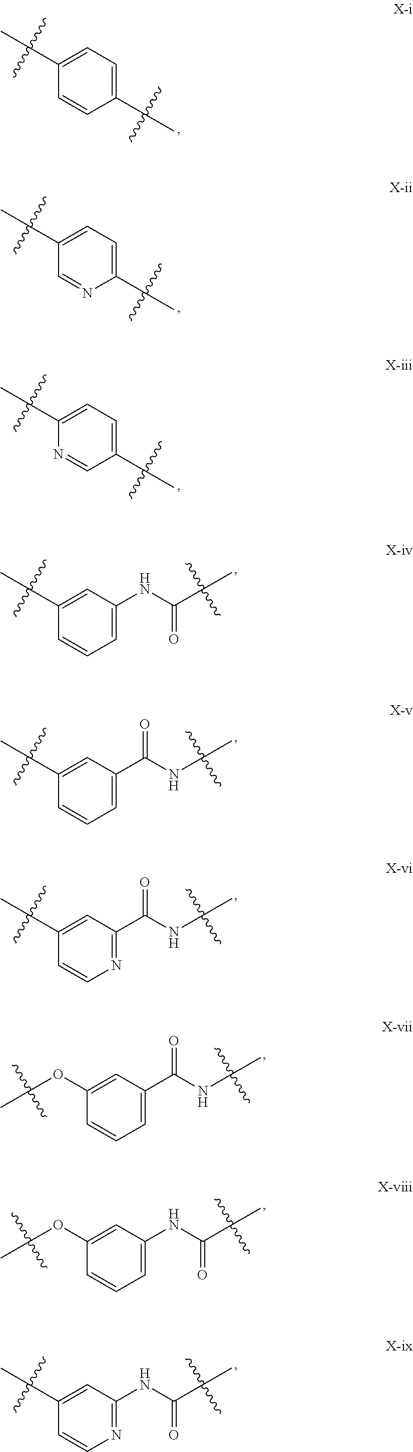

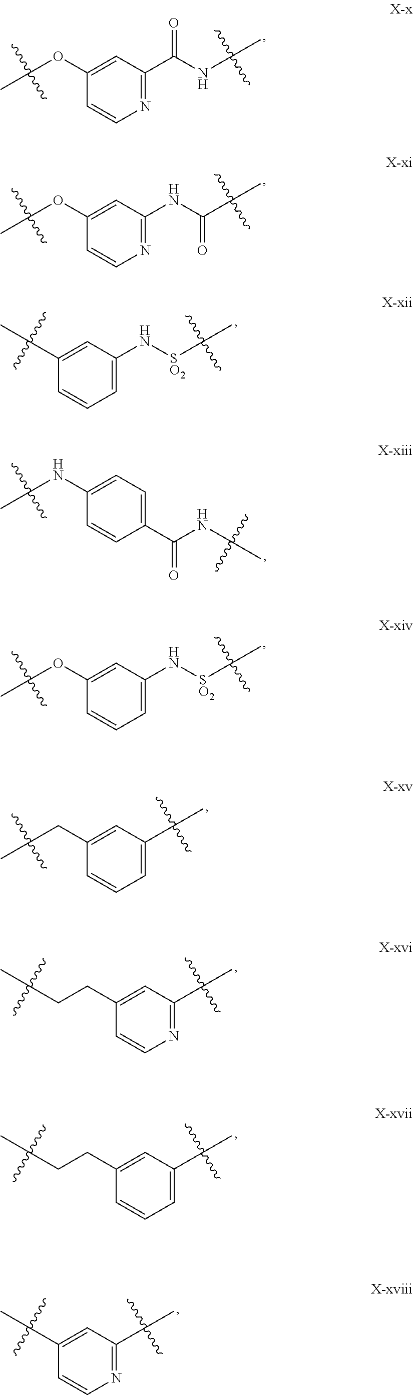

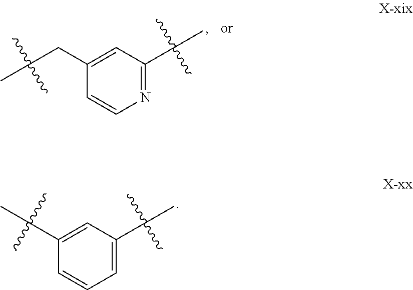 [SMILES: *Cc1ccnc(*)c1 ; *c1cccc(*)c1]

In certain embodiments, X is NH—C(O)—, X-iv, X-vi, X-vii, X-viii, X-ix, or X-x.

The variable V₂ is a bond, NH—C(O)—, —C(O)—NH—, —NH—, or —O—. In some embodiments, V₂ is a bond, NH—C(O)— or —O—. In certain embodiments, V₂ is a bond. In certain embodiments, V₂ is —O—. In certain embodiments, V₂ is NH—C(O)—.

The variable L₂ is a bond or unsubstituted or substituted C₁₋₃ alkylene chain. In some embodiments, L₂ is a bond, —CH₂—, —CH₂CH₂—, or —CH₂CH₂CH₂—. In certain embodiments, L₂ is a bond. In certain embodiments, L₂ is CH₂—. In certain embodiments, L₂ is —CH₂CH₂—.

Ring C is a 4-7 membered heterocyclic ring containing one nitrogen atom, wherein the nitrogen atom is not the atom bound to X, and wherein the nitrogen atom in Ring C is substituted with R^{9bb} and Ring C is unsubstituted or substituted by 1-4 occurrences of R^{5b}; wherein R^{9bb}, X, and R^{5b} have the values described herein. In some embodiments, Ring C is a 4-7 membered heterocyclic ring containing one nitrogen atom, wherein the nitrogen atom is not the atom bound to X, and wherein the nitrogen atom in Ring C is substituted with R^{9bb} and Ring C is unsubstituted or substituted by 1-2 occurrences of R^{5b}; wherein R^{9bb}, X, and R^{5b} have the values described herein.

In certain embodiments, Ring C is:

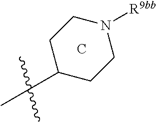

wherein Ring C is unsubstituted or substituted with 1 occurrence of R^{5b}, wherein R^{9bb} and R^{5b} have the values described herein. In certain embodiments, Ring C is:

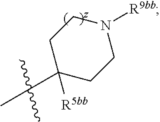

wherein R^{9bb}, z and R^{5bb} have the values described herein.

The variable V₃ is a bond, NH—C(O)—, —C(O)—NH—, —NH—S(O)₂—, or NH—C(O)—NH—. In some embodiments, V₃ is a bond, —C(O)—NH—, or NH—C(O)—. In certain embodiments, V₃ is a bond. In certain embodiments, V₃ is NH—C(O)—. In certain embodiments, V₃ is —C(O)—NH—.

The variable t is 0-2. In some embodiments, t is 0-1. In certain embodiments, t is 0. In certain embodiments, t is 1. In certain embodiments, t is 2.

The variable R^{3aa} is a 6-membered aromatic ring containing 0-2 nitrogen atoms which is unsubstituted or substituted with 1-2 independent occurrences of R^{4c}, wherein R^{4c} has the values described herein. In some embodiments, R^{3aa} is phenyl or pyridyl, each of which is unsubstituted or substituted with 1-2 independent occurrences of R^{4c}, wherein R^{4c} has the values described herein. In some embodiments, R^{3aa} is:

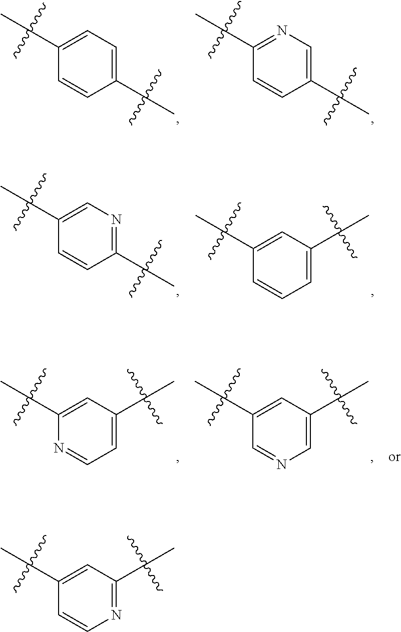

wherein each ring is unsubstituted or substituted with 1-2 independent occurrences of R^{4c}.

The variable R^{4c} is chloro, fluoro, cyano, hydroxy, methoxy, ethoxy, trifluoromethoxy, trifluoromethyl, methyl, or ethyl. In some embodiments, R^{4c} is chloro, fluoro, methyl or ethyl.

The variable z is 0-1. In some embodiments, z is 0. In some embodiments, z is 1.

Each occurrence of the variable R^{5b} is independently chloro, fluoro, hydroxy, methyl, ethyl, methoxy, ethoxy, trifluoromethyl, trifluoromethoxy, —C(O)NH₂, or CO₂H. In some embodiments, each occurrence of the variable R^{5b} is independently chloro, fluoro, hydroxy, methyl, or ethyl. In certain embodiments, each occurrence of the variable R^{5b} is methyl.

The variable R^{5bb} is hydrogen or methyl. In some embodiments, R^{5bb} is hydrogen. In some embodiments, R^{5bb} is methyl.

The variable R^{9bb} is hydrogen, unsubstituted C(O)—O—C₁₋₆ aliphatic, unsubstituted C(O)—C₁₋₆ aliphatic, unsubstituted C(O)—C₃₋₁₀ cycloaliphatic, or unsubstituted C₁₋₆ aliphatic. In some embodiments, R^{9bb} is hydrogen, methyl, ethyl, isopropyl, or tert-butoxycarbonyl. In some embodiments, R^{9bb} is methyl, ethyl, or isopropyl. In certain embodiments, R^{9bb} is hydrogen.

In certain embodiments for the compounds of formulas (I), (II), (III) and (IV):

G is -V₁-R³, -L₁-R³, or —R³;

L₁ is CH₂— or CH₂CH₂—; and

V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—;

wherein R³ and R^{4a} have the values contained herein.

In certain embodiments, for the compound of formula (I):

each occurrence of R^{1a} is independently hydrogen, fluoro, or methyl;

each occurrence of R^{1b} is independently hydrogen, fluoro, or methyl; and

each occurrence of R¹ is independently hydrogen, chloro, fluoro, cyano, hydroxy, methoxy, ethoxy, trifluoromethoxy, trifluoromethyl, methyl, or ethyl.

In certain embodiments, for the compounds of formula (II):

each occurrence of R^{1a} is independently hydrogen, fluoro, trifluoromethyl, or methyl; and

each occurrence of R^{1b} is independently hydrogen, chloro, fluoro, cyano, hydroxy, methoxy, ethoxy, trifluoromethoxy, trifluoromethyl, methyl, or ethyl.

In certain embodiments, the compound of formula (I) is represented by:

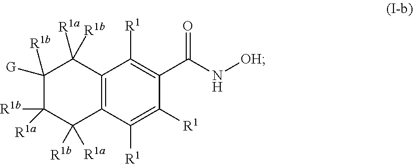

wherein:

each occurrence of R^{1a} is independently hydrogen, fluoro, or methyl;

each occurrence of R^{1b} is independently hydrogen, fluoro, or methyl; and

each occurrence of R¹ is independently hydrogen, chloro, fluoro, cyano, hydroxy, methoxy, ethoxy, trifluoromethoxy, trifluoromethyl, methyl, or ethyl;

G is -V₁-R³, -L₁-R³, or —R³;

L₁ is CH₂— or CH₂CH₂—; and

V₁ is —N(R^{4a})—, —N(R^{4a})—C(O)—, —N(R^{4a})—SO₂—, —O—, —C(O)—O—, —N(R^{4a})—C(O)—O—, or —N(R^{4a})—C(O)—N(R^{4a})—;

wherein R³ and R^{4a} have the values contained herein.

In certain embodiments, the compound of formula (I) is represented by:

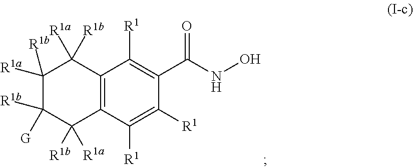

wherein:

each occurrence of R^{1a} is independently hydrogen, fluoro, or methyl;

each occurrence of R^{1b} is independently hydrogen, fluoro, or methyl; and

each occurrence of R¹ is independently hydrogen, chloro, fluoro, cyano, hydroxy, methoxy, ethoxy, trifluoromethoxy, trifluoromethyl, methyl, or ethyl;

G is -V₁-R³, or —R³;

L₁ is CH₂— or CH₂CH₂—; and

V₁ is —N(R^{4a})—, —N(R^{4a})—C(O)—, —N(R^{4a})—SO₂—, —O—, —C(O)—O—, —N(R^{4a})—C(O)—O—, or —N(R^{4a})—C(O)—N(R^{4a})—;

wherein R³ and R^{4a} have the values contained herein.

In certain embodiments, the compound of formula (I) is represented by:

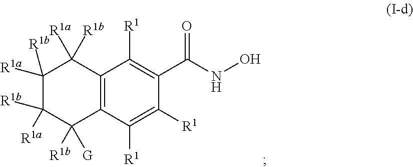

wherein:

each occurrence of R^{1a} is independently hydrogen, fluoro, or methyl;

each occurrence of R^{1b} is independently hydrogen, fluoro, or methyl; and

each occurrence of R¹ is independently hydrogen, chloro, fluoro, cyano, hydroxy, methoxy, ethoxy, trifluoromethoxy, trifluoromethyl, methyl, or ethyl;

G is -V₁—R³, -L₁-R³, or —R³;

L₁ is CH₂— or CH₂CH₂—; and

V₁ is —N(R^{4a})—, —N(R^{4a})—C(O)—, —N(R^{4a})—SO₂—, —O—, —C(O)—O—, —N(R^{4a})—C(O)—O—, or —N(R^{4a})—C(O)—N(R^{4a})—;

wherein R³ and R^{4a} have the values contained herein.

In certain embodiments, the compound of formula (I) is represented by:

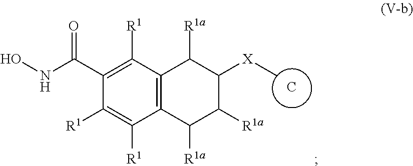

wherein:

each occurrence of R^{1a} is independently hydrogen, fluoro, or methyl;

each occurrence of R^{1b} is independently hydrogen, chloro, fluoro, cyano, hydroxy, methoxy, ethoxy, trifluoromethoxy, trifluoromethyl, methyl, or ethyl; and

X and Ring C have the values described herein.

In certain such embodiments:

R¹ is H; and

R^{1a} is H.

In certain embodiments, the compound of formula (I) is represented by:

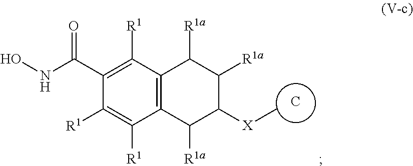

wherein:

each occurrence of R^{1a} is independently hydrogen, fluoro, or methyl;

each occurrence of R¹ is independently hydrogen, chloro, fluoro, cyano, hydroxy, methoxy, ethoxy, trifluoromethoxy, trifluoromethyl, methyl, or ethyl; and

X and Ring C have the values described herein.

In certain such embodiments:

R¹ is H; and

R^{1a} is H.

In certain embodiments, the compound of formula (I) is represented by:

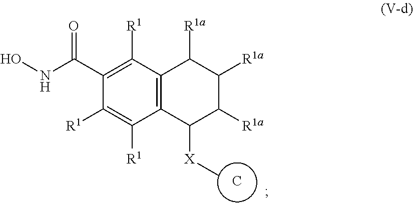

wherein:

each occurrence of R^{1a} is independently hydrogen, fluoro, or methyl;

each occurrence of R¹ is independently hydrogen, chloro, fluoro, cyano, hydroxy, methoxy, ethoxy, trifluoromethoxy, trifluoromethyl, methyl, or ethyl; and

X and Ring C have the values described herein.

In certain such embodiments:

R¹ is H; and

R^{1a} is H.

In certain embodiments, the compound of formula (I) is represented by:

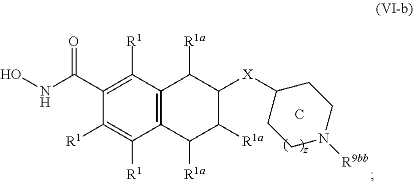

wherein:

each occurrence of R^{1a} is independently hydrogen, fluoro, or methyl;

each occurrence of R¹ is independently hydrogen, chloro, fluoro, cyano, hydroxy, methoxy, ethoxy, trifluoromethoxy, trifluoromethyl, methyl, or ethyl;

R^{9bb} is hydrogen, methyl, ethyl, isopropyl, or tert-butoxycarbonyl;

Ring C is unsubstituted or substituted with one occurrence of R^{5b};

X is a bond, NH—C(O)—, —C(O)—NH—, X-a, X-b, X-c, X-d, X-e, X-f, or X-g; and

z, R^{5b}, t, V₂, and V₃ have the values described herein.

In certain such embodiments:

R^{5b} is methyl;

R¹ is H; and

R^{1a} is H.

In certain embodiments, the compound of formula (I) is represented by:

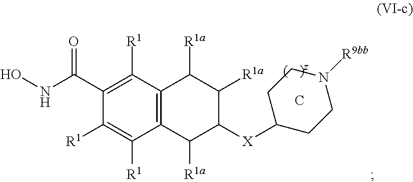

wherein:

each occurrence of R^{1a} is independently hydrogen, fluoro, or methyl;

each occurrence of R¹ is independently hydrogen, chloro, fluoro, cyano, hydroxy, methoxy, ethoxy, trifluoromethoxy, trifluoromethyl, methyl, or ethyl;

R^{9bb} is hydrogen, methyl, ethyl, isopropyl, or tert-butoxycarbonyl;

Ring C is unsubstituted or substituted with one occurrence of R^{5b};

X is a bond, NH—C(O)—, —C(O)—NH—, X-a, X-b, X-c, X-d, X-e, X-f, or X-g; and

z, R^{5b}, t, V₂, and V₃ have the values described herein.

In certain such embodiments:

R^{5b} is methyl;

R¹ is H; and

R^{1a} is H.

In certain embodiments, the compound of formula (I) is represented by:

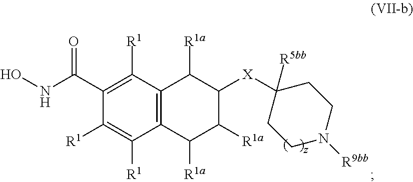

wherein:

each occurrence of R^{1a} is independently hydrogen, fluoro, or methyl;

each occurrence of R¹ is independently hydrogen, chloro, fluoro, cyano, hydroxy, methoxy, ethoxy, trifluoromethoxy, trifluoromethyl, methyl, or ethyl;

R^{9bb} is hydrogen, methyl, ethyl, isopropyl, or tert-butoxycarbonyl;

R^{5bb} is hydrogen or methyl;

X is NH—C(O)—, X-iv, X-vi, X-vii, X-viii, X-ix, or X-x; and

z has the values described herein.

In certain such embodiments:

R^{5bb} is methyl;

z is 1;

R¹ is H; and

R^{1a} is H.

In certain embodiments, the compound of formula (I) is represented by:

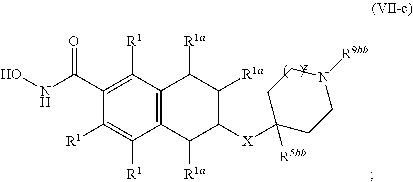

wherein:

each occurrence of R^{1a} is independently hydrogen, fluoro, or methyl;

each occurrence of R¹ is independently hydrogen, chloro, fluoro, cyano, hydroxy, methoxy, ethoxy, trifluoromethoxy, trifluoromethyl, methyl, or ethyl;

R^{9bb} is hydrogen, methyl, ethyl, isopropyl, or tert-butoxycarbonyl;

R^{5bb} is hydrogen or methyl;

X is NH—C(O)—, X-iv, X-vi, X-vii, X-viii, X-ix, or X-x; and

z has the values described herein.

In certain such embodiments:

R^{5bb} is methyl;

Z is 1;

R¹ is H; and

R^{1a} is H.

In some embodiments, the compound of formula (I) is represented by formula (II-a)-(II-d):

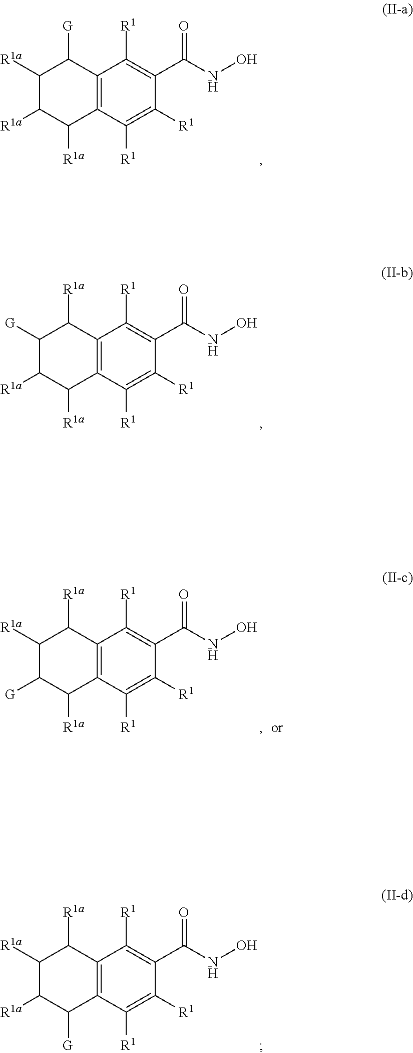

wherein:

G is -V₁-R³, -L₁-R³, or —R³;

L₁ is CH₂— or CH₂CH₂—;

V₁ is —NH—, —NH—C(O)—, —C(O)—NH—, or —O—;

each occurrence of R^{1a} is independently hydrogen, fluoro, trifluoromethyl, or methyl; and

each occurrence of R¹ is independently hydrogen, chloro, fluoro, cyano, hydroxy, methoxy, ethoxy, trifluoromethoxy, trifluoromethyl, methyl, or ethyl;

wherein R³ has the values described herein.

In some such embodiments, the compound of formula (I) is represented by formula (II-a). In certain such embodiments, the compound of formula (I) is represented by formula (II-b). In certain such embodiments, the compound of formula (I) is represented by formula (II-c). In certain such embodiments, the compound of formula (I) is represented by formula (II-d).

In some embodiments, the compound of formula (I) is represented by formula (II-a)-(II-d):

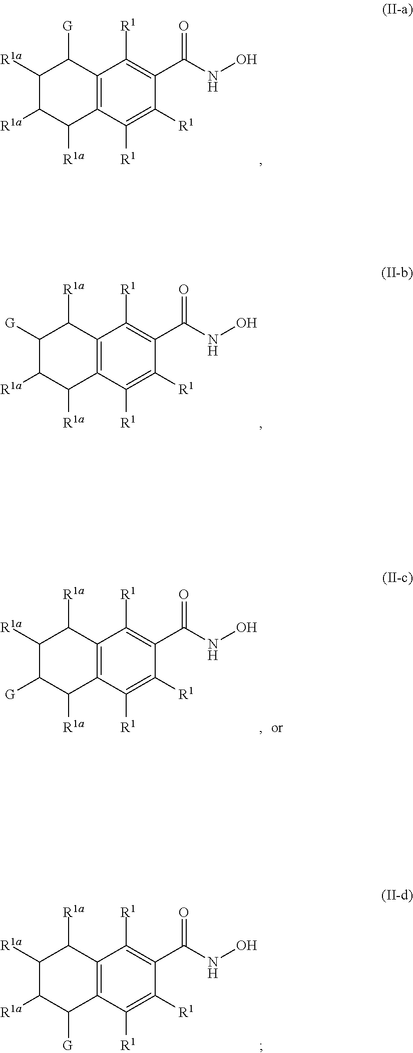

wherein:

G is -V₁-R³, -L₁-R³, or —R³;

L₁ is CH₂— or CH₂CH₂—; and

V₁ is —NH—, —NH—C(O)—, —C(O)—NH—, or —O—;

each occurrence of R^{1a} is hydrogen; and

each occurrence of R¹ is hydrogen.

wherein R³ has the values described herein.

In some such embodiments, the compound of formula (I) is represented by formula (II-a). In certain such embodiments, the compound of formula (I) is represented by formula (II-b). In certain such embodiments, the compound of formula (I) is represented by formula (II-c). In certain such embodiments, the compound of formula (I) is represented by formula (II-d).

Representative examples of compounds of formula (I) are shown in Table 1:

| | |
|---|---|
| 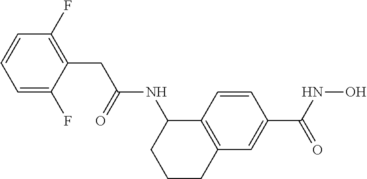 [SMILES: O=C(Cc1c(F)cccc1F)NC1CCCc2cc(C(=O)NO)ccc21] | I-1 |
| 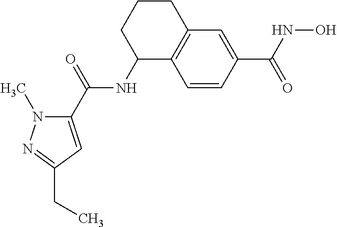 [SMILES: CCc1cc(C(=O)NC2CCCc3cc(C(=O)NO)ccc32)n(C)n1] | I-2 |
| 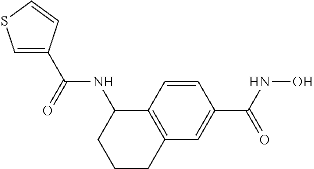 [SMILES: O=C(NO)c1ccc2c(c1)CCCC2NC(=O)c1ccsc1] | I-3 |
| 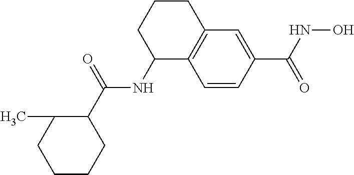 [SMILES: CC1CCCCC1C(=O)NC1CCCc2cc(C(=O)NO)ccc21] | I-4 |
| 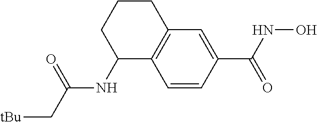 [SMILES: CC(C)(C)CC(=O)NC1CCCc2cc(C(=O)NO)ccc21] | I-5 |
| 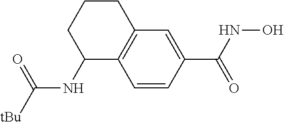 [SMILES: CC(C)(C)C(=O)NC1CCCc2cc(C(=O)NO)ccc21] | I-6 |
| 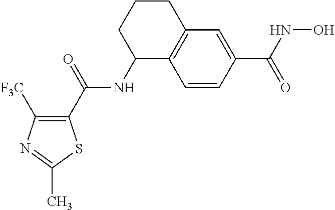 [SMILES: Cc1nc(C(F)(F)F)c(C(=O)NC2CCCc3cc(C(=O)NO)ccc32)s1] | I-7 |
| 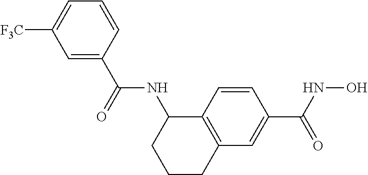 [SMILES: O=C(NO)c1ccc2c(c1)CCCC2NC(=O)c1cccc(C(F)(F)F)c1] | I-8 |
| 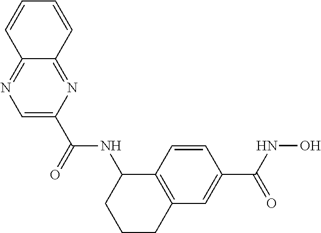 [SMILES: O=C(NO)c1ccc2c(c1)CCCC2NC(=O)c1cnc2ccccc2n1] | I-9 |
| 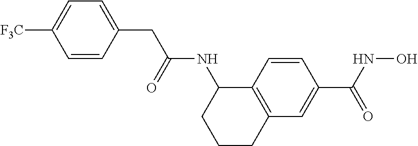 [SMILES: O=C(Cc1ccc(C(F)(F)F)cc1)NC1CCCc2cc(C(=O)NO)ccc21] | I-10 |
| 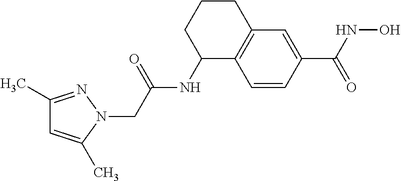 [SMILES: Cc1cc(C)n(CC(=O)NC2CCCc3cc(C(=O)NO)ccc32)n1] | I-11 |
| 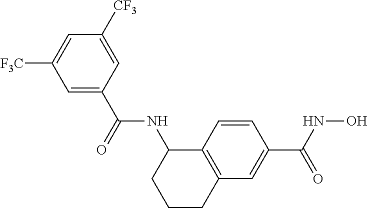 [SMILES: O=C(NO)c1ccc2c(c1)CCCC2NC(=O)c1cc(C(F)(F)F)cc(C(F)(F)F)c1] | I-12 |
| 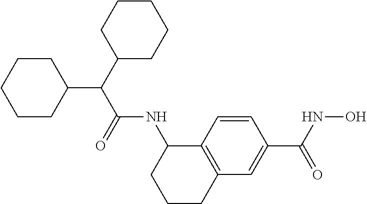 [SMILES: O=C(NO)c1ccc2c(c1)CCCC2NC(=O)C(C1CCCCC1)C1CCCCC1] | I-13 |
| 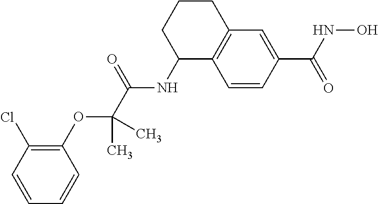 [SMILES: CC(C)(Oc1ccccc1Cl)C(=O)NC1CCCc2cc(C(=O)NO)ccc21] | I-14 |
| 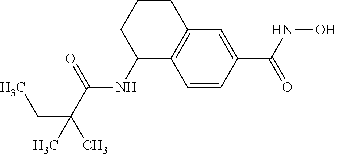 [SMILES: CCC(C)(C)C(=O)NC1CCCc2cc(C(=O)NO)ccc21] | I-15 |
| 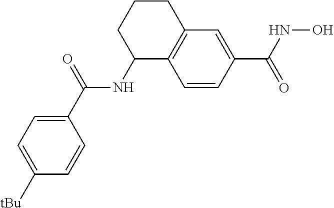 [SMILES: CC(C)(C)c1ccc(C(=O)NC2CCCc3cc(C(=O)NO)ccc32)cc1] | I-16 |
| 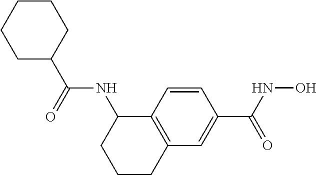 [SMILES: O=C(NO)c1ccc2c(c1)CCCC2NC(=O)C1CCCCC1] | I-17 |
| 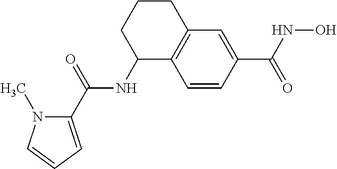 [SMILES: Cn1cccc1C(=O)NC1CCCc2cc(C(=O)NO)ccc21] | I-18 |
| 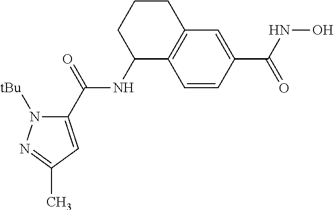 [SMILES: Cc1cc(C(=O)NC2CCCc3cc(C(=O)NO)ccc32)n(C(C)(C)C)n1] | I-19 |
| 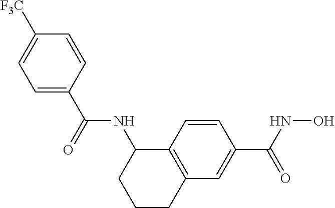 [SMILES: O=C(NO)c1ccc2c(c1)CCCC2NC(=O)c1ccc(C(F)(F)F)cc1] | I-20 |
| 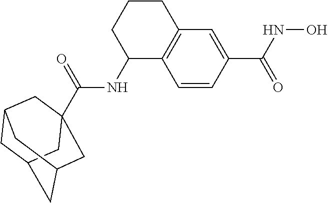 [SMILES: O=C(NO)c1ccc2c(c1)CCCC2NC(=O)C12CC3CC(CC(C3)C1)C2] | I-21 |
| 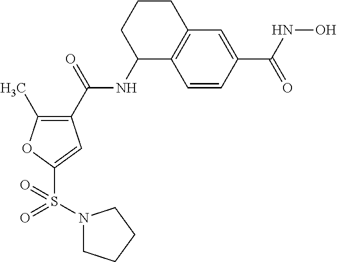 [SMILES: Cc1oc(S(=O)(=O)N2CCCC2)cc1C(=O)NC1CCCc2cc(C(=O)NO)ccc21] | I-22 |
| 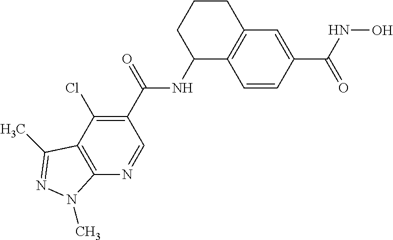 [SMILES: Cc1nn(C)c2ncc(C(=O)NC3CCCc4cc(C(=O)NO)ccc43)c(Cl)c12] | I-23 |
| 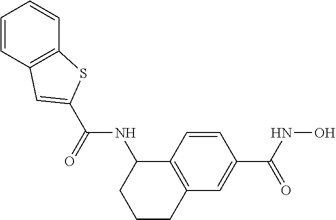 [SMILES: O=C(NO)c1ccc2c(c1)CCCC2NC(=O)c1cc2ccccc2s1] | I-24 |
| 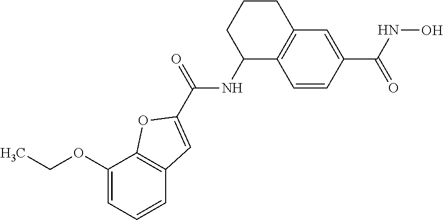 [SMILES: CCOc1cccc2cc(C(=O)NC3CCCc4cc(C(=O)NO)ccc43)oc12] | I-25 |
| 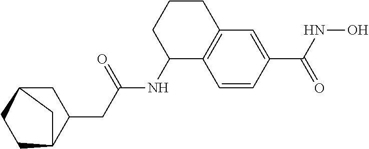 [SMILES: O=C(CC1C[C@@H]2CC[C@H]1C2)NC1CCCc2cc(C(=O)NO)ccc21] | I-26 |
| 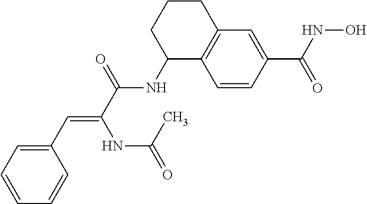 [SMILES: CC(=O)N/C(=C\c1ccccc1)C(=O)NC1CCCc2cc(C(=O)NO)ccc21] | I-27 |
| 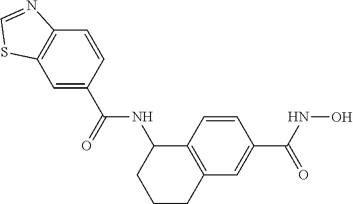 [SMILES: O=C(NO)c1ccc2c(c1)CCCC2NC(=O)c1ccc2ncsc2c1] | I-28 |
| 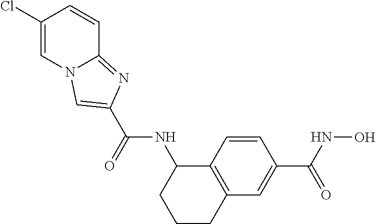 [SMILES: O=C(NO)c1ccc2c(c1)CCCC2NC(=O)c1cn2cc(Cl)ccc2n1] | I-29 |
| 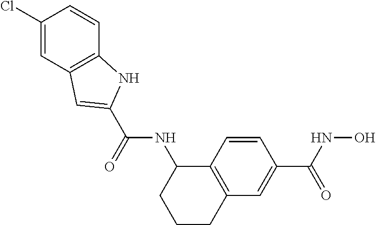 [SMILES: O=C(NO)c1ccc2c(c1)CCCC2NC(=O)c1cc2cc(Cl)ccc2[nH]1] | I-30 |
| 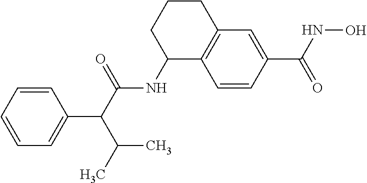 [SMILES: CC(C)C(C(=O)NC1CCCc2cc(C(=O)NO)ccc21)c1ccccc1] | I-31 |
| 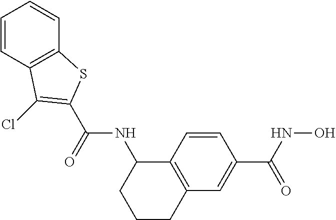 [SMILES: O=C(NO)c1ccc2c(c1)CCCC2NC(=O)c1sc2ccccc2c1Cl] | I-32 |
| 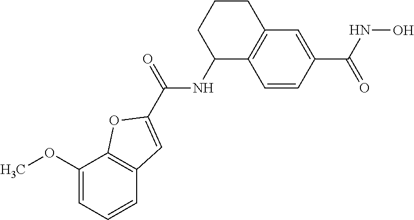 [SMILES: COc1cccc2cc(C(=O)NC3CCCc4cc(C(=O)NO)ccc43)oc12] | I-33 |
| 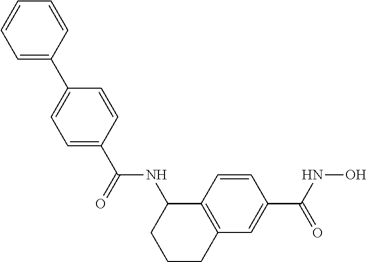 [SMILES: O=C(NO)c1ccc2c(c1)CCCC2NC(=O)c1ccc(-c2ccccc2)cc1] | I-34 |
| 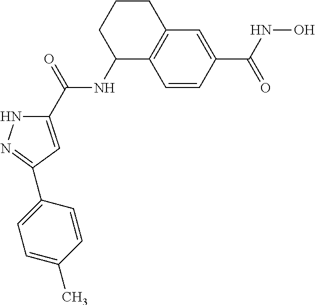 [SMILES: Cc1ccc(-c2cc(C(=O)NC3CCCc4cc(C(=O)NO)ccc43)[nH]n2)cc1] | I-35 |
| 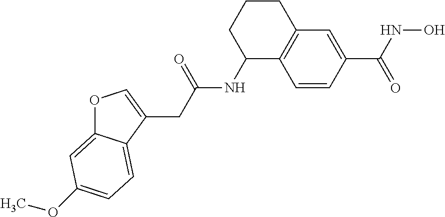 [SMILES: COc1ccc2c(CC(=O)NC3CCCc4cc(C(=O)NO)ccc43)coc2c1] | I-36 |
| 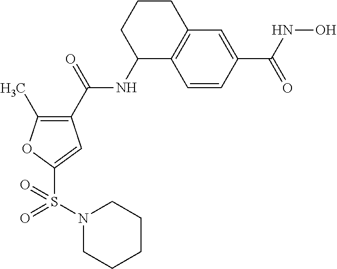 [SMILES: Cc1oc(S(=O)(=O)N2CCCCC2)cc1C(=O)NC1CCCc2cc(C(=O)NO)ccc21] | I-37 |
| 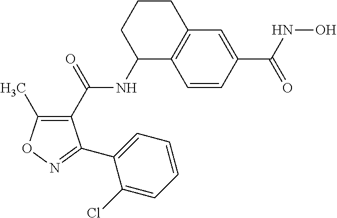 [SMILES: Cc1onc(-c2ccccc2Cl)c1C(=O)NC1CCCc2cc(C(=O)NO)ccc21] | I-38 |
| 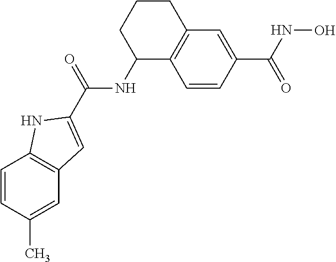 [SMILES: Cc1ccc2[nH]c(C(=O)NC3CCCc4cc(C(=O)NO)ccc43)cc2c1] | I-39 |
| 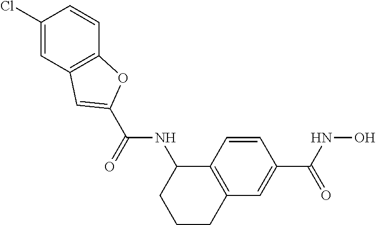 [SMILES: O=C(NO)c1ccc2c(c1)CCCC2NC(=O)c1cc2cc(Cl)ccc2o1] | I-40 |
| 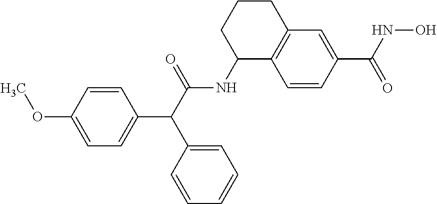 [SMILES: COc1ccc(C(C(=O)NC2CCCc3cc(C(=O)NO)ccc32)c2ccccc2)cc1] | I-41 |
| 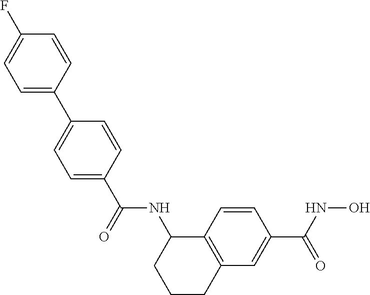 [SMILES: O=C(NO)c1ccc2c(c1)CCCC2NC(=O)c1ccc(-c2ccc(F)cc2)cc1] | I-42 |
| 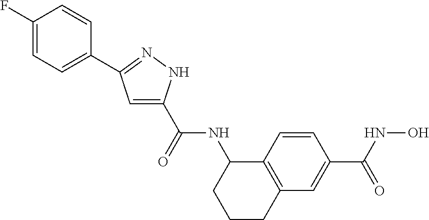 [SMILES: O=C(NO)c1ccc2c(c1)CCCC2NC(=O)c1cc(-c2ccc(F)cc2)n[nH]1] | I-43 |
| 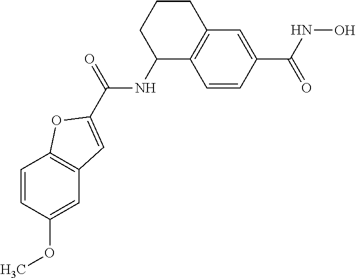 [SMILES: COc1ccc2oc(C(=O)NC3CCCc4cc(C(=O)NO)ccc43)cc2c1] | I-44 |
| 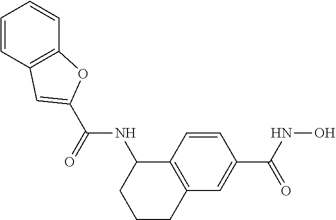 [SMILES: O=C(NO)c1ccc2c(c1)CCCC2NC(=O)c1cc2ccccc2o1] | I-45 |
| 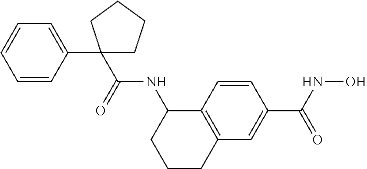 [SMILES: O=C(NO)c1ccc2c(c1)CCCC2NC(=O)C1(c2ccccc2)CCCC1] | I-46 |
| 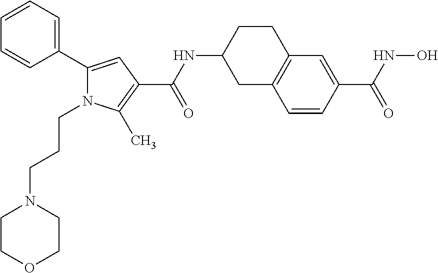 [SMILES: Cc1c(C(=O)NC2CCc3cc(C(=O)NO)ccc3C2)cc(-c2ccccc2)n1CCCN1CCOCC1] | I-47 |
| 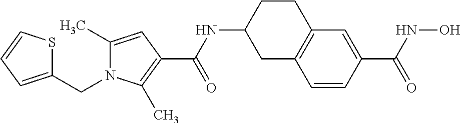 [SMILES: Cc1cc(C(=O)NC2CCc3cc(C(=O)NO)ccc3C2)c(C)n1Cc1cccs1] | I-48 |
| 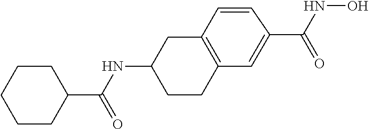 [SMILES: O=C(NO)c1ccc2c(c1)CCC(NC(=O)C1CCCCC1)C2] | I-49 |
| 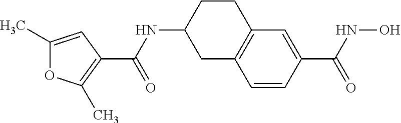 [SMILES: Cc1cc(C(=O)NC2CCc3cc(C(=O)NO)ccc3C2)c(C)o1] | I-50 |
| 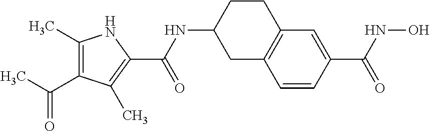 [SMILES: CC(=O)c1c(C)[nH]c(C(=O)NC2CCc3cc(C(=O)NO)ccc3C2)c1C] | I-51 |
| 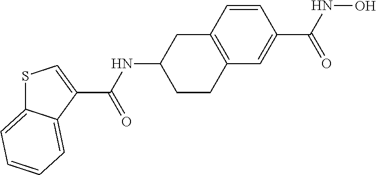 [SMILES: O=C(NO)c1ccc2c(c1)CCC(NC(=O)c1csc3ccccc13)C2] | I-52 |
| 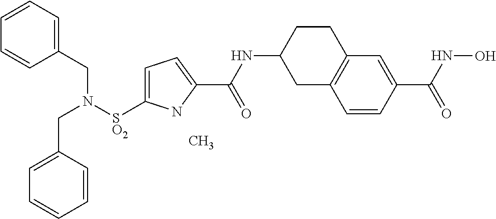 | I-53 |
| 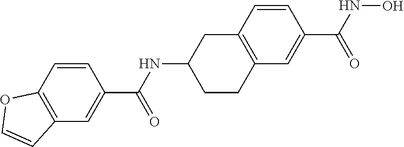 [SMILES: O=C(NO)c1ccc2c(c1)CCC(NC(=O)c1ccc3occc3c1)C2] | I-54 |
| 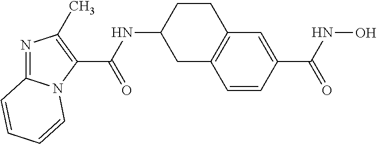 [SMILES: Cc1nc2ccccn2c1C(=O)NC1CCc2cc(C(=O)NO)ccc2C1] | I-55 |
| 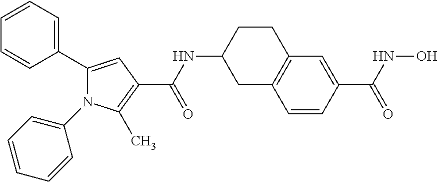 [SMILES: Cc1c(C(=O)NC2CCc3cc(C(=O)NO)ccc3C2)cc(-c2ccccc2)n1-c1ccccc1] | I-56 |
| 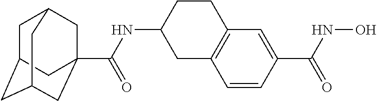 [SMILES: O=C(NO)c1ccc2c(c1)CCC(NC(=O)C13CC4CC(CC(C4)C1)C3)C2] | I-57 |
| 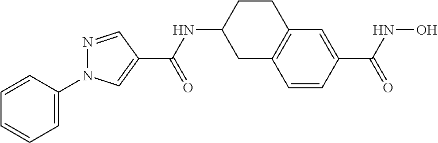 [SMILES: O=C(NO)c1ccc2c(c1)CCC(NC(=O)c1cnn(-c3ccccc3)c1)C2] | I-58 |
| 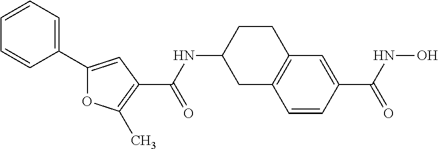 [SMILES: Cc1oc(-c2ccccc2)cc1C(=O)NC1CCc2cc(C(=O)NO)ccc2C1] | I-59 |
| 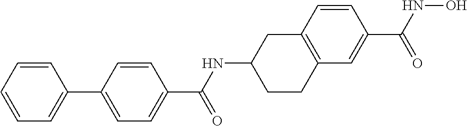 [SMILES: O=C(NO)c1ccc2c(c1)CCC(NC(=O)c1ccc(-c3ccccc3)cc1)C2] | I-60 |
| 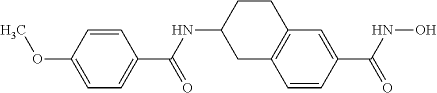 [SMILES: COc1ccc(C(=O)NC2CCc3cc(C(=O)NO)ccc3C2)cc1] | I-61 |
| 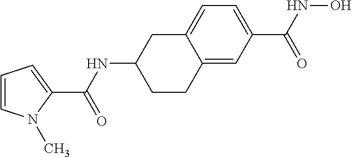 [SMILES: Cn1cccc1C(=O)NC1CCc2cc(C(=O)NO)ccc2C1] | I-62 |
| 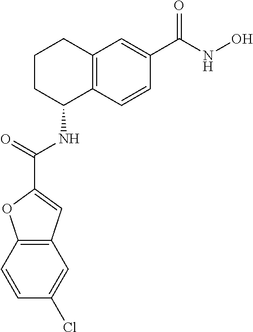 [SMILES: O=C(NO)c1ccc2c(c1)CCC[C@H]2NC(=O)c1cc2cc(Cl)ccc2o1] | I-63 |
| 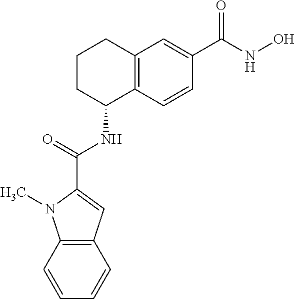 [SMILES: Cn1c(C(=O)N[C@@H]2CCCc3cc(C(=O)NO)ccc32)cc2ccccc21] | I-64 |
| 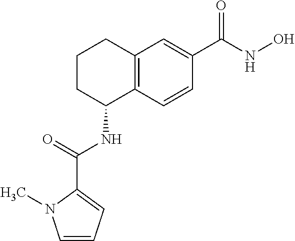 [SMILES: Cn1cccc1C(=O)N[C@@H]1CCCc2cc(C(=O)NO)ccc21] | I-65 |
| 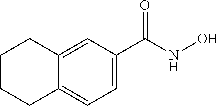 [SMILES: O=C(NO)c1ccc2c(c1)CCCC2] | I-66 |
| 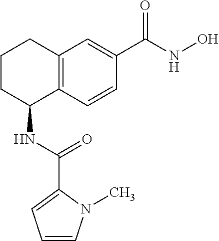 [SMILES: Cn1cccc1C(=O)N[C@H]1CCCc2cc(C(=O)NO)ccc21] | I-67 |
| 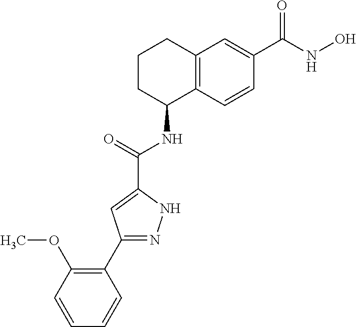 [SMILES: COc1ccccc1-c1cc(C(=O)N[C@H]2CCCc3cc(C(=O)NO)ccc32)[nH]n1] | I-68 |
| 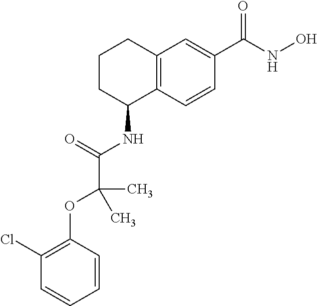 [SMILES: CC(C)(Oc1ccccc1Cl)C(=O)N[C@H]1CCCc2cc(C(=O)NO)ccc21] | I-69 |
| 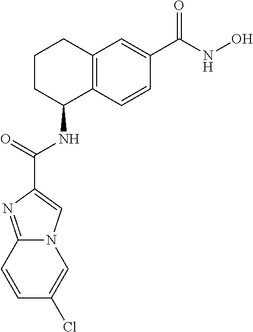 [SMILES: O=C(NO)c1ccc2c(c1)CCC[C@@H]2NC(=O)c1cn2cc(Cl)ccc2n1] | I-70 |
| 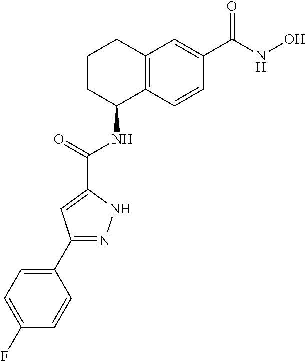 [SMILES: O=C(NO)c1ccc2c(c1)CCC[C@@H]2NC(=O)c1cc(-c2ccc(F)cc2)n[nH]1] | I-71 |
| 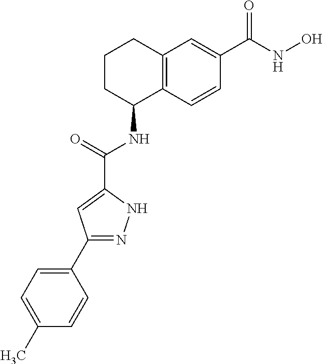 [SMILES: Cc1ccc(-c2cc(C(=O)N[C@H]3CCCc4cc(C(=O)NO)ccc43)[nH]n2)cc1] | I-72 |
| 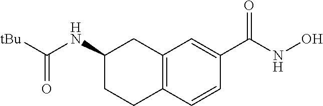 [SMILES: CC(C)(C)C(=O)N[C@@H]1CCc2ccc(C(=O)NO)cc2C1] | I-73 |
| 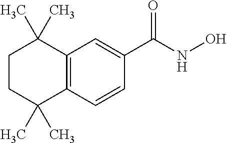 [SMILES: CC1(C)CCC(C)(C)c2cc(C(=O)NO)ccc21] | I-74 |
| 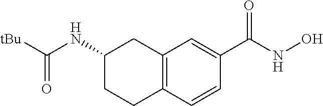 [SMILES: CC(C)(C)C(=O)N[C@H]1CCc2ccc(C(=O)NO)cc2C1] | I-75 |
| 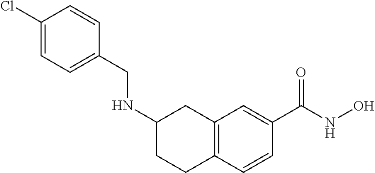 [SMILES: O=C(NO)c1ccc2c(c1)CC(NCc1ccc(Cl)cc1)CC2] | I-76 |
| 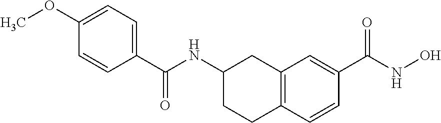 [SMILES: COc1ccc(C(=O)NC2CCc3ccc(C(=O)NO)cc3C2)cc1] | I-77 |
| 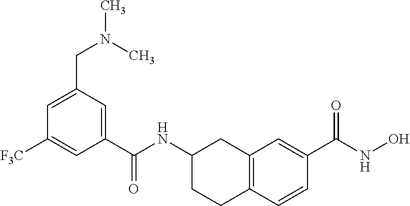 [SMILES: CN(C)Cc1cc(C(=O)NC2CCc3ccc(C(=O)NO)cc3C2)cc(C(F)(F)F)c1] | I-78 |
| 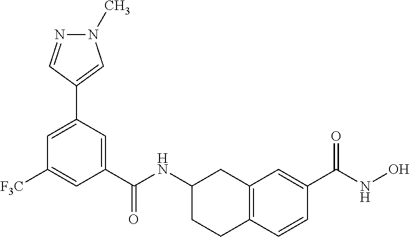 [SMILES: Cn1cc(-c2cc(C(=O)NC3CCc4ccc(C(=O)NO)cc4C3)cc(C(F)(F)F)c2)cn1] | I-79 |
| 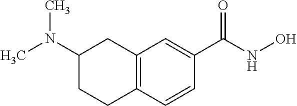 [SMILES: CN(C)C1CCc2ccc(C(=O)NO)cc2C1] | I-80 |
| 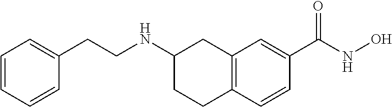 [SMILES: O=C(NO)c1ccc2c(c1)CC(NCCc1ccccc1)CC2] | I-81 |
| 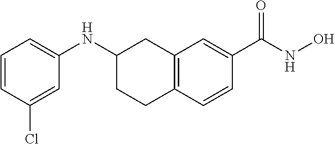 [SMILES: O=C(NO)c1ccc2c(c1)CC(Nc1cccc(Cl)c1)CC2] | I-82 |
| 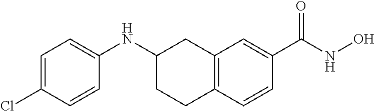 [SMILES: O=C(NO)c1ccc2c(c1)CC(Nc1ccc(Cl)cc1)CC2] | I-83 |
| 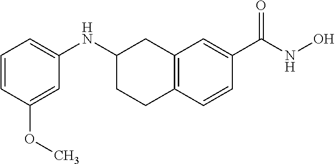 [SMILES: COc1cccc(NC2CCc3ccc(C(=O)NO)cc3C2)c1] | I-84 |
| 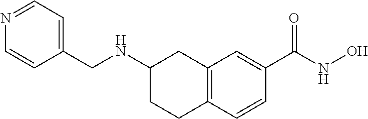 [SMILES: O=C(NO)c1ccc2c(c1)CC(NCc1ccncc1)CC2] | I-85 |
| 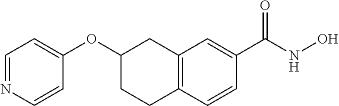 [SMILES: O=C(NO)c1ccc2c(c1)CC(Oc1ccncc1)CC2] | I-86 |
| 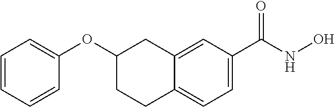 [SMILES: O=C(NO)c1ccc2c(c1)CC(Oc1ccccc1)CC2] | I-87 |
| 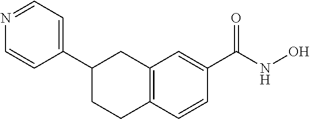 [SMILES: O=C(NO)c1ccc2c(c1)CC(c1ccncc1)CC2] | I-88 |
| 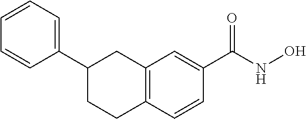 [SMILES: O=C(NO)c1ccc2c(c1)CC(c1ccccc1)CC2] | I-89 |
| 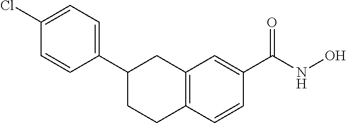 [SMILES: O=C(NO)c1ccc2c(c1)CC(c1ccc(Cl)cc1)CC2] | I-90 |
| 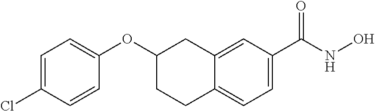 [SMILES: O=C(NO)c1ccc2c(c1)CC(Oc1ccc(Cl)cc1)CC2] | I-91 |
| 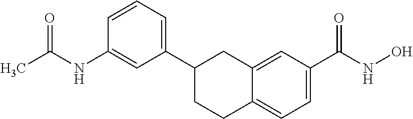 [SMILES: CC(=O)Nc1cccc(C2CCc3ccc(C(=O)NO)cc3C2)c1] | I-92 |
| 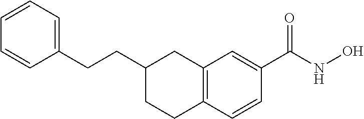 [SMILES: O=C(NO)c1ccc2c(c1)CC(CCc1ccccc1)CC2] | I-93 |
| 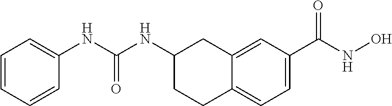 [SMILES: O=C(Nc1ccccc1)NC1CCc2ccc(C(=O)NO)cc2C1] | I-94 |
| 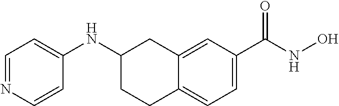 [SMILES: O=C(NO)c1ccc2c(c1)CC(Nc1ccncc1)CC2] | I-95 |
| 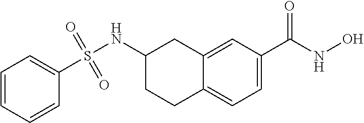 [SMILES: O=C(NO)c1ccc2c(c1)CC(NS(=O)(=O)c1ccccc1)CC2] | I-96 |
| 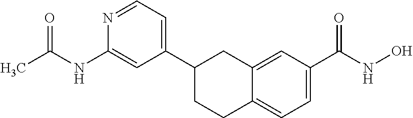 [SMILES: CC(=O)Nc1cc(C2CCc3ccc(C(=O)NO)cc3C2)ccn1] | I-97 |
| 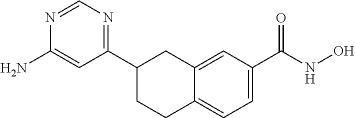 [SMILES: Nc1cc(C2CCc3ccc(C(=O)NO)cc3C2)ncn1] | I-98 |
| 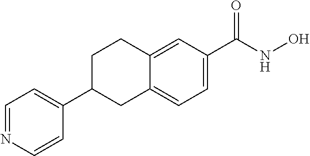 [SMILES: O=C(NO)c1ccc2c(c1)CCC(c1ccncc1)C2] | I-99 |
| 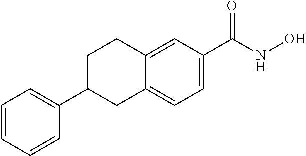 [SMILES: O=C(NO)c1ccc2c(c1)CCC(c1ccccc1)C2] | I-100 |
| 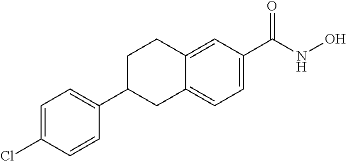 [SMILES: O=C(NO)c1ccc2c(c1)CCC(c1ccc(Cl)cc1)C2] | I-101 |
| 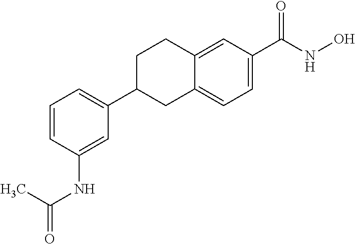 [SMILES: CC(=O)Nc1cccc(C2CCc3cc(C(=O)NO)ccc3C2)c1] | I-102 |
| 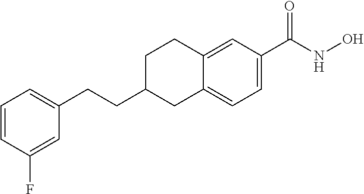 [SMILES: O=C(NO)c1ccc2c(c1)CCC(CCc1cccc(F)c1)C2] | I-103 |
| 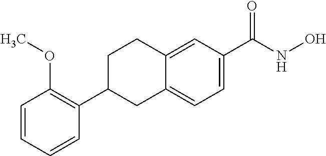 [SMILES: COc1ccccc1C1CCc2cc(C(=O)NO)ccc2C1] | I-104 |
| 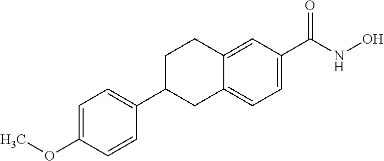 [SMILES: COc1ccc(C2CCc3cc(C(=O)NO)ccc3C2)cc1] | I-105 |
| 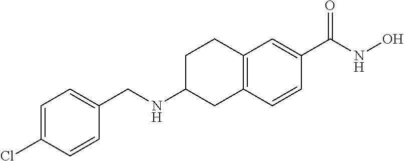 [SMILES: O=C(NO)c1ccc2c(c1)CCC(NCc1ccc(Cl)cc1)C2] | I-106 |
| 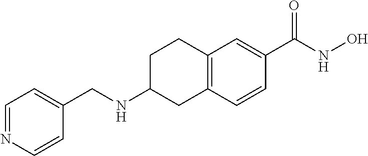 [SMILES: O=C(NO)c1ccc2c(c1)CCC(NCc1ccncc1)C2] | I-107 |
| 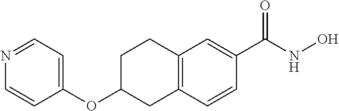 [SMILES: O=C(NO)c1ccc2c(c1)CCC(Oc1ccncc1)C2] | I-108 |
| 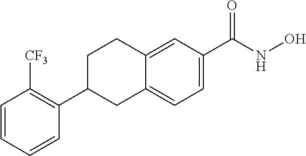 [SMILES: O=C(NO)c1ccc2c(c1)CCC(c1ccccc1C(F)(F)F)C2] | I-109 |
| 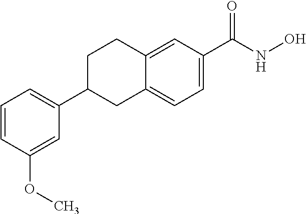 [SMILES: COc1cccc(C2CCc3cc(C(=O)NO)ccc3C2)c1] | I-110 |
| 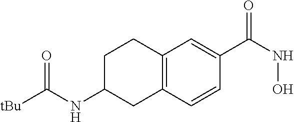 [SMILES: CC(C)(C)C(=O)NC1CCc2cc(C(=O)NO)ccc2C1] | I-111 |
| 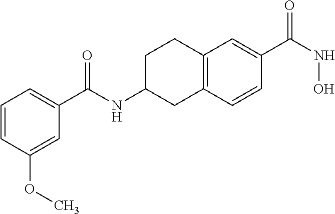 [SMILES: COc1cccc(C(=O)NC2CCc3cc(C(=O)NO)ccc3C2)c1] | I-112 |
| 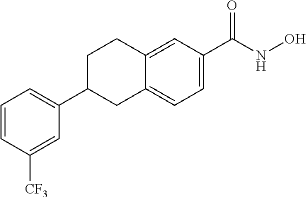 [SMILES: O=C(NO)c1ccc2c(c1)CCC(c1cccc(C(F)(F)F)c1)C2] | I-113 |
| 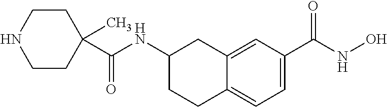 [SMILES: CC1(C(=O)NC2CCc3ccc(C(=O)NO)cc3C2)CCNCC1] | I-114 |
| 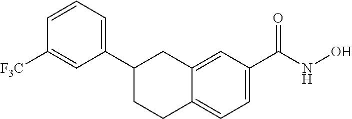 [SMILES: O=C(NO)c1ccc2c(c1)CC(c1cccc(C(F)(F)F)c1)CC2] | I-115 |
| 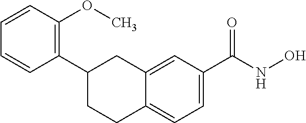 [SMILES: COc1ccccc1C1CCc2ccc(C(=O)NO)cc2C1] | I-116 |
| 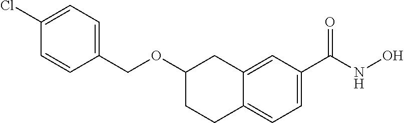 [SMILES: O=C(NO)c1ccc2c(c1)CC(OCc1ccc(Cl)cc1)CC2] | I-117 |
| 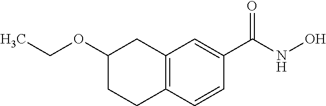 [SMILES: CCOC1CCc2ccc(C(=O)NO)cc2C1] | I-118 |
| 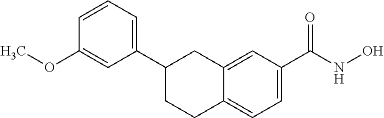 [SMILES: COc1cccc(C2CCc3ccc(C(=O)NO)cc3C2)c1] | I-119 |
| 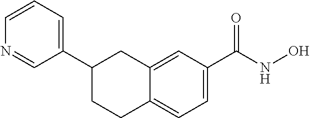 [SMILES: O=C(NO)c1ccc2c(c1)CC(c1cccnc1)CC2] | I-120 |
| 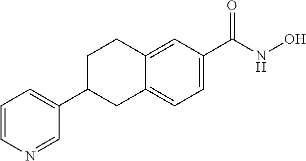 [SMILES: O=C(NO)c1ccc2c(c1)CCC(c1cccnc1)C2] | I-121 |
| 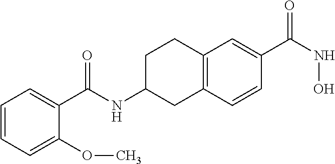 [SMILES: COc1ccccc1C(=O)NC1CCc2cc(C(=O)NO)ccc2C1] | I-122 |
| 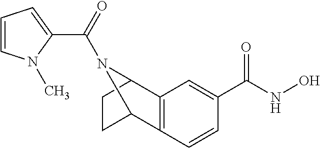 [SMILES: Cn1cccc1C(=O)N1C2CCC1c1cc(C(=O)NO)ccc12] | I-123 |
| 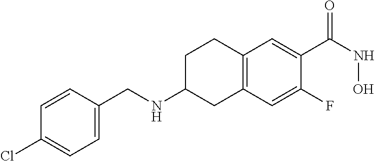 [SMILES: O=C(NO)c1cc2c(cc1F)CC(NCc1ccc(Cl)cc1)CC2] | I-124 |
| 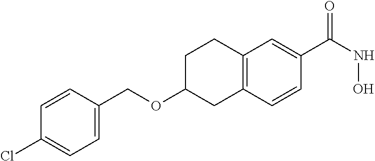 [SMILES: O=C(NO)c1ccc2c(c1)CCC(OCc1ccc(Cl)cc1)C2] | I-125 |
| 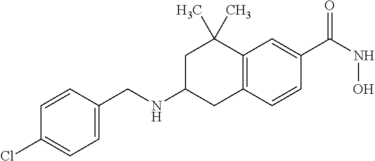 [SMILES: CC1(C)CC(NCc2ccc(Cl)cc2)Cc2ccc(C(=O)NO)cc21] | I-126 |
| 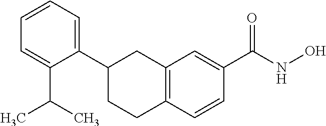 [SMILES: CC(C)c1ccccc1C1CCc2ccc(C(=O)NO)cc2C1] | I-127 |
| 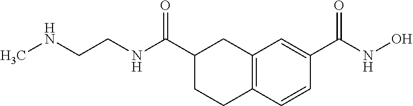 [SMILES: CNCCNC(=O)C1CCc2ccc(C(=O)NO)cc2C1] | I-128 |
| 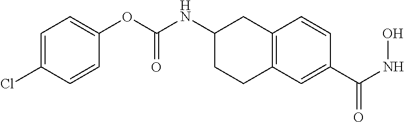 [SMILES: O=C(NC1CCc2cc(C(=O)NO)ccc2C1)Oc1ccc(Cl)cc1] | I-129 |
| 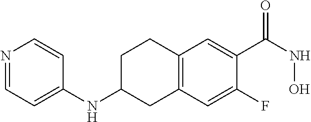 [SMILES: O=C(NO)c1cc2c(cc1F)CC(Nc1ccncc1)CC2] | I-130 |
| 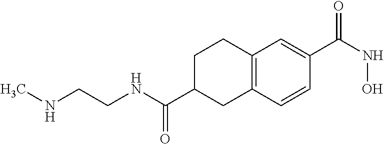 [SMILES: CNCCNC(=O)C1CCc2cc(C(=O)NO)ccc2C1] | I-131 |
| 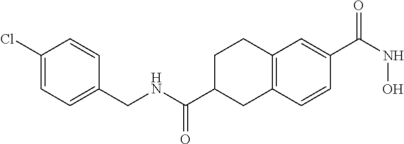 [SMILES: O=C(NO)c1ccc2c(c1)CCC(C(=O)NCc1ccc(Cl)cc1)C2] | I-132 |
| 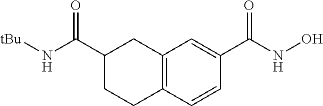 [SMILES: CC(C)(C)NC(=O)C1CCc2ccc(C(=O)NO)cc2C1] | I-133 |
| 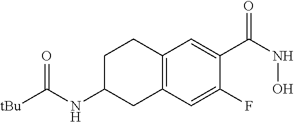 [SMILES: CC(C)(C)C(=O)NC1CCc2cc(C(=O)NO)c(F)cc2C1] | I-134 |
| 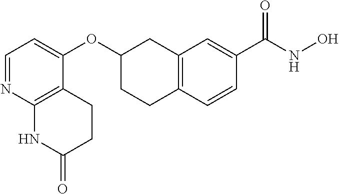 [SMILES: O=C1CCc2c(OC3CCc4ccc(C(=O)NO)cc4C3)ccnc2N1] | I-135 |
| 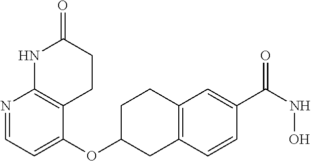 [SMILES: O=C1CCc2c(OC3CCc4cc(C(=O)NO)ccc4C3)ccnc2N1] | I-136 |
| 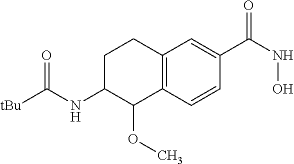 [SMILES: COC1c2ccc(C(=O)NO)cc2CCC1NC(=O)C(C)(C)C] | I-137 |
| 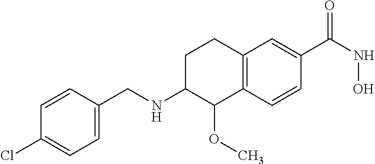 [SMILES: COC1c2ccc(C(=O)NO)cc2CCC1NCc1ccc(Cl)cc1] | I-138 |
| 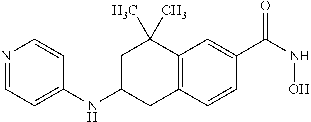 [SMILES: CC1(C)CC(Nc2ccncc2)Cc2ccc(C(=O)NO)cc21] | I-139 |
| 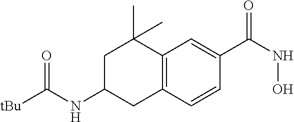 [SMILES: CC(C)(C)C(=O)NC1Cc2ccc(C(=O)NO)cc2C(C)(C)C1] | I-140 |
| 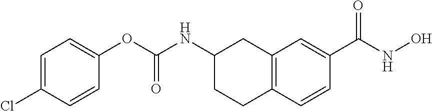 [SMILES: O=C(NC1CCc2ccc(C(=O)NO)cc2C1)Oc1ccc(Cl)cc1] | I-141 |
| 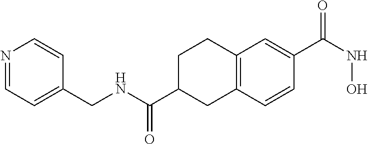 [SMILES: O=C(NO)c1ccc2c(c1)CCC(C(=O)NCc1ccncc1)C2] | I-142 |
| 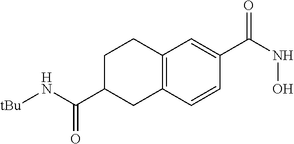 [SMILES: CC(C)(C)NC(=O)C1CCc2cc(C(=O)NO)ccc2C1] | I-143 |
| 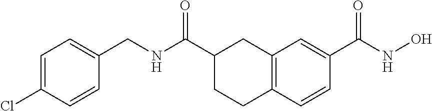 [SMILES: O=C(NO)c1ccc2c(c1)CC(C(=O)NCc1ccc(Cl)cc1)CC2] | I-144 |
| 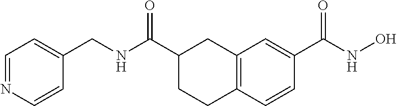 [SMILES: O=C(NO)c1ccc2c(c1)CC(C(=O)NCc1ccncc1)CC2] | I-145 |
| 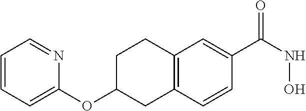 [SMILES: O=C(NO)c1ccc2c(c1)CCC(Oc1ccccn1)C2] | I-146 |
| 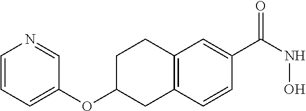 [SMILES: O=C(NO)c1ccc2c(c1)CCC(Oc1cccnc1)C2] | I-147 |
| 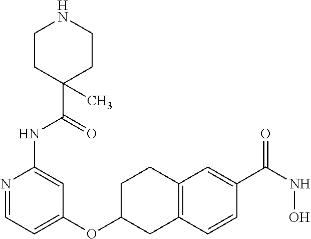 [SMILES: CC1(C(=O)Nc2cc(OC3CCc4cc(C(=O)NO)ccc4C3)ccn2)CCNCC1] | I-148 |
| 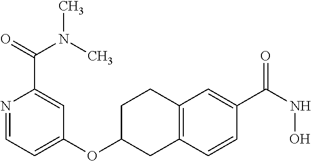 [SMILES: CN(C)C(=O)c1cc(OC2CCc3cc(C(=O)NO)ccc3C2)ccn1] | I-149 |
| 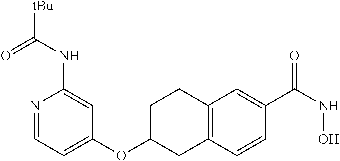 [SMILES: CC(C)(C)C(=O)Nc1cc(OC2CCc3cc(C(=O)NO)ccc3C2)ccn1] | I-150 |
| 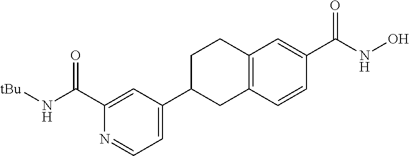 [SMILES: CC(C)(C)NC(=O)c1cc(C2CCc3cc(C(=O)NO)ccc3C2)ccn1] | I-151 |
| 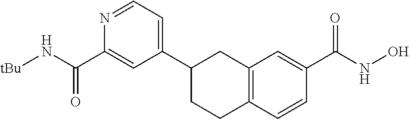 [SMILES: CC(C)(C)NC(=O)c1cc(C2CCc3ccc(C(=O)NO)cc3C2)ccn1] | I-152 |
| 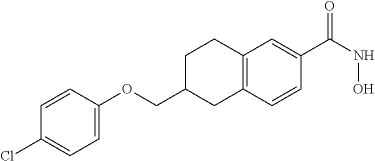 [SMILES: O=C(NO)c1ccc2c(c1)CCC(COc1ccc(Cl)cc1)C2] | I-153 |
| 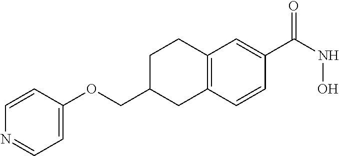 [SMILES: O=C(NO)c1ccc2c(c1)CCC(COc1ccncc1)C2] | I-154 |
| 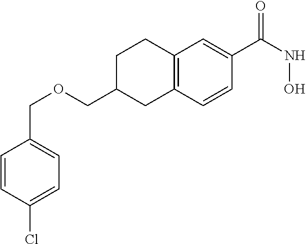 [SMILES: O=C(NO)c1ccc2c(c1)CCC(COCc1ccc(Cl)cc1)C2] | I-155 |
| 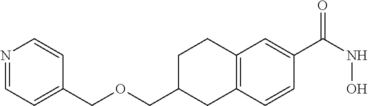 [SMILES: O=C(NO)c1ccc2c(c1)CCC(COCc1ccncc1)C2] | I-156 |
| 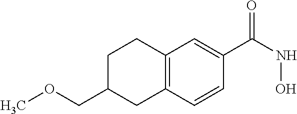 [SMILES: COCC1CCc2cc(C(=O)NO)ccc2C1] | I-157 |
| 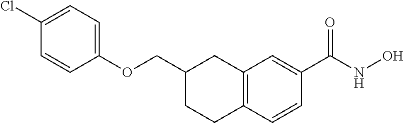 [SMILES: O=C(NO)c1ccc2c(c1)CC(COc1ccc(Cl)cc1)CC2] | I-158 |
| 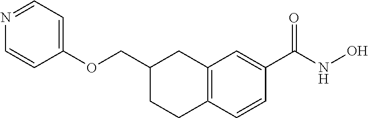 [SMILES: O=C(NO)c1ccc2c(c1)CC(COc1ccncc1)CC2] | I-159 |
| 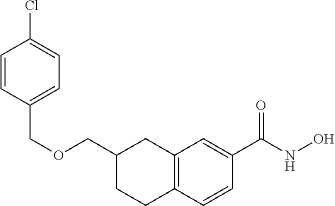 [SMILES: O=C(NO)c1ccc2c(c1)CC(COCc1ccc(Cl)cc1)CC2] | I-160 |
| 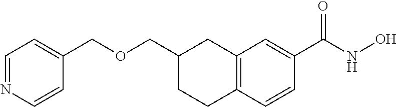 [SMILES: O=C(NO)c1ccc2c(c1)CC(COCc1ccncc1)CC2] | I-161 |
| 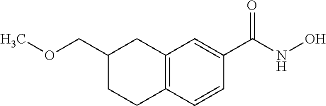 [SMILES: COCC1CCc2ccc(C(=O)NO)cc2C1] | I-162 |
| 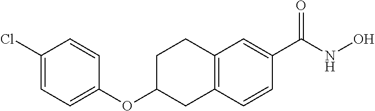 [SMILES: O=C(NO)c1ccc2c(c1)CCC(Oc1ccc(Cl)cc1)C2] | I-163 |
| 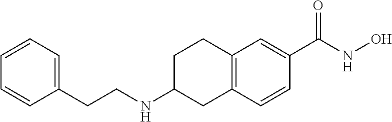 [SMILES: O=C(NO)c1ccc2c(c1)CCC(NCCc1ccccc1)C2] | I-164 |
| 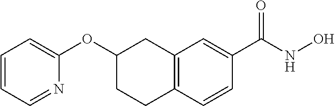 [SMILES: O=C(NO)c1ccc2c(c1)CC(Oc1ccccn1)CC2] | I-165 |
| 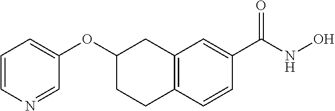 [SMILES: O=C(NO)c1ccc2c(c1)CC(Oc1cccnc1)CC2] | I-166 |
| 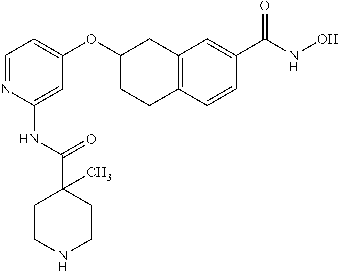 [SMILES: CC1(C(=O)Nc2cc(OC3CCc4ccc(C(=O)NO)cc4C3)ccn2)CCNCC1] | I-167 |
| 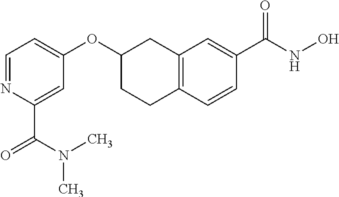 [SMILES: CN(C)C(=O)c1cc(OC2CCc3ccc(C(=O)NO)cc3C2)ccn1] | I-168 |
| 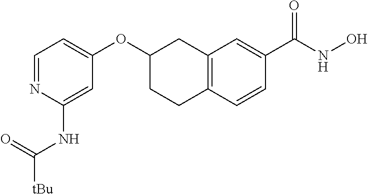 [SMILES: CC(C)(C)C(=O)Nc1cc(OC2CCc3ccc(C(=O)NO)cc3C2)ccn1] | I-169 |
| 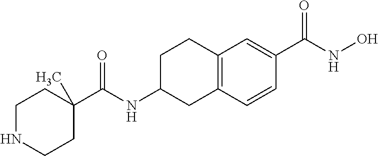 [SMILES: CC1(C(=O)NC2CCc3cc(C(=O)NO)ccc3C2)CCNCC1] | I-170 |
| 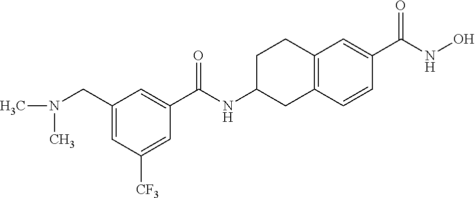 [SMILES: CN(C)Cc1cc(C(=O)NC2CCc3cc(C(=O)NO)ccc3C2)cc(C(F)(F)F)c1] | I-171 |
| 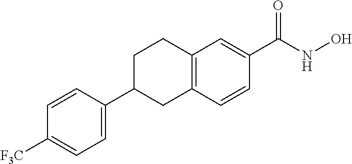 [SMILES: O=C(NO)c1ccc2c(c1)CCC(c1ccc(C(F)(F)F)cc1)C2] | I-172 |
| 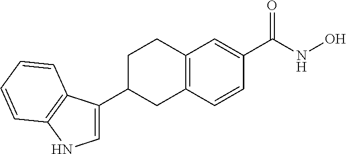 [SMILES: O=C(NO)c1ccc2c(c1)CCC(c1c[nH]c3ccccc13)C2] | I-173 |
| 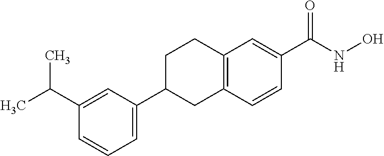 [SMILES: CC(C)c1cccc(C2CCc3cc(C(=O)NO)ccc3C2)c1] | I-174 |
| 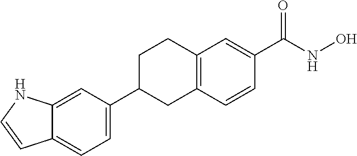 [SMILES: O=C(NO)c1ccc2c(c1)CCC(c1ccc3cc[nH]c3c1)C2] | I-175 |
| 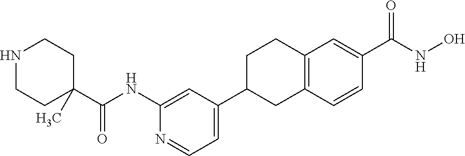 [SMILES: CC1(C(=O)Nc2cc(C3CCc4cc(C(=O)NO)ccc4C3)ccn2)CCNCC1] | I-176 |
| 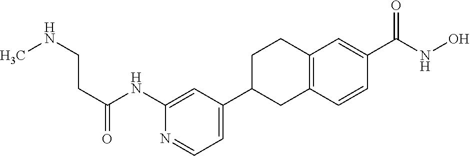 [SMILES: CNCCC(=O)Nc1cc(C2CCc3cc(C(=O)NO)ccc3C2)ccn1] | I-177 |
| 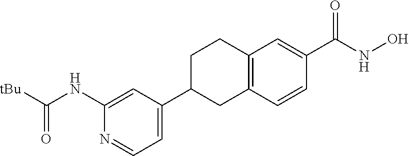 [SMILES: CC(C)(C)C(=O)Nc1cc(C2CCc3cc(C(=O)NO)ccc3C2)ccn1] | I-178 |
| 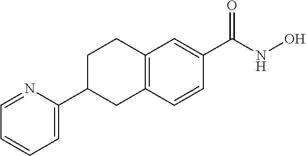 [SMILES: O=C(NO)c1ccc2c(c1)CCC(c1ccccn1)C2] | I-179 |
| 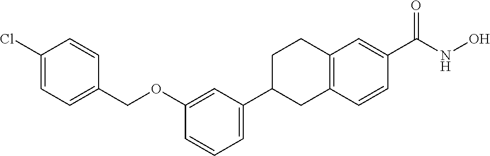 [SMILES: O=C(NO)c1ccc2c(c1)CCC(c1cccc(OCc3ccc(Cl)cc3)c1)C2] | I-180 |
| 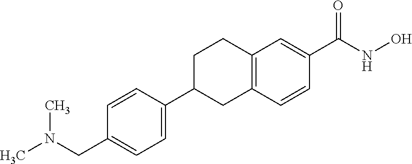 [SMILES: CN(C)Cc1ccc(C2CCc3cc(C(=O)NO)ccc3C2)cc1] | I-181 |
| 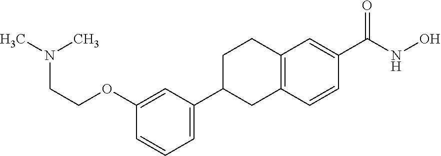 [SMILES: CN(C)CCOc1cccc(C2CCc3cc(C(=O)NO)ccc3C2)c1] | I-182 |
| 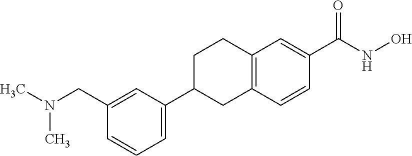 [SMILES: CN(C)Cc1cccc(C2CCc3cc(C(=O)NO)ccc3C2)c1] | I-183 |
| 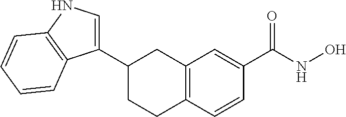 [SMILES: O=C(NO)c1ccc2c(c1)CC(c1c[nH]c3ccccc13)CC2] | I-184 |
| 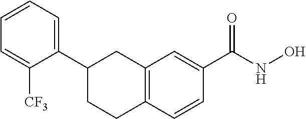 [SMILES: O=C(NO)c1ccc2c(c1)CC(c1ccccc1C(F)(F)F)CC2] | I-185 |
| 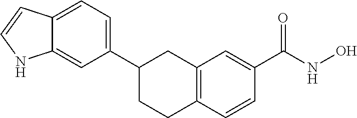 [SMILES: O=C(NO)c1ccc2c(c1)CC(c1ccc3cc[nH]c3c1)CC2] | I-186 |
| 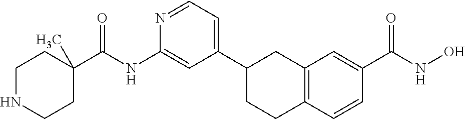 [SMILES: CC1(C(=O)Nc2cc(C3CCc4ccc(C(=O)NO)cc4C3)ccn2)CCNCC1] | I-187 |
| 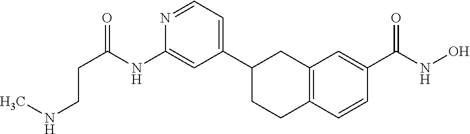 [SMILES: CNCCC(=O)Nc1cc(C2CCc3ccc(C(=O)NO)cc3C2)ccn1] | I-188 |
| 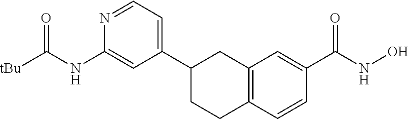 [SMILES: CC(C)(C)C(=O)Nc1cc(C2CCc3ccc(C(=O)NO)cc3C2)ccn1] | I-189 |
| 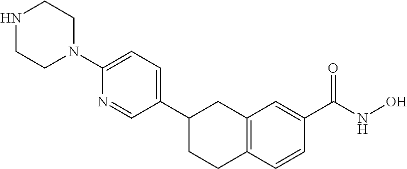 [SMILES: O=C(NO)c1ccc2c(c1)CC(c1ccc(N3CCNCC3)nc1)CC2] | I-190 |
| 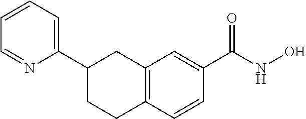 [SMILES: O=C(NO)c1ccc2c(c1)CC(c1ccccn1)CC2] | I-191 |
| 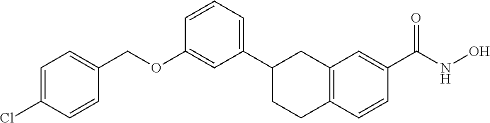 [SMILES: O=C(NO)c1ccc2c(c1)CC(c1cccc(OCc3ccc(Cl)cc3)c1)CC2] | I-192 |
| 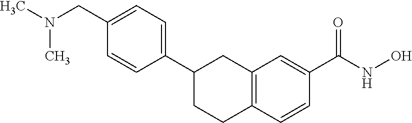 [SMILES: CN(C)Cc1ccc(C2CCc3ccc(C(=O)NO)cc3C2)cc1] | I-193 |
| 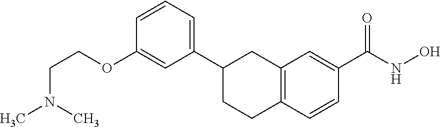 [SMILES: CN(C)CCOc1cccc(C2CCc3ccc(C(=O)NO)cc3C2)c1] | I-194 |
| 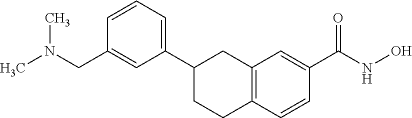 [SMILES: CN(C)Cc1cccc(C2CCc3ccc(C(=O)NO)cc3C2)c1] | I-195 |
| 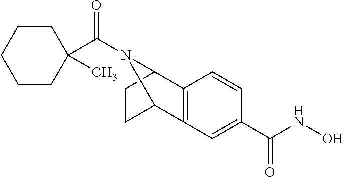 [SMILES: CC1(C(=O)N2C3CCC2c2cc(C(=O)NO)ccc23)CCCCC1] | I-196 |
| 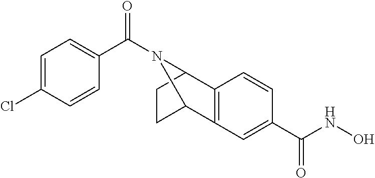 [SMILES: O=C(NO)c1ccc2c(c1)C1CCC2N1C(=O)c1ccc(Cl)cc1] | I-197 |
| 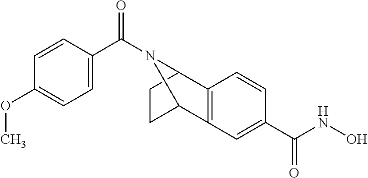 [SMILES: COc1ccc(C(=O)N2C3CCC2c2cc(C(=O)NO)ccc23)cc1] | I-198 |
| 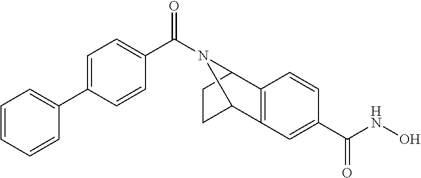 [SMILES: O=C(NO)c1ccc2c(c1)C1CCC2N1C(=O)c1ccc(-c2ccccc2)cc1] | I-199 |
| 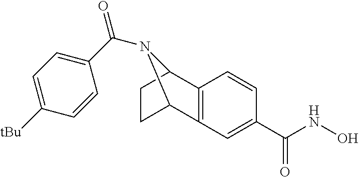 [SMILES: CC(C)(C)c1ccc(C(=O)N2C3CCC2c2cc(C(=O)NO)ccc23)cc1] | I-200 |
| 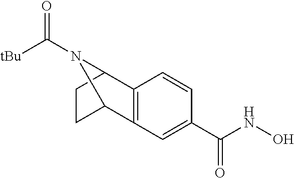 [SMILES: CC(C)(C)C(=O)N1C2CCC1c1cc(C(=O)NO)ccc12] | I-201 |
| 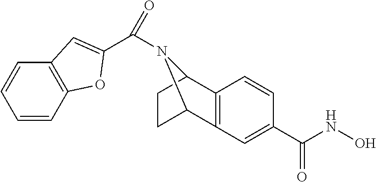 [SMILES: O=C(NO)c1ccc2c(c1)C1CCC2N1C(=O)c1cc2ccccc2o1] | I-202 |
| 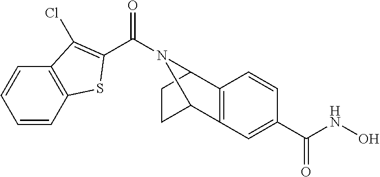 [SMILES: O=C(NO)c1ccc2c(c1)C1CCC2N1C(=O)c1sc2ccccc2c1Cl] | I-203 |
| 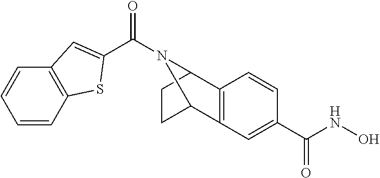 [SMILES: O=C(NO)c1ccc2c(c1)C1CCC2N1C(=O)c1cc2ccccc2s1] | I-204 |
| 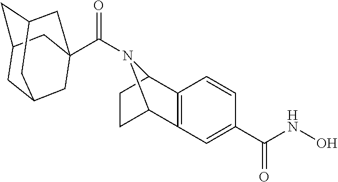 [SMILES: O=C(NO)c1ccc2c(c1)C1CCC2N1C(=O)C12CC3CC(CC(C3)C1)C2] | I-205 |
| 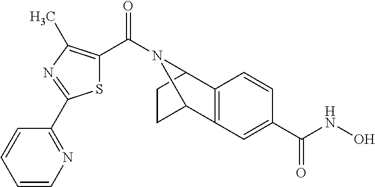 [SMILES: Cc1nc(-c2ccccn2)sc1C(=O)N1C2CCC1c1cc(C(=O)NO)ccc12] | I-206 |
| 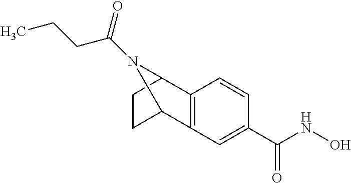 [SMILES: CCCC(=O)N1C2CCC1c1cc(C(=O)NO)ccc12] | I-207 |
| 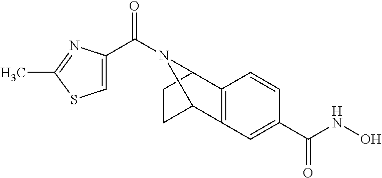 [SMILES: Cc1nc(C(=O)N2C3CCC2c2cc(C(=O)NO)ccc23)cs1] | I-208 |
| 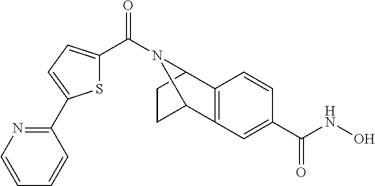 [SMILES: O=C(NO)c1ccc2c(c1)C1CCC2N1C(=O)c1ccc(-c2ccccn2)s1] | I-209 |
| 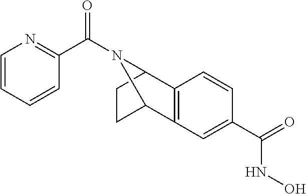 [SMILES: O=C(NO)c1ccc2c(c1)C1CCC2N1C(=O)c1ccccn1] | I-210 |
| 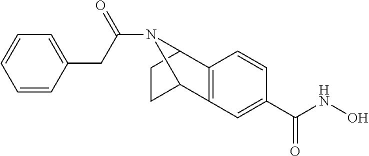 [SMILES: O=C(NO)c1ccc2c(c1)C1CCC2N1C(=O)Cc1ccccc1] | I-211 |
| 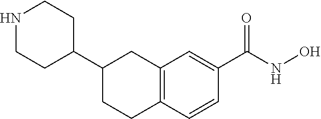 [SMILES: O=C(NO)c1ccc2c(c1)CC(C1CCNCC1)CC2] | I-212 |
| 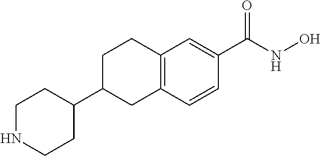 [SMILES: O=C(NO)c1ccc2c(c1)CCC(C1CCNCC1)C2] | I-213 |
| 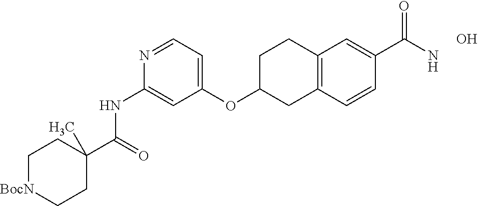 | I-214 |
| 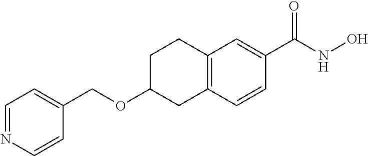 [SMILES: O=C(NO)c1ccc2c(c1)CCC(OCc1ccncc1)C2] | I-215 |
| 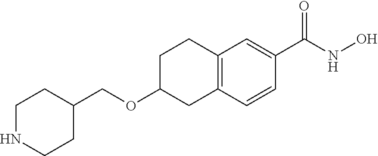 [SMILES: O=C(NO)c1ccc2c(c1)CCC(OCC1CCNCC1)C2] | I-216 |
| 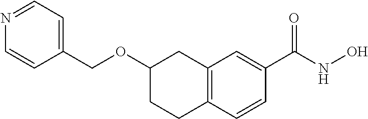 [SMILES: O=C(NO)c1ccc2c(c1)CC(OCc1ccncc1)CC2] | I-217 |
| 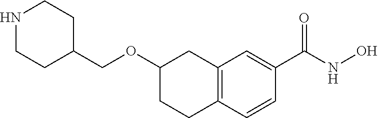 [SMILES: O=C(NO)c1ccc2c(c1)CC(OCC1CCNCC1)CC2] | I-218 |
| 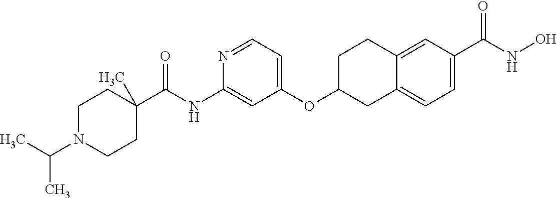 [SMILES: CC(C)N1CCC(C)(C(=O)Nc2cc(OC3CCc4cc(C(=O)NO)ccc4C3)ccn2)CC1] | I-219 |
| 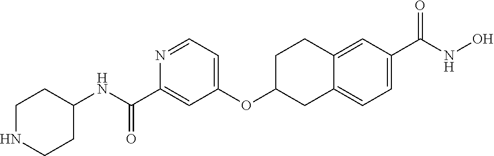 [SMILES: O=C(NO)c1ccc2c(c1)CCC(Oc1ccnc(C(=O)NC3CCNCC3)c1)C2] | I-220 |
| 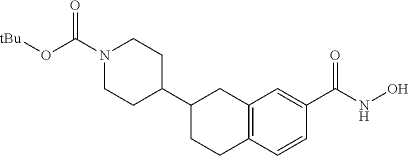 [SMILES: CC(C)(C)OC(=O)N1CCC(C2CCc3ccc(C(=O)NO)cc3C2)CC1] | I-221 |
| 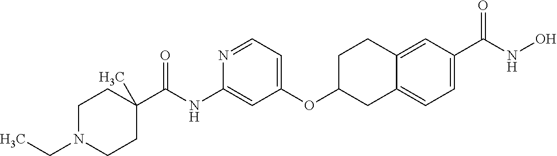 [SMILES: CCN1CCC(C)(C(=O)Nc2cc(OC3CCc4cc(C(=O)NO)ccc4C3)ccn2)CC1] | I-222 |
| 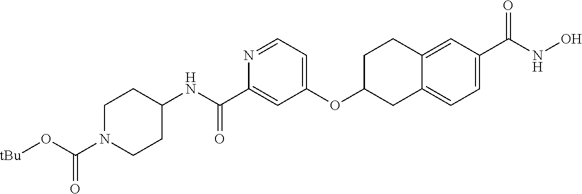 [SMILES: CC(C)(C)OC(=O)N1CCC(NC(=O)c2cc(OC3CCc4cc(C(=O)NO)ccc4C3)ccn2)CC1] | I-223 |
| 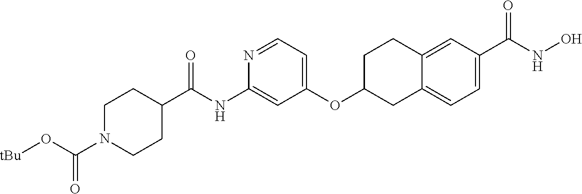 [SMILES: CC(C)(C)OC(=O)N1CCC(C(=O)Nc2cc(OC3CCc4cc(C(=O)NO)ccc4C3)ccn2)CC1] | I-224 |
| 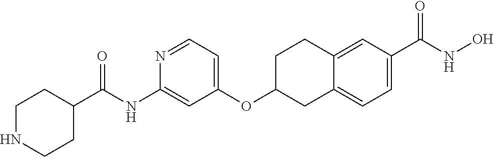 [SMILES: O=C(NO)c1ccc2c(c1)CCC(Oc1ccnc(NC(=O)C3CCNCC3)c1)C2] | I-225 |
| 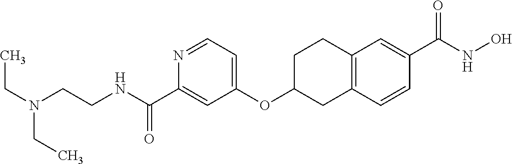 [SMILES: CCN(CC)CCNC(=O)c1cc(OC2CCc3cc(C(=O)NO)ccc3C2)ccn1] | I-226 |
| 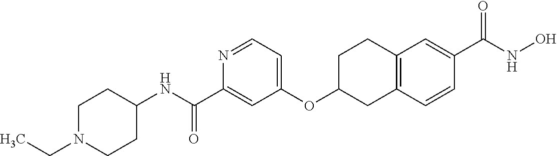 [SMILES: CCN1CCC(NC(=O)c2cc(OC3CCc4cc(C(=O)NO)ccc4C3)ccn2)CC1] | I-227 |
| 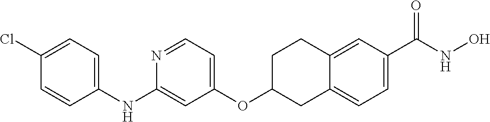 [SMILES: O=C(NO)c1ccc2c(c1)CCC(Oc1ccnc(Nc3ccc(Cl)cc3)c1)C2] | I-228 |
| 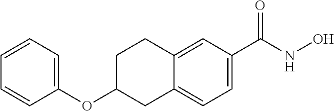 [SMILES: O=C(NO)c1ccc2c(c1)CCC(Oc1ccccc1)C2] | I-229 |
| 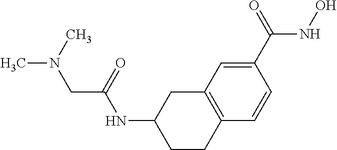 [SMILES: CN(C)CC(=O)NC1CCc2ccc(C(=O)NO)cc2C1] | I-230 |
| 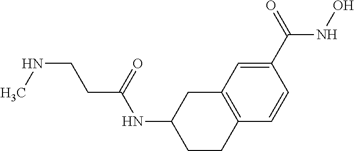 [SMILES: CNCCC(=O)NC1CCc2ccc(C(=O)NO)cc2C1] | I-231 |
| 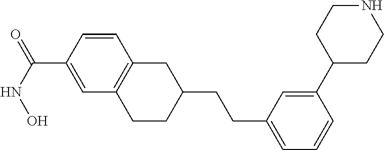 [SMILES: O=C(NO)c1ccc2c(c1)CCC(CCc1cccc(C3CCNCC3)c1)C2] | I-232 |
| 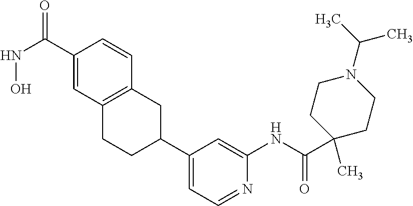 [SMILES: CC(C)N1CCC(C)(C(=O)Nc2cc(C3CCc4cc(C(=O)NO)ccc4C3)ccn2)CC1] | I-233 |
| 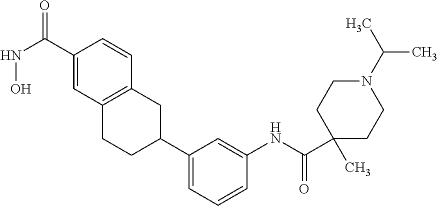 [SMILES: CC(C)N1CCC(C)(C(=O)Nc2cccc(C3CCc4cc(C(=O)NO)ccc4C3)c2)CC1] | I-234 |
| 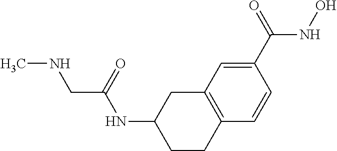 [SMILES: CNCC(=O)NC1CCc2ccc(C(=O)NO)cc2C1] | I-235 |
| 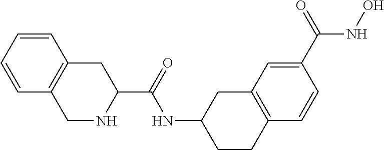 [SMILES: O=C(NO)c1ccc2c(c1)CC(NC(=O)C1Cc3ccccc3CN1)CC2] | I-236 |
| 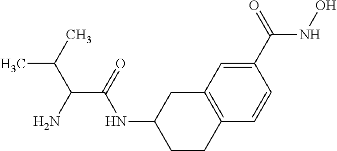 [SMILES: CC(C)C(N)C(=O)NC1CCc2ccc(C(=O)NO)cc2C1] | I-237 |
| 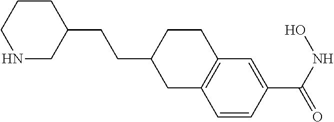 [SMILES: O=C(NO)c1ccc2c(c1)CCC(CCC1CCCNC1)C2] | I-238 |
| 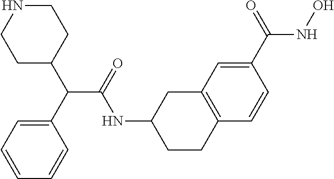 [SMILES: O=C(NO)c1ccc2c(c1)CC(NC(=O)C(c1ccccc1)C1CCNCC1)CC2] | I-239 |
| 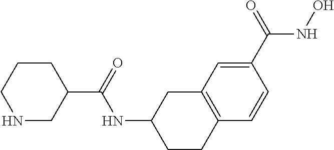 [SMILES: O=C(NO)c1ccc2c(c1)CC(NC(=O)C1CCCNC1)CC2] | I-240 |
| 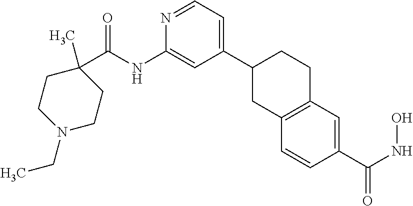 [SMILES: CCN1CCC(C)(C(=O)Nc2cc(C3CCc4cc(C(=O)NO)ccc4C3)ccn2)CC1] | I-241 |
| 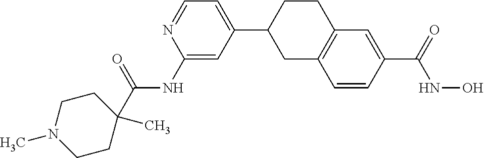 [SMILES: CN1CCC(C)(C(=O)Nc2cc(C3CCc4cc(C(=O)NO)ccc4C3)ccn2)CC1] | I-242 |
| 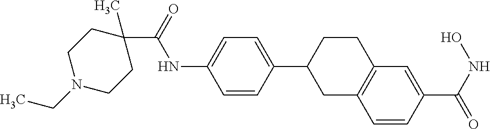 [SMILES: CCN1CCC(C)(C(=O)Nc2ccc(C3CCc4cc(C(=O)NO)ccc4C3)cc2)CC1] | I-243 |
| 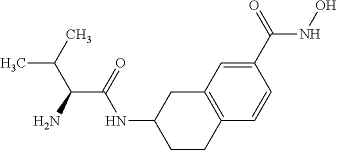 [SMILES: CC(C)[C@H](N)C(=O)NC1CCc2ccc(C(=O)NO)cc2C1] | I-244 |
| 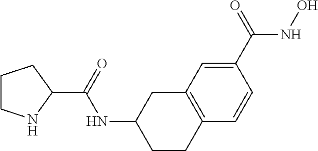 [SMILES: O=C(NO)c1ccc2c(c1)CC(NC(=O)C1CCCN1)CC2] | I-245 |

The compounds in Table 1 above may also be identified by the following chemical names:

| | |
|---|---|
| I-1 | 5-{[(2,6-difluorophenyl)acetyl]amino}-N-hydroxy-5,6,7,8- |
| | tetrahydronaphthalene-2-carboxamide |
| I-2 | 3-ethyl-N-{6-[(hydroxyamino)carbonyl]-1,2,3,4- |
| | tetrahydronaphthalen-1-yl}-1-methyl-1H-pyrazole-5-carboxamide |
| I-3 | N-{6-[(hydroxyamino)carbonyl]-1,2,3,4-tetrahydronaphthalen-1- |
| | yl}thiophene-3-carboxamide |
| I-4 | N-hydroxy-5-{[(1-methylcyclohexyl)carbonyl]amino}-5,6,7,8- |
| | tetrahydronaphthalene-2-carboxamide |
| I-5 | 5-[(3,3-dimethylbutanoyl)amino]-N-hydroxy-5,6,7,8- |
| | tetrahydronaphthalene-2-carboxamide |
| I-6 | 5-[(2,2-dimethylpropanoyl)amino]-N-hydroxy-5,6,7,8- |
| | tetrahydronaphthalene-2-carboxamide |
| I-7 | N-{6-[(hydroxyamino)carbonyl]-1,2,3,4-tetrahydronaphthalen-1- |
| | yl}-2-methyl-4-(trifluoromethyl)-1,3-thiazole-5-carboxamide |
| I-8 | N-hydroxy-5-{[3-(trifluoromethyl)benzoyl]amino}-5,6,7,8- |
| | tetrahydronaphthalene-2-carboxamide |
| I-9 | N-{6-[(hydroxyamino)carbonyl]-1,2,3,4-tetrahydronaphthalen-1- |
| | yl}quinoxaline-2-carboxamide |
| I-10 | N-hydroxy-5-({[4-(trifluoromethyl)phenyl]acetyl}amino)- |
| | 5,6,7,8-tetrahydronaphthalene-2-carboxamide |
| I-11 | 5-{[(3,5-dimethyl-1H-pyrazol-1-yl)acetyl]amino}-N-hydroxy- |
| | 5,6,7,8-tetrahydronaphthalene-2-carboxamide |
| I-12 | 5-{[3,5-bis(trifluoromethyl)benzoyl]amino}-N-hydroxy-5,6,7,8- |
| | tetrahydronaphthalene-2-carboxamide |
| I-13 | 5-[(dicyclohexylacetyl)amino]-N-hydroxy-5,6,7,8- |
| | tetrahydronaphthalene-2-carboxamide |
| I-14 | 5-{[2-(2-chlorophenoxy)-2-methylpropanoyl]amino}-N-hydroxy- |
| | 5,6,7,8-tetrahydronaphthalene-2-carboxamide |
| I-15 | 5-[(2,2-dimethylbutanoyl)amino]-N-hydroxy-5,6,7,8- |
| | tetrahydronaphthalene-2-carboxamide |
| I-16 | 5-[(4-tert-butylbenzoyl)amino]-N-hydroxy-5,6,7,8- |
| | tetrahydronaphthalene-2-carboxamide |
| I-17 | 5-[(cyclohexylcarbonyl)amino]-N-hydroxy-5,6,7,8- |
| | tetrahydronaphthalene-2-carboxamide |
| I-18 | N-{6-[(hydroxyamino)carbonyl]-1,2,3,4-tetrahydronaphthalen-1- |
| | yl}-1-methyl-1H-pyrrole-2-carboxamide |
| I-19 | 1-tert-butyl-N-{6-[(hydroxyamino)carbonyl]-1,2,3,4- |
| | tetrahydronaphthalen-1-yl}-3-methyl-1H-pyrazole-5-carboxamide |
| I-20 | N-hydroxy-5-{[4-(trifluoromethyl)benzoyl]amino}-5,6,7,8- |
| | tetrahydronaphthalene-2-carboxamide |
| I-21 | N-{6-[(hydroxyamino)carbonyl]-1,2,3,4-tetrahydronaphthalen-1- |
| | yl}adamantane-1-carboxamide |
| I-22 | N-{6-[(hydroxyamino)carbonyl]-1,2,3,4-tetrahydronaphthalen-1- |
| | yl}-2-methyl-5-(pyrrolidin-1-ylsulfonyl)-3-furamide |
| I-23 | 4-chloro-N-{6-[(hydroxyamino)carbonyl]-1,2,3,4- |
| | tetrahydronaphthalen-1-yl}-1,3-dimethyl-1H-pyrazolo |
| | [3,4-b]pyridine-5-carboxamide |
| I-24 | N-{6-[(hydroxyamino)carbonyl]-1,2,3,4-tetrahydronaphthalen-1- |
| | yl}-1-benzothiophene-2-carboxamide |
| I-25 | 7-ethoxy-N-{6-[(hydroxyamino)carbonyl]-1,2,3,4- |
| | tetrahydronaphthalen-1-yl}-1-benzofuran-2-carboxamide |
| I-26 | 5-{[(1S,4R)-bicyclo[2.2.1]hept-2-ylacetyl]amino}-N-hydroxy- |
| | 5,6,7,8-tetrahydronaphthalene-2-carboxamide |
| I-27 | 5-{[(2Z)-2-(acetylamino)-3-phenylprop-2-enoyl]amino}-N- |
| | hydroxy-5,6,7,8-tetrahydronaphthalene-2-carboxamide |
| I-28 | N-{6-[(hydroxyamino)carbonyl]-1,2,3,4-tetrahydronaphthalen-1- |
| | yl}-1,3-benzothiazole-6-carboxamide |
| I-29 | 6-chloro-N-{6-[(hydroxyamino)carbonyl]-1,2,3,4- |
| | tetrahydronaphthalen-1-yl}imidazo[1,2-a]pyridine-2-carboxamide |
| I-30 | 5-chloro-N-{6-[(hydroxyamino)carbonyl]-1,2,3,4- |
| | tetrahydronaphthalen-1-yl}-1H-indole-2-carboxamide |
| I-31 | N-hydroxy-5-[(3-methyl-2-phenylbutanoyl)amino]-5,6,7,8- |
| | tetrahydronaphthalene-2-carboxamide |
| I-32 | 3-chloro-N-{6-[(hydroxyamino)carbonyl]-1,2,3,4- |
| | tetrahydronaphthalen-1-yl}-1-benzothiophene-2-carboxamide |
| I-33 | N-{6-[(hydroxyamino)carbonyl]-1,2,3,4-tetrahydronaphthalen-1- |
| | yl}-7-methoxy-1-benzofuran-2-carboxamide |
| I-34 | N-{6-[(hydroxyamino)carbonyl]-1,2,3,4-tetrahydronaphthalen-1- |
| | yl}biphenyl-4-carboxamide |
| I-35 | N-{6-[(hydroxyamino)carbonyl]-1,2,3,4-tetrahydronaphthalen-1- |
| | yl}-3-(4-methylphenyl)-1H-pyrazole-5-carboxamide |
| I-36 | N-hydroxy-5-{[(6-methoxy-1-benzofuran-3-yl)acetyl]amino}- |
| | 5,6,7,8-tetrahydronaphthalene-2-carboxamide |
| I-37 | N-{6-[(hydroxyamino)carbonyl]-1,2,3,4-tetrahydronaphthalen-1- |
| | yl}-2-methyl-5-(piperidin-1-ylsulfonyl)-3-furamide |
| I-38 | 3-(2-chlorophenyl)-N-{6-[(hydroxyamino)carbonyl]-1,2,3,4- |
| | tetrahydronaphthalen-1-yl}-5-methylisoxazole-4-carboxamide |
| I-39 | N-{6-[(hydroxyamino)carbonyl]-1,2,3,4-tetrahydronaphthalen-1- |
| | yl}-5-methyl-1H-indole-2-carboxamide |
| I-40 | 5-chloro-N-{6-[(hydroxyamino)carbonyl]-1,2,3,4- |
| | tetrahydronaphthalen-1-yl}-1-benzofuran-2-carboxamide |
| I-41 | N-hydroxy-5-{[(4-methoxyphenyl)(phenyl)acetyl]amino}-5,6,7,8- |
| | tetrahydronaphthalene-2-carboxamide |
| I-42 | 4′-fluoro-N-{6-[(hydroxyamino)carbonyl]-1,2,3,4- |
| | tetrahydronaphthalen-1-yl}biphenyl-4-carboxamide |
| I-43 | 3-(4-fluorophenyl)-N-{6-[(hydroxyamino)carbonyl]-1,2,3,4- |
| | tetrahydronaphthalen-1-yl}-1H-pyrazole-5-carboxamide |
| I-44 | N-{6-[(hydroxyamino)carbonyl]-1,2,3,4-tetrahydronaphthalen-1- |
| | yl}-5-methoxy-1-benzofuran-2-carboxamide |
| I-45 | N-{6-[(hydroxyamino)carbonyl]-1,2,3,4-tetrahydronaphthalen-1- |
| | yl}-1-benzofuran-2-carboxamide |
| I-46 | N-hydroxy-5-{[(1-phenylcyclopentyl)carbonyl]amino}-5,6,7,8- |
| | tetrahydronaphthalene-2-carboxamide |
| I-47 | N-{6-[(hydroxyamino)carbonyl]-1,2,3,4-tetrahydronaphthalen-2- |
| | yl}-2-methyl-1-(3-morpholin-4-ylpropyl)-5-phenyl-1H-pyrrole-3- |
| | carboxamide |
| I-48 | N-{6-[(hydroxyamino)carbonyl]-1,2,3,4-tetrahydronaphthalen-2- |
| | yl}-2,5-dimethyl-1-(2-thienylmethyl)-1H-pyrrole-3-carboxamide |
| I-49 | 6-[(cyclohexylcarbonyl)amino]-N-hydroxy-5,6,7,8- |
| | tetrahydronaphthalene-2-carboxamide |
| I-50 | N-{6-[(hydroxyamino)carbonyl]-1,2,3,4-tetrahydronaphthalen-2- |
| | yl}-2,5-dimethyl-3-furamide |
| I-51 | 4-acetyl-N-{6-[(hydroxyamino)carbonyl]-1,2,3,4- |
| | tetrahydronaphthalen-2-yl}-3,5-dimethyl-1H-pyrrole-2- |
| | carboxamide |
| I-52 | N-{6-[(hydroxyamino)carbonyl]-1,2,3,4-tetrahydronaphthalen-2- |
| | yl}-1-benzothiophene-3-carboxamide |
| I-53 | 5-[(dibenzylamino)sulfonyl]-N-{6-[(hydroxyamino)carbonyl]- |
| | 1,2,3,4-tetrahydronaphthalen-2-yl}-1-methyl-1H-pyrrole-2- |
| | carboxamide |
| I-54 | N-{6-[(hydroxyamino)carbonyl]-1,2,3,4-tetrahydronaphthalen-2- |
| | yl}-1-benzofuran-5-carboxamide |
| I-55 | N-{6-[(hydroxyamino)carbonyl]-1,2,3,4-tetrahydronaphthalen-2- |
| | yl}-2-methylimidazo[1,2-a]pyridine-3-carboxamide |
| I-56 | N-{6-[(hydroxyamino)carbonyl]-1,2,3,4-tetrahydronaphthalen-2- |
| | yl}-2-methyl-1,5-diphenyl-1H-pyrrole-3-carboxamide |
| I-57 | N-{6-[(hydroxyamino)carbonyl]-1,2,3,4-tetrahydronaphthalen-2- |
| | yl}adamantane-1-carboxamide |
| I-58 | N-{6-[(hydroxyamino)carbonyl]-1,2,3,4-tetrahydronaphthalen-2- |
| | yl}-1-phenyl-1H-pyrazole-4-carboxamide |
| I-59 | N-{6-[(hydroxyamino)carbonyl]-1,2,3,4-tetrahydronaphthalen-2- |
| | yl}-2-methyl-5-phenyl-3-furamide |
| I-60 | N-{6-[(hydroxyamino)carbonyl]-1,2,3,4-tetrahydronaphthalen-2- |
| | yl}biphenyl-4-carboxamide |
| I-61 | N-hydroxy-6-[(4-methoxybenzoyl)amino]-5,6,7,8- |
| | tetrahydronaphthalene-2-carboxamide |
| I-62 | N-{6-[(hydroxyamino)carbonyl]-1,2,3,4-tetrahydronaphthalen-2- |
| | yl}-1-methyl-1H-pyrrole-2-carboxamide |
| I-63 | 5-chloro-N-{(1R)-6-[(hydroxyamino)carbonyl]-1,2,3,4- |
| | tetrahydronaphthalen-1-yl}-1-benzofuran-2-carboxamide |
| I-64 | N-{(1R)-6-[(hydroxyamino)carbonyl]-1,2,3,4- |
| | tetrahydronaphthalen-1-yl}-1-methyl-1H-indole-2-carboxamide |
| I-65 | N-{(1R)-6-[(hydroxyamino)carbonyl]-1,2,3,4- |
| | tetrahydronaphthalen-1-yl}-1-methyl-1H-pyrrole-2-carboxamide |
| I-66 | N-hydroxy-5,6,7,8-tetrahydronaphthalene-2-carboxamide |
| I-67 | N-{(1S)-6-[(hydroxyamino)carbonyl]-1,2,3,4- |
| | tetrahydronaphthalen-1-yl}-1-methyl-1H-pyrrole-2-carboxamide |
| I-68 | N-{(1S)-6-[(hydroxyamino)carbonyl]-1,2,3,4- |
| | tetrahydronaphthalen-1-yl}-3-(2-methoxyphenyl)-1H-pyrazole-5- |
| | carboxamide |
| I-69 | (5S)-5-{[2-(2-chlorophenoxy)-2-methylpropanoyl]amino}-N- |
| | hydroxy-5,6,7,8-tetrahydronaphthalene-2-carboxamide |
| I-70 | 6-chloro-N-{(1S)-6-[(hydroxyamino)carbonyl]-1,2,3,4- |
| | tetrahydronaphthalen-1-yl}imidazo[1,2-a]pyridine-2-carboxamide |
| I-71 | 3-(4-fluorophenyl)-N-{(1S)-6-[(hydroxyamino)carbonyl]-1,2,3,4- |
| | tetrahydronaphthalen-1-yl}-1H-pyrazole-5-carboxamide |
| I-72 | N-{(1S)-6-[(hydroxyamino)carbonyl]-1,2,3,4- |
| | tetrahydronaphthalen-1-yl}-3-(4-methylphenyl)-1H-pyrazole-5- |
| | carboxamide |
| I-73 | (7R)-7-[(2,2-dimethylpropanoyl)amino]-N-hydroxy-5,6,7,8- |
| | tetrahydronaphthalene-2-carboxamide |
| I-74 | N-hydroxy-5,5,8,8-tetramethyl-5,6,7,8-tetrahydronaphthalene-2- |
| | carboxamide |
| I-75 | (7S)-7-[(2,2-dimethylpropanoyl)amino]-N-hydroxy-5,6,7,8- |
| | tetrahydronaphthalene-2-carboxamide |
| I-76 | 7-[(4-chlorobenzyl)amino]-N-hydroxy-5,6,7,8- |
| | tetrahydronaphthalene-2-carboxamide |
| I-77 | N-hydroxy-7-[(4-methoxybenzoyl)amino]-5,6,7,8- |
| | tetrahydronaphthalene-2-carboxamide |
| I-78 | 7-{[3-[(dimethylamino)methyl]-5- |
| | (trifluoromethyl)benzoyl]amino}-N-hydroxy-5,6,7,8- |
| | tetrahydronaphthalene-2-carboxamide |
| I-79 | N-hydroxy-7-{[3-(1-methyl-1H-pyrazol-4-yl)-5- |
| | (trifluoromethyl)benzoyl]amino}-5,6,7,8-tetrahydronaphthalene-2- |
| | carboxamide |
| I-80 | 7-(dimethylamino)-N-hydroxy-5,6,7,8-tetrahydronaphthalene-2- |
| | carboxamide |
| I-81 | N-hydroxy-7-[(2-phenylethyl)amino]-5,6,7,8- |
| | tetrahydronaphthalene-2-carboxamide |
| I-82 | 7-[(3-chlorophenyl)amino]-N-hydroxy-5,6,7,8- |
| | tetrahydronaphthalene-2-carboxamide |
| I-83 | 7-[(4-chlorophenyl)amino]-N-hydroxy-5,6,7,8- |
| | tetrahydronaphthalene-2-carboxamide |
| I-84 | N-hydroxy-7-[(3-methoxyphenyl)amino]-5,6,7,8- |
| | tetrahydronaphthalene-2-carboxamide |
| I-85 | N-hydroxy-7-[(pyridin-4-ylmethyl)amino]-5,6,7,8- |
| | tetrahydronaphthalene-2-carboxamide |
| I-86 | N-hydroxy-7-(pyridin-4-yloxy)-5,6,7,8-tetrahydronaphthalene-2- |
| | carboxamide |
| I-87 | N-hydroxy-7-phenoxy-5,6,7,8-tetrahydronaphthalene-2- |
| | carboxamide |
| I-88 | N-hydroxy-7-pyridin-4-yl-5,6,7,8-tetrahydronaphthalene-2- |
| | carboxamide |
| I-89 | N-hydroxy-7-phenyl-5,6,7,8-tetrahydronaphthalene-2-carboxamide |
| I-90 | 7-(4-chlorophenyl)-N-hydroxy-5,6,7,8-tetrahydronaphthalene-2- |
| | carboxamide |
| I-91 | 7-(4-chlorophenoxy)-N-hydroxy-5,6,7,8-tetrahydronaphthalene-2- |
| | carboxamide |
| I-92 | 7-[3-(acetylamino)phenyl]-N-hydroxy-5,6,7,8- |
| | tetrahydronaphthalene-2-carboxamide |
| I-93 | N-hydroxy-7-(2-phenylethyl)-5,6,7,8-tetrahydronaphthalene-2- |
| | carboxamide |
| I-94 | 7-[(anilinocarbonyl)amino]-N-hydroxy-5,6,7,8- |
| | tetrahydronaphthalene-2-carboxamide |
| I-95 | N-hydroxy-7-(pyridin-4-ylamino)-5,6,7,8-tetrahydronaphthalene-2- |
| | carboxamide |
| I-96 | N-hydroxy-7-[(phenylsulfonyl)amino]-5,6,7,8- |
| | tetrahydronaphthalene-2-carboxamide |
| I-97 | 7-[2-(acetylamino)pyridin-4-yl]-N-hydroxy-5,6,7,8- |
| | tetrahydronaphthalene-2-carboxamide |
| I-98 | 7-(6-aminopyrimidin-4-yl)-N-hydroxy-5,6,7,8- |
| | tetrahydronaphthalene-2-carboxamide |
| I-99 | N-hydroxy-6-pyridin-4-yl-5,6,7,8-tetrahydronaphthalene-2- |
| | carboxamide |
| I-100 | N-hydroxy-6-phenyl-5,6,7,8-tetrahydronaphthalene-2-carboxamide |
| I-101 | 6-(4-chlorophenyl)-N-hydroxy-5,6,7,8-tetrahydronaphthalene-2- |
| | carboxamide |
| I-102 | 6-[3-(acetylamino)phenyl]-N-hydroxy-5,6,7,8- |
| | tetrahydronaphthalene-2-carboxamide |
| I-103 | 6-[2-(3-fluorophenyl)ethyl]-N-hydroxy-5,6,7,8- |
| | tetrahydronaphthalene-2-carboxamide |
| I-104 | N-hydroxy-6-(2-methoxyphenyl)-5,6,7,8-tetrahydronaphthalene-2- |
| | carboxamide |
| I-105 | N-hydroxy-6-(4-methoxyphenyl)-5,6,7,8-tetrahydronaphthalene-2- |
| | carboxamide |
| I-106 | 6-[(4-chlorobenzyl)amino]-N-hydroxy-5,6,7,8- |
| | tetrahydronaphthalene-2-carboxamide |
| I-107 | N-hydroxy-6-[(pyridin-4-ylmethyl)amino]-5,6,7,8- |
| | tetrahydronaphthalene-2-carboxamide |
| I-108 | N-hydroxy-6-(pyridin-4-yloxy)-5,6,7,8-tetrahydronaphthalene-2- |
| | carboxamide |
| I-109 | N-hydroxy-6-[2-(trifluoromethyl)phenyl]-5,6,7,8- |
| | tetrahydronaphthalene-2-carboxamide |
| I-110 | N-hydroxy-6-(3-methoxyphenyl)-5,6,7,8-tetrahydronaphthalene-2- |
| | carboxamide |
| I-111 | 6-[(2,2-dimethylpropanoyl)amino]-N-hydroxy-5,6,7,8- |
| | tetrahydronaphthalene-2-carboxamide |
| I-112 | N-hydroxy-6-[(3-methoxybenzoyl)amino]-5,6,7,8- |
| | tetrahydronaphthalene-2-carboxamide |
| I-113 | N-hydroxy-6-[3-(trifluoromethyl)phenyl]-5,6,7,8- |
| | tetrahydronaphthalene-2-carboxamide |
| I-114 | N-{7-[(hydroxyamino)carbonyl]-1,2,3,4-tetrahydronaphthalen-2- |
| | yl}-4-methylpiperidine-4-carboxamide |
| I-115 | N-hydroxy-7-[3-(trifluoromethyl)phenyl]-5,6,7,8- |
| | tetrahydronaphthalene-2-carboxamide |
| I-116 | N-hydroxy-7-(2-methoxyphenyl)-5,6,7,8-tetrahydronaphthalene-2- |
| | carboxamide |
| I-117 | 7-[(4-chlorobenzyl)oxy]-N-hydroxy-5,6,7,8-tetrahydronaphthalene- |
| | 2-carboxamide |
| I-118 | 7-ethoxy-N-hydroxy-5,6,7,8-tetrahydronaphthalene-2-carboxamide |
| I-119 | N-hydroxy-7-(3-methoxyphenyl)-5,6,7,8-tetrahydronaphthalene-2- |
| | carboxamide |
| I-120 | N-hydroxy-7-pyridin-3-yl-5,6,7,8-tetrahydronaphthalene-2- |
| | carboxamide |
| I-121 | N-hydroxy-6-pyridin-3-yl-5,6,7,8-tetrahydronaphthalene-2- |
| | carboxamide |
| I-122 | N-hydroxy-6-[(2-methoxybenzoyl)amino]-5,6,7,8- |
| | tetrahydronaphthalene-2-carboxamide |
| I-123 | N-hydroxy-9-[(1-methyl-1H-pyrrol-2-yl)carbonyl]-1,2,3,4- |
| | tetrahydro-1,4-epiminonaphthalene-6-carboxamide |
| I-124 | 6-[(4-chlorobenzyl)amino]-3-fluoro-N-hydroxy-5,6,7,8- |
| | tetrahydronaphthalene-2-carboxamide |
| I-125 | 6-[(4-chlorobenzyl)oxy]-N-hydroxy-5,6,7,8-tetrahydronaphthalene- |
| | 2-carboxamide |
| I-126 | 6-[(4-chlorobenzyl)amino]-N-hydroxy-8,8-dimethyl-5,6,7,8- |
| | tetrahydronaphthalene-2-carboxamide |
| I-127 | N-hydroxy-7-(2-isopropylphenyl)-5,6,7,8-tetrahydronaphthalene-2- |
| | carboxamide |
| I-128 | N-7-hydroxy-N-2-[2-(methylamino)ethyl]-1,2,3,4- |
| | tetrahydronaphthalene-2,7-dicarboxamide |
| I-129 | 4-chlorophenyl {6-[(hydroxyamino)carbonyl]-1,2,3,4- |
| | tetrahydronaphthalen-2-yl}carbamate |
| I-130 | 3-fluoro-N-hydroxy-6-(pyridin-4-ylamino)-5,6,7,8- |
| | tetrahydronaphthalene-2-carboxamide |
| I-131 | N-6-hydroxy-N-2-[2-(methylamino)ethyl]-1,2,3,4- |
| | tetrahydronaphthalene-2,6-dicarboxamide |
| I-132 | N-2-(4-chlorobenzyl)-N-6-hydroxy-1,2,3,4-tetrahydronaphthalene- |
| | 2,6-dicarboxamide |
| I-133 | N-2-(tert-butyl)-N-7-hydroxy-1,2,3,4-tetrahydronaphthalene-2,7- |
| | dicarboxamide |
| I-134 | 6-[(2,2-dimethylpropanoyl)amino]-3-fluoro-N-hydroxy-5,6,7,8- |
| | tetrahydronaphthalene-2-carboxamide |
| I-135 | N-hydroxy-7-[(7-oxo-5,6,7,8-tetrahydro-1,8-naphthyridin-4- |
| | yl)oxy]-5,6,7,8-tetrahydronaphthalene-2-carboxamide |
| I-136 | N-hydroxy-6-[(7-oxo-5,6,7,8-tetrahydro-1,8-naphthyridin-4- |
| | yl)oxy]-5,6,7,8-tetrahydronaphthalene-2-carboxamide |
| I-137 | 6-[(2,2-dimethylpropanoyl)amino]-N-hydroxy-5-methoxy-5,6,7,8- |
| | tetrahydronaphthalene-2-carboxamide |
| I-138 | 6-[(4-chlorobenzyl)amino]-N-hydroxy-5-methoxy-5,6,7,8- |
| | tetrahydronaphthalene-2-carboxamide |
| I-139 | N-hydroxy-8,8-dimethyl-6-(pyridin-4-ylamino)-5,6,7,8- |
| | tetrahydronaphthalene-2-carboxamide |
| I-140 | 6-[(2,2-dimethylpropanoyl)amino]-N-hydroxy-8,8-dimethyl- |
| | 5,6,7,8-tetrahydronaphthalene-2-carboxamide |
| I-141 | 4-chlorophenyl {7-[(hydroxyamino)carbonyl]-1,2,3,4- |
| | tetrahydronaphthalen-2-yl}carbamate |
| I-142 | N-6-hydroxy-N-2-(pyridin-4-ylmethyl)-1,2,3,4- |
| | tetrahydronaphthalene-2,6-dicarboxamide |
| I-143 | N-2-(tert-butyl)-N-6-hydroxy-1,2,3,4-tetrahydronaphthalene-2,6- |
| | dicarboxamide |
| I-144 | N-2-(4-chlorobenzyl)-N-7-hydroxy-1,2,3,4-tetrahydronaphthalene- |
| | 2,7-dicarboxamide |
| I-145 | N-7-hydroxy-N-2-(pyridin-4-ylmethyl)-1,2,3,4- |
| | tetrahydronaphthalene-2,7-dicarboxamide |
| I-146 | N-hydroxy-6-(pyridin-2-yloxy)-5,6,7,8-tetrahydronaphthalene-2- |
| | carboxamide |
| I-147 | N-hydroxy-6-(pyridin-3-yloxy)-5,6,7,8-tetrahydronaphthalene-2- |
| | carboxamide |
| I-148 | N-[4-({6-[(hydroxyamino)carbonyl]-1,2,3,4- |
| | tetrahydronaphthalen-2-yl}oxy)pyridin-2-yl]-4- |
| | methylpiperidine-4-carboxamide |
| I-149 | 4-({6-[(hydroxyamino)carbonyl]-1,2,3,4- |
| | tetrahydronaphthalen-2-yl}oxy)-N,N-dimethylpyridine-2- |
| | carboxamide |
| I-150 | 6-({2-[(2,2-dimethylpropanoyl)amino]pyridin-4-yl}oxy)- |
| | N-hydroxy-5,6,7,8-tetrahydronaphthalene-2-carboxamide |
| I-151 | N-(tert-butyl)-4-{6-[(hydroxyamino)carbonyl]-1,2,3,4- |
| | tetrahydronaphthalen-2-yl}pyridine-2-carboxamide |
| I-152 | N-(tert-butyl)-4-{7-[(hydroxyamino)carbonyl]-1,2,3,4- |
| | tetrahydronaphthalen-2-yl}pyridine-2-carboxamide |
| I-153 | 6-[(4-chlorophenoxy)methyl]-N-hydroxy-5,6,7,8- |
| | tetrahydronaphthalene-2-carboxamide |
| I-154 | N-hydroxy-6-[(pyridin-4-yloxy)methyl]-5,6,7,8- |
| | tetrahydronaphthalene-2-carboxamide |
| I-155 | 6-{[(4-chlorobenzyl)oxy]methyl}-N-hydroxy-5,6,7,8- |
| | tetrahydronaphthalene-2-carboxamide |
| I-156 | N-hydroxy-6-[(pyridin-4-ylmethoxy)methyl]-5,6,7,8- |
| | tetrahydronaphthalene-2-carboxamide |
| I-157 | N-hydroxy-6-(methoxymethyl)-5,6,7,8-tetrahydronaphthalene-2- |
| | carboxamide |
| I-158 | 7-[(4-chlorophenoxy)methyl]-N-hydroxy-5,6,7,8- |
| | tetrahydronaphthalene-2-carboxamide |
| I-159 | N-hydroxy-7-[(pyridin-4-yloxy)methyl]-5,6,7,8- |
| | tetrahydronaphthalene-2-carboxamide |
| I-160 | 7-{[(4-chlorobenzyl)oxy]methyl}-N-hydroxy-5,6,7,8- |
| | tetrahydronaphthalene-2-carboxamide |
| I-161 | N-hydroxy-7-[(pyridin-4-ylmethoxy)methyl]-5,6,7,8- |
| | tetrahydronaphthalene-2-carboxamide |
| I-162 | N-hydroxy-7-(methoxymethyl)-5,6,7,8-tetrahydronaphthalene-2- |
| | carboxamide |
| I-163 | 6-(4-chlorophenoxy)-N-hydroxy-5,6,7,8-tetrahydronaphthalene-2- |
| | carboxamide |
| I-164 | N-hydroxy-6-[(2-phenylethyl)amino]-5,6,7,8- |
| | tetrahydronaphthalene-2-carboxamide |
| I-165 | N-hydroxy-7-(pyridin-2-yloxy)-5,6,7,8-tetrahydronaphthalene-2- |
| | carboxamide |
| I-166 | N-hydroxy-7-(pyridin-3-yloxy)-5,6,7,8-tetrahydronaphthalene-2- |
| | carboxamide |
| I-167 | N-[4-({7-[(hydroxyamino)carbonyl]-1,2,3,4- |
| | tetrahydronaphthalen-2-yl}oxy)pyridin-2-yl]-4- |
| | methylpiperidine-4-carboxamide |
| I-168 | 4-({7-[(hydroxyamino)carbonyl]-1,2,3,4- |
| | tetrahydronaphthalen-2-yl}oxy)-N,N-dimethylpyridine-2- |
| | carboxamide |
| I-169 | 7-({2-[(2,2-dimethylpropanoyl)amino]pyridin-4-yl}oxy)- |
| | N-hydroxy-5,6,7,8-tetrahydronaphthalene-2-carboxamide |
| I-170 | N-{6-[(hydroxyamino)carbonyl]-1,2,3,4-tetrahydronaphthalen- |
| | 2-yl}-4-methylpiperidine-4-carboxamide |
| I-171 | 6-{[3-[(dimethylamino)methyl]-5-(trifluoromethyl) |
| | benzoyl]amino}-N-hydroxy-5,6,7,8-tetrahydronaphthalene-2- |
| | carboxamide |
| I-172 | N-hydroxy-6-[4-(trifluoromethyl)phenyl]-5,6,7,8- |
| | tetrahydronaphthalene-2-carboxamide |
| I-173 | N-hydroxy-6-(1H-indol-3-yl)-5,6,7,8-tetrahydronaphthalene-2- |
| | carboxamide |
| I-174 | N-hydroxy-6-(3-isopropylphenyl)-5,6,7,8-tetrahydronaphthalene-2- |
| | carboxamide |
| I-175 | N-hydroxy-6-(1H-indol-6-yl)-5,6,7,8-tetrahydronaphthalene-2- |
| | carboxamide |
| I-176 | N-(4-{6-[(hydroxyamino)carbonyl]-1,2,3,4-tetrahydronaphthalen-2- |
| | yl}pyridin-2-yl)-4-methylpiperidine-4-carboxamide |
| I-177 | N-hydroxy-6-(2-{[3-(methylamino)propanoyl]amino}pyridin-4-yl)- |
| | 5,6,7,8-tetrahydronaphthalene-2-carboxamide |
| I-178 | 6-{2-[(2,2-dimethylpropanoyl)amino]pyridin-4-yl}-N-hydroxy- |
| | 5,6,7,8-tetrahydronaphthalene-2-carboxamide |
| I-179 | N-hydroxy-6-pyridin-2-yl-5,6,7,8-tetrahydronaphthalene-2- |
| | carboxamide |
| I-180 | 6-{3-[(4-chlorobenzyl)oxy]phenyl}-N-hydroxy-5,6,7,8- |
| | tetrahydronaphthalene-2-carboxamide |
| I-181 | 6-{4-[(dimethylamino)methyl]phenyl}-N-hydroxy-5,6,7,8- |
| | tetrahydronaphthalene-2-carboxamide |
| I-182 | 6-{3-[2-(dimethylamino)ethoxy]phenyl}-N-hydroxy-5,6,7,8- |
| | tetrahydronaphthalene-2-carboxamide |
| I-183 | 6-{3-[(dimethylamino)methyl]phenyl}-N-hydroxy-5,6,7,8- |
| | tetrahydronaphthalene-2-carboxamide |
| I-184 | N-hydroxy-7-(1H-indol-3-yl)-5,6,7,8-tetrahydronaphthalene-2- |
| | carboxamide |
| I-185 | N-hydroxy-7-[2-(trifluoromethyl)phenyl]-5,6,7,8- |
| | tetrahydronaphthalene-2-carboxamide |
| I-186 | N-hydroxy-7-(1H-indol-6-yl)-5,6,7,8-tetrahydronaphthalene-2- |
| | carboxamide |
| I-187 | N-(4-{7-[(hydroxyamino)carbonyl]-1,2,3,4-tetrahydronaphthalen-2- |
| | yl}pyridin-2-yl)-4-methylpiperidine-4-carboxamide |
| I-188 | N-hydroxy-7-(2-{[3-(methylamino)propanoyl]amino}pyridin-4-yl)- |
| | 5,6,7,8-tetrahydronaphthalene-2-carboxamide |
| I-189 | 7-{2-[(2,2-dimethylpropanoyl)amino]pyridin-4-yl}-N-hydroxy- |
| | 5,6,7,8-tetrahydronaphthalene-2-carboxamide |
| I-190 | N-hydroxy-7-(6-piperazin-1-ylpyridin-3-yl)-5,6,7,8- |
| | tetrahydronaphthalene-2-carboxamide |
| I-191 | N-hydroxy-7-pyridin-2-yl-5,6,7,8-tetrahydronaphthalene-2- |
| | carboxamide |
| I-192 | 7-{3-[(4-chlorobenzyl)oxy]phenyl}-N-hydroxy-5,6,7,8- |
| | tetrahydronaphthalene-2-carboxamide |
| I-193 | 7-{4-[(dimethylamino)methyl]phenyl}-N-hydroxy-5,6,7,8- |
| | tetrahydronaphthalene-2-carboxamide |
| I-194 | 7-{3-[2-(dimethylamino)ethoxy]phenyl}-N-hydroxy-5,6,7,8- |
| | tetrahydronaphthalene-2-carboxamide |
| I-195 | 7-{3-[(dimethylamino)methyl]phenyl}-N-hydroxy-5,6,7,8- |
| | tetrahydronaphthalene-2-carboxamide |
| I-196 | N-hydroxy-9-[(1-methylcyclohexyl)carbonyl]-1,2,3,4-tetrahydro- |
| | 1,4-epiminonaphthalene-6-carboxamide |
| I-197 | 9-(4-chlorobenzoyl)-N-hydroxy-1,2,3,4-tetrahydro-1,4- |
| | epiminonaphthalene-6-carboxamide |
| I-198 | N-hydroxy-9-(4-methoxybenzoyl)-1,2,3,4-tetrahydro-1,4- |
| | epiminonaphthalene-6-carboxamide |
| I-199 | 9-(biphenyl-4-ylcarbonyl)-N-hydroxy-1,2,3,4-tetrahydro-1,4- |
| | epiminonaphthalene-6-carboxamide |
| I-200 | 9-(4-tert-butylbenzoyl)-N-hydroxy-1,2,3,4-tetrahydro-1,4- |
| | epiminonaphthalene-6-carboxamide |
| I-201 | 9-(2,2-dimethylpropanoyl)-N-hydroxy-1,2,3,4-tetrahydro-1,4- |
| | epiminonaphthalene-6-carboxamide |
| I-202 | 9-(1-benzofuran-2-ylcarbonyl)-N-hydroxy-1,2,3,4-tetrahydro-1,4- |
| | epiminonaphthalene-6-carboxamide |
| I-203 | 9-[(3-chloro-1-benzothien-2-yl)carbonyl]-N-hydroxy-1,2,3,4- |
| | tetrahydro-1,4-epiminonaphthalene-6-carboxamide |
| I-204 | 9-(1-benzothien-2-ylcarbonyl)-N-hydroxy-1,2,3,4-tetrahydro-1,4- |
| | epiminonaphthalene-6-carboxamide |
| I-205 | 9-(1-adamantylcarbonyl)-N-hydroxy-1,2,3,4-tetrahydro-1,4- |
| | epiminonaphthalene-6-carboxamide |
| I-206 | N-hydroxy-9-[(4-methyl-2-pyridin-2-yl-1,3-thiazol-5-yl)carbonyl]- |
| | 1,2,3,4,4a,8a-hexahydro-1,4-epiminonaphthalene-6-carboxamide |
| I-207 | 9-butyryl-N-hydroxy-1,2,3,4-tetrahydro-1,4-epiminonaphthalene-6- |
| | carboxamide |
| I-208 | N-hydroxy-9-[(2-methyl-1,3-thiazol-4-yl)carbonyl]-1,2,3,4- |
| | tetrahydro-1,4-epiminonaphthalene-6-carboxamide |
| I-209 | N-hydroxy-9-[(5-pyridin-2-yl-2-thienyl)carbonyl]-1,2,3,4- |
| | tetrahydro-1,4-epiminonaphthalene-6-carboxamide |
| I-210 | N-hydroxy-9-(pyridin-2-ylcarbonyl)-1,2,3,4-tetrahydro-1,4- |
| | epiminonaphthalene-6-carboxamide |
| I-211 | N-hydroxy-9-(phenylacetyl)-1,2,3,4-tetrahydro-1,4- |
| | epiminonaphthalene-6-carboxamide |
| I-212 | N-hydroxy-7-piperidin-4-yl-5,6,7,8-tetrahydronaphthalene-2- |
| | carboxamide |
| I-213 | N-hydroxy-6-piperidin-4-yl-5,6,7,8-tetrahydronaphthalene-2- |
| | carboxamide |
| I-214 | tert-butyl 4-({[4-({6-[(hydroxyamino)carbonyl]-1,2,3,4- |
| | tetrahydronaphthalen-2-yl}oxy)pyridin-2-yl]amino}carbonyl)-4- |
| | methylpiperidine-1-carboxylate |
| I-215 | N-hydroxy-6-(pyridin-4-ylmethoxy)-5,6,7,8- |
| | tetrahydronaphthalene-2-carboxamide |
| I-216 | N-hydroxy-6-(piperidin-4-ylmethoxy)-5,6,7,8- |
| | tetrahydronaphthalene-2-carboxamide |
| I-217 | N-hydroxy-7-(pyridin-4-ylmethoxy)-5,6,7,8-tetrahydronaphthalene- |
| | 2-carboxamide |
| I-218 | N-hydroxy-7-(piperidin-4-ylmethoxy)-5,6,7,8- |
| | tetrahydronaphthalene-2-carboxamide |
| -219 | N-[4-({6-[(hydroxyamino)carbonyl]-1,2,3,4- |
| | tetrahydronaphthalen-2-yl}oxy)pyridin-2-yl]-1-isopropyl-4- |
| | methylpiperidine-4-carboxamide |
| I-220 | 4-({6-[(hydroxyamino)carbonyl]-1,2,3,4- |
| | tetrahydronaphthalen-2-yl}oxy)-N-piperidin-4-ylpyridine-2- |
| | carboxamide |
| I-221 | tert-butyl 4-{7-[(hydroxyamino)carbonyl]-1,2,3,4- |
| | tetrahydronaphthalen-2-yl}piperidine-1-carboxylate |
| I-222 | 1-ethyl-N-[4-({6-[(hydroxyamino)carbonyl]-1,2,3,4- |
| | tetrahydronaphthalen-2-yl}oxy)pyridin-2-yl]-4-methylpiperidine-4- |
| | carboxamide |
| I-223 | tert-butyl 4-({[4-({6-[(hydroxyamino)carbonyl]-1,2,3,4- |
| | tetrahydronaphthalen-2-yl}oxy)pyridin-2- |
| | yl]carbonyl}amino)piperidine-1-carboxylate |
| I-224 | tert-butyl 4-({[4-({6-[(hydroxyamino)carbonyl]-1,2,3,4- |
| | tetrahydronaphthalen-2-yl}oxy)pyridin-2- |
| | yl]amino}carbonyl)piperidine-1-carboxylate |
| I-225 | N-[4-({6-[(hydroxyamino)carbonyl]-1,2,3,4- |
| | tetrahydronaphthalen-2-yl}oxy)pyridin-2-yl]piperidine-4- |
| | carboxamide |
| I-226 | N-[2-(diethylamino)ethyl]-4-({6-[(hydroxyamino)carbonyl]- |
| | 1,2,3,4-tetrahydronaphthalen-2-yl}oxy)pyridine-2- |
| | carboxamide |
| I-227 | N-(1-ethylpiperidin-4-yl)-4-({6-[(hydroxyamino)carbonyl]-1,2,3,4- |
| | tetrahydronaphthalen-2-yl}oxy)pyridine-2-carboxamide |
| I-228 | 6-({2-[(4-chlorophenyl)amino]pyridin-4-yl}oxy)-N- |
| | hydroxy-5,6,7,8-tetrahydronaphthalene-2-carboxamide |
| I-229 | N-hydroxy-6-phenoxy-5,6,7,8-tetrahydronaphthalene-2- |
| | carboxamide |
| I-230 | 7-{[(dimethylamino)acetyl]amino}-N-hydroxy-5,6,7,8- |
| | tetrahydronaphthalene-2-carboxamide |
| I-231 | N-hydroxy-7-{[3-(methylamino)propanoyl]amino}-5,6,7,8- |
| | tetrahydronaphthalene-2-carboxamide |
| I-232 | N-hydroxy-6-[2-(3-piperidin-4-ylphenyl)ethyl]-5,6,7,8- |
| | tetrahydronaphthalene-2-carboxamide |
| I-233 | N-(4-{6-[(hydroxyamino)carbonyl]-1,2,3,4-tetrahydronaphthalen-2- |
| | yl}pyridin-2-yl)-1-isopropyl-4-methylpiperidine-4-carboxamide |
| I-234 | N-(3-{6-[(hydroxyamino)carbonyl]-1,2,3,4-tetrahydronaphthalen-2- |
| | yl}phenyl)-1-isopropyl-4-methylpiperidine-4-carboxamide |
| I-235 | N-hydroxy-7-{[(methylamino)acetyl]amino}-5,6,7,8- |
| | tetrahydronaphthalene-2-carboxamide |
| I-236 | N-{7-[(hydroxyamino)carbonyl]-1,2,3,4-tetrahydronaphthalen-2- |
| | yl}-1,2,3,4-tetrahydroisoquinoline-3-carboxamide |
| I-237 | 7-[(2-amino-3-methylbutanoyl)amino]-N-hydroxy-5,6,7,8- |
| | tetrahydronaphthalene-2-carboxamide |
| I-238 | N-hydroxy-6-(2-piperidin-3-ylethyl)-5,6,7,8-tetrahydronaphthalene- |
| | 2-carboxamide |
| I-239 | N-hydroxy-7-{[phenyl(piperidin-4-yl)acetyl]amino}-5,6,7,8- |
| | tetrahydronaphthalene-2-carboxamide |
| I-240 | N-{7-[(hydroxyamino)carbonyl]-1,2,3,4-tetrahydronaphthalen-2- |
| | yl}piperidine-3-carboxamide |
| I-241 | 1-ethyl-N-(4-{6-[(hydroxyamino)carbonyl]-1,2,3,4- |
| | tetrahydronaphthalen-2-yl}pyridin-2-yl)-4-methylpiperidine-4- |
| | carboxamide |
| I-242 | N-(4-{6-[(hydroxyamino)carbonyl]-1,2,3,4-tetrahydronaphthalen-2- |
| | yl}pyridin-2-yl)-1,4-dimethylpiperidine-4-carboxamide |
| I-243 | 1-ethyl-N-(4-{6-[(hydroxyamino)carbonyl]-1,2,3,4- |
| | tetrahydronaphthalen-2-yl}phenyl)-4-methylpiperidine-4- |
| | carboxamide |
| I-244 | 7-{[(2S)-2-amino-3-methylbutanoyl]amino}-N-hydroxy-5,6,7,8- |
| | tetrahydronaphthalene-2-carboxamide |
| I-245 | N-{7-[(hydroxyamino)carbonyl]-1,2,3,4-tetrahydronaphthalen-2- |
| | yl}pyrrolidine-2-carboxamide |

### 4. General Synthetic Methods and Intermediates

The compounds of the present invention can be prepared by methods known to one of ordinary skill in the art and/or by reference to the schemes shown below and the synthetic examples. Exemplary synthetic routes are set forth in Schemes below and in the Examples.

It will be appreciated that unless otherwise stated any one of the four available positions on the saturated ring of the tetralin can be functionalized as shown in Schemes below and the other three positions are substituted with R^{1a}, wherein R^{1a} has the values described herein. It will further be appreciated that similar transformations shown in Schemes below can be carried out on tetralins with substitution on either of the two rings in the R^{1a}, R^{1b}, an R¹ positions, as shown in Formula (I) herein, wherein R^{1a}, R^{1b}, and R¹ have the values described herein.

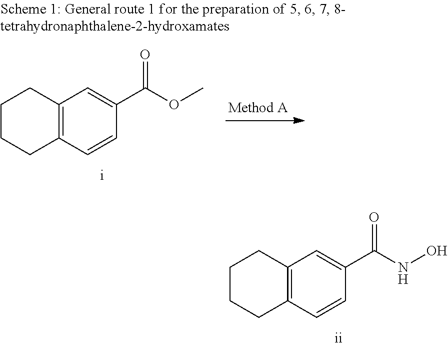

Scheme 1 shows a general route for the conversion of methyl ester i to the corresponding hydroxamate by reaction with the potassium salt of hydroxylamine (Method A; Huang et al., J. Med. Chem. 2009, 52(21):6757) leading to the formation of compounds of formula II. Methyl ester i may be commercially available or synthetically derived as described in the schemes below.

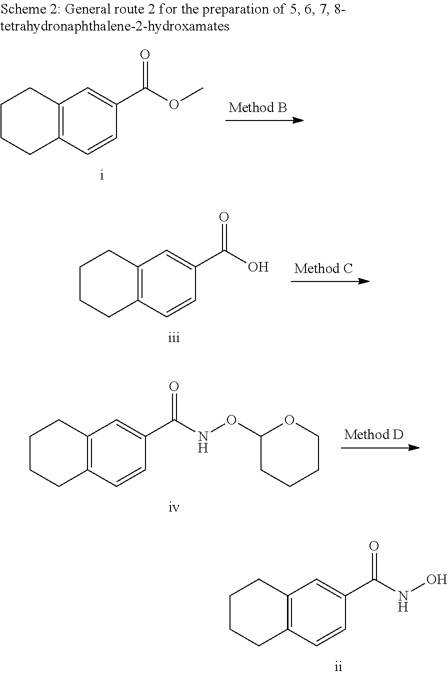 [SMILES: COC(=O)c1ccc2c(c1)CCCC2 ; O=C(O)c1ccc2c(c1)CCCC2 ; O=C(NOC1CCCCO1)c1ccc2c(c1)CCCC2 ; O=C(NO)c1ccc2c(c1)CCCC2]

Scheme 2 shows a general route for the conversion of methyl ester i to the corresponding hydroxamate. Methyl ester i may be converted to the corresponding carboxylic acid iii through the use of standard saponification chemistry using an aqueous hydroxide base such as sodium hydroxide or lithium hydroxide (Method B). The resulting carboxylic acid iii may be coupled to THP-protected hydroxylamine under standard amide coupling conditions (Method C, e.g. Carpino et al. J. Am. Chem. Soc. 1995, 117(19):5401). The THP acetal group may be hydrolyzed under treatment with mild acid (Method D, Secrist et al. J. Org. Chem. 1979, 44(9):1434) to afford the hydroxamate ii. Methyl ester i may be commercially available or synthetically derived as described in the schemes below.

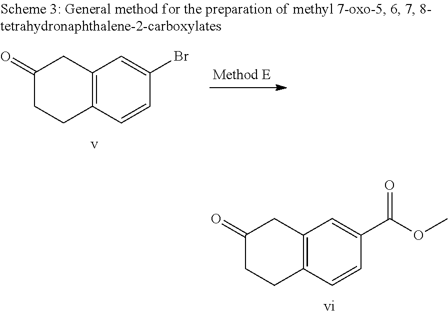

Scheme 3 shows a general method for conversion of commercially available bromide v to methyl ester vi. Bromide v may be carbonylated on the presence of carbon monoxide gas, methanol and a suitable palladium catalyst and ligand (Method E, Buchwald et al. J. Org. Chem., 2008 73: 7102) to afford the methyl ester vi.

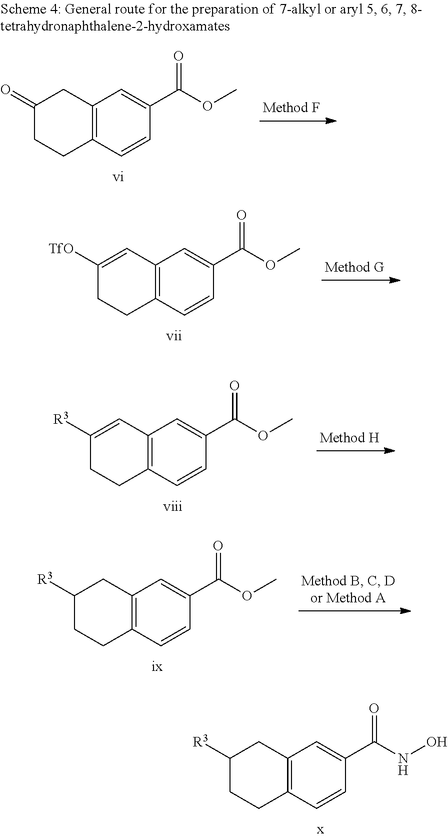

Scheme 4 depicts the preparation of analogs of formula x where the R³ substituent is directly attached to the tetrahydronapthalene ring. In this scheme the reactive substituent on the tetrahydronapthalene ring is shown on the 7-position but can also be in the 6-position. Tetralone vi (commercially available; prepared by Method E, or as described by Okumura et al., J. Med. Chem. 1998, 41 (21): 4036-4052) may be converted to the corresponding enol triflate vii by reaction with a suitable base and triflating agent (Method F; McMurry et al., Tetrahedron Lett. 1983, 24 (10): 979). Enol triflate vii may be arylated or alkylated by a Suzuki type reaction with a suitable arylboronic or alkylboronic acid or ester, by a Heck-type reaction with an alkene or acetylene, or by a Stille-type reaction with an organostannane (Method G; Molander et al., Tetrahedron 2002, 58: 1465; Ritter Synthesis 1993, 8: 735-62; Martinez et al., Organometallics 2001, 20 (5): 1020). The dihydronaphalene product viii can be reduced using standard palladium catalyzed hydrogenation chemistry (Method H). The methyl ester may be converted to the corresponding hydroxamate using Method A or Methods B-D.

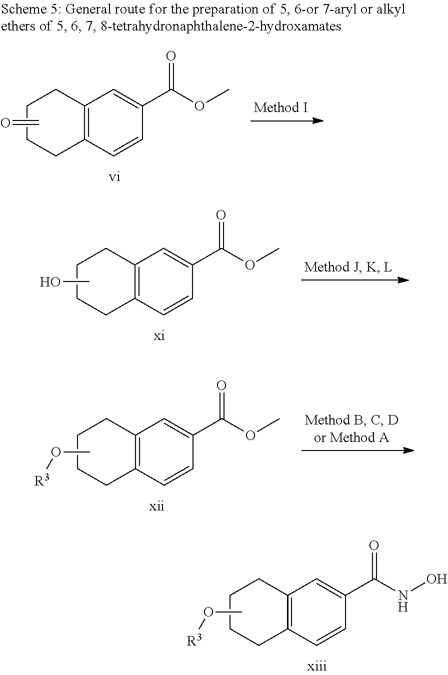

The preparation of O-arylated or alkylated tetrahydronapthalenes is shown in Scheme 5. Intermediate vi may be converted to the corresponding alcohol using standard reduction chemistry, using a hydride source such as sodium borohydride (Method I). The alcohol xi may be converted to its aryl ether by standard Mitsonobu reaction conditions (Method J; Swarmy et al., Chem. Rev. 2009, 109(6): 2551) or by standard nucleophilic aromatic substitution of a suitable electrophile such as 2-chloro-4-nitropyridine, in the presence of suitable base such as cesium carbonate in DMF at elevated temperature (Method K). The alcohol xi may also be converted to its alkyl ether in the presence of an appropriate alkyl iodide or bromide. This type of reaction can be carried out though silver (I) oxide mediated coupling in the presence of a phase transfer catalyst such as TBAI, or in the presence of a base such as sodium hydride or cesium carbonate at elevated temperatures. (Method L; Stauffer et al., J. Org. Chem., 2008, 73: 4166). The methyl ester may be converted to the hydroxamate xiii using Method A or Methods B-D.

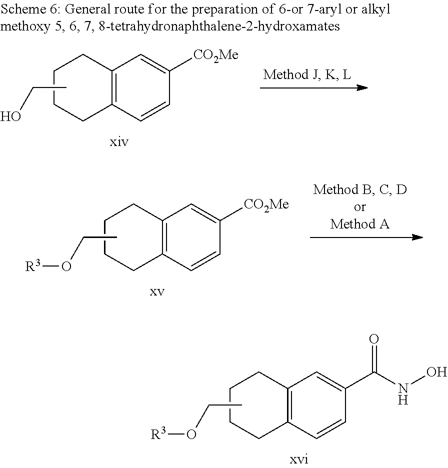

Scheme 6 shows a general route for the preparation of 6- or 7-aryl or alkyl methoxy-5,6,7,8-tetrahydronaphthalene-2-hydroxamates. Alcohol xiv may be prepared as described by Kanao et al, J. Med. Chem. 1989, 32, 1326. The alcohol may be alkylated or arylated using Methods J, K or L to afford intermediate xv, and converted to the corresponding hydroxamates using Method A, or Methods B-D.

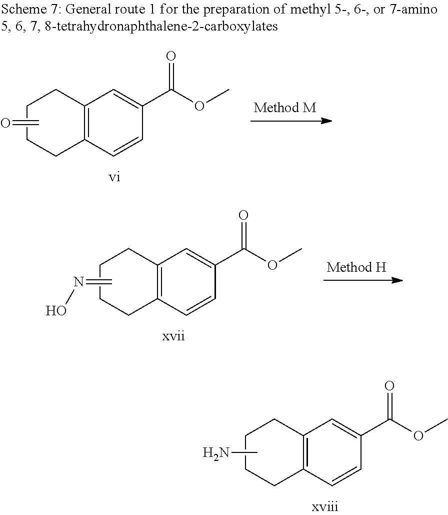

Scheme 7 shows a general method for the preparation of 5-, 6-, or 7-amino tetrahydronapthalenes xviii. Tetralone vi can be converted to an oxime xvii by treatment with hydroxylamine hydrochloride in the presence of sodium acetate and methanol (Method M; PCT Int. Appl. Publ. WO 06/002928). The oxime can be reduced to the amine xviii under standard palladium catalysed hydrogenation conditions (Method H).

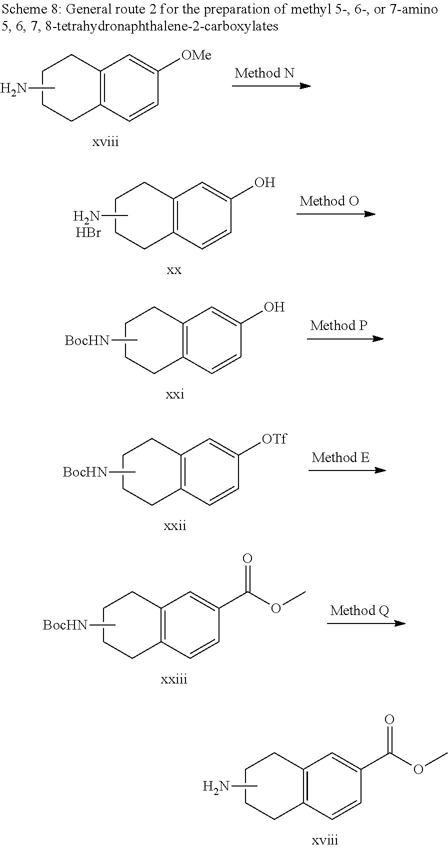

Scheme 8 shows an alternative general method for the preparation of 5-, 6-, or 7-amino tetrahydronapthalenes xviii. Amine xix (commercially available or prepared as described in European Patent Appl. Publ. EP 375560) may be demethylated under standard conditions by treatment with HBr at elevated temperature (Method N). Protection of the amine under standard conditions (Method O) and triflation of the phenol in the presence of a suitable base and triflic anhydride provides the triflate xxii (Method P). Carbonylation in the presence of carbon monoxide gas, methanol and a suitable palladium catalyst and ligand (Method E) affords the methyl ester xxiii. Boc deprotection is carried out in the presence of a suitable acid such as HCl (Method Q) to afford the desired amine xviii.

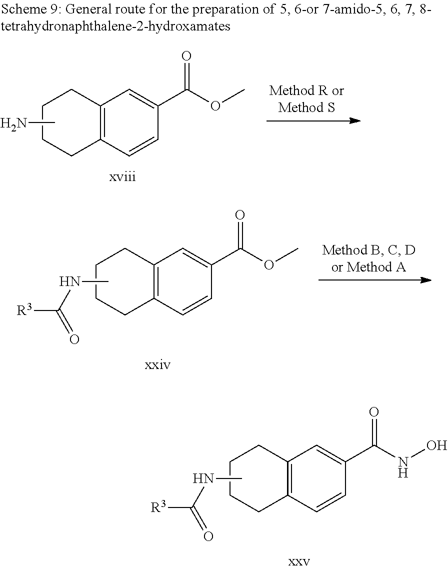

Scheme 9 shows a general method for the preparation of 5-, 6-, or 7-amido tetrahydronapthalenes xxv. Acylation of xviii may be achieved through a number of standard procedures, including reaction with an acid chloride in the presence of an amine base (Method R) or coupling with a carboxylic acid in the presence of a suitable coupling agent such as HATU or TFFH (Method S). The methyl ester may be converted to the hydroxamate using Method A or Methods B-D. 9-tert-Butyl 6-methyl 1,2,3,4-tetrahydro-1,4-epiminonaphthalene-6,9-dicarboxylate (prepared as described by Kitamura et al., Synlett 1999 6: 731-732 and PCT Int. Appl. Publ. WO 05/094251) may also be acylated and converted to the desired hydroxamate xxv using Methods R or S followed by Method A, or Methods B-D.

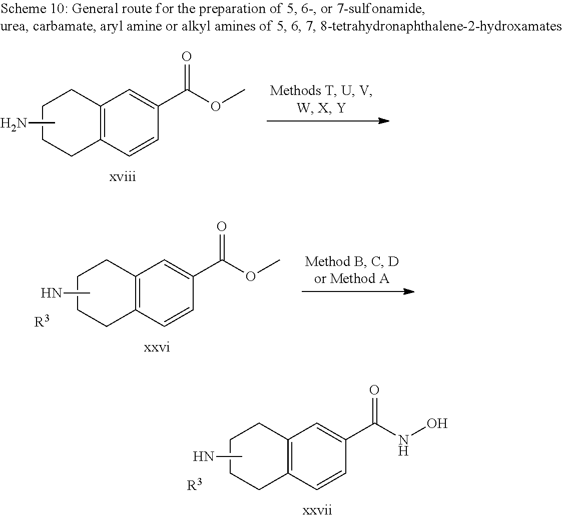

Scheme 10 depicts how amine xviii can be converted to ureas, sulfonamides, carbamates, alkylamines and arylamines. Treatment of amine xviii with an aldehyde under standard reductive amination conditions affords the alkylamine xxvi (Method T). Amine xviii can also be arylated using standard nucleophilic aromatic substitution of a suitable electrophile such as 2-chloro-4-nitropyridine, in the presence of suitable base such as DIPEA at elevated temperature (Method U) Amine xviii may also be N-arylated through a copper(II) acetate mediated coupling with a suitable arylboronic acid (Method V, Chan et al. Tetrahedron Lett. 1998, 39(19):2933) Amine xviii may be converted under standard reaction conditions to a sulfonamide by treatment with a sulfonyl chloride (Method W); to a urea by treatment with an isocyanate (Method X) or to a carbamate by treatment with an anhydride (Method Y). Methyl ester xxvii may be converted to the corresponding hydroxamate using Method A or Methods B-D.

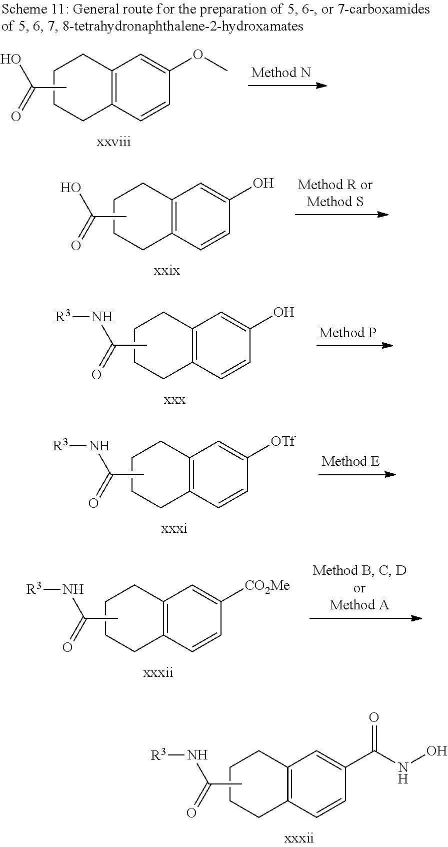

Scheme 11 shows a general method for the preparation of 5,6-, or 7-carboxamides of 5, 6, 7, 8-tetrahydronaphthalene-2-hydroxamates. Commercially available acids xxviii may be deprotected (Method N) and coupled to a suitable amine using Method S or by first preparing the acid chloride using standard conditions, then coupling to an amine using method R. Formation of the triflate and carbonylation may be carried out using Methods P and E. Methyl ester xxxii may be converted to the corresponding hydroxamate using Method A or Methods B-D.

### 5. Uses, Formulation and Administration

As discussed above, the present invention provides compounds and pharmaceutical compositions that are useful as inhibitors of HDAC enzymes, particularly HDAC6, and thus the present compounds are useful for treating proliferative, inflammatory, infectious, neurological or cardiovascular disorders.

The compounds and pharmaceutical compositions of the invention are particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

In some embodiments, therefore, the invention provides the compound of formula (I), or a pharmaceutically acceptable salt thereof, for use in treating cancer. In some embodiments, the invention provides a pharmaceutical composition (as described herein) for the treatment of cancer comprising the compound of formula (I), or a pharmaceutically acceptable salt thereof. In some embodiments, the invention provides the use of the compound of formula (I), or a pharmaceutically acceptable salt thereof, for the preparation of a pharmaceutical composition (as described herein) for the treatment of cancer. In some embodiments, the invention provides the use of an effective amount of the compound of formula (I), or a pharmaceutically acceptable salt thereof, for the treatment of cancer.

Non-limiting examples of solid tumors that can be treated with the disclosed inhibitors include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

Non-limiting examples of hematologic malignancies that can be treated with the disclosed inhibitors include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In some embodiments, compounds of the invention are suitable for the treatment of breast cancer, lung cancer, ovarian cancer, multiple myeloma, acute myeloid leukemia or acute lymphoblastic leukemia.

In other embodiments, compounds of the invention are suitable for the treatment of inflammatory and cardiovascular disorders including, but not limited to, allergies/anaphylaxis, acute and chronic inflammation, rheumatoid arthritis; autoimmunity disorders, thrombosis, hypertension, cardiac hypertrophy, and heart failure.

Accordingly, in another aspect of the present invention, pharmaceutical compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable prodrugs, salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of HDAC6.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, a method for treating a proliferative, inflammatory, infectious, neurological or cardiovascular disorder is provided comprising administering an effective amount of a compound, or a pharmaceutical composition to a subject in need thereof. In certain embodiments of the present invention an “effective amount” of the compound or pharmaceutical composition is that amount effective for treating a proliferative, inflammatory, infectious, neurological or cardiovascular disorder, or is that amount effective for treating cancer. In other embodiments, an “effective amount” of a compound is an amount which inhibits binding of HDAC6, and thereby blocks the resulting signaling cascades that lead to the abnormal activity of growth factors, receptor tyrosine kinases, protein serine/threonine kinases, G protein coupled receptors and phospholipid kinases and phosphatases.

The compounds and compositions, according to the method of the present invention, may be administered using any amount and any route of administration effective for treating the disease. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, a compound of formula (I) or a pharmaceutical composition thereof is administered in conjunction with an anticancer agent. As used herein, the term “anticancer agent” refers to any agent that is administered to a subject with cancer for purposes of treating the cancer. Combination therapy includes administration of the therapeutic agents concurrently or sequentially. Alternatively, the therapeutic agents can be combined into one composition which is administered to the patient.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which down-regulates cell replication. In certain embodiments, a compound of the invention is administered in conjunction with a proteasome inhibitor.

Another aspect of the invention relates to inhibiting HDAC6, activity in a biological sample or a patient, which method comprises administering to the patient, or contacting said biological sample with a compound of formula (I), or a composition comprising said compound. The term “biological sample”, as used herein, generally includes in vivo, in vitro, and ex vivo materials, and also includes, without limitation, cell cultures or extracts thereof; biopsied material obtained from a mammal or extracts thereof; and blood, saliva, urine, feces, semen, tears, or other body fluids or extracts thereof.

Still another aspect of this invention is to provide a kit comprising separate containers in a single package, wherein the inventive pharmaceutical compounds, compositions and/or salts thereof are used in combination with pharmaceutically acceptable carriers to treat disorders, symptoms and diseases where HDAC6 plays a role.

### 5. Preparation of Exemplary Compounds

### Experimental Procedures

### Definitions

ATP adenosine triphosphate
DCE dichloroethane
DCM dichloromethane
DIPEA diisopropylethyl amine
DMF N,N-dimethylformamide
DMSO dimethylsulfoxide
EDTA ethylenediaminetetraacetic acid
EtOAc ethyl acetate
EtOH ethanol
FA formic acid
FBS fetal bovine serum
h hours
HATU N,N,N′,N′-tetramethyl-o-(7-azabenzotriazole-1-yl)uronium hexafluorophosphate
HEPES N-(2-hydroxyethyl)piperazine-N′-(2-ethanesulfonic acid)
HRMS high resolution mass spectrum
IPA isopropyl alcohol
LC-MS liquid chromatography mass spectrum
m/z mass to charge
MTBE methyl tert-butyl ether
Me methyl
MEM minimum essential media
MeOH methanol
min minutes
MS mass spectrum
MWI microwave irradiation
NMM N-methyl morpholine
PBS phosphate buffered saline
rt room temperature
TEA triethylamine
TFA trifluoroacetic acid
TFAA trifluoroacetic anhydride
TFFH 1,1,3,3-tetramethylfluoroformamidinium hexafluorophosphate
THF tetrahydrofuran
TMEDA IV, N,N′,N′-tetramethyl-ethane-1,2-diamine
Xantphos 4,5-Bis(diphenylphosphino)-9,9-dimethylxanthene

Analytical Methods

NMR: 1H NMR spectra are run on a 400 MHz Bruker unless otherwise stated.

LC-MS: LC-MS spectra are run using an Agilent 1100 LC fitted with a Waters Symmetry® C18 3.5 μm, 4.6×100 mm column, interfaced to a micromass Waters® Micromass® Zspray™ Mass Detector (ZMD) using the following gradients:
Formic Acid (FA): Acetonitrile containing zero to 100 percent 0.1% formic acid in water.
Ammonium Acetate (AA): Acetonitrile containing zero to 100 percent 10 mM ammonium acetate in water.

HPLC: Preparative HPLC are conducted using 18×150 mm Sunfire C-18 columns eluting with water-MeCN gradients using a Gilson instrument operated by 322 pumps with the UV/visible 155 detector triggered fraction collection set to between 200 nm and 400 nm. Mass gated fraction collection is conducted on an Agilent 1100 LC/MSD instrument.

### Example 1

### methyl 6-[(trifluoromethyl)sulfonyl]-7,8-dihydronaphthalene-2-carboxylate. Intermediate 2

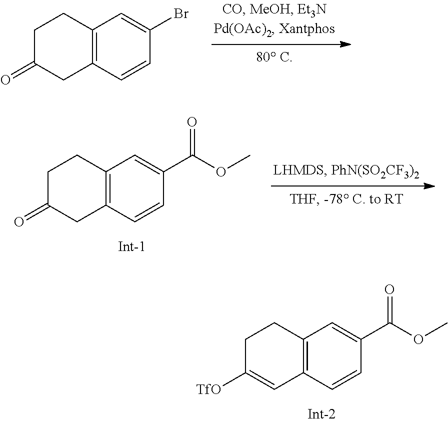 [SMILES: O=C1CCc2cc(Br)ccc2C1 ; COC(=O)c1ccc2c(c1)CCC(=O)C2 ; COC(=O)c1ccc2c(c1)CCC(OS(=O)(=O)C(F)(F)F)=C2]

### Step 1: Methyl 6-oxo-5,6,7,8-tetrahydronaphthalene-2-carboxylate Intermediate 1

A solution of 6-bromo-3,4-dihydronaphthalen-2(1H)-one (5 g, 22.2 mmol) in triethylamine (44.8 mL) and methanol (9 mL, 222 mmol) was degassed with nitrogen and palladium (II) acetate (99.7 mg, 0.45 mmol) and Xantphos (514 mg, 0.89 mmol) were added. The solution was degassed again and CO gas was bubbled through the solution for approximately 2 min. The reaction flask was fitted with a condenser and a CO balloon and the reaction mixture was heated at 70° C. for 3.5 h. The reaction mixture was cooled to rt, diluted with EtOAc and filtered through Celite. The filtrate was evaporated and the residue was purified by silica gel chromatography (5% to 20% EtOAc/hexanes) to afford methyl 6-oxo-5,6,7,8-tetrahydronaphthalene-2-carboxylate (1.00 g, 22%). ¹H NMR (300 MHz, CDCl₃) δ ppm 7.93 (s, 1H), 7.90 (dd, J=8.0, 1.5 Hz, 1H), 7.21 (d, J=7.9 Hz, 1H), 3.93 (s, 3H), 3.64 (s, 2H), 3.13 (t, J=6.7 Hz, 2H), 2.60-2.55 (m, 2H).

### Step 2: Methyl 6-[(trifluoromethyl)sulfonyl]-7,8-dihydronaphthalene-2-carboxylate. Intermediate 2

A solution of methyl 6-oxo-5,6,7,8-tetrahydronaphthalene-2-carboxylate (1.0 g, 4.9 mmol) in THF (16 mL) was cooled to −78° C. and lithium hexamethyldisilazide (1.0 M in THF, 5.88 mL, 5.88 mmol) was added drop-wise. The reaction mixture was allowed to stir at −78° C. for 1 h, and then a solution of N-phenylbis(trifluoromethanesulphonimide) (2.1 g, 5.88 mmol) in THF (9.4 mL) was added drop-wise. The reaction mixture was allowed to warm to rt and was stirred overnight. Water was added and the reaction mixture was extracted with Et₂O (2×). The organic phases were washed with 1N HCl, sat. NaHCO₃ solution and brine, dried over Na₂SO₄ and evaporated. The residue was purified by silica gel chromatography (5% to 20% EtOAc/hexane) to afford methyl 6-[(trifluoromethyl)sulfonyl]-7,8-dihydronaphthalene-2-carboxylate (885 mg, 54%). ¹H NMR (300 MHz, CDCl₃) δ ppm 7.87 (dd, J=7.9, 1.6 Hz, 1H), 7.82 (s, 1H), 7.14 (d, J=7.9 Hz, 1H), 6.53 (s, 1H), 3.11 (t, J=8.4 Hz, 2H), 2.73 (t, J=8.5 Hz, 2H).

### Example 2

### Methyl 7-{[(trifluoromethyl)sulfonyl]oxy}-5,6-dihydronaphthalene-2-carboxylate Intermediate 9

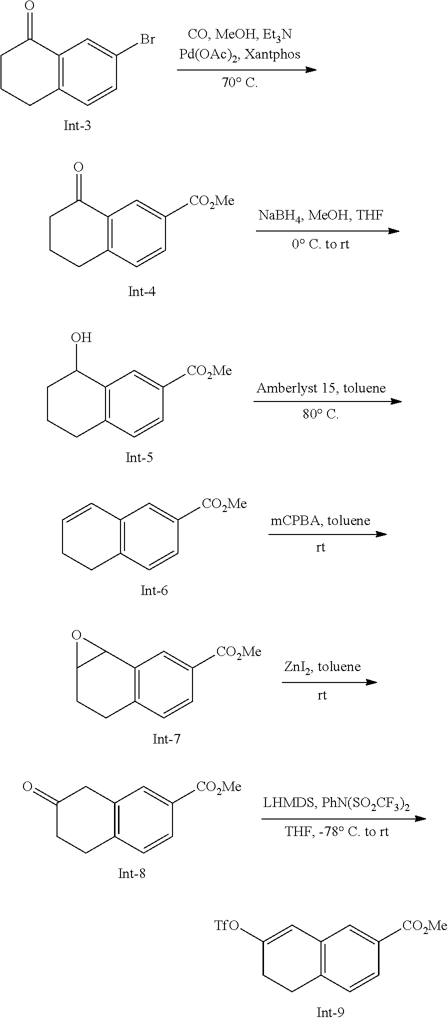

### Step 1: methyl 8-oxo-5,6,7,8-tetrahydronaphthalene-2-carboxylate Intermediate 4

The title compound was prepared from 7-bromo-3,4-dihydronaphthalen-1(2H)-one following the procedure outlined in Example 1, step 1 (88%) ¹H NMR (400 MHz, CDCl₃) δ ppm 8.68 (d, J=1.7 Hz, 1H), 8.12 (dd, J=7.9, 1.9 Hz, 1H), 7.34 (d, J=8.0 Hz, 1H), 3.92 (s, 3H), 3.02 (t, J=6.1 Hz, 2H), 2.71-2.67 (m, 2H), 2.17 (td, J=12.7, 6.4 Hz, 2H).

### Step 2: methyl 8-hydroxy-5,6,7,8-tetrahydronaphthalene-2-carboxylate Intermediate 5

Into a round bottom flask was added methyl 8-oxo-5,6,7,8-tetrahydronaphthalene-2-carboxylate (4.45 g, 21.8 mmol), THF (90 mL) and methanol (40 mL). The reaction mixture was cooled to 0° C. and sodium borohydride (0.495 g, 13.1 mmol) was added portion-wise. The reaction mixture was stirred at 0° C. for 30 minutes then allowed to warm to rt. After 2 h, the reaction mixture was neutralized with 1N HCl and extracted with EtOAc (2×). The organic phase was washed with brine, dried over anhydrous sodium sulfate, filtered and concentrated to give methyl 8-hydroxy-5,6,7,8-tetrahydronaphthalene-2-carboxylate (4.91 g, quantitative). ¹H NMR (400 MHz, CDCl₃) δ ppm 8.12 (d, J 1.6 Hz, 1H), 7.84 (dd, J=8.0, 1.8 Hz, 1H), 7.16 (d, J=8.0 Hz, 1H), 4.84-4.80 (m, 1H), 3.90 (s, 3H), 2.88 (td, J=17.0, 5.4 Hz, 1H), 2.76 (td, J=17.6, 6.6 Hz, 1H), 2.03-1.74 (m, 5H).

### Step 3: methyl 5,6-dihydronaphthalene-2-carboxylate Intermediate 6

Into a round bottom flask was added methyl 8-hydroxy-5,6,7,8-tetrahydronaphthalene-2-carboxylate (4.48 g, 21.7 mmol), toluene (82 mL) and 500 mg of Amberlyst 15. The reaction mixture was heated at 80° C. After 2 h, the reaction mixture was cooled to rt, filtered, and the filtrate was evaporated. The residue was purified by silica gel chromatography (0% to 30% EtOAc/hexane) to afford methyl 5,6-dihydronaphthalene-2-carboxylate (3.84 g, 94%). ¹H NMR (400 MHz, CDCl₃) δ ppm 7.79 (dd, J=7.8, 1.8 Hz, 1H), 7.68 (d, J=1.6 Hz, 1H), 7.15 (d, J=7.8 Hz, 1H), 6.50 (td, J=9.6, 1.7 Hz, 1H), 6.11-6.06 (m, 1H), 3.90 (s, 3H), 2.85 (t, J=8.2 Hz, 2H), 2.37-2.31 (m, 2H).

### Step 4: methyl 1a,2,3,7b-tetrahydronaphtho[1,2-b]oxirene-6-carboxylate Intermediate 7

To a solution of methyl 5,6-dihydronaphthalene-2-carboxylate (1.19 g, 6.32 mmol) in toluene (17.8 mL) was added m-chloroperbenzoic acid (1.42 g, 8.22 mmol) and the reaction mixture was stirred at rt for 2 h. After 2 h, the reaction mixture was filtered and the organic phase was washed with sat. NaHCO₃ solution (4×) then brine, dried over anhydrous Na₂SO₄, filtered and evaporated. The residue was purified by silica gel chromatography (0-20% EtOAc/hexane) to give methyl 1a,2,3,7b-tetrahydronaphtho[1,2-b]oxirene-6-carboxylate (1.11 g, 86%). ¹H NMR (400 MHz, CDCl₃) δ ppm 8.08 (d, J=1.7 Hz, 1H), 7.92 (dd, J=7.8, 1.8 Hz, 1H), 7.16 (d, J=7.8 Hz, 1H), 3.92 (s, 3H), 3.91 (d, J=4.2 Hz, 1H), 3.76 (t, J=3.4 Hz, 1H), 2.87-2.77 (m, 1H), 2.61 (dd, J=15.9, 5.5 Hz, 1H), 2.48-2.41 (m, 1H), 1.78 (dt, J=13.9, 5.6 Hz, 1H).

### Step 5: methyl 7-oxo-5,6,7,8-tetrahydronaphthalene-2-carboxylate Intermediate 8

Into a solution of methyl 1a,2,3,7b-tetrahydronaphtho[1,2-b]oxirene-6-carboxylate (3.5 g, 17.1 mmol) in toluene (100 mL) was added zinc diiodide (6.56 g, 20.6 mmol) and the reaction mixture was stirred for 3 h. The reaction mixture was quenched with water and extracted with EtOAc (2×) and washed with brine, dried over anhydrous Na₂SO₄, filtered and evaporated. The residue was purified by silica gel chromatography (0-50% EtOAc/hexane) to give methyl 7-oxo-5,6,7,8-tetrahydronaphthalene-2-carboxylate (3.48 g, 99%). ¹H NMR (400 MHz, CDCl₃) δ ppm 7.89 (dd, J=7.9, 1.6 Hz, 1H), 7.81 (s, 1H), 7.31 (d, J=7.7 Hz, 1H), 3.91 (s, 3H), 3.64 (s, 2H), 3.12 (t, J=6.7 Hz, 2H), 2.59-2.55 (m, 2H).

### Step 6: methyl 7-{[(trifluoromethyl)sulfonyl]oxy}-5,6-dihydronaphthalene-2-carboxylate Intermediate 9

A solution of methyl 7-oxo-5,6,7,8-tetrahydronaphthalene-2-carboxylate (3.48 g, 17 mmol) in THF (54.4 mL) was cooled to −78° C. and lithium hexamethyldisilazide (1.0 M in THF, 20.4 mL, 20.4 mmol) was added drop-wise. The reaction mixture was allowed to stir at −78° C. for 1 h, and then a solution of N-phenylbis(trifluoromethanesulphonimide) (7.3 g, 20.45 mmol) in THF (32.6 mL) was added drop-wise. The reaction mixture was allowed to warm to rt and was stirred overnight. Water was added and the reaction mixture was extracted with Et₂O (2×). The organic phases were washed with 1N HCl, 1N NaOH and brine, dried over anhydrous Na₂SO₄, filtered and evaporated. The residue was purified by silica gel chromatography (0% to 50% EtOAc/hexane) to afford 7-{[(trifluoromethyl)sulfonyl]oxy}-5,6-dihydronaphthalene-2-carboxylate (3.45 g, 73%). ¹H NMR (400 MHz, CDCl₃) δ ppm 7.88 (dd, J=7.8, 1.73 Hz, 1H), 7.76 (d, J=1.6 Hz, 1H), 7.22 (d, J=7.8 Hz, 1H), 6.53 (s, 1H), 3.92-3.91 (m, 3H), 3.12 (t, J=8.4 Hz, 2H), 2.75-2.70 (m, 2H).

### Example 3

### N-hydroxy-6-(4-methoxyphenyl)-5,6,7,8-tetrahydronaphthalene-2-carboxamide Compound I-105

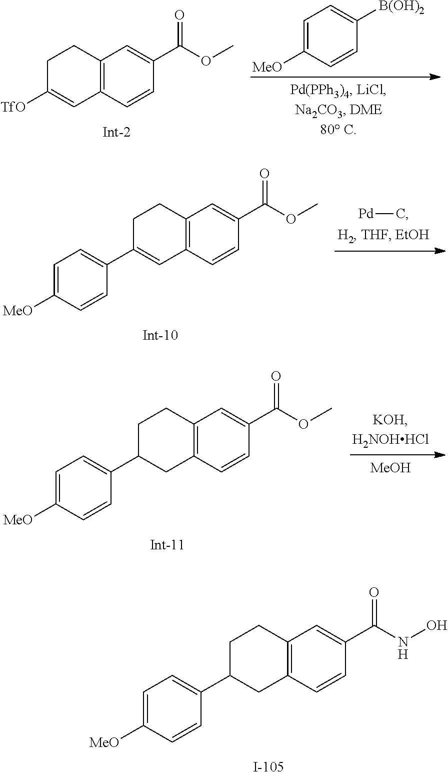

### Step 1: methyl 6-(4-methoxyphenyl)-7,8-dihydronaphthalene-2-carboxylate Intermediate 10

To a solution of methyl 6-[(trifluoromethyl)sulfonyl]-7,8-dihydronaphthalene-2-carboxylate (181 mg, 0.54 mmol) in dimethoxyethane (4.5 mL) was added sodium carbonate (2.0 M in water, 0.81 mL, 1.61 mmol), lithium chloride (68.4 mg, 1.61 mmol) and 4-methoxybenzene boronic acid (98.1 mg, 0.65 mmol). The reaction mixture was degassed with argon and tetrakis(triphenylphosphine)palladium(0) (12.4 mg, 10.8 mmol) was added. The reaction mixture was heated at 80° C. overnight. The reaction mixture was cooled to rt, diluted with water and extracted with EtOAc (2×). The organic phase was washed with brine, dried over Na₂SO₄ and evaporated. The residue was purified by silica gel chromatography (0% to 20% EtOAc/hexane) to afford methyl 6-(4-methoxyphenyl)-7,8-dihydronaphthalene-2-carboxylate (66 mg, 42%). ¹H NMR (400 MHz, CDCl₃) δ ppm 7.85 (dd, J=7.8, 1.7 Hz, 1H), 7.82 (s, 1H), 7.53 (d, J=2.1 Hz, 1H), 7.51 (d, J=2.1 Hz, 1H), 7.15 (d, J=7.8 Hz, 1H), 6.94 (d, J=2.1 Hz, 1H), 6.92 (d, J=2.1 Hz, 1H), 6.81 (s, 1H), 3.91 (s, 3H), 3.85 (s, 3H), 2.99 (t, J=8.1 Hz, 2H), 2.79-2.73 (m, 2H).

### Step 2: methyl 6-(4-methoxyphenyl)-5,6,7,8-tetrahydronaphthalene-2-carboxylate Intermediate 11

To a solution of methyl 6-(4-methoxyphenyl)-7,8-dihydronaphthalene-2-carboxylate (66 mg, 0.22 mmol) in ethanol (1.1 mL) and THF (3 mL), was added 10% palladium on carbon (24 mg). The mixture was stirred under an atmosphere of H₂ gas overnight. The reaction mixture was filtered through Celite, washed with ethyl acetate and the filtrate was evaporated. The residue was purified by chromatography on silica (0% to 20% EtOAc/hexane) to afford methyl 6-(4-methoxyphenyl)-5,6,7,8-tetrahydronaphthalene-2-carboxylate (54 mg, 81%). ¹H NMR (400 MHz, CDCl₃) δ ppm 7.82 (s, 1H), 7.77 (dd, J=8.0, 1.6 Hz, 1H), 7.20 (d, J=1.9 Hz, 1H), 7.18 (d, J=2.0 Hz, 1H), 7.15 (d, J=8.0 Hz, 1H), 6.89 (d, J=2.0 Hz, 1H), 6.88 (d, J=2.0 Hz, 1H), 3.91 (s, 3H), 3.81 (s, 3H), 3.07 (d, J=13.0 Hz, 1H), 3.01-2.89 (m, 4H), 2.17-2.10 (m, 1H), 1.97-1.85 (m, 1H).

### Step 3: N-Hydroxy-6-(4-methoxyphenyl)-5,6,7,8-tetrahydronaphthalene-2-carboxamide Compound I-105

A mixture of hydroxylamine hydrochloride (2.0 g, 29 mmol) in methanol (10 mL) was heated at 90° C. under a dry nitrogen atmosphere until homogeneous. To this heated solution was added a solution of potassium hydroxide (2.85 g, 50.8 mmol) in methanol (6 mL). A precipitate formed on mixing. After heating at 90° C. for 30 minutes, the mixture was cooled to rt and the solids were allowed to settle. The resulting solution was assumed to contain 1.7 M hydroxylamine.potassium salt and was carefully removed by syringe to exclude solids. An aliquot of the above solution (1.02 mL, 1.74 mmol) was added to a solution of methyl 6-(4-methoxyphenyl)-5,6,7,8-tetrahydronaphthalene-2-carboxylate (0.051 g, 0.17 mmol) in methanol (0.7 mL). After stirring for 1 h at rt, excess reagent was quenched by the addition of acetic acid (0.098 mL, 1.72 mmol). The mixture was concentrated to dryness and the residue was twice co-evaporated with toluene. The crude product was purified by preparative HPLC to afford N-hydroxy-6-(4-methoxyphenyl)-5,6,7,8-tetrahydronaphthalene-2-carboxamide (27 mg, 59%). LC-MS (FA) ES+ 298; ¹H NMR (400 MHz, CD₃OD) δ ppm 7.50 (s, 1H), 7.46 (d, J=7.9 Hz, 1H), 7.21-7.14 (m, 3H), 6.86 (d, J=2.1 Hz, 1H), 6.85 (d, J=1.9 Hz, 1H), 3.76 (s, 3H), 3.06-2.84 (m, 5H), 2.13-2.05 (m, 1H), 1.97-1.85 (m, 1H).

### Example 4

The following compounds were prepared in a fashion analogous to that described in Example 3 starting from the intermediates which were prepared as described above and the corresponding boronic acids or esters. Where the boronic acid or ester used contained an N-Boc group, this was removed in the final step following the procedure outlined in Example 30, step 3.

| Compound | LC-MS |
|---|---|
| I-104 | ES+ 298 (FA) |
| I-102 | ES+ 325 (FA) |
| I-101 | ES+ 302 (FA) |
| I-100 | ES+ 268 (FA) |
| I-99 | ES+ 269 (AA) |
| I-92 | ES+ 325 (FA) |
| I-90 | ES+ 302 (FA) |
| I-89 | ES+ 268 (FA) |
| I-88 | ES+ 269 (FA) |
| I-110 | ES+ 298 (FA) |
| I-109 | ES+ 336 (FA) |
| I-116 | ES+ 298 (FA) |
| I-115 | ES+ 336 (FA) |
| I-113 | ES+ 336 (FA) |
| I-120 | ES+ 269 (AA) |
| I-119 | ES+ 298 (FA) |
| I-121 | ES+ 269 (AA) |
| I-185 | ES+ 336 (FA) |
| I-190 | ES+ 353 (FA) |
| I-183 | ES+ 325 (FA) |

### Example 5

### 6-[2-(3-fluorophenyl)ethyl]-N-hydroxy-5,6,7,8-tetrahydronaphthalene-2-carboxamide Compound I-103

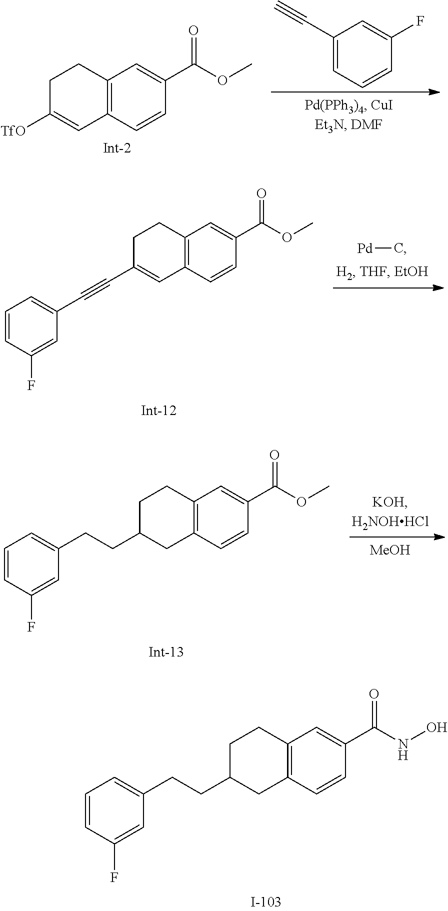

### Step 1: methyl 6-[(3-fluorophenyl)ethynyl]-7,8-dihydronaphthalene-2-carboxylate Intermediate 12

A mixture of methyl 6-{[(trifluoromethyl)sulfonyl]oxy}-7,8-dihydronaphthalene-2-carboxylate (0.22 g, 0.65 mmol) in DMF (8.8 mL), copper(I) iodide (12.4 mg, 0.065 mmol), tetrakis(triphenylphosphine)palladium(0) (75.6 mg, 0.065 mmol) and triethylamine (0.182 mL, 1.31 mmol) was degassed with argon. The reaction mixture was stirred for 1 h at rt. 1-Ethynyl-3-fluorobenzene (0.25 mL, 2.16 mmol) was added and the reaction mixture was heated at 60° C. overnight. After cooling to rt, the reaction mixture was diluted with water and extracted with EtOAc (2×). The organic phases were washed with brine, dried over anhydrous sodium sulfate, filtered and concentrated. The residue was purified by silica gel chromatography (0-10% EtOAc/hexane) to afford methyl 6-[(3-fluorophenyl)ethynyl]-7,8-dihydronaphthalene-2-carboxylate (166 mg, 83%). ¹H NMR (400 MHz, CDCl₃) δ ppm 7.85 (dd, J=7.8, 1.7 Hz, 1H), 7.81 (s, 1H), 7.34-7.29 (m, 1H), 7.28-7.26 (m, 1H), 7.20-7.15 (m, 1H), 7.12 (d, J=7.9 Hz, 1H), 7.07-7.00 (m, 1H), 6.91 (s, 1H), 3.91 (s, 3H), 2.94 (t, J=8.2 Hz, 2H), 2.59-2.54 (m, 2H).

### Step 2: methyl 6-[2-(3-fluorophenyl)ethyl]-5,6,7,8-tetrahydronaphthalene-2-carboxylate Intermediate 13

The title compound was prepared from methyl 6-[(3-fluorophenyl)ethynyl]-7,8-dihydronaphthalene-2-carboxylate following the procedure outlined in Example 3, step 2 (157 mg, 95%). ¹H NMR (400 MHz, CDCl₃) δ ppm 7.78-7.73 (m, 2H), 7.25-7.21 (m, 1H), 7.12 (d, J=8.0 Hz, 1H), 6.98 (d, J=7.7 Hz, 1H), 6.94-6.85 (m, 2H), 3.89 (s, 3H), 2.95 (dd, J=17.4, 4.3 Hz, 1H), 2.90-2.76 (m, 2H), 2.75-2.70 (m, 2H), 2.50 (dd, J=17.0, 10.1 Hz, 1H), 2.05-1.97 (m, 1H), 1.83-1.74 (m, 1H), 1.73-1.66 (m, 2H), 1.51-1.40 (m, 1H).

### Step 3: 6-[2-(3-Fluorophenyl)ethyl]-N-hydroxy-5,6,7,8-tetrahydronaphthalene-2-carboxamide Compound I-103

The title compound was prepared from methyl 6-[2-(3-fluorophenyl)ethyl]-5,6,7,8-tetrahydronaphthalene-2-carboxylate following the procedure outlined in Example 3, step 3. (97 mg, 62%) LC-MS (FA) ES+ 314; ¹H NMR (400 MHz, CD₃OD) δ ppm 7.43 (d, J=8.4 Hz, 2H), 7.25 (dt, J=7.9, 6.3 Hz, 1H), 7.13 (d, J=7.8 Hz, 1H), 7.02 (d, J=7.6 Hz, 1H), 6.95 (d, J=10.2 Hz, 1H), 6.87 (dt, J=8.4, 2.0 Hz, 1H), 2.94 (dd, J=17.0, 3.6 Hz, 1H), 2.90-2.78 (m, 2H), 2.77-2.72 (m, 2H), 2.47 (dd, J=16.9, 9.7 Hz, 1H), 2.02 (d, J=12.7 Hz, 1H), 1.80-1.64 (m, 3H), 1.50-1.38 (m, 1H).

### Example 6

### N-hydroxy-7-(2-phenylethyl)-5,6,7,8-tetrahydronaphthalene-2-carboxamide Compound I-93

The title compound was prepared from methyl 7-{[(trifluoromethyl)sulfonyl]oxy}-5,6-dihydronaphthalene-2-carboxylate following the procedures outlined in Example 5, using ethynylbenzene in the place of 1-ethynyl-3-fluorobenzene. LC-MS (FA) ES+ 296; ¹H NMR (400 MHz, CD₃OD) δ ppm 7.46-7.41 (m, 2H), 7.27-7.18 (m, 4H), 7.16-7.10 (m, 2H), 2.95 (dd, J=16.4, 3.8 Hz, 1H), 2.90-2.77 (m, 2H), 2.76-2.70 (m, 2H), 2.48 (dd, J=16.4, 9.8 Hz, 1H), 2.05-1.98 (m, 1H), 1.79-1.64 (m, 3H), 1.49-1.38 (m, 1H).

### Example 7

### 7-(6-aminopyrimidin-4-yl)-N-hydroxy-5,6,7,8-tetrahydronaphthalene-2-carboxamide Compound I-98

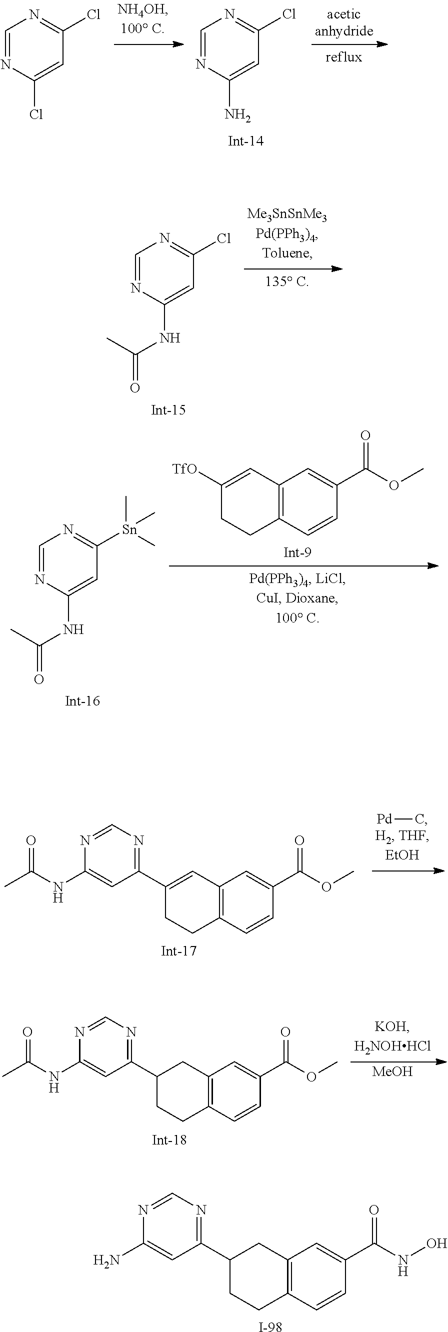

### Step 1: 6-chloropyrimidin-4-amine Intermediate 14

4,6-Dichloropyrimidine (7.5 g, 50 mmol) was suspended in ammonium hydroxide (64 mL) in a sealed tube. The tube was sealed and heated at 100° C. in an oil bath overnight. The reaction mixture was cooled to it. The solid was removed by filtration, washed with water and dried under high vacuum to afford 6-chloropyrimidin-4-amine (5.23 g, 80%) LC-MS (AA) ES+ 130.

### Step 2: N-(6-chloropyrimidin-4-yl)acetamide Intermediate 15

6-Chloropyrimidin-4-amine (1.47 g, 11.3 mmol) was suspended in acetic anhydride (22.7 mL, 241 mmol) and the reaction mixture was heated at reflux for 5 h. The solvent was removed under reduced pressure. The residue was suspended in toluene, concentrated to dryness (2×) and dried under high vacuum to afford N-(6-chloropyrimidin-4-yl)acetamide (2.00 g, 98%) LC-MS (AA) ES+ 172.

### Step 3: N-[6-(trimethylstannyl)pyrimidin-4-yl]acetamide Intermediate 16

To a 500 mL round bottom flask fitted with a stir bar, reflux condenser and 3-way valve was added N-(6-chloropyrimidin-4-yl)acetamide (2.88 g, 16.8 mmol) and tetrakis(triphenylphosphine)palladium(0) (0.81 g, 0.7 mmol). The flask was flushed with argon and toluene (173 mL, 1630 mmol) was added. Hexamethylditin (3.84 mL, 18.5 mmol) was added by syringe and the resulting mixture was heated at 135° C. in an oil bath for 5 h under an atmosphere of argon. The reaction mixture was cooled to it and 12.5 g of Celite was added and the mixture was concentrated to dryness under reduced pressure. The residue was purified by silica gel chromatography (30-90% EtOAc/hexane) to afford N-[6-(trimethylstannyl)pyrimidin-4-yl]acetamide (2.9 g, 56%) LC-MS (AA) ES+ 302.

### Step 4: methyl 7-[6-(acetylamino)pyrimidin-4-yl]-5,6-dihydronaphthalene-2-carboxylate Intermediate 17

A mixture of methyl 7-{[(trifluoromethyl)sulfonyl]oxy}-5,6-dihydronaphthalene-2-carboxylate (0.169 g, 0.502 mmol), N-[6-(trimethylstannyl)pyrimidin-4-yl]acetamide (0.196 g, 0.653 mmol), 1,4-dioxane (16.9 mL), lithium chloride (31.9 mg, 0.753 mmol), copper(I) iodide (14.4 mg, 0.075 mmol) and tetrakis(triphenylphosphine)palladium(0) (29 mg, 0.025 mmol) was degassed under argon and heated at 100° C. for 3 h. The reaction mixture was cooled to it and filtered through a pad of Celite. The filtrate was concentrated under reduced pressure. The residue was purified by silica gel chromatography (0-50% of 10% MeOH DCM/hexane) to afford methyl 7-[6-(acetylamino)pyrimidin-4-yl]-5,6-dihydronaphthalene-2-carboxylate (131 mg, 81%) LC-MS (FA) ES+ 324.

### Step 5: methyl 7-[6-(acetylamino)pyrimidin-4-yl]-5,6,7,8-tetrahydronaphthalene-2-carboxylate Intermediate 18

A mixture of methyl 7-[6-(acetylamino)pyrimidin-4-yl]-5,6-dihydronaphthalene-2-carboxylate (0.131 g, 0.405 mmol), ethanol (1.94 mL), ethyl acetate (1.94 mL), THF (3 mL) and 10% Pd on carbon (43.1 mg, 0.04 mmol) was stirred at rt under an atmosphere of H₂ for 3 days. The reaction mixture was diluted with EtOAc and filtered through Celite. The filtrate was concentrated under reduced pressure. The residue was purified by silica gel chromatography (0-50% EtOAc/DCM) to give methyl 7-[6-(acetylamino)pyrimidin-4-yl]-5,6,7,8-tetrahydronaphthalene-2-carboxylate (60 mg, 45%) LC-MS (FA) ES+ 326.

### Step 6: 7-(6-Aminopyrimidin-4-yl)-N-hydroxy-5,6,7,8-tetrahydronaphthalene-2-carboxamide Compound I-98

The title compound was prepared from methyl 7-[6-(acetylamino)pyrimidin-4-yl]-5,6,7,8-tetrahydronaphthalene-2-carboxylate following the procedure outlined in Example 3, step 3. LC-MS (AA) ES+ 285; ¹H NMR (400 MHz, CD₃OD) δ ppm 8.31 (d, J=6.8 Hz, 2H), 7.51 (s, 1H), 7.49 (d, J=8.0 Hz, 1H), 7.20 (d, J=7.9 Hz, 1H), 6.46 (s, 1H), 3.11-2.98 (m, 2H), 2.98-2.91 (m, 3H), 2.19-2.11 (m, 1H), 2.02-1.89 (m, 1H).

### Example 8

### 7-[2-(Acetylamino)pyridin-4-yl]-N-hydroxy-5,6,7,8-tetrahydronaphthalene-2-carboxamide Compound I-97

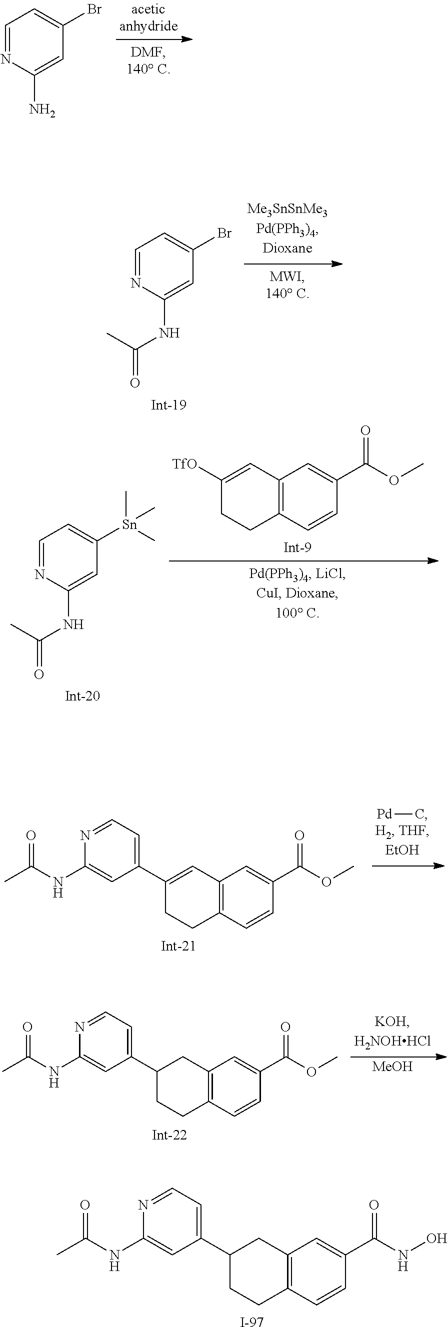

### Step 1: N-(4-bromopyridin-2-yl)acetamide Intermediate 19

To a solution of 4-bromopyridin-2-amine (0.9 g, 5.2 mmol) in acetic anhydride (18 mL, 191 mmol) was added N,N-dimethylaminopyridine (0.006 g, 0.052 mmol). The reaction mixture was stirred at 140° C. for 3 h. The reaction mixture was cooled to rt and ice water (50 mL) was added followed by conc. NH₄OH (˜60 mL) to adjust the pH to 8.5. A white solid precipitated and was isolated by filtration. This solid was washed with cold water and then hexanes. The solid was dried under high vacuum to afford N-(4-bromopyridin-2-yl)acetamide (858 mg, 77%) LC-MS (FA) ES+ 216.

### Step 2: N-[4-(trimethylstannyl)pyridin-2-yl]acetamide Intermediate 20

A mixture of N-(4-bromopyridin-2-yl)acetamide (12.6 g, 58.7 mmol), hexamethylditin (25 g, 76.3 mmol) and tetrakis(triphenylphosphine)palladium(0) (3.39 g, 2.93 mmol) in 1,4-dioxane (300 mL) was heated at 95° C. for 3 h. The solvents were removed under reduced pressure. DCM was added and the resulting black precipitate was removed by filtration and washed with DCM. The filtrate was evaporated. The residue was purified by silica gel chromatography (25-40% EtOAc/hexanes) to afford N-[4-(trimethylstannyl)pyridin-2-yl]acetamide (12.6 g, 72%). ¹H NMR (400 MHz, CDCl₃) δ ppm 8.33 (s, 1H), 8.22 (s, 1H), 8.17 (dd, J=4.7, 0.9 Hz, 1H), 7.14 (dd, J=4.7, 0.6 Hz, 1H), 2.20 (s, 3H), 0.34 (s, 9H).

### Step 3: 7-[2-(acetylamino)pyridin-4-yl]-N-hydroxy-5,6,7,8-tetrahydronaphthalene-2-carboxamide Compound I-97

The title compound was prepared from 7-[2-(acetylamino)pyridin-4-yl]-5,6,7,8-tetrahydronaphthalene-2-carboxylate following the procedures outlined in Example 7, Steps 4-6. LC-MS (FA) ES+ 326; ¹H NMR (400 MHz, CD₃OD) δ ppm 8.20 (d, J=5.1 Hz, 1H), 8.05 (s, 1H), 7.49 (d, J=6.7 Hz, 2H), 7.20 (d, J=8.5 Hz, 1H), 7.08 (d, J=4.3 Hz, 1H), 3.14-2.92 (m, 5H), 2.20-2.12 (m, 4H), 2.03-1.90 (m, 1H).

### Example 9

### N-hydroxy-6-(pyridin-4-yloxy)-5,6,7,8-tetrahydronaphthalene-2-carboxamide Compound I-108

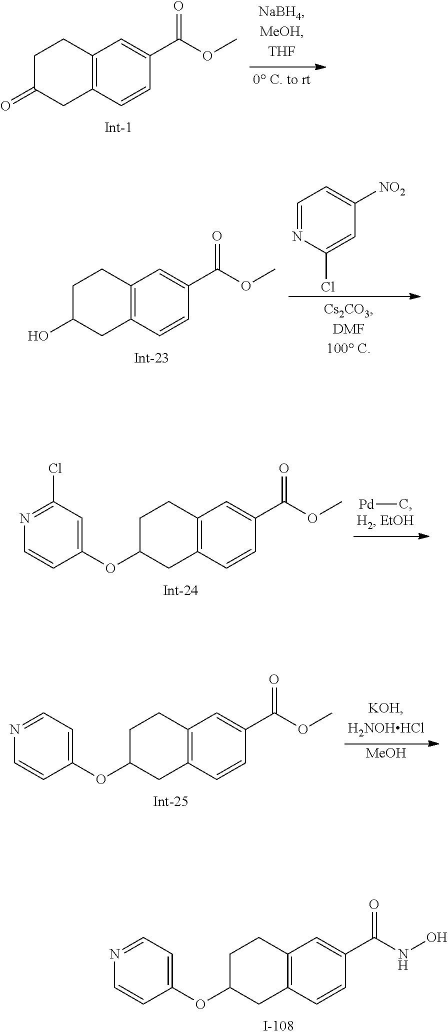

### Step 1: methyl 6-hydroxy-5,6,7,8-tetrahydronaphthalene-2-carboxylate Intermediate 23

A solution of methyl 6-oxo-5,6,7,8-tetrahydronaphthalene-2-carboxylate (300 mg, 1.47 mmol) in methanol (5 mL) and THF (5 mL) was cooled to 0° C. and sodium borohydride (33.3 mg, 0.89 mmol) was added portion-wise. The reaction mixture was stirred at 0° C. for 30 min, and then allowed to warm to it. After 90 min, the reaction mixture was neutralized with a few drops of 1N HCl solution and extracted with Et₂O (2×). The organic phases were washed with water (2×) and brine, dried over Na₂SO₄ and evaporated. The residue was purified by silica gel chromatography (12% to 50% EtOAc/hexane) to afford methyl 6-hydroxy-5,6,7,8-tetrahydronaphthalene-2-carboxylate (275 mg, 91%). ¹H NMR (400 MHz, CDCl₃) δ ppm 7.81-7.74 (m, 2H), 7.15 (d, J=7.9 Hz, 1H), 4.21 (dq, J=8.1, 4.8 Hz, 1H), 3.90 (s, 3H), 3.13 (dd, J=16.8, 4.8 Hz, 1H), 3.02 (td, J=16.8, 6.0 Hz, 1H), 2.85 (ddd, J=24.5, 13.0, 7.0 Hz, 2H), 2.14-2.01 (m, 1H), 1.91-1.80 (m, 1H), 1.58 (d, J=4.5 Hz, 1H).

### Step 2: methyl 6-[(2-chloropyridin-4-yl)oxy]-5,6,7,8-tetrahydronaphthalene-2-carboxylate Intermediate 24

Methyl 6-hydroxy-5,6,7,8-tetrahydronaphthalene-2-carboxylate (178 mg, 0.86 mmol), 2-chloro-4-nitropyridine (274 mg 1.73 mmol), cesium carbonate (844 mg, 2.59 mmol) and DMF (8 mL) were combined in a microwave vial. The vial was sealed and heated to 100° C. in an oil bath. After 2 hours a further portion of 2-chloro-4-nitropyridine (136 mg, 0.86 mmol) was added and heating continued overnight. The reaction mixture was cooled to rt, water was added and the mixture was extracted with EtOAc. The organic phase was washed with water (2×) and then with brine, dried over Na₂SO₄ and evaporated. The residue was purified by silica gel chromatography (5% to 20% EtOAc/hexane) to afford methyl 6-[(2-chloropyridin-4-yl)oxy]-5,6,7,8-tetrahydronaphthalene-2-carboxylate (126 mg, 56%). ¹H NMR (400 MHz, CDCl₃) δ ppm 8.20 (d, J=5.8 Hz, 1H), 7.86-7.75 (m, 2H), 7.16 (d, J=7.9 Hz, 1H), 6.86 (d, J=2.2 Hz, 1H), 6.76 (dd, J=5.8, 2.3 Hz, 1H), 4.92-4.80 (m, 1H), 3.91 (s, 3H), 3.25 (dd, J=17.2, 4.7 Hz, 1H), 3.11-3.01 (m, 2H), 2.91 (td, J=17.0, 6.7 Hz, 1H), 2.23-2.09 (m, 2H).

### Step 3: methyl 6-(pyridin-4-yloxy)-5,6,7,8-tetrahydronaphthalene-2-carboxylate Intermediate 25

A mixture of methyl 6-[(2-chloropyridin-4-yl)oxy]-5,6,7,8-tetrahydronaphthalene-2-carboxylate (126 mg, 0.4 mmol) and 10% palladium on carbon (10 mg) in ethanol (4 mL) was hydrogenated at atmospheric pressure (hydrogen balloon) over 48 h. The mixture was diluted with EtOAc and filtered through Celite. The filtrate was evaporated and purified by silica gel chromatography (2% to 8% MeOH/DCM) to afford methyl 6-(pyridin-4-yloxy)-5,6,7,8-tetrahydronaphthalene-2-carboxylate (63 mg, 56%). ¹H NMR (400 MHz, CDCl₃) δ ppm 8.48-8.38 (m, 2H), 7.87-7.74 (m, 2H), 7.16 (d, J=8.0 Hz, 1H), 6.86-6.79 (m, 2H), 4.92-4.84 (m, 1H), 3.91 (s, 3H), 3.26 (dd, J=17.0, 4.8 Hz, 1H), 3.12-3.00 (m, 2H), 2.95-2.85 (m, 1H), 2.25-2.05 (m, 2H).

### Step 4: N-hydroxy-6-(pyridin-4-yloxy)-5,6,7,8-tetrahydronaphthalene-2-carboxamide Compound I-108

The title compound was prepared from methyl 6-(pyridin-4-yloxy)-5,6,7,8-tetrahydronaphthalene-2-carboxylate following the procedure outlined in Example 3, step 3 (24%). LC-MS: (AA) ES+ 285; ¹H NMR (400 MHz, CD₃OD) δ ppm 8.37-8.29 (m, 2H), 7.52 (s, 1H), 7.49 (d, J=7.9 Hz, 1H), 7.18 (d, J=7.9 Hz, 1H), 7.03-7.01 (m, 2H), 5.08-5.01 (m, 1H), 3.08-2.88 (m, 4H), 2.23-2.07 (m, 2H).

### Example 10

### 7-(4-chlorophenoxy)-N-hydroxy-5,6,7,8-tetrahydronaphthalene-2-carboxamide I-91

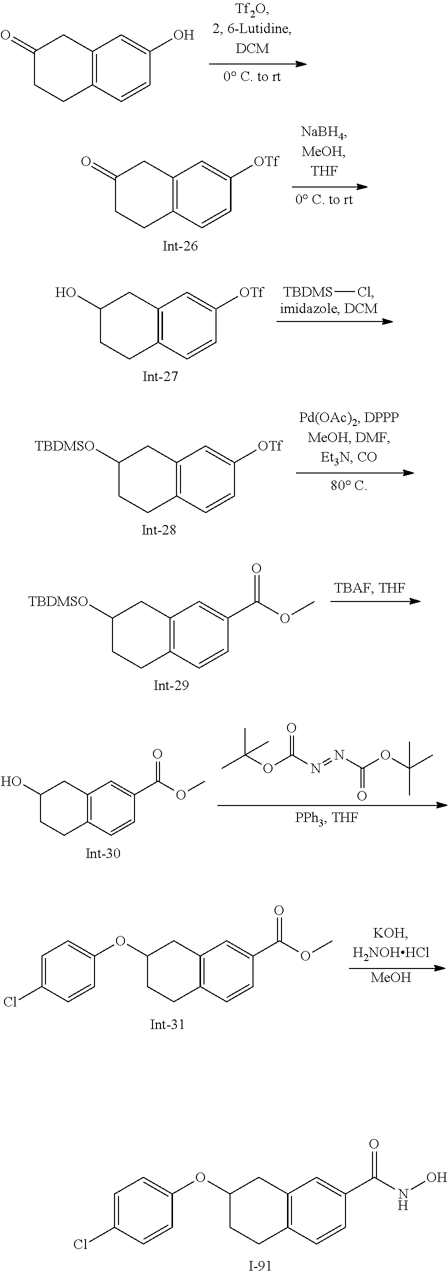

### Step 1: 7-oxo-5,6,7,8-tetrahydronaphthalen-2-yl trifluoromethanesulfonate Intermediate 26

To a solution of 7-hydroxy-3,4-dihydronaphthalen-2(1H)-one (3.82 g, 23.6 mmol) in DCM (100 mL) and 2,6-lutidine (3.27 mL, 28.3 mmol) at 0° C. was added trifluoromethanesulfonic anhydride (4.76 mL, 28.3 mmol) drop-wise. The reaction mixture was stirred at 0° C. for 30 min, and then allowed to warm to rt. After 30 min, the reaction mixture was diluted with DCM and washed with 1N HCl and then brine. The organic phase was dried over Na₂SO₄ and evaporated. The residue was purified by silica gel chromatography (7% to 30% EtOAc/hexane) to afford 7-oxo-5,6,7,8-tetrahydronaphthalen-2-yl trifluoromethanesulfonate (5.96 g, 86%). ¹H NMR (400 MHz, CDCl₃) δ ppm 7.32 (d, J=8.3 Hz, 1H), 7.14 (dd, J=8.3, 2.6 Hz, 1H), 7.06 (d, J=2.5 Hz, 1H), 3.62 (s, 2H), 3.10 (t, J=6.7 Hz, 2H), 2.60-2.56 (m, 2H).

### Step 2: 7-hydroxy-5,6,7,8-tetrahydronaphthalen-2-yl trifluoromethanesulfonate Intermediate 27

The title compound was prepared from methyl 7-oxo-5,6,7,8-tetrahydronaphthalen-2-yl trifluoromethanesulfonate following the procedure outlined in Example 9, step 1. ¹H NMR (400 MHz, CDCl₃) δ ppm 7.16 (d, J=8.4 Hz, 1H), 7.05-6.97 (m, 2H), 4.21 (dq, J=8.1, 8.1, 7.8, 4.8 Hz, 1H), 3.10 (dd, J=16.8, 4.8 Hz, 1H), 2.99 (td, J=17.2, 6.0 Hz, 1H), 2.88-2.75 (m, 2H), 2.10-1.99 (m, 1H), 1.87 (dtd, J=14.3, 8.6, 5.8 Hz, 1H), 1.58 (d, J=4.3 Hz, 1H).

### Step 3: 7-{[tert-butyl(dimethyl)silyl]oxy}-5,6,7,8-tetrahydronaphthalen-2-yl trifluoromethanesulfonate Intermediate 28

To a solution of 7-hydroxy-5,6,7,8-tetrahydronaphthalen-2-yl trifluoromethanesulfonate (2.23 g, 7.53 mmol) in DCM (35 mL), was added 1H-imidazole (769 mg, 11.3 mmol) and tert-butyldimethylsilyl chloride (1.7 g, 11.3 mmol). The reaction mixture was stirred at rt overnight. The mixture was diluted with DCM and washed with water and then brine. The organic phase was dried over Na₂SO₄ and evaporated. The residue was purified by silica gel chromatography (0% to 5% EtOAc/hexane) to afford 7-{[tert-butyl(dimethyl)silyl]oxy}-5,6,7,8-tetrahydronaphthalen-2-yl trifluoromethanesulfonate (2.40 g, 78%). ¹H NMR (400 MHz, CDCl₃) δ ppm 7.13 (d, J=8.4 Hz, 1H), 7.01-6.94 (m, 2H), 4.16-4.08 (m, 1H), 3.01-2.91 (m, 2H), 2.81-2.71 (m, 2H), 1.98-1.88 (m, 1H), 1.86-1.75 (m, 1H), 0.88 (s, 9H), 0.10 (s, 3H), 0.09 (s, 3H).

### Step 4: methyl 7-{[tert-butyl(dimethyl)silyl]oxy}-5,6,7,8-tetrahydronaphthalene-2-carboxylate Intermediate 29

A solution of 7-{[tert-butyl(dimethyl)silyl]oxy}-5,6,7,8-tetrahydronaphthalen-2-yl trifluoromethanesulfonate (2.4 g, 5.85 mmol) in DMF (23 mL) and methanol (35 mL) was degassed with nitrogen. Palladium(II) acetate (328 mg, 1.46 mmol), 1,3-bis(diphenylphosphino)propane (603 mg, 1.46 mmol) and triethylamine (8.15 mL, 58.5 mmol) were added. The solution was degassed again and CO gas was bubbled through the solution for approximately 2 min. The reaction flask was fitted with a condenser and a CO balloon and the reaction mixture was heated at 80° C. for 2 h, the methanol was evaporated and water was added. The mixture was extracted with EtOAc and the organic phase was washed with water (2 x) and then brine, dried over Na₂SO₄ and evaporated. The residue was purified twice by silica gel chromatography (0% to 8% EtOAc/hexane, then 2% to 8% EtOAc/hexane) to afford methyl 7-{[tert-butyl)dimethyl)silyl]oxy}-5,6,7,8-tetrahydronaphthalene-2-carboxylate (929 mg, 50%). ¹H NMR (400 MHz, CDCl₃) δ ppm 7.77-7.72 (m, 2H), 7.13 (d, J=8.5 Hz, 1H), 4.17-4.06 (m, 1H), 3.89 (s, 3H), 3.05-2.94 (m, 2H), 2.86-2.73 (m, 2H), 2.02-1.90 (m, 1H), 1.86-1.74 (m, 1H), 0.89 (s, 9H), 0.09 (s, 3H), 0.09 (s, 3H).

### Step 5: methyl 7-hydroxy-5,6,7,8-tetrahydronaphthalene-2-carboxylate Intermediate 30

To a solution of methyl 7-{[tert-butyl(dimethyl)silyl]oxy}-5,6,7,8-tetrahydronaphthalene-2-carboxylate (162 mg, 0.51 mmol) in THF (5 mL), was added tetra-n-butylammonium fluoride (1.0M in THF, 0.61 mL, 0.61 mmol) drop-wise. The reaction mixture was stirred at rt overnight. The solvents were evaporated and the residue was purified by silica gel chromatography (12% to 50% EtOAc/hexane) to afford methyl 7-hydroxy-5,6,7,8-tetrahydronaphthalene-2-carboxylate (85 mg, 82%). ¹H NMR (400 MHz, CDCl₃) δ ppm 7.79-7.76 (m, 2H), 7.16 (d, J=8.5 Hz, 1H), 4.25-4.17 (m, 1H), 3.90 (s, 3H), 3.13 (dd, J=16.3, 5.0 Hz, 1H), 3.02 (td, J=17.4, 5.9 Hz, 1H), 2.92-2.76 (m, 2H), 2.12-2.02 (m, 1H), 1.92-1.81 (m, 1H), 1.59 (d, J=4.4 Hz, 1H).

### Step 6: methyl 7-(4-chlorophenoxy)-5,6,7,8-tetrahydronaphthalene-2-carboxylate Intermediate 31

To a mixture of methyl 7-hydroxy-5,6,7,8-tetrahydronaphthalene-2-carboxylate (100 mg, 0.48 mmol), 4-chlorophenol (102 mg, 0.8 mmol) and triphenylphosphine (209 mg, 0.8 mmol) in THF (2.5 mL) was added a solution of di-tert-butyl azodicarboxylate (184.2 mg, 0.8 mmol) in THF (1 mL) at 0° C. The reaction mixture was allowed to warm to rt and stirred for 2 h. The solvents were evaporated and the residue was purified by silica gel chromatography (0% to 10% EtOAc/hexane) to afford methyl 7-(4-chlorophenoxy)-5,6,7,8-tetrahydronaphthalene-2-carboxylate (50 mg, 32%). ¹H NMR (400 MHz, CDCl₃) δ ppm 7.82-7.77 (m, 2H), 7.26-7.20 (m, 2H), 7.18 (d, J=8.0 Hz, 1H), 6.88-6.83 (m, 2H), 4.76-4.68 (m, 1H), 3.90 (s, 3H), 3.20 (dd, J=16.8, 4.7 Hz, 1H), 3.11-2.99 (m, 2H), 2.92-2.81 (m, 1H), 2.15-2.02 (m, 2H).

### Step 7: 7-(4-chlorophenoxy)-N-hydroxy-5,6,7,8-tetrahydronaphthalene-2-carboxamide Compound I-91

The title compound was prepared from methyl 7-(4-chlorophenoxy)-5,6,7,8-tetrahydronaphthalene-2-carboxylate following the procedure outlined in Example 3, step 3 (58%). LC-MS: (AA) ES+ 318; ¹H NMR (400 MHz, d₆-DMSO) δ ppm 7.51-7.47 (m, 2H), 7.34-7.29 (m, 2H), 7.17 (d, J=8.2 Hz, 1H), 7.03-6.99 (m, 2H), 4.89-4.82 (m, 1H), 3.17 (dd, J=16.5, 4.2 Hz, 1H), 2.93-2.80 (m, 3H), 2.10-2.01 (m, 1H), 1.98-1.89 (m, 1H).

### Example 11

### N-hydroxy-7-phenoxy-5,6,7,8-tetrahydronaphthalene-2-carboxamide Compound I-87

The title compound was prepared following the procedures outlined in Example 10, substituting phenol for 4-chlorophenol. LC-MS: (AA) ES+ 284; ¹H NMR (400 MHz, d₆-DMSO) δ ppm 7.51-7.47 (m, 2H), 7.31-7.25 (m, 2H), 7.17 (d, J=8.5 Hz, 1H), 6.99-6.89 (m, 3H), 4.90-4.82 (m, 1H), 3.17 (dd, J=16.8, 4.6 Hz, 1H), 2.95-2.76 (m, 3H), 2.11-2.02 (m, 1H), 1.98-1.89 (m, 1H).

### Example 12

### N-hydroxy-7-(pyridin-4-yloxy)-5,6,7,8-tetrahydronaphthalene-2-carboxamide Compound I-86

The title compound was prepared from methyl 7-hydroxy-5,6,7,8-tetrahydronaphthalene-2-carboxylate following the procedures outlined in Example 9. LC-MS: (AA) ES+ 285; ¹H NMR (400 MHz, d₆-DMSO) δ ppm 8.38-8.35 (m, 2H), 7.51-7.48 (m, 2H), 7.16 (d, J=8.6 Hz, 1H), 7.03-6.99 (m, 2H), 5.04-4.97 (m, 1H), 3.21 (dd, J=16.6, 4.7 Hz, 1H), 2.96-2.84 (m, 3H), 2.15-2.04 (m, 1H), 2.02-1.91 (m, 1H).

### Example 13

### 7-[(4-chlorobenzyl)oxy]-N-hydroxy-5,6,7,8-tetrahydronaphthalene-2-carboxamide Compound I-117

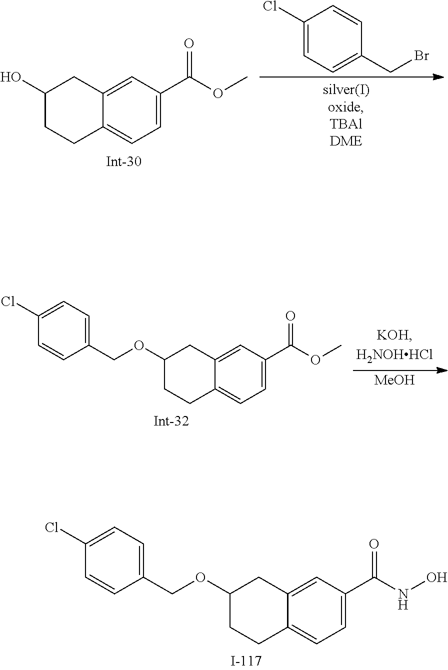 [SMILES: "MeOH" ; COC(=O)c1ccc2c(c1)CC(O)CC2 ; Clc1ccc(CBr)cc1 ; COC(=O)c1ccc2c(c1)CC(OCc1ccc(Cl)cc1)CC2 ; O=C(NO)c1ccc2c(c1)CC(OCc1ccc(Cl)cc1)CC2]

### Step 1: methyl 7-[(4-chlorobenzyl)oxy]-5,6,7,8-tetrahydronaphthalene-2-carboxylate Intermediate 32

To a solution of methyl 7-hydroxy-5,6,7,8-tetrahydronaphthalene-2-carboxylate (100 mg, 0.48 mmol) and 4-chlorobenzyl bromide (299 mg, 1.45 mmol) in 1,2-dimethoxyethane (10 mL) was added tetra-n-butylammonium iodide (358 mg, 0.97 mmol) and silver(I) oxide (337 mg, 1.45 mmol). The reaction mixture was stirred at rt overnight. The solid was removed by filtration and the resulting filtrate was diluted with ethyl acetate. The organic phases were washed with 10% sodium thiosulfate solution, water, and brine, dried (Na₂SO₄) and evaporated. The residue was purified by silica gel chromatography (0% to 10% EtOAc/hexane) to afford methyl 7-[(4-chlorobenzyl)oxy]-5,6,7,8-tetrahydronaphthalene-2-carboxylate (98 mg, 61%). ¹H NMR (400 MHz, CDCl₃) δ ppm 7.79-7.74 (m, 2H), 7.33-7.27 (m, 4H), 7.15 (d, J=8.5 Hz, 1H), 4.60 (dd, J=15.8, 12.3 Hz, 2H), 3.92-3.84 (m, 4H), 3.11 (dd, J=16.6, 4.5 Hz, 1H), 3.06-2.97 (m, 1H), 2.91 (dd, J=16.6, 7.2 Hz, 1H), 2.86-2.77 (m, 1H), 2.13-2.05 (m, 1H), 1.98-1.88 (m, 1H).

### Step 2: 7-[(4-chlorobenzyl)oxy]-N-hydroxy-5,6,7,8-tetrahydronaphthalene-2-carboxamide Compound I-117

The title compound was prepared from methyl 7-[(4-chlorobenzyl)oxy]-5,6,7,8-tetrahydronaphthalene-2-carboxylate following the procedure outlined in Example 3, step 3 (61%). LC-MS: (AA) ES+ 332; ¹H NMR (400 MHz, CDCl₃) δ ppm 7.48-7.40 (m, 2H), 7.32-7.27 (m, 4H), 7.14 (d, J=8.0 Hz, 1H), 4.58 (dd, J=18.6, 12.3 Hz, 2H), 3.91-3.84 (m, 1H), 3.11-2.95 (m, 2H), 2.86 (dd, J=16.7, 7.1 Hz, 1H), 2.84-2.74 (m, 1H), 2.11-2.01 (m, 1H), 1.98-1.89 (m, 1H).

### Example 14

### 7-ethoxy-N-hydroxy-5,6,7,8-tetrahydronaphthalene-2-carboxamide Compound I-118

The title compound was prepared following the procedures outlined in Example 13, substituting iodoethane for 4-chlorobenzyl bromide. LC-MS: (AA) ES+ 236; ¹H NMR (400 MHz, CDCl₃) δ ppm 7.42-7.36 (m, 2H), 7.03 (d, J=7.7 Hz, 1H), 3.76-3.68 (m, 1H), 3.63-3.50 (m, 2H), 2.98 (dd, J=16.6, 4.8 Hz, 1H), 2.94-2.85 (m, 1H), 2.78-2.67 (m, 2H), 2.04-1.95 (m, 1H), 1.86-1.75 (m, 1H), 1.20 (t, J=7.1 Hz, 3H).

### Example 15

### methyl 6-amino-5,6,7,8-tetrahydronaphthalene-2-carboxylate hydrochloride Intermediate 34

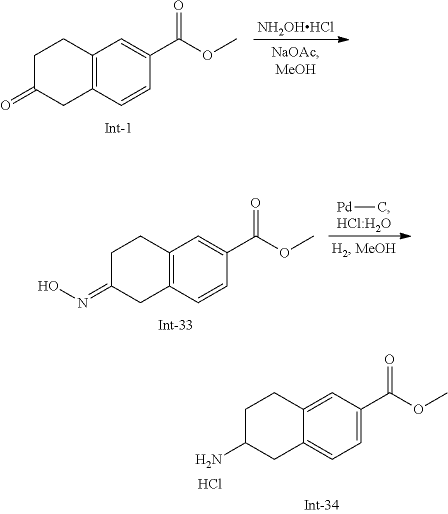

### Step 1: Methyl 6-(hydroxyimino)-5,6,7,8-tetrahydronaphthalene-2-carboxylate Intermediate 33

A mixture of methyl 6-oxo-5,6,7,8-tetrahydronaphthalene-2-carboxylate (1.92 g, 9.4 mmol), hydroxylamine hydrochloride (3.92 g, 56.4 mmol), and sodium acetate (4.63 g, 56.4 mmol) in MeOH (90 mL) was heated to 50° C. for 2 h. The solution was then concentrated and EtOAc (250 mL) and water (50 mL) were added. After separation, the organic phase was washed with water (50 mL) and the combined aqueous phases were extracted with EtOAc (3×250 mL). The combined organic phases were washed with brine (50 mL), dried (MgSO₄), and concentrated. Purification by silica gel chromatography (EtOAc: hexanes, 1:9 to 1:4) yielded methyl 6-(hydroxyimino)-5,6,7,8-tetrahydronaphthalene-2-carboxylate (0.972 g, 47.2%). LC-MS: (FA) ES+ 220.

### Step 2: methyl 6-amino-5,6,7,8-tetrahydronaphthalene-2-carboxylate hydrochloride Intermediate 34

Methyl 6-(hydroxyimino)-5,6,7,8-tetrahydronaphthalene-2-carboxylate was dissolved in MeOH (100 mL) and the solution was degassed with nitrogen. Palladium on carbon (0.301 g, 10 wt. %) and hydrochloric acid (6.27 mL, 75.2 mmol, 12.0 M in water) were quickly added to the solution. The mixture was purged with H₂ twice and then stirred under 1 atm of H₂ gas for 16 h at rt. The suspension was then filtered through a pad of Celite and concentrated to give methyl 6-amino-5,6,7,8-tetrahydronaphthalene-2-carboxylate hydrochloride (1.01 g, 44.4%). LC-MS: (FA) ES+ 206; ¹H NMR (400 MHz, CD₃OD) δ ppm 7.81 (s, 1H), 7.78 (d, J=8.0 Hz, 1H), 7.23 (d, J=8.0 Hz, 1H), 3.87 (s, 3 H), 3.58 (m, 1H), 3.25 (dd, J=16.0, 5.3 Hz, 1H), 3.00 (m, 2H), 2.88 (dd, J=16.6, 9.8 Hz, 1H), 2.25 (m, 1H), 1.86 (m, 1H).

### Example 16

### methyl 7-amino-5,6,7,8-tetrahydronaphthalene-2-carboxylate. HCl Intermediate 42

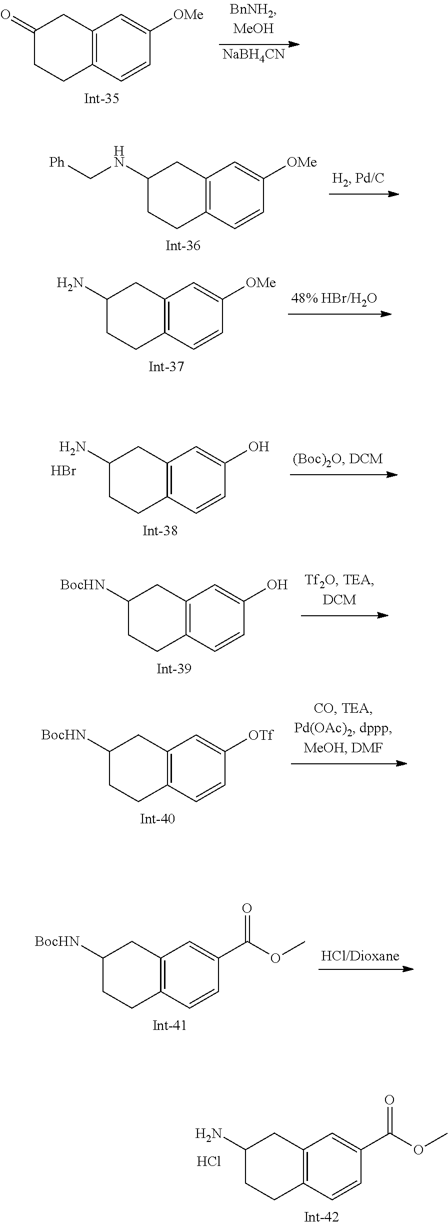

### Step 1: N-benzyl-7-methoxy-1,2,3,4-tetrahydronaphthalen-2-amine Intermediate 36

Into a round bottom flask was added 7-methoxy-3,4-dihydronaphthalen-2(1H)-one (5.75 g, 32.6 mmol), benzylamine (3.56 mL, 32.6 mmol), methanol (200 mL) and acetic acid (3.71 mL, 65.2 mmol). The reaction mixture was stirred at rt for 30 minutes and then cooled to 0° C. Sodium cyanoborohydride (3.07 g, 48.9 mmol) was carefully and the reaction mixture was allowed to warm to rt and stirred overnight. Approximately ⅔ of the solvent was evaporated and sat. NaHCO₃ solution was added. The aqueous phase was extracted with EtOAc (2×). The combined organic phases were washed with brine, dried over anhydrous sodium sulfate, filtered and concentrated. The residue was purified by silica gel chromatography (0-5% MeOH/DCM) to afford N-benzyl-7-methoxy-1,2,3,4-tetrahydronaphthalen-2-amine (8.48 g, 97%). LC-MS (FA) ES+ 268.

### Step 2: 7-methoxy-1,2,3,4-tetrahydronaphthalen-2-amine Intermediate 37

A mixture of N-benzyl-7-methoxy-1,2,3,4-tetrahydronaphthalen-2-amine (7.23 g, 27 mmol), ethanol (90 mL), acetic acid (24 mL) and palladium hydroxide (0.9 g, 6.4 mmol) was stirred at rt under an atmosphere of H₂ for 4 days. The reaction mixture was filtered through a pad of Celite. The solids were washed with EtOAc several times and the filtrate was concentrated. The crude compound was purified by silica gel chromatography (0-5% MeOH/DCM and 20-100% of 1% NH₄OH/10% MeOH/DCM in DCM) to give 7-methoxy-1,2,3,4-tetrahydronaphthalen-2-amine (3.93 g, 82%). LC-MS (AA) ES+ 178.

### Step 3: 7-amino-5,6,7,8-tetrahydronaphthalen-2-ol.HBr Intermediate 38

A mixture of 7-methoxy-1,2,3,4-tetrahydronaphthalen-2-amine (3.93 g, 22.2 mmol) in 48% HBr/water (20 mL) and acetic acid (10 mL) was heated at 110° C. overnight. The reaction mixture was cooled to rt and the solvent was removed. The residue was suspended in toluene, concentrated and dried under high vacuum to afford 7-amino-5,6,7,8-tetrahydronaphthalen-2-ol.HBr (5.39 g, 99%) LC-MS (AA) ES+ 164.

### Step 4: tert-butyl (7-hydroxy-1,2,3,4-tetrahydronaphthalen-2-yl)carbamate Intermediate 39

To a mixture of 7-amino-5,6,7,8-tetrahydronaphthalen-2-ol.HBr (1.01 g, 4.14 mmol) in DMF (4.8 mL) was added TEA (2.02 mL, 14.4 mmol) followed by di-tert-butyldicarbonate (1.17 g, 5.38 mmol) and the reaction mixture was stirred for 6 h. Water was added and the mixture was extracted with EtOAc (2×). The organic phases were washed with brine, dried over anhydrous sodium sulfate, filtered and concentrated. The residue was purified by silica gel chromatography (0-50% EtOAc/Hexanes) to give tert-butyl (7-hydroxy-1,2,3,4-tetrahydronaphthalen-2-yl)carbamate (930 mg, 85%). LC-MS (FA) ES+ 264.

### Step 5: 7-[(tert-butoxycarbonyl)amino]-5,6,7,8-tetrahydronaphthalen-2-yl trifluoromethanesulfonate Intermediate 40

Into a solution of tert-butyl (7-hydroxy-1,2,3,4-tetrahydronaphthalen-2-yl)carbamate (1.1 g, 4.18 mmol) in DCM (20 mL) was added triethylamine (1.75 mL, 10.2 mmol). The resulting reaction mixture was cooled to 0° C. then trifluoromethanesulfonic anhydride (0.84 mL, 5 mmol) was added drop-wise. The reaction mixture was stirred at 0° C. for 1 h and rt for 2 h. The mixture was poured into ice and extracted with DCM (2×). The extracts were washed with sat.NaHCO₃ solution and brine, dried over anhydrous sodium sulfate, filtered and concentrated. The residue was purified by silica gel chromatography (20-50% EtOAc/hexanes) to afford 7-[(tert-butoxycarbonyl)amino]-5,6,7,8-tetrahydronaphthalen-2-yl trifluoromethanesulfonate (1.46 g, 88%). ¹H NMR (400 MHz, CDCl₃) δ ppm 7.15 (d, J=8.5 Hz, 1H), 7.02 (dd, J=8.5, 2.6 Hz, 1H), 6.97 (d, J=2.4 Hz, 1H), 4.58 (s, 1H), 3.96 (s, 1H), 3.14 (dd, J=16.5, 4.7 Hz, 1H), 2.88 (dd, J=12.5, 6.0 Hz, 2H), 2.65 (dd, J=16.6, 8.5 Hz, 1H), 2.12-2.04 (m, 1H), 1.80-1.68 (m, 1H), 1.46 (s, 9H).

### Step 6: methyl 7-[(tert-butoxycarbonyl)amino]-5,6,7,8-tetrahydronaphthalene-2-carboxylate Intermediate 41

A mixture of 7-[(tert-butoxycarbonyl)amino]-5,6,7,8-tetrahydronaphthalen-2-yl trifluoromethanesulfonate (0.992 g, 2.51 mmol), triethylamine (3.497 mL, 25.09 mmol), methanol (15 mL), and DMF (10 mL) was degassed under nitrogen for 15 min. To the solution was added 1,3-bis(diphenylphosphino)propane (0.259 g, 0.627 mmol) and palladium (II) acetate (0.141 g, 0.627 mmol). CO was bubbled through the solution for 5 min and the mixture was heated at 80° C. overnight under an atmosphere of carbon monoxide. The solvents were evaporated and suspended in EtOAc. Water was added and the mixture was extracted with EtOAc (2×). The organic phases were washed with brine, dried over anhydrous sodium sulfate, filtered and concentrated. The residue was purified by silica gel chromatography (0-30% EtOAc/hexanes) to give methyl 7-[(tert-butoxycarbonyl)amino]-5,6,7,8-tetrahydronaphthalene-2-carboxylate (420 mg, 55%). ¹H NMR (300 MHz, CDCl₃) δ ppm 7.77 (d, J=8.4 Hz, 2H), 7.15 (d, J=7.8 Hz, 1H), 4.56 (s, 1H), 3.98 (s, 1H), 3.89 (s, 3H), 3.14 (dd, J=16.4, 5.0 Hz, 1H), 2.91 (t, J=6.6 Hz, 2H), 2.66 (dd, J=16.4, 8.4 Hz, 1H), 2.16-2.04 (m, 1H), 1.82-1.65 (m, 1H), 1.46 (s, 9H).

### Step 7: methyl 7-amino-5,6,7,8-tetrahydronaphthalene-2-carboxylate.HCl Intermediate 42

Methyl 7-[(tert-butoxycarbonyl)amino]-5,6,7,8-tetrahydronaphthalene-2-carboxylate (0.42 g, 1.38 mmol) was dissolved in DCM (6 mL) and 4.0 M hydrochloric acid in 1,4-dioxane (3 mL, 12 mmol) was added. The reaction mixture was stirred at rt for 4 h. After 4 h, the reaction mixture was concentrated and dried under vacuum to afford methyl 7-amino-5,6,7,8-tetrahydronaphthalene-2-carboxylate.HCl (326 mg, 98%). LC-MS (FA) ES+ 206; ¹H NMR (300 MHz, d₆-DMSO) δ ppm 8.34 (s, 1H), 7.71 (d, J=7.5 Hz, 1H), 7.25 (d, J=8.1 Hz, 1H), 3.82 (s, 3H), 3.52-3.39 (m, 1H), 3.16 (dd, J=16.4, 5.0 Hz, 1H), 3.00-2.80 (m, 3H), 2.14 (d, J=14.6 Hz, 1H), 1.86-1.70 (m, 1H).

### Example 17

### methyl (7R)-7-amino-5,6,7,8-tetrahydronaphthalene-2-carboxylate.HCl Intermediate 48a

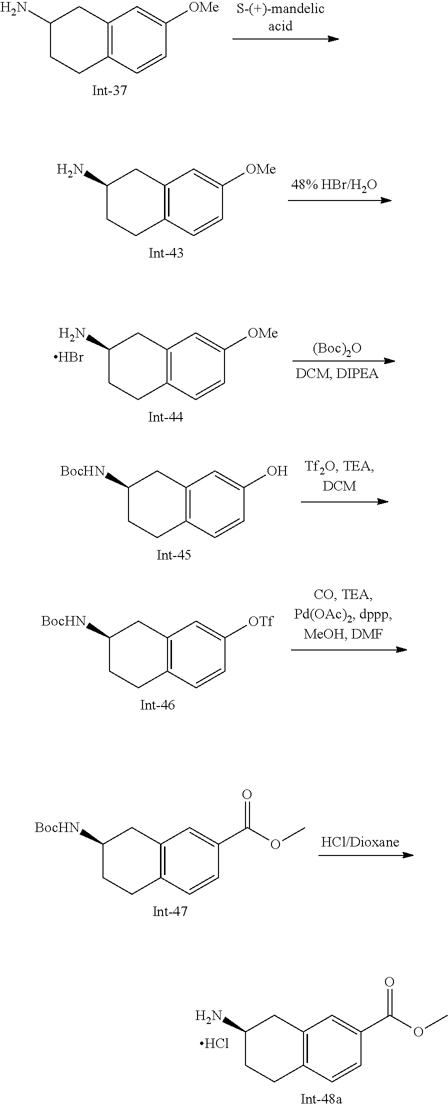

### Step 1: (2R)-7-methoxy-1,2,3,4-tetrahydronaphthalen-2-amine Intermediate 43

To a stirred solution of S-(+)-mandelic acid (15.4 g, 101 mmol), isopropyl alcohol (78 mL) and 80/20 methanol/water (51 mL) was added a solution of 7-methoxy-1,2,3,4-tetrahydronaphthalen-2-amine (17.7 g, 109 mmol) in toluene (10 mL) and 80/20 methanol/water (40 mL) via a dropping funnel. After addition was completed, the mixture was stirred at reflux for 30 min. The mixture was then cooled to rt. The mixture was allowed to stand at rt over the weekend. The resulting solids (16.95 g) were isolated by filtration, washed with minimal ethyl acetate and dried in vacuo. The salt was then suspended in an 80/20 methanol/water solution (55 mL) and warmed to reflux. Additional 80/20 methanol/water solution was added until the solution became homogeneous (about 10 mL). Upon complete dissolution, the solution was stirred at reflux 30 min, cooled to rt and allowed to stand undisturbed overnight. The resulting white solids which precipitated were collected by suction filtration (11.94 g) and dried in vacuo. The solids were recrystallized as above from 80/20 methanol/water (ca. 60 mL) to afford 10.05 g of the S-(+)-mandelate salt (Cecchi et al. Eur. J. Med. Chem. 1994, 29:259) ([α]=+90°, c=0.5, MeOH). The salt was partitioned between 4.00 M of sodium hydroxide in water (30 mL) and ethyl acetate (100 mL). The phases were separated and the aqueous phase was extracted with ethyl acetate (2×100 mL). The extracts were combined, washed with brine (35 mL), dried over sodium sulfate, filtered and concentrated to afford (2R)-7-methoxy-1,2,3,4-tetrahydronaphthalen-2-amine (5.39 g, 60% of theoretical) as an oil. LC-MS: (AA) ES+ 178; ¹H NMR (300 MHz, CDCl₃) δ ppm 7.00 (d, J=8.4 Hz, 1H), 6.69 (dd, J=8.4, 2.6 Hz, 1H), 6.64-6.57 (m, 1H), 3.21 (ddt, J=9.8, 5.0, 3.2 Hz, 1H), 2.99 (dd, J=16.1, 4.7 Hz, 1H), 2.91-2.69 (m, 2H), 2.60 (dd, J=16.1, 9.5 Hz, 1H), 2.41-2.33 (m, 2H), 2.10-1.95 (m, 1H), 1.62 (dtd, J=12.6, 10.1, 6.7 Hz, 1H).

### Step 2: (7R)-7-amino-5,6,7,8-tetrahydronaphthalen-2-ol.HBr Intermediate 44

A suspension of (2R)-7-methoxy-1,2,3,4-tetrahydronaphthalen-2-amine (5.92 g, 33.7 mmol) in hydrobromic acid (48% in water, 80 mL) was warmed to reflux. After 1.75 h, the reaction solution was cooled to P. The solvent was removed under reduced pressure. The oily residue was twice dissolved in ethanol (100 mL) and concentrated to dryness. The resulting oil was further dried in vacuo, affording (7R)-7-amino-5,6,7,8-tetrahydronaphthalen-2-ol hydrobromide (Cecchi et al. Eur. J. Med. Chem. 1994, 29:259) as a brown waxy solid (9.13 g, 99% yield), ([α]=+91°, c=0.5, MeOH). LC-MS: (AA) ES+ 164; ¹H NMR (400 MHz, d₆-DMSO) δ ppm 8.76 (s, 1H), 7.60 (s, 3H), 6.49 (d, J=8.3 Hz, 1H), 6.17 (dd, J=8.2, 2.5 Hz, 1H), 6.10 (d, J=2.4 Hz, 1H), 3.12-2.87 (m, 2H), 2.56 (dd, J=16.1, 5.0 Hz, 1H), 2.39-2.24 (m, 2H), 1.75-1.58 (m, 1H), 1.29 (dq, J=11.3, 11.3, 10.9, 6.6 Hz, 1H).

### Step 3: methyl (7R)-7-amino-5,6,7,8-tetrahydronaphthalene-2-carboxylate.HCl Intermediate 48a

The title compound was prepared from (7R)-7-amino-5,6,7,8-tetrahydronaphthalen-2-ol hydrobromide following the procedure outlined in Example 16 steps 4-7. ¹H NMR (400 MHz, d₆-DMSO) δ ppm 8.26 (br s, 3H), 7.76-7.71 (m, 2H), 7.27 (d, J=7.9 Hz, 1H), 3.82 (s, 3H), 3.36 (s, 1H), 3.17 (dd, J=16.4, 5.0 Hz, 1H), 2.96-2.81 (m, 3H), 2.18-2.10 (m, 1H), 1.84-1.72 (m, 1H).

### Example 18

### methyl (7S)-7-amino-5,6,7,8-tetrahydronaphthalene-2-carboxylate.HCl Intermediate 48b

The title compound was prepared from 7-methoxy-1,2,3,4-tetrahydronaphthalen-2-amine following the procedure outlined in Example 17. R-(−)-Mandelic acid was used in place of S-(+)-mandelic acid in Step 1.

### Example 19

### methyl 5-amino-5,6,7,8-tetrahydronaphthalene-2-carboxylate hydrochloride Intermediate 52

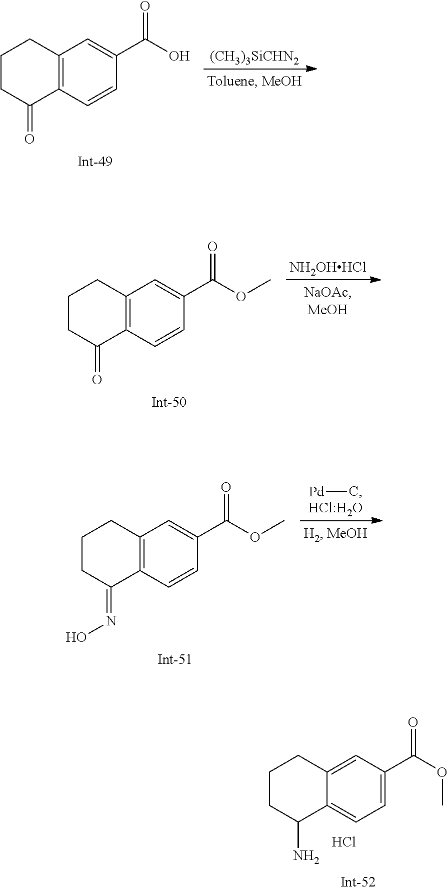

### Step 1: methyl 5-oxo-5,6,7,8-tetrahydronaphthalene-2-carboxylate Intermediate 50

A solution of 5,6,7,8-tetrahydro-5-oxo-2-naphthalenecarboxylic acid (2.1 g, 11 mmol) in MeOH (22 mL) and toluene (22 mL) was cooled to 0° C. Trimethylsilyldiazomethane (7.5 mL, 15 mmol, 2 M in ether) was added drop-wise to the stirred solution. When addition was complete, the solution was allowed to stir for 16 h at rt. Afterwards, the reaction mixture was concentrated. The residue was then diluted with EtOAc (200 mL) and washed with aqueous NaHCO₃ (2×20 mL) and brine (20 mL), dried (MgSO₄), and concentrated to yield methyl 5-oxo-5,6,7,8-tetrahydronaphthalene-2-carboxylate (2.25 g, 99.8%) as a slightly yellow solid. ¹H NMR (400 MHz, CDCl₃) δ ppm 8.10-8.07 (m, 1H), 7.96-7.89 (m, 2H), 3.94 (s, 3H), 3.03 (t, J=6.1 2H), 2.72-2.68 (m, 2H), 2.17 (td, J=12.7, 6.4 Hz, 2H)

### Step 2: 5-amino-5,6,7,8-tetrahydronaphthalene-2-carboxylate hydrochloride Intermediate 52

The title compound was prepared from methyl 5-oxo-5,6,7,8-tetrahydronaphthalene-2-carboxylate following the procedures outlined in Example 15. ¹H NMR (400 MHz, CD₃OD) δ ppm 8.09-7.77 (m, 2H), 7.54 (d, J=7.9 Hz, 1H), 4.61 (t, J=5.5, 5.5 Hz, 1H), 3.93 (s, 3H), 3.05-2.87 (m, 2H), 2.32-2.20 (m, 1H), 2.11-1.89 (m, 3H).

### Example 20

### (R)-ethyl 5-((R)-1,1-dimethylethylsulfinamido)-5,6,7,8-tetrahydronaphthalene-2-carboxylate and (S)-ethyl 5-((R)-1,1-dimethylethylsulfinamido)-5,6,7,8-tetrahydronaphthalene-2-carboxylate Intermediates 55 and 56

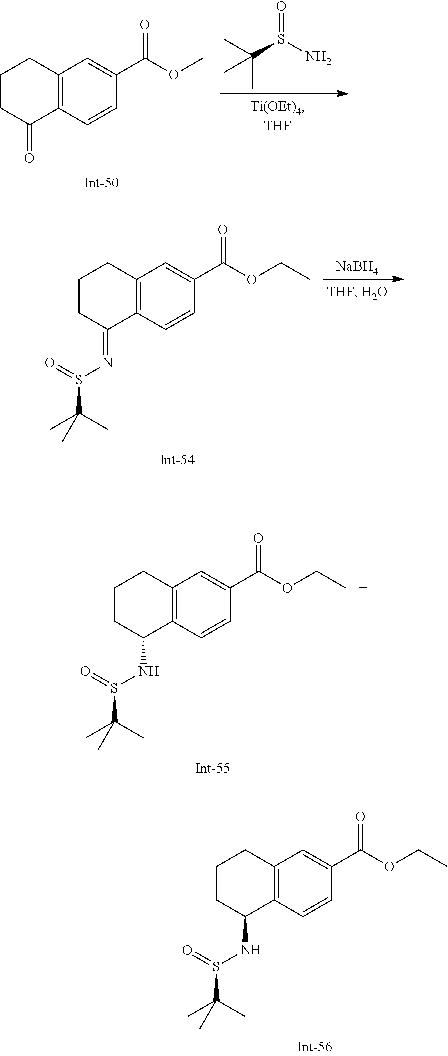

### Step 1: ethyl (5E)-5-{[(R)-tert-butylsulfinyl]imino}-5,6,7,8-tetrahydronaphthalene-2-carboxylate Intermediate 54

To a solution of (R)-(+)-2-methyl-2-propanesulfinamide (0.57 g, 4.71 mmol) and methyl 5-oxo-5,6,7,8-tetrahydronaphthalene-2-carboxylate (0.801 g, 3.92 mmol) in THF (50 mL) at 0° C. was added titanium(IV) ethoxide (5.0 mL, 24 mmol). Upon complete addition, the solution was warmed to rt and further at 85° C. overnight. The reaction mixture was cooled to rt diluted with DCM (50 mL) and cooled to 0° C. Water (5 mL) was added drop-wise with vigorous stirring, resulting in the formation of a thick precipitate. The mixture was stirred at rt for 30 min, and then filtered to remove the solids. Concentration of the filtrate afforded a yellow oil which was purified by silica gel chromatography (15-40% EtOAc/hexane) to afford a viscous yellow oil (0.82 g, 65%). LC-MS (FA): 322; ¹H NMR (400 MHz, CDCl₃) δ ppm 8.19 (d, J=8.9 Hz, 1H), 7.92-7.84 (m, 2H), 4.39 (q, J=7.1 Hz, 2H), 3.31 (ddd, J=17.7, 9.3, 4.8 Hz, 1H), 3.09 (ddd, J=17.6, 7.3, 4.5 Hz, 1H), 2.92 (t, J=6.1 Hz, 2H), 2.10-1.91 (m, 2H), 1.40 (t, J=7.2 Hz, 3H), 1.33 (s, 9H).

### Step 2: (R)-ethyl 5-((R)-1,1-dimethylethylsulfinamido)-5,6,7,8-tetrahydronaphthalene-2-carboxylate and (S)-ethyl 5-((R)-1,1-dimethylethylsulfinamido)-5,6,7,8-tetrahydronaphthalene-2-carboxylate Intermediates 55 and 56

A solution of ethyl (5E)-5-{[(R)-tert-butylsulfinyl]imino}-5,6,7,8-tetrahydronaphthalene-2-carboxylate (0.823 g, 2.56 mmol) in THF (6.3 mL) and water (0.13 mL, 7.1 mmol) was cooled in an CH₃CN/dry ice bath to −45° C. Upon equilibration to bath temperature, solid sodium borohydride (0.29 g, 7.68 mmol) was added. The resulting mixture was allowed to warm to rt over 4 h. The solvent was evaporated and the residue taken up in DCM, dried over anhydrous MgSO₄, the insoluble material was removed via filtration and the solvent was evaporated to afford a colorless oil. Purification by silica chromatography (25-55% EtOAc/hexane) afforded the major product (R)-ethyl 5-((R)-1,1-dimethylethylsulfinamido)-5,6,7,8-tetrahydronaphthalene-2-carboxylate (0.648 g, 78%). LC-MS: (FA) ES+ 324; ¹H NMR (400 MHz, CDCl₃) δ 7.83 (d, J=8.1 Hz, 1H), 7.78 (s, 1H), 7.53 (d, J=8.1 Hz, 1H), 4.58 (d, J=4.1 Hz, 1H), 4.38-4.31 (m, 2H), 3.25 (d, J=3.9 Hz, 1H), 2.90-2.68 (m, 2H), 2.05-1.86 (m, 3H), 1.77 (td, J=11.7, 5.7 Hz, 1H), 1.40-1.32 (m, 3H), 1.21 (s, 9H), and the minor product (S)-ethyl 5-((R)-1,1-dimethylethylsulfinamido)-5,6,7,8-tetrahydronaphthalene-2-carboxylate (0.179 g, 21%). (FA) ES+ 324; ¹H NMR (400 MHz, CDCl₃) δ 7.80 (d, J=8.1 Hz, 1H), 7.75 (s, 1H), 7.46 (d, J=8.1 Hz, 1H), 4.47 (t, J=10.9 Hz, 1H), 4.34 (q, J=7.1 Hz, 2H), 3.42 (d, J=9.9 Hz, 1H), 2.89-2.70 (m, 2H), 2.38-2.26 (m, 1H), 2.03-1.88 (m, 2H), 1.82 (s, 1H), 1.36 (dd, J=14.3, 7.0 Hz, 3H), 1.25 (s, 9H) as colorless oils. (Colyer et al. J. Org. Chem., 2006, 71(18): 6859).

### Example 21

### (R)-ethyl 5-amino-5,6,7,8-tetrahydronaphthalene-2-carboxylate hydrochloride Intermediate 57

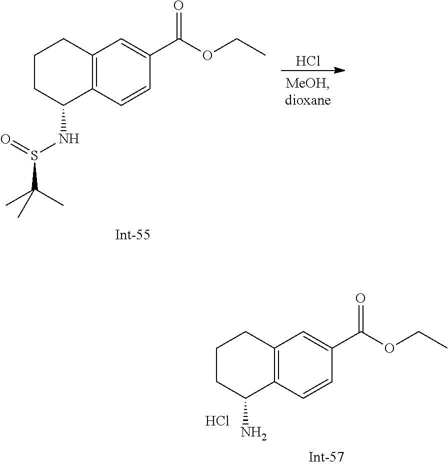 [SMILES: CCOC(=O)c1ccc2c(c1)CCC[C@H]2N[S@](=O)C(C)(C)C ; Cl ; Cl ; CCOC(=O)c1ccc2c(c1)CCC[C@H]2N]

To a solution of ethyl (5R)-5-{[(R)-tert-butylsulfinyl]amino}-5,6,7,8-tetrahydronaphthalene-2-carboxylate (0.641 g, 1.98 mmol) in methanol (20 mL) was added 4.0 M of hydrochloric acid in 1,4-dioxane (0.991 mL, 3.96 mmol). The reaction mixture was stirred at rt for 90 minutes. The reaction mixture was concentrated and the residue obtained was washed with Et₂O/hexane (1:1) and collected by vacuum filtration to afford white solid (0.464 g, 91%). LC-MS: (FA) ES+ 220; ¹H NMR (400 MHz, CDCl₃) δ 8.77 (br s, 3H), 7.86 (dd, J=8.1, 1.5 Hz, 1H), 7.80 (s, 1H), 7.65 (d, J=8.1 Hz, 1H), 4.46 (s, HA), 4.36 (q, J=7.1 Hz, 2H), 2.90 (dt, J=16.8, 5.4 Hz, 1H), 2.81-2.68 (m, 1H), 2.17-2.08 (m, 2H), 2.05-1.92 (m, 1H), 1.79 (dd, J=14.6, 9.6 Hz, 1H), 1.38 (t, J=7.1 Hz, 3H).

### Example 22

### (S)-ethyl 5-amino-5,6,7,8-tetrahydronaphthalene-2-carboxylate hydrochloride Intermediate 58

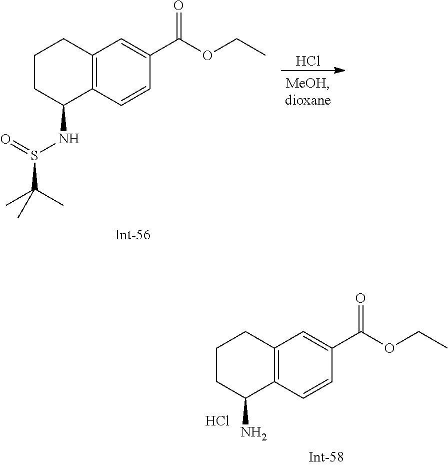 [SMILES: CCOC(=O)c1ccc2c(c1)CCC[C@@H]2N[S@](=O)C(C)(C)C ; Cl ; Cl ; CCOC(=O)c1ccc2c(c1)CCC[C@@H]2N]

The title compound was prepared from (S)-ethyl 5-((R)-1,1-dimethylethylsulfinamido)-5,6,7,8-tetrahydronaphthalene-2-carboxylate following the procedure outlined in Example 21. LC-MS: (FA) ES+ 220; ¹H NMR (400 MHz, CDCl₃) δ 8.79 (br s, 3H), 7.86 (d, J=7.9 Hz, 1H), 7.81 (s, 1H), 7.64 (d, J=8.0 Hz, 1H), 4.46 (s, 1H), 4.37 (q, J=7.0 Hz, 2H), 2.96-2.84 (m, 1H), 2.81-2.68 (m, 1H), 2.20-2.06 (m, 2H), 2.06-1.92 (m, 1H), 1.87-1.72 (m, 1H), 1.39 (t, J=7.0 Hz, 3H).

### Example 23

### 6-[(4-chlorobenzyl)amino]-N-hydroxy-5,6,7,8-tetrahydronaphthalene-2-carboxamide Compound I-106

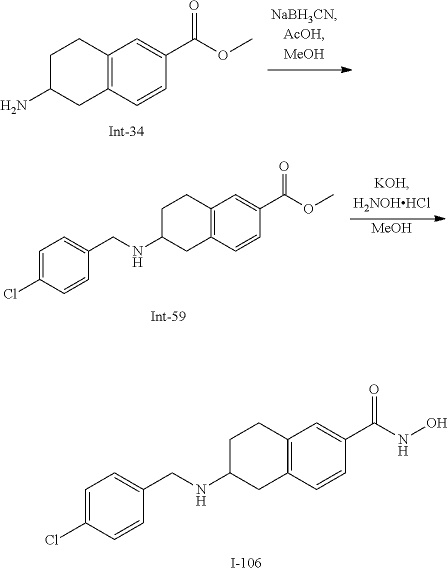 [SMILES: "MeOH" ; COC(=O)c1ccc2c(c1)CCC(N)C2 ; COC(=O)c1ccc2c(c1)CCC(NCc1ccc(Cl)cc1)C2 ; O=C(NO)c1ccc2c(c1)CCC(NCc1ccc(Cl)cc1)C2]

### Step 1: methyl 6-[(4-chlorobenzyl)amino]-5,6,7,8-tetrahydronaphthalene-2-carboxylate Intermediate 59

To a solution of methyl 6-amino-5,6,7,8-tetrahydronaphthalene-2-carboxylate (200 mg, 0.97 mmol) in methanol (10 mL) was added 4-chlorobenzaldehyde (151 mg, 1.07 mmol) and acetic acid (0.11 mL, 1.95 mmol). The reaction mixture was stirred at rt for 45 min after which time sodium cyanoborohydride (73.5 mg, 1.17 mmol) was added. The reaction mixture was allowed to stir overnight after which time LC-MS showed complete reaction. Saturated sodium bicarbonate solution was added and the mixture was extracted with EtOAc (2×). The organic phases were washed with water (2×) then with brine, dried over Na₂SO₄ and evaporated. The residue was purified by silica gel chromatography (1.2% to 5% MeOH/DCM) to afford methyl 6-[(4-chlorobenzyl)amino]-5,6,7,8-tetrahydronaphthalene-2-carboxylate (286 mg, 89%). ¹H NMR (400 MHz, CDCl₃) 5 ppm 7.78-7.73 (m, 2H), 7.30-7.28 (m, 4H), 7.13 (d, J=7.9 Hz, 1H), 3.89 (s, 3H), 3.87 (s, 2H), 3.12-2.91 (m, 3H), 2.88-2.78 (m, 1H), 2.67 (dd, J=16.2, 8.3 Hz, 1H), 2.14-2.04 (m, 1H), 1.74-1.60 (m, 1H).

### Step 2: 6-[(4-chlorobenzyl)amino]-N-hydroxy-5,6,7,8-tetrahydronaphthalene-2-carboxamide Compound I-106

The title compound was prepared from methyl 6-[4-chlorobenzyl)amino]-5,6,7,8-tetrahydronaphthalene-2-carboxylate following the procedure outlined in Example 3, step 3 (45%). LC-MS: (AA) ES+ 331; ¹H NMR (400 MHz, d₆-DMSO) δ ppm 7.47-7.41 (m, 2H), 7.40-7.32 (m, 4H), 7.09 (d, J=7.7 Hz, 1H), 3.77 (s, 2H), 2.98 (dd, J=16.7, 4.7 Hz, 1H), 2.90-2.77 (m, 2H), 2.74-2.63 (m, 1H), 2.58-2.52 (m, 1H), 2.02-1.94 (m, 1H), 1.60-1.47 (m, 1H).

### Example 24

The following compounds were prepared in a fashion analogous to that described in Example 23 starting from the intermediates which were prepared as described above, and the corresponding aldehydes.

| Compound | LC-MS |
|---|---|
| I-107 | ES+ 298 (AA) |
| I-85 | ES+ 298 (FA) |
| I-81 | ES+ 311 (FA) |
| I-80 | ES+ 235 (FA) |
| I-76 | ES+ 331 (FA) |

### Example 25

### N-hydroxy-7-[(3-methoxyphenyl)amino]-5,6,7,8-tetrahydronaphthalene-2-carboxamide Compound I-84

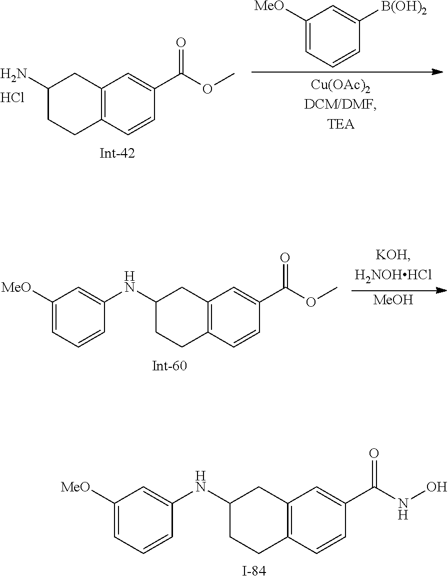

### Step 1: methyl 7-[(3-methoxyphenyl)amino]-5,6,7,8-tetrahydronaphthalene-2-carboxylate Intermediate 60

To a mixture of methyl 7-amino-5,6,7,8-tetrahydronaphthalene-2-carboxylate.HCl (0.1 g, 0.414 mmol) in DMF (4 mL) and triethylamine (0.577 mL, 4.14 mmol) was added 3-methoxyphenylboronic acid (0.288 g, 1.9 mmol), copper(II) acetate (0.348 g, 0.1.91 mmol) and 4 Å molecular sieves. The reaction mixture was stirred at rt for 2 days. The solid was removed by filtration and the filtrate was diluted with EtOAc, washed with NH₄OH (2×) and brine, dried over anhydrous sodium sulfate, filtered and concentrated. The residue was purified by silica gel chromatography (0-40% EtOAc/hexanes) to afford methyl 7-[(3-methoxyphenyl)amino]-5,6,7,8-tetrahydronaphthalene-2-carboxylate (106 mg, 82%). LC-MS (FA) ES+ 312.

### Step 2: N-hydroxy-7-[(3-methoxyphenyl)amino]-5,6,7,8-tetrahydronaphthalene-2-carboxamide Compound I-84

The title compound was prepared from methyl 7-[(3-methoxyphenyl)amino]-5,6,7,8-tetrahydronaphthalene-2-carboxylate following the procedure outlined in Example 3, step 3 (99%). LC-MS (AA) ES+ 313; ¹H NMR (400 MHz, CD₃OD) δ ppm 7.48 (d, J=8.6 Hz, 2H), 7.18 (d, J=7.8 Hz, 1H), 7.02 (t, J=8.0 Hz, 1H), 6.36-6.22 (m, 3H), 3.72 (s, 4H), 3.18 (dd, J=16.1, 3.8 Hz, 1H), 3.02-2.85 (m, 2H), 2.73 (dd, J=16.3, 9.2 Hz, 1H), 2.24-2.15 (m, 1H), 1.78-1.62 (m, 1H).

### Example 26

The following compounds were prepared in a fashion analogous to that described in Example 25 starting from methyl 7-amino-5,6,7,8-tetrahydronaphthalene-2-carboxylate.HCl, and the corresponding boronic acid.

| Compound | LC-MS |
|---|---|
| I-82 | ES+ 317 (AA) |
| I-83 | ES+ 317 (FA) |

### Example 27

### N-hydroxy-7-(pyridin-4-ylamino)-5,6,7,8-tetrahydronaphthalene-2-carboxamide Compound I-95

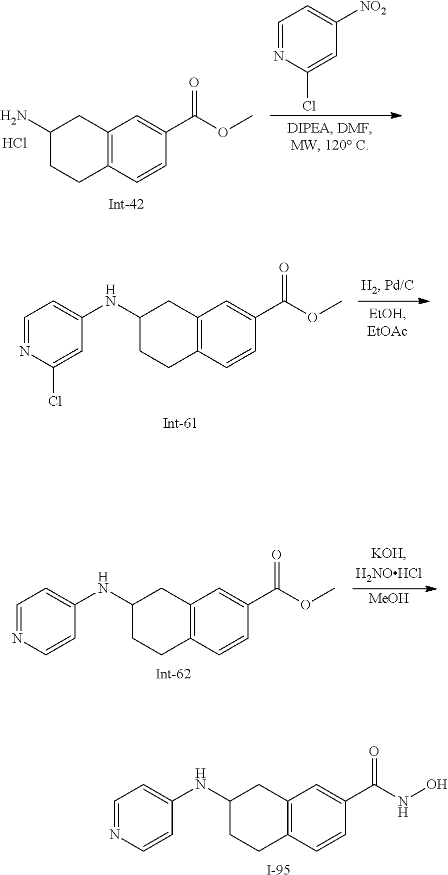

### Step 1: methyl 7-[(2-chloropyridin-4-yl)amino]-5,6,7,8-tetrahydronaphthalene-2-carboxylate Intermediate 61

Into a microwave vial was added methyl 7-amino-5,6,7,8-tetrahydronaphthalene-2-carboxylate.HCl (0.01 g, 0.041 mmol), 2-chloro-4-nitro-pyridine (0.007 g, 0.045 mmol), N,N-diisopropylethylamine (0.028 mL, 0.165 mmol) and DMF (0.1 mL). The reaction mixture was heated in the microwave at 120° C. for 2 h. The reaction mixture was diluted with EtOAc and washed with water (2×). The organic phase was washed with brine, dried over anhydrous sodium sulfate, filtered and concentrated. The residue was purified by silica gel chromatography (0-50% EtOAc/DCM) to give methyl 7-[2-chloropyridin-4-yl)amino]-5,6,7,8-tetrahydronaphthalene-2-carboxylate (27 mg, with impurities). LC-MS (FA) ES+ 317.

### Step 2: methyl 7-(pyridin-4-ylamino)-5,6,7,8-tetrahydronaphthalene-2-carboxylate Intermediate 62

To a solution of methyl 7-[(2-chloropyridin-4-yl)amino]-5,6,7,8-tetrahydronaphthalene-2-carboxylate (63 mg, 0.2 mmol) in ethanol (1 mL) and EtOAc (1 mL) was added 10% palladium on carbon (21 mg). The mixture was stirred under an atmosphere of hydrogen for 3 h. The reaction mixture was then filtered through Celite, washed with ethyl acetate and the filtrate was evaporated. The residue was purified by chromatography on silica (0% to 10% MeOH/DCM) to afford methyl 7-(pyridin-4-ylamino)-5,6,7,8-tetrahydronaphthalene-2-carboxylate (15 mg, 27%). LC-MS (FA) ES+ 283.

### Step 3: N-hydroxy-7-(pyridin-4-ylamino)-5,6,7,8-tetrahydronaphthalene-2-carboxamide Compound I-95

The title compound was prepared from methyl 7-(pyridin-4-ylamino)-5,6,7,8-tetrahydronaphthalene-2-carboxylate following the procedure outlined in Example 3, step 3 (80%) LC-MS (AA) ES+ 284; ¹H NMR (400 MHz, d₆-DMSO) δ ppm 8.01 (d, J=6.3 Hz, 2H), 7.49 (d, J=6.4 Hz, 2H), 7.16 (d, J=8.5 Hz, 1H), 6.60-6.53 (m, 3H), 3.82-3.67 (m, 1H), 3.10 (dd, J=16.7, 4.8 Hz, 1H), 2.94-2.86 (m, 2H), 2.69 (dd, J=16.2, 8.9 Hz, 1H), 2.11-2.02 (m, 1H), 1.68-1.58 (m, 1H).

### Example 28

### 3-(1-methyl-1H-pyrazol-4-yl)-5-(trifluoromethyl)benzoic acid Intermediate-64

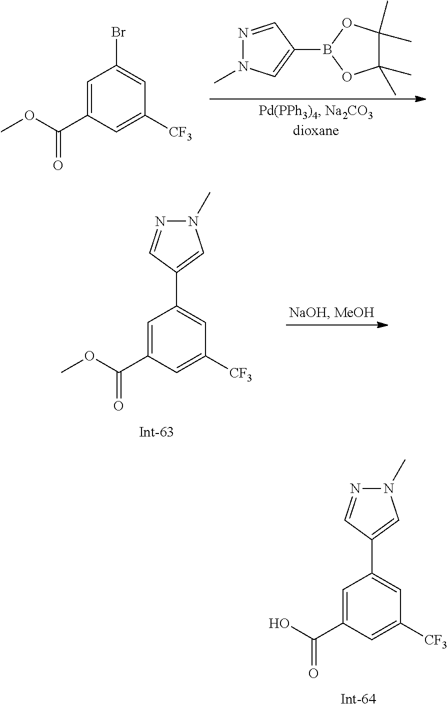

### Step 1: methyl 3-(1-methyl-1H-pyrazol-4-yl)-5-(trifluoromethyl)benzoate Intermediate 63

To a solution of methyl 3-bromo-5-(trifluoromethyl)benzoate (21.8 g, 77 mmol) in 1,4-dioxane (218 mL), and water (131 mL) was added 1-methyl-4-(4,4,5,5-tetramethyl-1,3,2-dioxaborolan-2-yl)-1H-pyrazole (24 g, 116 mmol), sodium carbonate (27.7 g, 261 mmol) and tetrakis(triphenyphosphine)palladium(0) (4.4 g, 3.8 mmol). The reaction mixture was heated at 80° C. for 3 h. The reaction mixture was cooled to rt and precipitated solids were removed by filtration. The filtrate was diluted with water and extracted twice with ethyl acetate. The extracts were washed with brine, dried over sodium sulfate, filtered and concentrated. The residue was purified by filtration through silica, eluting with 0% to 40% ethyl acetate in hexanes to provide methyl 3-(1-methyl-1H-pyrazol-4-yl)-5-(trifluoromethyl)benzoate as a pale yellow solid (22.3 g, 100%). LC-MS (FA): ES+ 285; ¹H NMR (400 MHz, CDCl₃) δ ppm 8.29 (s, 1H), 8.14 (s, 1H), 7.87 (s, 1H), 7.86 (s, 1H), 7.76 (s, 1H), 4.01 (s, 3H), 3.98 (s, 3H).

### Step 2: 3-(1-methyl-1H-pyrazol-4-yl)-5-(trifluoromethyl)benzoic acid Intermediate 64

To a solution of methyl 3-(1-methyl-1H-pyrazol-4-yl)-5-(trifluoromethyl)benzoate (22.3 g, 78.5 mmol) in methanol (375 mL), was added 1 N NaOH solution (314 mL, 314 mmol). The reaction mixture was stirred at rt for 2 h. The methanol was removed by concentration under reduced pressure and the resulting aqueous mixture was acidified to pH 2 with 1 N HCl. The product was isolated by suction filtration, washed with water and hexane and dried under vacuum to provide 3-(1-methyl-1H-pyrazol-4-yl)-5-(trifluoromethyl)benzoic acid as a white solid (20.3 g, 95.7%). LC-MS: (FA) ES+ 271; ¹H NMR (400 MHz, d₆-DMSO) δ ppm 13.53 (s, 1H), 8.44 (s, 1H), 8.33 (s, 1H), 8.16 (s, 1H), 8.09 (d, J=0.7 Hz, 1H), 7.95 (s, 1H), 3.87 (s, 3H).

### Example 29

### 3-[(dimethylamino)methyl]-5-(trifluoromethyl)benzoic acid.HCL Intermediate 66

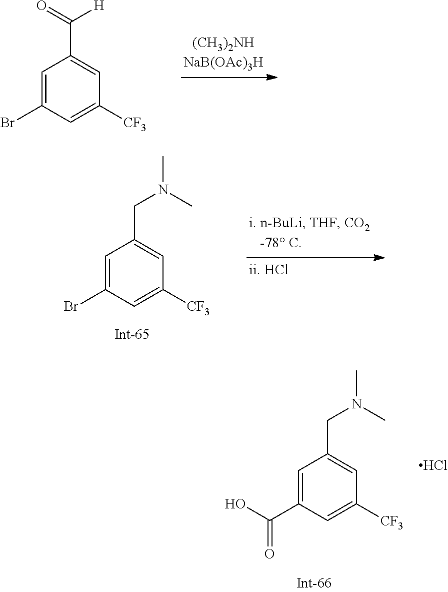 [SMILES: O=Cc1cc(Br)cc(C(F)(F)F)c1 ; CN(C)Cc1cc(Br)cc(C(F)(F)F)c1 ; CN(C)Cc1cc(C(=O)O)cc(C(F)(F)F)c1 ; Cl]

### Step 1: 1-[3-bromo-5-(trifluoromethyl)phenyl]-N,N-dimethylmethanamine Intermediate 65

To a solution of 3-bromo-5-(trifluoromethyl)benzaldehyde (30 g, 118.6 mmol) in methylene chloride (150 mL) was added dimethylamine (2.0 M in THF, 118 mL) and the reaction mixture was stirred at rt for 15 min. The reaction mixture was cooled to 0° C. and sodium triacetoxyborohydride (37.7 g, 178 mmol) was added. The resulting mixture was warmed to it and stirred for 3 h. The solvents were removed under reduced pressure and saturated sodium bicarbonate solution was added. The resulting mixture was extracted three times with ethyl acetate. The combined extracts were washed with brine, dried over sodium sulfate, filtered and concentrated under reduced pressure. Silica gel chromatography (10% to 60% ethyl acetate/hexanes gradient) provided 1-[3-bromo-5-(trifluoromethyl)phenyl]-N,N-dimethylmethanamine as a colorless oil (24.9 g, 74% yield). LC-MS: (FA) ES+ 282; ¹H NMR (300 MHz, CDCl₃) δ ppm 7.68 (s, 1H), 7.65 (s, 1H), 7.52 (s, 1H), 3.44 (s, 2H), 2.25 (s, 6H).

### Step 2: 3-[(dimethylamino)methyl]-5-(trifluoromethyl)benzoic acid.HCl Intermediate 66

To a solution of 1-[3-bromo-5-(trifluoromethyl)phenyl]-N,N-dimethylmethanamine (2.0 g, 7.1 mmol) in THF (40 mL) at −78° C. was added a solution of n-butyllithium (2.50 M in hexane, 3.12 mL, 7.81 mmol) dropwise. The resulting mixture was stirred at −78° C. for 20 min. Excess crushed solid CO₂ was added and the mixture was stirred at −78° C. for another 15 min. The reaction mixture was quenched by the addition of water (0.156 mL), and allowed to warm to rt. The solvents were evaporated and the solid was dried overnight under vacuum to give 3-[(dimethylamino)methyl]-5-(trifluoromethyl)benzoic acid.Li salt as a white solid contaminated with valeric acid. The crude acid was dissolved in aqueous hydrochloric acid (1 M in water, 5 eq) and water (20 vols). Dissolution was not complete. The solids were removed by suction filtration, washed with methylene chloride and set aside. The resulting aqueous solution was washed with DCM (3×). The washed aqueous phase was transferred to a round bottom flask. The filtered solids were added to the aqueous phase. The mixture was concentrated to dryness under reduced pressure. On concentration, the solution afforded a gummy solid, which was azeotropically dried in toluene to become a free-flowing solid. The resulting powder was suspended in ether and filtered. The filter cake was briefly dried under suction. The product was transferred to a round bottom flask and dried under high vacuum at 40° C. overnight. LC-MS: (AA) ES+ 248; ¹H NMR (400 MHz, CD₃OD) δ ppm 8.46 (s, 1H), 8.39 (s, 1H), 8.14 (s, 1H), 4.51 (s, 2H), 2.89 (s, 6H).

### Example 30

### N-{7-[(hydroxyamino)carbonyl]-1,2,3,4-tetrahydronaphthalen-2-yl}-4-methylpiperidine-4-carboxamide Compound I-114

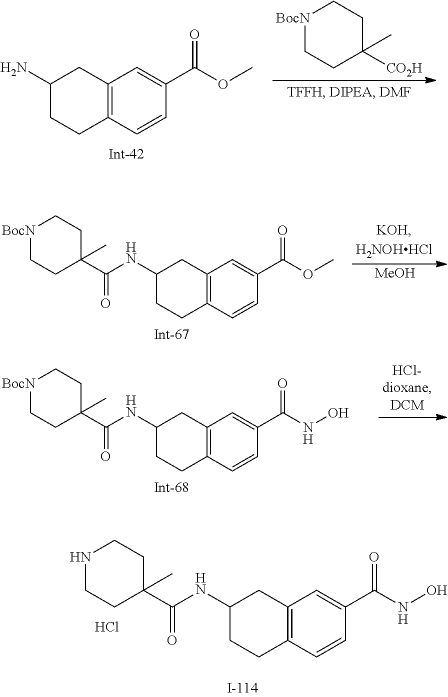

### Step 1: tert-butyl 4-({[7-(methoxycarbonyl)-1,2,3,4-tetrahydronaphthalen-2-yl]amino}carbonyl)-4-methylpiperidine-1-carboxylate Intermediate 67

To a solution of 1-(tert-butoxycarbonyl)-4-methylpiperidine-4-carboxylic (0.141 g, 0.579 mmol) in DMF (1.5 mL) was added N,N-diisopropylethylamine (0.216 mL, 1.24 mmol) and fluoro-N,N,N′,N′-tetramethylformamidinium hexafluorophosphate (0.164 g, 0.62 mmol). The reaction mixture was stirred for 15 min. Methyl 7-amino-5,6,7,8-tetrahydronaphthalene-2-carboxylate.HCl (0.067 g, 0.28 mmol) was added and the reaction mixture was stirred at rt overnight. Water was added and the mixture was extracted with EtOAc (2×). The organic phases were washed with brine, dried over anhydrous sodium sulfate, filtered and concentrated. The residue was purified by silica gel chromatography (0-50% EtOAc/hexanes) to afford tert-butyl 4-({[7-(methoxycarbonyl)-1,2,3,4-tetrahydronaphthalen-2-yl]amino}carbonyl)-4-methylpiperidine-1-carboxylate as a colorless oil (94 mg, 79%). LC-MS (FA) ES+ 431.

### Step 2: tert-butyl 4-[({7-[(hydroxyamino)carbonyl]-1,2,3,4-tetrahydronaphthalen-2-yl}amino)carbonyl]-4-methylpiperidine-1-carboxylate Intermediate 68

The title compound was prepared from methyl tert-butyl 4-({[7-(methoxycarbonyl)-1,2,3,4-tetrahydronaphthalen-2-yl]amino}carbonyl)-4-methylpiperidine-1-carboxylate following the procedure outlined in Example 3, step 3. The crude compound was taken up in toluene and concentrated to a residue which was taken into water (5 mL) and sat. NaHCO₃ solution (5 mL). The white suspension was sonicated for 5 min, filtered and washed with water. The impure compound was dissolved into MeOH and dry loaded onto Celite. The compound was purified on an amine functionalized silica gel column (0-50% EtOH/EtOAc) to afford tert-butyl 4-[({7-[(hydroxyamino)carbonyl]-1,2,3,4-tetrahydronaphthalen-2-yl}amino)carbonyl]-4-methylpiperidine-1-carboxylate as a white solid (38 mg, 41%) LC-MS (FA) ES+ 432.

### Step 3: N-[7-[(hydroxyamino)carbonyl]-1,2,3,4-tetrahydronaphthalen-2-yl]-4-methylpiperidine-4-carboxamide Compound I-114

Into a solution of tert-butyl 4-[({7-[(hydroxyamino)carbonyl]-1,2,3,4-tetrahydronaphthalen-2-yl}amino)carbonyl]-4-methylpiperidine-1-carboxylate (0.038 g, 0.088 mmol in methylene chloride (1.2 mL) was added 4.0 M of hydrochloric acid in 1,4-dioxane (1.2 mL). Upon addition of HCl solution, a white solid precipitated. The reaction mixture was stirred at rt for 2 h. The white solid was filtered, washed with DCM and concentrated from EtOH (4×). The salt was dried under high vacuum at 40° C. for 2 days to afford N-{7-[(hydroxyamino)carbonyl]-1,2,3,4-tetrahydronaphthalen-2-yl}-4-methylpiperidine-4-carboxamide as a white solid (31 mg, 95%). LC-MS (AA) ES+ 332; ¹H NMR (400 MHz, d₆-DMSO) δ ppm 11.11 (s, 1H), 8.96 (s, 1H), 8.67 (s, 2H), 7.79 (d, J=7.6 Hz, 1H), 7.48 (d, J=7.3 Hz, 2H), 7.15 (d, J=8.2 Hz, 1H), 4.01 (s, 1H), 3.15 (d, J=12.5 Hz, 2H), 2.94 (dd, J=16.4, 5.4 Hz, 1H), 2.89-2.77 (m, 4H), 2.72 (dd, J=16.4, 10.3 Hz, 1H), 2.18 (d, J=14.5 Hz, 2H), 1.91 (d, J=12.6 Hz, 1H), 1.74-1.51 (m, 3H), 1.17 (s, 3H).

### Example 31

The following compounds were prepared in a fashion analogous to that described in Example 30, Steps 1 and 2 starting from the appropriate amine intermediates which were prepared as described above, and the corresponding carboxylic acids which were either commercially available or prepared as described above. Where the carboxylic acid used contained an N-Boc group, this was removed in the final step following the procedure outlined in Example 30, step 3.

| Compound | LC-MS |
|---|---|
| I-79 | ES+ 459 (FA) |
| I-78 | ES+ 436 (FA) |
| I-77 | ES+ 341 (FA) |
| I-112 | ES+ 341 (FA) |
| I-122 | ES+ 341 (AA) |
| I-170 | ES+ 332 (AA) |
| I-171 | ES+ 436 (FA) |

### Example 32

### N-(6-(hydroxycarbamoyl)-1,2,3,4-tetrahydronaphthalen-2-yl)-1-methyl-1H-pyrrole-2-carboxamide Compound I-62

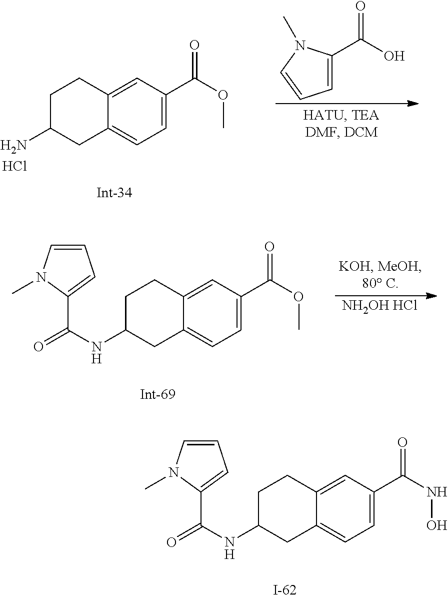

### Step 1: methyl 6-(1-methyl-1H-pyrrole-2-carboxamido)-5,6,7,8-tetrahydronaphthalene-2-carboxylate Intermediate 69

A mixture of N-methylpyrrole-2-carboxylic acid (0.0489 g, 0.391 mmol), HATU (0.149 g, 0.391 mmol), triethylamine (0.208 mL, 1.49 mmol) and DMF (2.5 mL) was stirred at rt for 25 min. Methyl 6-amino-5,6,7,8-tetrahydronaphthalene-2-carboxylate hydrochloride (0.09 g, 0.372 mmol) in DCM (0.3 mL) was added, and the solution was allowed to stir at rt for 16 h. The solution was concentrated and DCM (3 mL) and water (1 mL) were added. After separation, the aqueous phase was extracted with DCM (2×3 mL). The combined organic phases were concentrated to give methyl 6-(1-methyl-1H-pyrrole-2-carboxamido)-5,6,7,8-tetrahydronaphthalene-2-carboxylate as a solid. LC-MS: (FA) ES+ 313.

### Step 2: N-(6-(hydroxycarbamoyl)-1,2,3,4-tetrahydronaphthalen-2-yl)-1-methyl-1H-pyrrole-2-carboxamide Compound I-62

To a vial containing crude methyl 6-(1-methyl-1H-pyrrole-2-carboxamido)-5,6,7,8-tetrahydronaphthalene-2-carboxylate was added hydroxylamine hydrochloride (0.0776 g, 1.12 mmol), potassium hydroxide (0.209 g, 3.72 mmol) and MeOH (4.1 mL). The vial was sealed and the solution was heated at 80° C. with vigorous stirring for 1 h before being cooled to rt. Acetic acid (0.212 mL, 3.72 mmol) was then slowly added to the solution and shaken at rt for 10 min to quench excess base. The solvent was then completely evaporated. The solution was purified by prep-HPLC after filtration to yield N-(6-(hydroxycarbamoyl)-1,2,3,4-tetrahydronaphthalen-2-yl)-1-methyl-1H-pyrrole-2-carboxamide (0.030 g, 26.0%). LC-MS: (FA) ES+ 314; ¹H NMR (400 MHz, CD₃OD) δ ppm 7.50 (s, 1H), 7.48 (d, J=7.9 Hz, 1H), 7.17 (d, J=8.0 Hz, 1H), 6.81-6.80 (m, 1H), 6.76 (dd, J=4.0, 1.7 Hz, 1H), 6.04 (dd, J=4.0, 2.6 Hz, 1H), 4.28-4.14 (m, 1H), 3.88 (s, 3H), 3.13 (dd, J=16.6, 5.4 Hz, 1H), 2.98 (dd, J=8.4, 4.3 Hz, 2H), 2.83 (dd, J=16.8, 10.7 Hz, 1H), 2.19-2.08 (m, 1H), 1.88-1.75 (m, 1H).

### Example 33

The following compounds were prepared in a fashion analogous to that described in Example 32 starting from the intermediates which were prepared as described above, and the corresponding carboxylic acids.

| Compound | LC-MS (FA) |
|---|---|
| I-61 | ES+ 341 |
| I-60 | ES+ 387 |
| I-59 | ES+ 391 |
| I-58 | ES+ 377 |
| I-57 | ES+ 369 |
| I-56 | ES+ 467 |
| I-55 | ES+ 365 |
| I-54 | ES+ 351 |
| I-53 | ES+ 574 |
| I-52 | ES+ 367 |
| I-51 | ES+ 370 |
| I-50 | ES+ 329 |
| I-49 | ES+ 317 |
| I-48 | ES+ 425 |
| I-47 | ES+ 518 |
| I-46 | ES+ 379 |
| I-45 | ES+ 351 |
| I-44 | ES+ 381 |
| I-43 | ES+ 395 |
| I-42 | ES+ 405 |
| I-41 | ES+ 432 |
| I-40 | ES+ 386 |
| I-39 | ES+ 364 |
| I-38 | ES+ 427 |
| I-37 | ES+ 463 |
| I-36 | ES+ 395 |
| I-35 | ES+ 391 |
| I-34 | ES+ 387 |
| I-33 | ES+ 381 |
| I-32 | ES+ 402 |
| I-31 | ES+ 367 |
| I-30 | ES+ 385 |
| I-29 | ES+ 386 |
| I-28 | ES+ 368 |
| I-27 | ES+ 394 |
| I-26 | ES+ 343 |
| I-25 | ES+ 395 |
| I-24 | ES+ 367 |
| I-23 | ES+ 415 |
| I-22 | ES+ 449 |
| I-20 | ES+ 379 |
| I-19 | ES+ 371 |
| I-18 | ES+ 314 |
| I-17 | ES+ 317 |
| I-16 | ES+ 367 |
| I-15 | ES+ 305 |
| I-14 | ES+ 404 |
| I-13 | ES+ 414 |
| I-12 | ES+ 447 |
| I-11 | ES+ 343 |
| I-10 | ES+ 393 |
| I-9 | ES+ 363 |
| I-8 | ES+ 379 |
| I-7 | ES+ 400 |
| I-6 | ES+ 291 |
| I-5 | ES+ 305 |
| I-4 | ES+ 331 |
| I-3 | ES+ 317 |
| I-2 | ES+ 343 |
| I-21 | ES+ 369 |
| I-1 | ES+ 360 |

### Example 34

### (R)-N-(6-(hydroxycarbamoyl)-1,2,3,4-tetrahydronaphthalen-1-yl)-1-methyl-1H-pyrrole-2-carboxamide Compound I-65

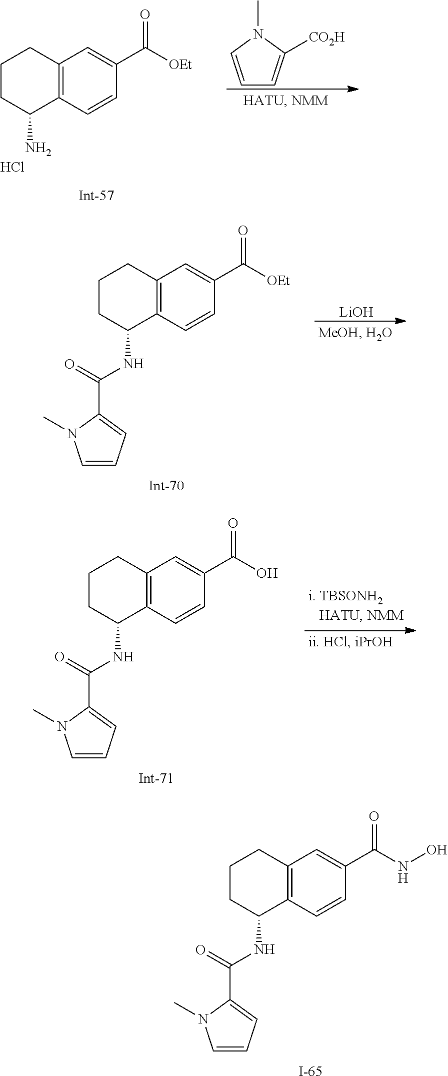

### Step 1: (R)-ethyl 5-(1-methyl-1H-pyrrole-2-carboxamido)-5,6,7,8-tetrahydronaphthalene-2-carboxylate Intermediate 70

To a solution of ethyl (5R)-5-amino-5,6,7,8-tetrahydronaphthalene-2-carboxylate hydrochloride (0.035 g, 0.14 mmol) and N-methylpyrrole-2-carboxylic acid (0.019 g, 0.15 mmol) in DCM (1.75 mL) was added N,N,N′,N′-tetramethyl-O-(7-azabenzotriazol-1-yl)uronium hexafluorophosphate (0.0598 g, 0.157 mmol). The sealed reaction mixture was stirred at rt overnight. The reaction solution was diluted with DCM (3 mL) and washed with saturated aqueous NaHCO₃. The aqueous phase was extracted with additional DCM (2 mL) and the combined organic phases were concentrated to afford oil residue. The material was used without further purification. LC-MS: (FA) ES+ 327.

### Step 2: (R)-5-(1-methyl-1H-pyrrole-2-carboxamido)-5,6,7,8-tetrahydronaphthalene-2-carboxylic acid Intermediate 71

To a solution of (R)-ethyl 5-(1-methyl-1H-pyrrole-2-carboxamido)-5,6,7,8-tetrahydronaphthalene-2-carboxylate in a mixture of tetrahydrofuran (2 mL) and methanol (0.3 mL) was added 1.0 M lithium hydroxide in water (0.4 mL, 0.4 mmol). The reaction mixture was stirred at rt for 5 h. The reaction mixture was neutralized with the addition of 1.0 M of hydrochloric acid in water (0.4 mL, 0.4 mmol) and concentrated. The material was used without further purification. LC-MS: (FA) ES+ 299.

### Step 3: (R)-N-(6-(hydroxycarbamoyl)-1,2,3,4-tetrahydronaphthalen-1-yl)-1-methyl-1H-pyrrole-2-carboxamide Compound I-65

To a mixture of (R)-5-(1-methyl-1H-pyrrole-2-carboxamido)-5,6,7,8-tetrahydronaphthalene-2-carboxylic acid obtained in step 2 and O-(tert-butyldimethylsilyl)hydroxylamine (0.04 g, 0.27 mmol) in DCM (1.5 mL, 23 mmol) was added N,N,N′,N′-tetramethyl-O-(7-azabenzotriazol-1-yl)uronium hexafluorophosphate (0.062 g, 0.164 mmol) and N-methylmorpholine (0.045 mL, 0.41 mmol). The reaction mixture was stirred at rt for 5 h. The solvent was removed and to the residue was added 3 mL (2% conc. HCl in IPA). The resulting mixture was stirred at rt for 1 h. The reaction mixture was concentrated and the material obtained was purified by prep-HPLC to afford the desired product as a white solid (17.3 mg, 40% over three steps). LC-MS: (FA) ES+ 314; ¹H NMR (400 MHz, DMSO) δ 11.14 (s, 1H), 8.96 (s, 1H), 8.27 (d, J=8.9 Hz, 1H), 7.51 (d, J=6.4 Hz, 2H), 7.21 (d, J=8.6 Hz, 1H), 6.94-6.88 (m, 1H), 6.84 (dd, J=3.9, 1.7 Hz, 1H), 5.99 (dd, J=3.9, 2.6 Hz, 1H), 5.15 (t, J=6.2 Hz, 1H), 3.88 (s, 3H), 2.78 (s, 2H), 2.00-1.89 (m, 2H), 1.83-1.69 (m, 2H).

### Example 35

The following compounds were prepared in a fashion analogous to that described in Example 34 starting from the intermediates which were prepared as described above and the corresponding carboxylic acids.

| Compound | LC-MS (FA) |
|---|---|
| I-71 | ES+ 395 |
| I-72 | ES+ 391 |
| I-70 | ES+ 386 |
| I-69 | ES+ 404 |
| I-68 | ES+ 407 |
| I-67 | ES+ 314 |
| I-63 | ES+ 385 |
| I-64 | ES+ 363 |

### Example 36

### 6-[(2,2-dimethylpropanoyl)amino]-N-hydroxy-5,6,7,8-tetrahydronaphthalene-2-carboxamide Compound I-111

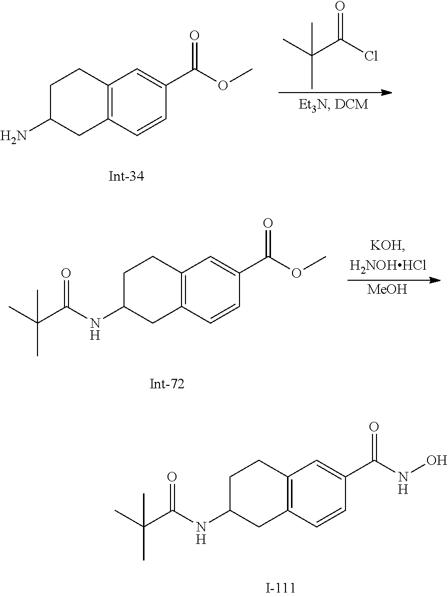

### Step 1: methyl 6-[(2,2-dimethylpropanoyl)amino]-5,6,7,8-tetrahydronaphthalene-2-carboxylate Intermediate 72

To a solution of methyl 6-amino-5,6,7,8-tetrahydronaphthalene-2-carboxylate (150 mg, 0.73 mmol) in DCM (6 mL), was added triethylamine (0.12 mL, 0.88 mmol), and the reaction mixture was cooled to 0° C. 2,2-Dimethylpropanoyl chloride (0.1 mL, 0.8 mmol) was added drop-wise as a solution in 0.3 mL DCM. After 10 min. the reaction mixture was allowed to warm to rt and was stirred for 30 min. The mixture was diluted with DCM and washed with water and brine. The organic phase was dried (Na₂SO₄) and evaporated. The residue was purified by silica gel chromatography (15% to 60% EtOAc/hexane) to afford the title compound (160 mg, 71%). ¹H NMR (400 MHz, CDCl₃) δ ppm 7.80-7.75 (m, 2H), 7.13 (d, J=7.9 Hz, 1H), 5.58 (d, J=7.3 Hz, 1H), 4.31-4.21 (m, 1H), 3.90 (s, 3H), 3.19 (dd, J=16.9, 5.2 Hz, 1H), 3.01-2.84 (m, 2H), 2.65 (dd, J=16.8, 8.5 Hz, 1H), 2.12-2.04 (m, 1H), 1.76 (dddd, J=12.7, 9.3, 9.2, 6.1 Hz, 1H), 1.19 (s, 9H).

### Step 2: 6-[(2,2-dimethylpropanoyl)amino]-N-hydroxy-5,6,7,8-tetrahydronaphthalene-2-carboxamide Compound I-111

The title compound was prepared from methyl 6-[(2,2-dimethylpropanoyl)amino]-5,6,7,8-tetrahydronaphthalene-2-carboxylate following the procedure outlined in Example 3, step 3 (41%). LC-MS: (FA) ES+ 291; ¹H NMR (400 MHz, CD₃OD) δ ppm 7.50-7.45 (m, 2H), 7.15 (d, J=7.9 Hz, 1H), 4.14-4.03 (m, 1H), 3.04 (dd, J=16.4, 4.9 Hz, 1H), 2.97-2.91 (m, 2H), 2.76 (dd, J=16.5, 10.5 Hz, 1H), 2.08-1.98 (m, 1H), 1.82-1.70 (m, 1H), 1.19 (s, 9H).

### Example 37

### (7S)-7-[(2,2-dimethylpropanoyl)amino]-N-hydroxy-5,6,7,8-tetrahydronaphthalene-2-carboxamide Compound I-75

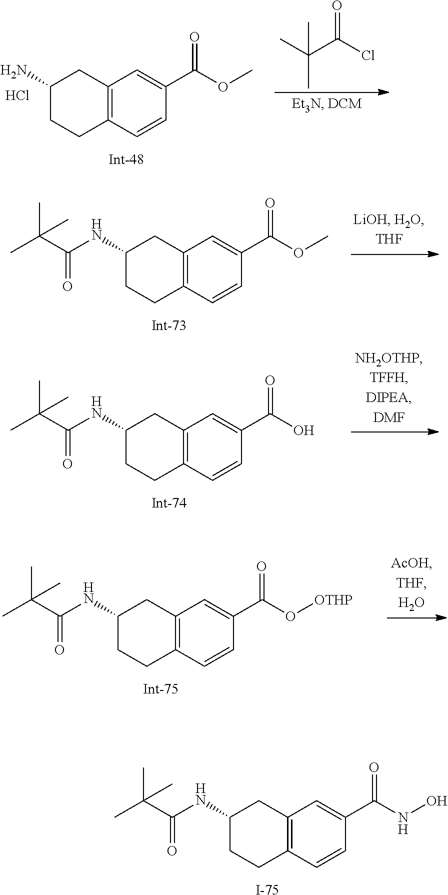

### Step 1: methyl (7S)-7-[(2,2-dimethylpropanoyl)amino]-5,6,7,8-tetrahydronaphthalene-2-carboxylate Intermediate 73

The title compound was prepared from methyl (7S)-7-amino-5,6,7,8-tetrahydronaphthalene-2-carboxylate.HCl following the procedure described in Example 36, Step 1 (89%). LC-MS (FA) ES+ 290.

### Step 2: (7S)-7-[(2,2-dimethylpropanoyl)amino]-5,6,7,8-tetrahydronaphthalene-2-carboxylic acid Intermediate 74

A mixture of methyl (7S)-7-[(2,2-dimethylpropanoyl)amino]-5,6,7,8-tetrahydronaphthalene-2-carboxylate (0.35 g, 1.2 mmol), 2 M lithium hydroxide in water (1.79 mL) and THF (5 mL) was stirred at 50° C. for 5 h. The THF was evaporated and the residue was diluted with water. The aqueous phase was acidified with 1N HCl to pH 2. The precipitated solid was isolated by suction filtration, washed with water and hexane and dried under high vacuum to afford (7S)-7-[(2,2-dimethylpropanoyl)amino]-5,6,7,8-tetrahydronaphthalene-2-carboxylic acid as a white solid (396 mg, quant.). LC-MS (FA) ES+ 276.

### Step 3: (7S)-7-[(2,2-dimethylpropanoyl)amino]-N-(tetrahydro-2H-pyran-2-yloxy)-5,6,7,8-tetrahydronaphthalene-2-carboxamide Intermediate 75

A mixture of (7S)-7-[(2,2-dimethylpropanoyl)amino]-5,6,7,8-tetrahydronaphthalene-2-carboxylic acid (0.375 g, 1.36 mmol), DMF (2.75 mL), N,N-diisopropylethylamine (0.712 mL, 4.08 mmol) and fluoro-N N,N′,N′-tetramethylformamidinium hexafluorophosphate (0.468 g, 1.77 mmol) was stirred for 15 min and O-(tetrahydropyran-2-yl)hydroxylamine (0.191 g, 1.63 mmol) was added. The reaction mixture was stirred at rt for 4 h. Water was added and the mixture was extracted with EtOAc (2×). The extracts were washed with brine, dried over anhydrous sodium sulfate, filtered and concentrated. The residue was purified by silica gel chromatography (0-5% MeOH/DCM) to afford (7S)-7-[(2,2-dimethylpropanoyl)amino]-N-(tetrahydro-2H-pyran-2-yloxy)-5,6,7,8-tetrahydronaphthalene-2-carboxamide as a white solid (482 mg, 94%). LC-MS (FA) ES+ 375.

### Step 4: (7S)-7-[(2,2-dimethylpropanoyl)amino]-N-hydroxy-5,6,7,8-tetrahydronaphthalene-2-carboxamide Compound I-75

A mixture of (7S)-7-[(2,2-dimethylpropanoyl)amino]-N-(tetrahydro-2H-pyran-2-yloxy)-5,6,7,8-tetrahydronaphthalene-2-carboxamide (0.32 g, 0.854 mmol) in THF (3 mL), acetic acid (6 mL) and water (1.5 mL) was heated at 60° C. for 3 h. The mixture was concentrated to dryness. The residue was purified by preparative HPLC to afford (7S)-7-[(2,2-dimethylpropanoyl)amino]-N-hydroxy-5,6,7,8-tetrahydronaphthalene-2-carboxamide (93 mg, 37%). LC-MS (AA) ES+ 291; ¹H NMR (300 MHz, CD₃OD) δ ppm 7.47 (d, J=7.2 Hz, 2H), 7.36 (d, J=7.3 Hz, 1H), 7.17 (d, J=8.4 Hz, 1H), 4.16-4.01 (m, 1H), 3.03 (dd, J=16.3, 4.8 Hz, 1H), 2.93 (dd, J=8.3, 4.4 Hz, 2H), 2.76 (dd, J=16.3, 10.6 Hz, 1H), 2.09-1.97 (m, 1H), 1.83-1.68 (m, 1H), 1.20 (s, 9H).

### Example 38

### (7R)-7-[(2,2-dimethylpropanoyl)amino]-N-hydroxy-5,6,7,8-tetrahydronaphthalene-2-carboxamide Compound I-73

The title compound was prepared from (7R)-7-amino-5,6,7,8-tetrahydronaphthalene-2-carboxylate HCl following the procedures outlined in Example 37. LC-MS (AA) ES+ 291; ¹H NMR (400 MHz, d₆-DMSO) δ ppm 11.08 (s, 1H), 8.95 (s, 1H), 7.46 (d, J=8.3 Hz, 2H), 7.35 (d, J=7.7 Hz, 1H), 7.13 (d, J=7.8 Hz, 1H), 3.99-3.88 (m, 1H), 2.92-2.79 (m, 3H), 2.70 (dd, J=16.3, 10.5 Hz, 1H), 1.87 (d, J=14.1 Hz, 1H), 1.71-1.60 (m, 1H), 1.11 (s, 9H).

### Example 39

### N-hydroxy-7-[(phenylsulfonyl)amino]-5,6,7,8-tetrahydronaphthalene-2-carboxamide Compound I-96

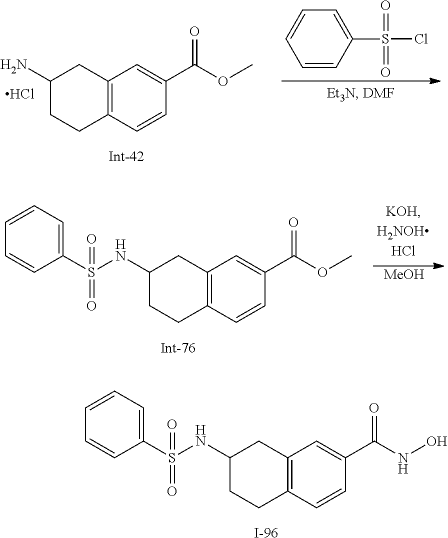

### Step 1: methyl 7-[(phenylsulfonyl)amino]-5,6,7,8-tetrahydronaphthalene-2-carboxylate Intermediate 76

Into a solution of methyl 7-amino-5,6,7,8-tetrahydronaphthalene-2-carboxylate.HCl (0.063 g, 0.26 mmol) in DMF (2.07 mL) was added triethylamine (0.109 mL, 0.782 mmol) and benzenesulfonyl chloride (0.0552 g, 0.313 mmol). The reaction mixture was stirred at rt for 3 h. The reaction mixture was quenched with water and extracted with EtOAc (3×). The organic phases were washed with brine, dried over anhydrous sodium sulfate, filtered and concentrated. The residue was purified by silica gel chromatography (0-20% EtOAc/hexanes) to afford methyl 7-[(phenylsulfonyl)amino]-5,6,7,8-tetrahydronaphthalene-2-carboxylate as a brown oil (59 mg, 65%). LC-MS (FA) ES+ 346.

### Step 2: N-hydroxy-7-[(phenylsulfonyl)amino]-5,6,7,8-tetrahydronaphthalene-2-carboxamide Compound I-96

The title compound was prepared from methyl 7-[(phenylsulfonyl)amino]-5,6,7,8-tetrahydronaphthalene-2-carboxylate following the procedure outlined in Example 3, step 3 (64%). LC-MS (FA) ES+ 347; ¹H NMR (400 MHz, CD₃OD) δ ppm 7.90 (d, J=7.2 Hz, 2H), 7.66-7.54 (m, 3H), 7.44 (d, J=7.8 Hz, 1H), 7.32 (s, 1H), 7.12 (d, J=8.0 Hz, 1H), 3.55-3.46 (m, 1H), 2.92-2.83 (m, 2H), 2.81-2.61 (m, 2H), 1.94-1.84 (m, 1H), 1.74-1.62 (m, 1H).

### Example 40

### 7-[(anilinocarbonyl)amino]-N-hydroxy-5,6,7,8-tetrahydronaphthalene-2-carboxamide Compound I-94

The title compound was prepared from methyl 7-[(anilinocarbonyl)amino]-5,6,7,8-tetrahydronaphthalene-2-carboxylate following the procedure outlined in Example 39, using phenyl isocyanate in the place of benzenesulfonyl chloride. LC-MS (FA) ES+ 326; ¹H NMR (400 MHz, CD₃OD) δ ppm 7.49 (d, J=7.1 Hz, 2H), 7.33 (d, J=7.6 Hz, 2H), 7.22 (dd, J=16.8, 8.2 Hz, 3H), 6.95 (t, J=7.4 Hz, 1H), 4.12-4.03 (m, 1H), 3.15 (dd, J=16.3, 4.8 Hz, 1H), 2.96 (t, J=6.2 Hz, 2H), 2.74 (dd, J=16.4, 8.3 Hz, 1H), 2.15-2.06 (m, 1H), 1.86-1.76 (m, 1H).

### Example 41

The following compounds were prepared from commercial carboxylic acids following the procedures outlined in Example 37, Steps 3 and 4:

| Compound | LC-MS |
|---|---|
| I-66 | ES+ 192 (FA) |
| I-74 | ES+ 248 (AA) |

### Example 42

### N-hydroxy-9-[(1-methyl-1H-pyrrol-2-yl)carbonyl]-1,2,3,4-tetrahydro-1,4-epiminonaphthalene-6-carboxamide Compound I-123

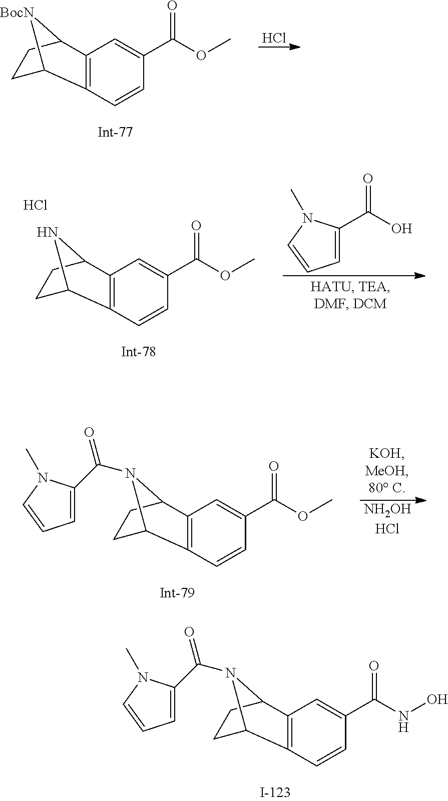

### Step 1: methyl 1,2,3,4-tetrahydro-1,4-epiminonaphthalene-6-carboxylate.HCl Intermediate 78

The title compound was prepared from 9-tert-butyl 6-methyl 1,2,3,4-tetrahydro-1,4-epiminonaphthalene-6,9-dicarboxylate (prepared as described by Kitamura et al. Synlett, 1999, 6: 731-732 and PCT Int. Appl. Publ. WO 05/094251), using the procedure described in Example 30, Step 3. LC-MS ES+ 204.

### Step 2: N-hydroxy-9-[(1-methyl-1H-pyrrol-2-yl)carbonyl]-1,2,3,4-tetrahydro-1,4-epiminonaphthalene-6-carboxamide Compound I-123

The title compound was prepared from methyl 1,2,3,4-tetrahydro-1,4-epiminonaphthalene-6-carboxylate HCl following the procedure outlined in Example 32. LC-MS (FA) ES+ 312; ¹H NMR (400 MHz, d₆-DMSO) δ ppm 11.12 (s, 1H), 9.00 (s, 1H), 7.69 (s, 1H), 7.55 (dd, J=7.6, 1.5 Hz, 1H), 7.40 (d, J=7.6 Hz, 1H), 6.94-6.92 (m, 1H), 6.55 (dd, J=3.8, 1.7 Hz, 1H), 6.06 (dd, J=3.8, 2.6 Hz, 1H), 5.48 (s, 1H), 3.63 (s, 3H), 2.13 (d, J=8.7 Hz, 2H), 1.25 (d, J=8.7 Hz, 2H).

### Example 43

The following compounds were prepared from 9-tert-butyl 6-methyl 1,2,3,4-tetrahydro-1,4-epiminonaphthalene-6,9-dicarboxylate and commercial carboxylic acids following the procedures outlined in Example 42, Step 2:

| Compound | LC-MS |
|---|---|
| I-196 | ES+ 328 (FA) |
| I-197 | ES+ 343 (FA) |
| I-198 | ES+ 339 (FA) |
| I-199 | ES+ 385 (FA) |
| I-200 | ES+ 365 (FA) |
| I-201 | ES+ 289 (FA) |
| I-202 | ES+ 349 (FA) |
| I-203 | ES+ 399 (FA) |
| I-204 | ES+ 365 (FA) |
| I-205 | ES+ 367 (FA) |
| I-206 | ES+ 407 (FA) |
| I-207 | ES+ 275 (FA) |
| I-208 | ES+ 330 (FA) |
| I-210 | ES+ 310 (FA) |
| I-211 | ES+ 323 (FA) |

### Example 44

### 7-{3-[2-(dimethylamino)ethoxy]phenyl}-N-hydroxy-5,6,7,8-tetrahydronaphthalene-2-carboxamide Compound I-194

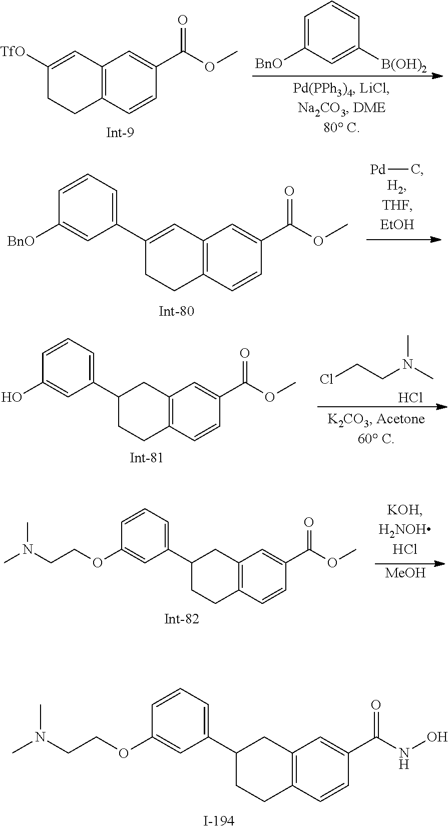

### Step 1: methyl 7-(3-hydroxyphenyl)-5,6,7,8-tetrahydronaphthalene-2-carboxylate Intermediate 81

The title compound was prepared from methyl 7-{[(trifluoromethyl)sulfonyl]oxy}-5,6-dihydronaphthalene-2-carboxylate and [3-(benzyloxy)phenyl]boronic acid following the procedures outlined in Example 3, Steps 1 and 2 LC-MS (FA) ES+ 283.

### Step 2: methyl 7-{3-[2-(dimethylamino)ethoxy]phenyl}-5,6,7,8-tetrahydronaphthalene-2-carboxylate Intermediate 82

To a solution of methyl 7-(3-hydroxyphenyl)-5,6,7,8-tetrahydronaphthalene-2-carboxylate (0.09 g, 0.3 mmol) in acetone (2.4 mL), was added potassium carbonate (0.132 g, 0.956 mmol) and 2-chloro-N,N-dimethylethanamine.HCl (0.055 g, 0.382 mmol). The mixture was heated at 60° C. overnight. The solvents were evaporated and water was added. The mixture was extracted with EtOAc (2×) and the organic phases were washed with water and then brine, dried (Na₂SO₄) and evaporated. The residue was purified by silica gel chromatography (0-10% MeOH/DCM) to afford the title compound as a colorless oil (65 mg, 60%) LC-MS (FA) ES+ 354.

### Step 3: 7-{3-[2-(dimethylamino)ethoxy]phenyl}-N-hydroxy-5,6,7,8-tetrahydronaphthalene-2-carboxamide Compound I-194

The title compound was prepared from methyl 7-{3-[2-(dimethylamino)ethoxy]phenyl}-5,6,7,8-tetrahydronaphthalene-2-carboxylate following the procedure outlined in Example 3, Step 3 (22 mg, 34%) LC-MS (FA) ES+ 355; ¹H NMR (400 MHz, d₆-DMSO) δ ppm 11.10 (s, 1H), 7.53-7.46 (m, 2H), 7.26-7.15 (m, 2H), 6.90-6.86 (m, 2H), 6.78 (dd, J=8.2, 1.6 Hz, 1H), 4.03 (t, J=5.8 Hz, 2H), 2.99-2.85 (m, 5H), 2.62 (t, J=5.8 Hz, 2H), 2.21 (s, 6H), 2.06-1.97 (m, 1H), 1.95-1.85 (m, 1H).

### Example 45

### 6-[(4-chlorobenzyl)oxy]-N-hydroxy-5,6,7,8-tetrahydronaphthalene-2-carboxamide methyl 6-hydroxy-5,6,7,8-tetrahydronaphthalene-2-carboxylate Compound I-125 [ML00750037]

The title compound was prepared from methyl 6-hydroxy-5,6,7,8-tetrahydronaphthalene-2-carboxylate and 4-chlorobenzyl bromide following the procedures described in Example 13 LC-MS: (AA) ES+ 332; ¹H NMR (400 MHz, d₆-DMSO) δ ppm 11.08 (s, 1H), 8.93 (s, 1H), 7.49-7.44 (m, 2H), 7.41-7.32 (m, 4H), 7.12 (d, J=7.9 Hz, 1H), 4.56 (q, J=12.3 Hz, 2H), 3.92-3.82 (m, 1H), 3.06 (dd, J=17.0, 4.6 Hz, 1H), 2.94-2.67 (m, 3H), 2.04-1.94 (m, 1H), 1.92-1.81 (m, 1H).

### Example 46

### 6-({2-[(2,2-dimethylpropanoyl)amino]pyridin-4-yl}oxy)-N-hydroxy-5,6,7,8-tetrahydronaphthalene-2-carboxamide Compound I-150

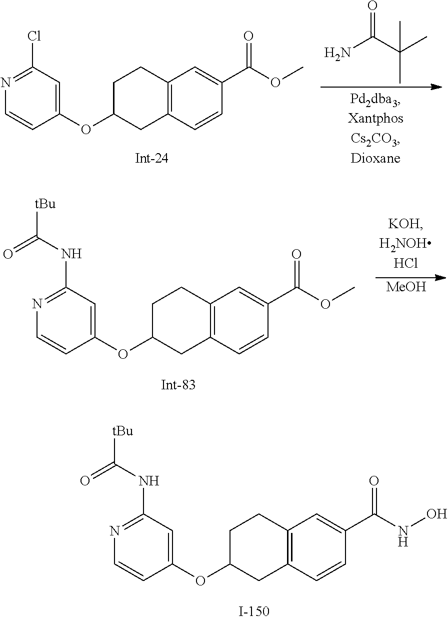

### Step 1: methyl 6-({2-[(2,2-dimethylpropanoyl)amino]pyridin-4-yl}oxy)-5,6,7,8-tetrahydronaphthalene-2-carboxylate Intermediate 83

A microwave vial was charged with pivalamide (68.3 mg, 0.68 mmol), tris(dibenzylideneacetone)dipalladium(0) (56.2 mg, 0.06 mmol), Xantphos (106.5 mg, 0.184 mmol) and cesium carbonate (400 mg, 1.23 mmol) and then sealed and placed under an argon atmosphere. A solution of methyl 6-[(2-chloropyridin-4-yl)oxy]-5,6,7,8-tetrahydronaphthalene-2-carboxylate (130 mg, 0.41 mmol) in 1,4-dioxane (10 mL) was degassed with argon and added to the vial by syringe. The reaction mixture was degassed with argon for a further 2 min and then heated in an oil bath at 100° C. overnight. The reaction mixture was filtered through Celite and the filter pad was washed several times with EtOAc. The filtrate was concentrated and the residue was purified by silica gel chromatography (0% to 40% EtOAc/hexane) to afford the title compound (95 mg, 61%) LC-MS: (AA) ES+ 383.

### Step 2: 6-({2-[(2,2-dimethylpropanoyl)amino]pyridin-4-yl}oxy)-N-hydroxy-5,6,7,8-tetrahydronaphthalene-2-carboxamide Compound I-150

The title compound was prepared from methyl 6-({2-[(2,2-dimethylpropanoyl)amino]pyridin-4-yl}oxy)-5,6,7,8-tetrahydronaphthalene-2-carboxylate following the procedure outlines in Example 3, Step 3 (56 mg, 60%) LC-MS: (AA) ES+ 384; ¹H NMR (400 MHz, CD₃OD) δ ppm 8.08 (d, J=6.0 Hz, 1H), 7.75 (d, J=2.2 Hz, 1H), 7.46-7.55 (m, 2H), 7.17 (d, J=8.1 Hz, 1H), 6.73 (dd, J=5.9, 2.4 Hz, 1H), 5.02-4.96 (m, 1H), 3.27-3.22 (m, 1H), 3.08-2.87 (m, 3H), 2.24-2.06 (m, 2H), 1.30 (s, 9H).

### Example 47

### tert-Butyl 4-({[4-({6-[(hydroxyamino)carbonyl]-1,2,3,4-tetrahydronaphthalen-2-yl}oxy)pyridin-2-yl]amino}carbonyl)-4-methylpiperidine-1-carboxylate Compound I-214 and N-[4-({6-[(hydroxyamino)carbonyl]-1,2,3,4-tetrahydronaphthalen-2-yl}oxy)pyridin-2-yl]-4-methylpiperidine-4-carboxamide Compound I-148

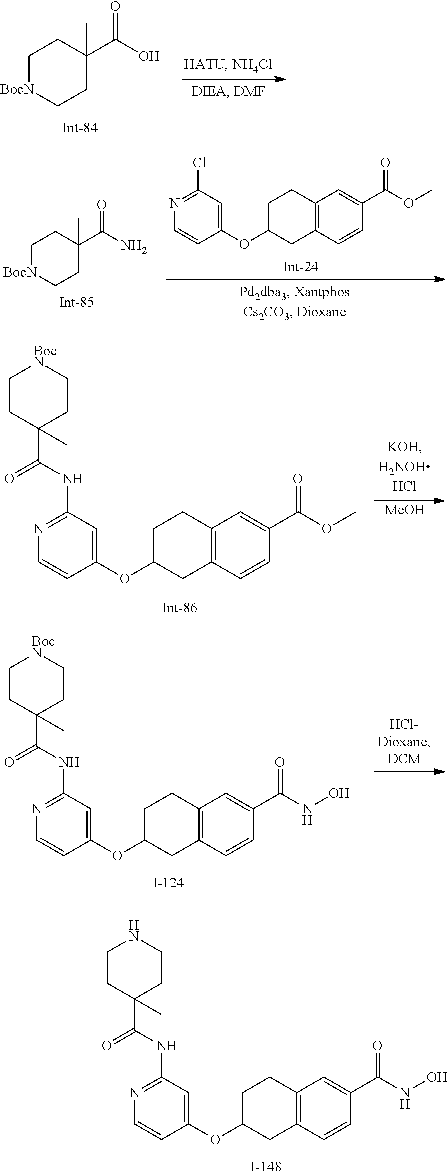

### Step 1: tert-Butyl 4-(aminocarbonyl)-4-methylpiperidine-1-carboxylate Intermediate 85

4-Methyl-4-carboxy-1-N-butoxycarbonyl-piperidine (0.486 g, 2 mmol) was dissolved in N,N-dimethylformamide (20 mL). N,N-diisopropylethylamine (1.4 mL, 8.2 mmol) was added followed by N,N,N′N′-tetramethyl-O-(7-azabenzotriazol-1-yl)uronium hexafluorophosphate (1.2 g, 3.1 mmol). The reaction solution was stirred at room temperature 20 min and then ammonium chloride (0.22 g, 4.1 mmol) was added. The reaction mixture was stirred over night at room temperature and then concentrated under reduced pressure. The residue was partitioned between ethyl acetate (100 mL) and water (150 mL). The phases were slow to settle. The phases were separated and the aqueous phase was extracted with additional ethyl acetate. The extracts were combined, washed with 1N HCl, saturated aqueous sodium bicarbonate solution, water and brine then dried over sodium sulfate, filtered and concentrated under reduced pressure. The crude reside was purified by silica gel chromatography (methylene chloride to 90/10 methylene chloride/methanol gradient) to afford the product as a light pink solid (333 mg, 68%).

### Step 2: tert-Butyl 4-({[4-({6-[(hydroxyamino)carbonyl]-1,2,3,4-tetrahydronaphthalen-2-yl}oxy)pyridin-2-yl]amino}carbonyl)-4-methylpiperidine-1-carboxylate Compound I-124

The title compound was prepared from methyl 6-[(2-chloropyridin-4-yl)oxy]-5,6,7,8-tetrahydronaphthalene-2-carboxylate and tert-butyl 4-(aminocarbonyl)-4-methylpiperidine-1-carboxylate, following the procedures outlined in Example 46 LC-MS: (AA) ES+ 525; ¹H NMR (400 MHz, CD₃OD) δ ppm 8.09 (d, J=6.0 Hz, 1H), 7.73 (d, J=2.2 Hz, 1H), 7.47-7.54 (m, 2H), 7.18 (d, J=8.0 Hz, 1H), 6.74 (dd, J=5.8, 2.3 Hz, 1H), 5.03-4.97 (m, 1H), 3.73-3.63 (m, 2H), 3.27-3.15 (m, 3H), 3.08-2.97 (m, 2H), 2.96-2.87 (m, 1H), 2.22-2.07 (m, 4H), 1.54-1.46 (m, 2H), 1.44 (s, 9H), 1.32 (s, 3H).

### Step 3: N-[4-({6-[(hydroxyamino)carbonyl]-1,2,3,4-tetrahydronaphthalen-2-yl}oxy)pyridin-2-yl]-4-methylpiperidine-4-carboxamide Compound I-148

The title compound was prepared from tert-butyl 4-({[4-({6-[(hydroxyamino)carbonyl]-1,2,3,4-tetrahydronaphthalen-2-yl}oxy)pyridin-2-yl]amino}carbonyl)-4-methylpiperidine-1-carboxylate following the procedure outlined in Example 30, Step 3 and was purified using preparative HPLC (AA method), (110 mg, 93%). LC-MS: (AA) ES+ 425; ¹H NMR (400 MHz, d₆-DMSO) δ ppm 8.13 (d, J=5.8 Hz, 1H), 7.70 (d, J=2.3 Hz, 1H), 7.57-7.46 (m, 2H), 7.17 (d, J=8.0 Hz, 1H), 6.79 (dd, J=5.8, 2.6 Hz, 1H), 5.02-4.95 (m, 1H), 3.26-3.17 (m, 1H), 3.01-2.79 (m, 5H), 2.73-2.61 (m, 2H), 2.17-1.97 (m, 4H), 1.51-1.36 (m, 2H), 1.23 (s, 3H).

### Example 48

### 1-ethyl-N-[4-({6-[(hydroxyamino)carbonyl]-1,2,3,4-tetrahydronaphthalen-2-yl}oxy)pyridin-2-yl]-4-methylpiperidine-4-carboxamide Compound I-222

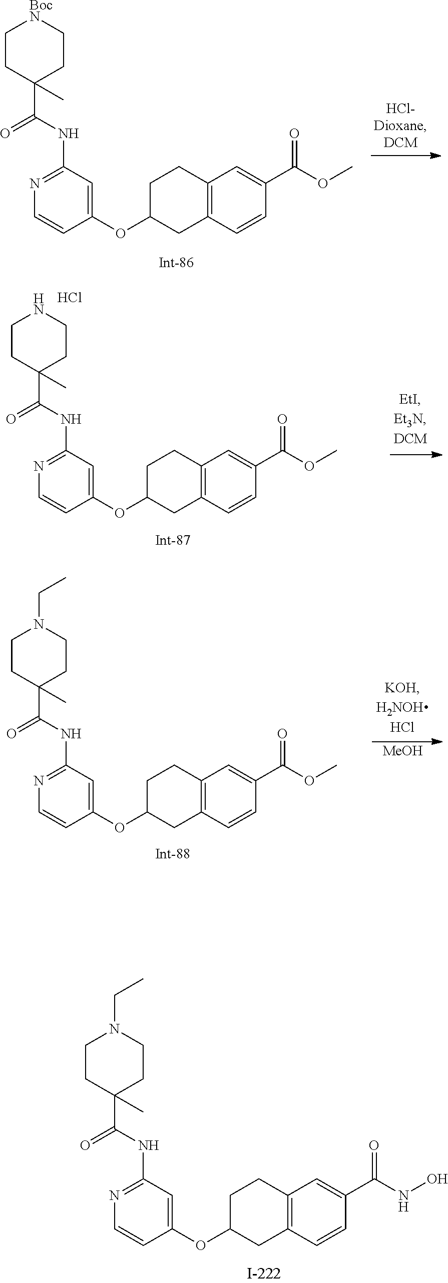

### Step 1: methyl 6-[(2-{[(4-methylpiperidin-4-yl)carbonyl]amino}pyridin-4-yl)oxy]-5,6,7,8-tetrahydronaphthalene-2-carboxylate.HCl Intermediate 87

Methyl 6-[(2-{[(4-methylpiperidin-4-yl)carbonyl]amino}pyridin-4-yl)oxy]-5,6,7,8-tetrahydronaphthalene-2-carboxylate was prepared from tert-butyl 4-{[(4-{[6-(methoxycarbonyl)-1,2,3,4-tetrahydronaphthalen-2-yl]oxy}pyridin-2-yl)amino]carbonyl}-4-methylpiperidine-1-carboxylate using the procedure described in Example 30, step 3. LC-MS: (AA) ES+ 424.

### Step 2: methyl 6-[(2-{[(1-ethyl-4-methylpiperidin-4-yl)carbonyl]amino}pyridin-4-yl)oxy]-5,6,7,8-tetrahydronaphthalene-2-carboxylate Intermediate 88

To a solution of methyl 6-[(2-{[(4-methylpiperidin-4-yl)carbonyl]amino}pyridin-4-yl)oxy]-5,6,7,8-tetrahydronaphthalene-2-carboxylate.HCl (0.100 g, 0.217 mmol) in DCM (5 mL) was added triethylamine (90.9 uL, 0.652 mmol) and iodoethane (52.2 uL, 0.652 mmol). The reaction was stirred at rt overnight after which time TLC (1:9 MeOH/DCM) showed complete reaction. The mixture was extracted with EtOAc (2×) and the organic phase was washed with water and then brine, dried (Na₂SO₄) and evaporated. The residue was purified by silica gel chromatography (0% to 10% MeOH/DCM) to afford the title compound (45 mg, 46%). LC-MS: (AA) ES+ 452.

### Step 3: 1-ethyl-N-[4-({6-[(hydroxyamino)carbonyl]-1,2,3,4-tetrahydronaphthalen-2-yl}oxy)pyridin-2-yl]-4-methylpiperidine-4-carboxamide Compound I-222

The title compound was prepared from methyl 6-[(2-{[(1-ethyl-4-methylpiperidin-4-yl)carbonyl]amino}pyridin-4-yl)oxy]-5,6,7,8-tetrahydronaphthalene-2-carboxylate following the procedure outlined in Example 3, step 3. LC-MS: (AA) ES+ 453; ¹H NMR (400 MHz, CD₃OD) δ ppm 8.47 (s, 1H), 8.12 (d, J=5.7 Hz, 1H), 7.68 (d, J=2.2 Hz, 1H), 7.54-7.47 (m, 2H), 7.18 (d, J=8.1 Hz, 1H), 6.78 (dd, J=6.1, 2.3 Hz, 1H), 5.04-4.96 (m, 1H), 3.46-3.35 (m, 2H), 3.25 (d, J=4.5 Hz, 1H), 3.15-2.98 (m, 6H), 2.96-2.87 (m, 1H), 2.53-2.40 (m, 2H), 2.22-2.08 (m, 2H), 1.90-1.76 (m, 2H), 1.39 (s, 3H), 1.31 (t, J=7.3 Hz, 3H).

### Example 49

### N-[4-({6-[(hydroxyamino)carbonyl]-1,2,3,4-tetrahydronaphthalen-2-yl}oxy)pyridin-2-yl]-1-isopropyl-4-methylpiperidine-4-carboxamide Compound I-219

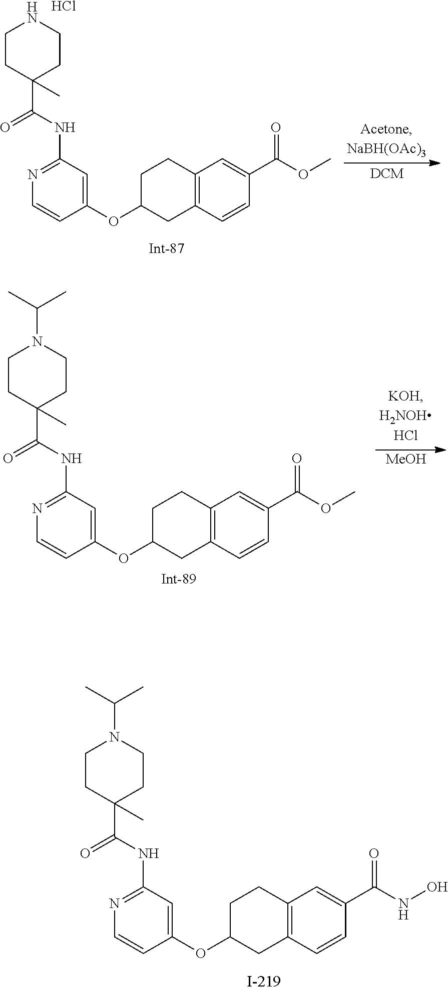 [SMILES: Cl ; COC(=O)c1ccc2c(c1)CCC(Oc1ccnc(NC(=O)C3(C)CCNCC3)c1)C2 ; "MeOH" ; COC(=O)c1ccc2c(c1)CCC(Oc1ccnc(NC(=O)C3(C)CCN(C(C)C)CC3)c1)C2 ; CC(C)N1CCC(C)(C(=O)Nc2cc(OC3CCc4cc(C(=O)NO)ccc4C3)ccn2)CC1]

### Step 1: methyl 6-[(2-{[(1-isopropyl-4-methylpiperidin-4-yl)carbonyl]amino}pyridin-4-yl)oxy]-5,6,7,8-tetrahydronaphthalene-2-carboxylate Intermediate 89

To a solution of methyl 6-[(2-{[(4-methylpiperidin-4-yl)carbonyl]amino}pyridin-4-yl)oxy]-5,6,7,8-tetrahydronaphthalene-2-carboxylate.HCl (0.100 g, 0.217 mmol) in DCM (8 mL) was added acetone (74.9 uL, 1.02 mmol). The reaction was stirred at rt for 4 hours and sodium triacetoxyborohydride (138 mg, 0.652 mmol) was added. The reaction was stirred at rt overnight after which time TLC (1:9 MeOH/DCM) showed complete reaction. The mixture was extracted with EtOAc (2×) and the organic phase was washed with water and then brine, dried (Na₂SO₄) and evaporated. The residue was purified by silica gel chromatography (0% to 10% MeOH/DCM) to afford methyl 6-[(2-{[(1-isopropyl-4-methylpiperidin-4-yl)carbonyl]amino}pyridin-4-yl)oxy]-5,6,7,8-tetrahydronaphthalene-2-carboxylate (71 mg, 72%). LC-MS: (AA) ES+ 466; ¹H NMR (300 MHz, CDCl₃) δ ppm 8.05 (d, J=5.8 Hz, 1H), 7.94 (d, J=2.3 Hz, 1H), 7.83-7.76 (m, 2H), 7.15 (d, J=7.9 Hz, 1H), 6.57 (dd, J=5.9, 2.3 Hz, 1H), 5.00-4.91 (m, 1H), 3.90 (s, 3H), 3.25 (dd, J=17.4, 4.7 Hz, 1H), 3.12-3.00 (m, 2H), 2.95-2.83 (m, 1H), 2.73-2.63 (m, 3H), 2.44-2.34 (m, 2H), 2.21-2.10 (m, 4H), 1.70-1.63 (m, 2H), 1.28 (s, 3H), 1.02 (t, J=6.4 Hz, 6H).

### Step 2: N-[4-({6-[(hydroxyamino)carbonyl]-1,2,3,4-tetrahydronaphthalen-2-yl}oxy)pyridin-2-yl]-1-isopropyl-4-methylpiperidine-4-carboxamide Compound I-219

The title compound was prepared from 6-[(2-{[(1-isopropyl-4-methylpiperidin-4-yl)carbonyl]amino}pyridin-4-yl)oxy]-5,6,7,8-tetrahydronaphthalene-2-carboxylate following the procedure outlined in Example 3, step 3. LC-MS: (AA) ES+ 467; ¹H NMR (400 MHz, CD₃OD) 8 ppm 8.49 (s, 1H), 8.13 (d, J=5.8 Hz, 1H), 7.67 (d, J=2.2 Hz, 1H), 7.54-7.47 (m, 2H), 7.18 (d, J=8.0 Hz, 1H), 6.79 (dd, J=6.1, 2.4 Hz, 1H), 5.04-4.96 (m, 1H), 3.48-3.33 (m, 3H), 3.25 (d, J=4.3 Hz, 1H), 3.16-2.87 (m, 5H), 2.58-2.39 (m, 2H), 2.22-2.10 (m, 2H), 1.90-1.73 (m, 2H), 1.40 (s, 3H), 1.32 (d, J=6.6 Hz, 6H).

### Example 50

The following compounds were prepared from methyl 6-[(2-chloropyridin-4-yl)oxy]-5,6,7,8-tetrahydronaphthalene-2-carboxylate and the appropriate amine or aniline, following the procedures outlined in Example 47.

| Compound | LC-MS |
|---|---|
| I-228 | ES+ 410 (AA) |
| I-225 | ES+ 411 (AA) |
| I-224 | ES+ 511 (AA) |

### Example 51

### N-hydroxy-6-phenoxy-5,6,7,8-tetrahydronaphthalene-2-carboxamide Compound I-229

The title compound was prepared from methyl 6-hydroxy-5,6,7,8-tetrahydronaphthalene-2-carboxylate and phenol, following the procedures outlined in Example 10, steps 6 and 7. Diethyl azodicarboxylate was used in place of di-tert-butyl azodicarboxylate in step 6. LC-MS: (AA) ES+ 284; ¹H NMR (400 MHz, CD₃OD) δ ppm 7.52-7.46 (m, 2H), 7.28-7.23 (m, 2H), 7.16 (d, J=8.0 Hz, 1H), 6.96-6.88 (m, 3H), 4.85-4.80 (m, 1H), 3.21 (dd, J=17.1, 4.5 Hz, 1H), 3.08-2.95 (m, 2H), 2.91-2.82 (m, 1H), 2.17-2.01 (m, 2H).

### Example 52

### tert-butyl 4-{7-[(hydroxyamino)carbonyl]-1,2,3,4-tetrahydronaphthalen-2-yl}piperidine-1-carboxylate Compound I-221

The title compound was prepared following from tert-butyl 4-(4,4,5,5-tetramethyl-1,3,2-dioxaborolan-2-yl)-3,6-dihydropyridine-1(2H)-carboxylate following the procedures outlined in Example 44, step 1 followed by step 3. LCMS (FA) ES+ 375; ¹H NMR (300 MHz, CD₃OD) δ ppm 7.47-7.40 (m, 2H), 7.13 (d, J=7.9 Hz, 1H), 4.15 (s, 1H), 4.10 (s, 1H), 2.94-2.67 (m, 5H), 2.57 (dd, J=16.7, 11.0 Hz, 1H), 2.07-1.96 (m, 1H), 1.81 (t, J=11.2 Hz, 2H), 1.66-1.35 (m, 12H), 1.32-1.15 (m, 2H).

### Example 53

### N-hydroxy-7-piperidin-4-yl-5,6,7,8-tetrahydronaphthalene-2-carboxamide. HCl Compound I-212

The title compound was prepared from tert-butyl 4-{7-[(hydroxyamino)carbonyl]-1,2,3,4-tetrahydronaphthalen-2-yl}piperidine-1-carboxylate following the procedure described in Example 30, step 3. LCMS (AA) ES+ 275; ¹H NMR (400 MHz, d₆-DMSO) δ ppm 11.09 (s, 1H), 8.94 (s, 1H), 8.59 (bs, 2H), 7.48 (s, 1H), 7.45 (dd, J=8.0, 1.5 Hz, 1H), 7.12 (d, J=8.0 Hz, 1H), 3.29 (d, J=12.6 Hz, 2H), 2.89-2.66 (m, 5H), 2.55-2.46 (m, 1H), 1.89 (t, J=13.7 Hz, 2H), 1.59-1.30 (m, 5H).

### Example 54

### tert-butyl 4-({[4-({6-[(hydroxyamino)carbonyl]-1,2,3,4-tetrahydronaphthalen-2-yl}oxy)pyridin-2-yl]carbonyl}amino)piperidine-1-carboxylate Compound I-223 and 4-({6-[(hydroxyamino)carbonyl]-1,2,3,4-tetrahydronaphthalen-2-yl}oxy)-N-piperidin-4-ylpyridine-2-carboxamide Compound I-220

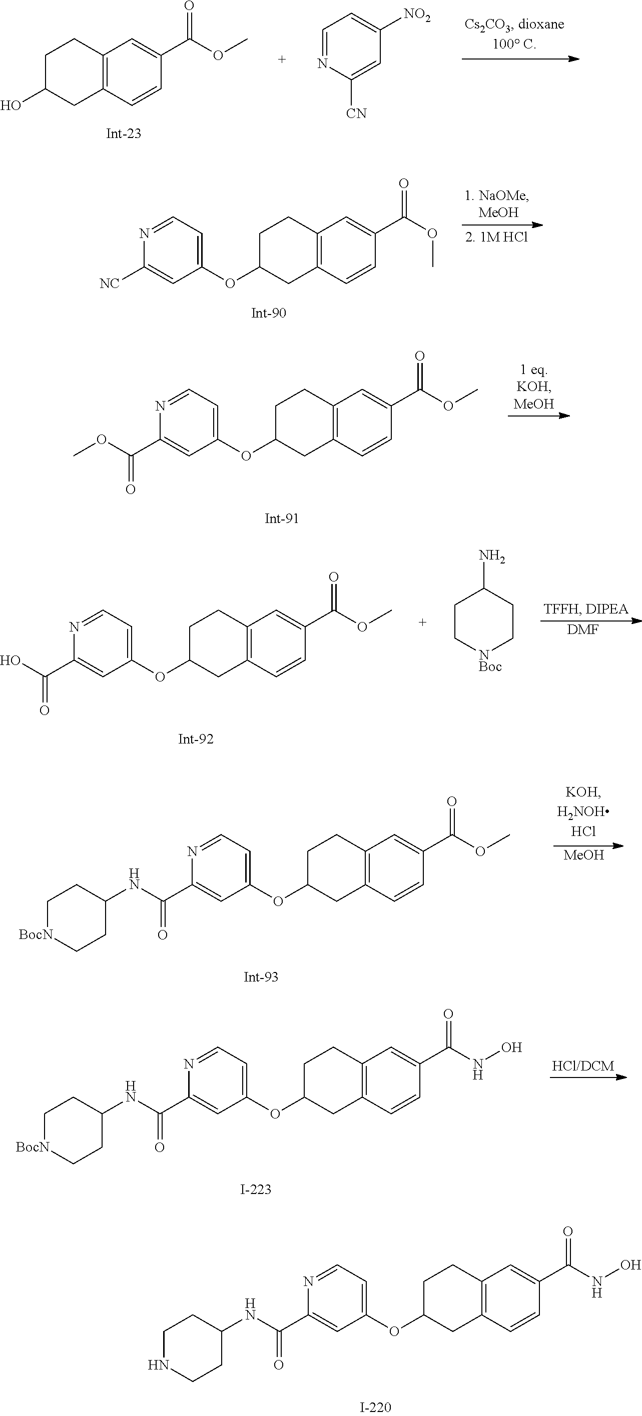

### Step 1: methyl 6-[(2-cyanopyridin-4-yl)oxy]-5,6,7,8-tetrahydronaphthalene-2-carboxylate Intermediate 90

A mixture of methyl 6-hydroxy-5,6,7,8-tetrahydronaphthalene-2-carboxylate (0.100 g, 0.48 mmol), 4-nitro-2-pyridinecarbonitrile (0.144 g, 0.969 mmol), cesium carbonate (0.473 g, 1.45 mmol) and 1,4-dioxane (2.00 mL) was heated in a sealed tube at 100° C. for 24 h. Water was added and the mixture was extracted into EtOAc (2×). The organic phases were washed with brine, dried over anhydrous Na₂SO₄, filtered and evaporated. The residue was purified by silica gel chromatography (0% to 50% EtOAc/hexane) to afford the title compound (142 mg, 95%). LCMS (FA) ES+ 309.

### Step 2: methyl 4-{[6-(methoxycarbonyl)-1,2,3,4-tetrahydronaphthalen-2-yl]oxy}pyridine-2-carboxylate Intermediate 91

To a solution of methyl 6-[(2-cyanopyridin-4-yl)oxy]-5,6,7,8-tetrahydronaphthalene-2-carboxylate (0.507 g, 1.64 mmol) in methanol (13.5 mL) was added 0.5 M sodium methoxide in methanol (1.60 mL, 0.82 mmol). The reaction was heated at 65° C. for 2 h, then hydrochloric acid (1 N, 8.75 mL, 8.75 mmol) was added at rt and the mixture was stirred for 2 h. The solvents were evaporated and the residue was basified by addition of sat. NaHCO₃ solution and extracted into EtOAc (2×). The organic phases were washed with brine, dried (Na₂SO₄) and evaporated to afford the title compound (569 mg, quant). ¹H NMR (400 MHz, CD₃OD) δ ppm 8.46 (d, J=5.7 Hz, 1H), 7.79 (s, 1H), 7.75 (d, J=8.0 Hz, 1H), 7.68 (d, J=2.5 Hz, 1H), 7.23 (dd, J=5.8, 2.6 Hz, 1H), 7.20 (d, J=8.1 Hz, 1H), 5.15-5.09 (m, 1H), 3.95 (s, 3H), 3.88 (s, 3H), 3.34-3.27 (m, 1H), 3.11-2.89 (m, 3H), 2.25-2.11 (m, 2H).

### Step 3: 4-{[6-(methoxycarbonyl)-1,2,3,4-tetrahydronaphthalen-2-yl]oxy}pyridine-2-carboxylic acid Intermediate 92

To a solution of methyl 4-{[6-(methoxycarbonyl)-1,2,3,4-tetrahydronaphthalen-2-yl]oxy}pyridine-2-carboxylate (0.569 g, 1.67 mmol) in methanol (17.2 mL) was slowly added 1.0 M potassium hydroxide in water (1.67 mL, 1.67 mmol) and the reaction mixture was stirred at rt for 3 h. 1.0 M potassium hydroxide in water (0.5 mL, 0.5 mmol) was added and the reaction was stirred for 3 h. The reaction was quenched with 1.0 M of hydrochloric acid in water and concentrated to afford the title compound (578 mg, quant). LCMS (FA) ES+ 328.

### Step 4: tert-butyl 4-{[(4-{[6-(methoxycarbonyl)-1,2,3,4-tetrahydronaphthalen-2-yl]oxy}pyridin-2-yl)carbonyl]amino}piperidine-1-carboxylate Intermediate 93

To a solution of 4-{[6-(methoxycarbonyl)-1,2,3,4-tetrahydronaphthalen-2-yl]oxy}pyridine-2-carboxylic acid (0.136 g, 0.415 mmol) was in N,N-dimethylformamide (2.25 mL, 29.0 mmol) and was added N,N-diisopropylethylamine (0.217 mL, 1.25 mmol) and fluoro-N,N,N′,N′-tetramethylformamidinium hexafluorophosphate (0.165 g, 0.623 mmol). The reaction mixture was stirred for 30 min. tert-Butyl 4-aminopiperidine-1-carboxylate (0.0915 g, 0.457 mmol) was added and the reaction was stirred at room temperature for 5 h. Water was added and extracted into ethyl acetate (2×). The organic phases were washed with brine, dried over anhydrous sodium sulfate, filtered and concentrated to give afford the title compound (161 mg, 76%). ¹H NMR (400 MHz, CDCl₃) δ ppm 8.32 (d, J=5.6 Hz, 1H), 8.00 (d, J=8.4 Hz, 1H), 7.82 (s, 1H), 7.80 (d, J=7.9 Hz, 1H), 7.75 (d, J=2.5 Hz, 1H), 7.14 (d, J=8.0 Hz, 1H), 6.91 (dd, J=5.6, 2.6 Hz, 1H), 5.02-4.96 (m, 1H), 4.16-3.99 (m, 3H), 3.91 (s, 3H), 3.27 (dd, J=17.3, 4.72 Hz, 1H), 3.11-2.86 (m, 5H), 2.23-2.10 (m, 2H), 2.03-1.95 (m, 2H), 1.55-1.42 (m, 11H).

### Step 5: tert-butyl 4-({[4-({6-[(hydroxyamino)carbonyl]-1,2,3,4-tetrahydronaphthalen-2-yl}oxy)pyridin-2-yl]carbonyl}amino)piperidine-1-carboxylate Compound I-223

The title compound was prepared from tert-butyl 4-{[(4-{[6-(methoxycarbonyl)-1,2,3,4-tetrahydronaphthalen-2-yl]oxy}pyridin-2-yl)carbonyl]amino}piperidine-1-carboxylate following the procedure outlined in Example 3, step 3. LCMS (AA) ES+ 511; NMR (400 MHz, CD₃OD) δ 8.41 (d, J=5.7 Hz, 1H), 7.63 (d, J=2.5 Hz, 1H), 7.52 (s, 1H), 7.49 (d, J=7.9 Hz, 1H), 7.18 (d, J=8.0 Hz, 1H), 7.10 (dd, J=5.7, 2.6 Hz, 1H), 5.11-5.03 (m, 1H), 4.12-3.99 (m, 3H), 3.29-3.25 (m, 1H), 3.09-2.88 (m, 5H), 2.24-2.09 (m, 2H), 1.96-1.89 (m, 2H), 1.54 (ddd, J=15.7, 12.2, 4.1 Hz, 2H), 1.46 (s, 9H).

### Step 6: 4-({6-[(hydroxyamino)carbonyl]-1,2,3,4-tetrahydronaphthalen-2-yl}oxy)-N-piperidin-4-ylpyridine-2-carboxamide Compound I-220

The title compound was prepared from tert-butyl 4-({[4-({6-[(hydroxyamino)carbonyl]-1,2,3,4-tetrahydronaphthalen-2-yl}oxy)pyridin-2-yl]carbonyl}amino)piperidine-1-carboxylate following the procedure outlined in Example 30, step 3. LCMS (AA) ES+ 411; ¹H NMR (400 MHz, d₆-DMSO) δ 11.15 (s, 1H), 9.10-9.00 (m, 1H), 8.95-8.85 (m, 1H), 8.73-8.63 (m, 1H), 8.51 (d, J=6.0 Hz, 1H), 7.78 (s, 1H), 7.55 (s, 1H), 7.50 (d, J=7.9 Hz, 1H), 7.39-7.34 (m, 1H), 7.17 (d, J=8.0 Hz, 1H), 5.31-5.20 (m, 1H), 4.16-4.01 (m, 2H), 3.35-3.21 (m, 3H), 3.07-2.84 (m, 6H), 2.19-1.99 (m, 2H), 1.99-1.77 (m, 4H).

### Example 55

The following compounds were prepared from 4-{[6-(methoxycarbonyl)-1,2,3,4-tetrahydronaphthalen-2-yl]oxy}pyridine-2-carboxylic acid and the appropriate amine, following the procedure outlined in Example 54, steps 4 and 5

| Compound | LC-MS |
|---|---|
| I-226 | ES+ 427 (FA) |
| I-227 | ES+ 439 (FA) |

### Example 56

### HDAC6 Enzyme Assay

To measure the inhibition of HDAC6 activity, purified human HDAC6 (BPS Bioscience; Cat. No. 5006) is incubated with substrate Ac-Arg-Gly-Lys(Ac)-AMC peptide (Bachem Biosciences; Cat. No. I-1925) for 1 hour at 30° C. in the presence of test compounds or vehicle DMSO control. The reaction is stopped with the HDAC inhibitor trichostatin A (Sigma; Cat. No. T8552) and the amount of Arg-Gly-Lys-AMC generated is quantitated by digestion with trypsin (Sigma; Cat. No. T1426) and subsequent measurement of the amount of AMC released using a fluorescent plate reader (Pherastar; BMG Technologies) set at Ex 340 nm and Em 460 nm. Concentration response curves are generated by calculating the fluorescence increase in test compound-treated samples relative to DMSO-treated controls, and enzyme inhibition (IC₅₀) values are determined from those curves.

### Example 57

### Nuclear Extract HDAC Assay

As a screen against Class I HDAC enzymes, HeLa nuclear extract (BIOMOL; Cat. No. KI-140) is incubated with Ac-Arg-Gly-Lys(Ac)-AMC peptide (Bachem Biosciences; Cat. No. 1-1925) in the presence of test compounds or vehicle DMSO control. The HeLa nuclear extract is enriched for Class I enzymes HDAC1, -2 and -3. The reaction is stopped with the HDAC inhibitor Trichostatin A (Sigma; Cat. No. T8552) and the amount of Arg-Gly-Lys-AMC generated is quantitated by digestion with trypsin (Sigma; Cat. No. T1426) and subsequent measurement of the amount of AMC released using a fluorescent plate reader (Pherastar; BMG Technologies) set at Ex 340 nm and Em 460 nm. Concentration response curves are generated by calculating the fluorescence increase in test compound-treated samples relative to DMSO-treated controls, and enzyme inhibition (IC₅₀) values are determined from those curves.

### Example 58

### Western Blot and Immunofluorescence Assays

Cellular potency and selectivity of compounds are determined using a published assay (Haggarty et al., Proc. Natl. Acad. Sci. USA 2003, 100 (8): 4389-4394) using Hela cells (ATCC cat# CCL-2™) which are maintained in MEM medium (Invitrogen) supplemented with 10% FBS; or multiple myeloma cells RPMI-8226 (ATCC cat# CCL-155™) which are maintained in RPMI 1640 medium (Invitrogen) supplemented with 10% FBS. Briefly, cells are treated with inhibitors for 6 or 24 h and either lysed for Western blotting, or fixed for immunofluorescence analyses. HDAC6 potency is determined by measuring K40 hyperacetylation of alpha-tubulin with an acetylation selective monoclonal antibody (Sigma cat# T7451) in IC50 experiments. Selectivity against Class I HDAC activity is determined similarly using an antibody that recognizes hyperacetylation of histone H4 (Upstate cat#06-866) in the Western blotting assay or nuclear acetylation (Abeam cat# ab21623) in the immunofluorescence assay.

### Example 59

### In Vivo Tumor Efficacy Model

Female NCr-Nude mice (age 6-8 weeks, Charles River Labs) are aseptically injected into the subcutaneous space in the right dorsal flank with 1.0-5.0×10⁶ cells (SKOV-3, HCT-116, BxPC3) in 100 μL of a 1:1 ratio of serum-free culture media (Sigma Aldrich) and BD Matrigel™ (BD Biosciences) using a 1 mL 26 ⅜ gauge needle (Becton Dickinson Ref#309625). Alternatively, some xenograft models require the use of more immunocompromised strains of mice such as CB-17 SCID (Charles River Labs) or NOD-SCID (Jackson Laboratory). Furthermore, some xenograft models require serial passaging of tumor fragments in which small fragments of tumor tissue (approximately 1 mm³) are implanted subcutaneously in the right dorsal flank of anesthetized (3-5% isoflourane/oxygen mixture) NCr-Nude, CB-17 SCID or NOD-SCID mice (age 5-8 weeks, Charles River Labs or Jackson Laboratory) via a 13-ga trocar needle (Popper & Sons 7927). Tumor volume is monitored twice weekly with Vernier calipers. The mean tumor volume is calculated using the formula V=W²×L/2. When the mean tumor volume is approximately 200 mm³, the animals are randomized into treatment groups of ten animals each. Drug treatment typically includes the test compound as a single agent, and may include combinations of the test compound and other anticancer agents. Dosing and schedules are determined for each experiment based on previous results obtained from pharmacokinetic/pharmacodynamic and maximum tolerated dose studies. The control group will receive vehicle without any drug. Typically, test compound (100-200 μL) is administered via intravenous (27-ga needle), oral (20-ga gavage needle) or subcutaneous (27-ga needle) routes at various doses and schedules. Tumor size and body weight are measured twice a week and the study is terminated when the control tumors reach approximately 2000 mm³, and/or if tumor volume exceeds 10% of the animal body weight or if the body weight loss exceeds 20%.

The differences in tumor growth trends over time between pairs of treatment groups are assessed using linear mixed effects regression models. These models account for the fact that each animal is measured at multiple time points. A separate model is fit for each comparison, and the areas under the curve (AUC) for each treatment group are calculated using the predicted values from the model. The percent decrease in AUC (dAUC) relative to the reference group is then calculated. A statistically significant P value suggests that the trends over time for the two treatment groups are different.

The tumor measurements observed on a date pre-specified by the researcher (typically the last day of treatment) are analyzed to assess tumor growth inhibition. For this analysis, a T/C ratio is calculated for each animal by dividing the tumor measurement for the given animal by the mean tumor measurement across all control animals. The T/C ratios across a treatment group are compared to the T/C ratios of the control group using a two-tailed Welch's t-test. To adjust for multiplicity, a False Discovery Rate (FDR) is calculated for each comparison using the approach described by Benjamini and Hochberg, J. R. Stat. Soc. B 1995, 57:289-300.

As detailed above, compounds of the invention inhibit HDAC6. In certain embodiments, compounds of the invention inhibit HDAC6 with the percent inhibition at a concentration of 0.412 μM shown in the table below.

| | Percent Inhibition | | Percent Inhibition |
|---|---|---|---|
| Compound | at 0.412 μM | Compound | at 0.412 μM |
| I-1 | 34 | I-79 | 94 |
| I-2 | 35 | I-80 | 43 |
| I-3 | 36 | I-81 | 84 |
| I-4 | 25 | I-82 | 85 |
| I-5 | 11 | I-83 | 86 |
| I-6 | 18 | I-84 | 82 |
| I-7 | 22 | I-85 | 75 |
| I-8 | 33 | I-86 | 91 |
| I-9 | 47 | I-87 | 92 |
| I-10 | 20 | I-88 | 89 |
| I-11 | 21 | I-89 | 84 |
| I-12 | 15 | I-90 | 82 |
| I-13 | 10 | I-91 | 93 |
| I-14 | 79 | I-92 | 92 |
| I-15 | 11 | I-93 | 74 |
| I-16 | 55 | I-94 | 96 |
| I-17 | 19 | I-95 | 71 |
| I-18 | 34 | I-96 | 92 |
| I-19 | 20 | I-97 | 92 |
| I-20 | 30 | I-98 | 93 |
| I-21 | 20 | I-99 | 77 |
| I-22 | 28 | I-100 | 62 |
| I-24 | 46 | I-101 | 55 |
| I-26 | 21 | I-102 | 85 |
| I-27 | 11 | I-103 | 63 |
| I-28 | −2 | I-104 | 56 |
| I-29 | 70 | I-105 | 43 |
| I-31 | 17 | I-106 | 90 |
| I-32 | 69 | I-107 | 84 |
| I-33 | 62 | I-108 | 95 |
| I-34 | 2 | I-109 | 53 |
| I-35 | 74 | I-110 | 42 |
| I-36 | 26 | I-111 | 75 |
| I-37 | 36 | I-112 | 87 |
| I-38 | 18 | I-113 | 53 |
| I-39 | 60 | I-114 | 82 |
| I-40 | 48 | I-115 | 81 |
| I-41 | 23 | I-116 | 81 |
| I-43 | 71 | I-117 | 92 |
| I-45 | 47 | I-118 | 75 |
| I-46 | 22 | I-119 | 74 |
| I-47 | 82 | I-120 | 85 |
| I-48 | 62 | I-121 | 80 |
| I-49 | 56 | I-122 | 88 |
| I-50 | 70 | I-123 | 90 |
| I-52 | 80 | I-125 | 87 |
| I-54 | 87 | I-150 | 86 |
| I-55 | 78 | I-170 | 73 |
| I-57 | 59 | I-171 | 77 |
| I-58 | 85 | I-183 | 69 |
| I-59 | 56 | I-185 | 85 |
| I-61 | 84 | I-190 | 90 |
| I-62 | 75 | I-194 | 86 |
| I-63 | 18 | I-196 | 32 |
| I-64 | 18 | I-197 | 55 |
| I-65 | 16 | I-198 | 60 |
| I-66 | 49 | I-199 | 70 |
| I-67 | 26 | I-200 | 67 |
| I-68 | 65 | I-201 | 29 |
| I-69 | 44 | I-202 | 19 |
| I-70 | 63 | I-203 | 53 |
| I-71 | 60 | I-204 | 23 |
| I-72 | 54 | I-205 | 17 |
| I-73 | 73 | I-206 | 18 |
| I-74 | 20 | I-207 | 25 |
| I-75 | 90 | I-208 | 34 |
| I-76 | 80 | I-210 | 28 |
| I-77 | 92 | I-211 | 51 |
| I-78 | 88 | I-214 | 88 |
| I-148 | 93 | I-224 | 94 |
| I-225 | 95 | I-228 | 90 |
| I-229 | 92 | I-220 | 92 |
| I-223 | 88 | I-222 | 91 |
| I-219 | 90 | I-227 | 95 |
| I-221 | 41 | I-226 | 93 |
| I-212 | 72 | | |

As detailed above, compounds of the invention are selective for HDAC6 over other Class I HDAC enzymes. In some embodiments, the ratio of HDAC IC50 (as obtained in the nuclear extract assay described above) to HDAC6 IC50 is less than 5 (HDAC IC50/HDAC6 IC50). In certain embodiments, the ratio of HDAC IC50 to HDAC6 IC50 is between 5 and 10. In certain embodiments, the ratio of HDAC IC50 to HDAC6 IC50 is between 10 and 100.

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of examples.

What is claimed is:

1. A compound of formula (I): 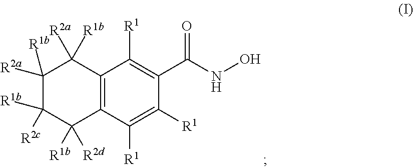
or a pharmaceutically acceptable salt thereof;
wherein:
each occurrence of R¹ is independently hydrogen, chloro, fluoro, —O—C₁₋₄ alkyl, cyano, hydroxy, C₁₋₄ alkyl, C₁₋₄ fluoroalkyl, —N(C₁₋₄ alkyl)₂, —NH(C₁₋₄ alkyl), —NH₂, or O—C₁₋₄ fluoroalkyl;
R^{2a} is G or R^{1a};
R^{2b} is G or R^{1a};
R^{2c} is G or R^{1a};
R^{2d} is G or R^{1a};
provided that one and only one of R^{2a}, R^{2b}, R^{2c}, and R^{2d} is G;
each occurrence of R^{1a} is independently hydrogen, fluoro, C₁₋₄ alkyl, or C₁₋₄ fluoroalkyl;
each occurrence of R^{1b} is independently hydrogen, fluoro, or C₁₋₄ alkyl;
or one occurrence of R^{1a} and one occurrence of R^{1b} on the same carbon atom can be taken together to form ═O or a 3-6 membered cycloaliphatic;
G is hydrogen, —R³, -V₁-R³, -V₁-L₁-R³, -L₁-V₁-R³, or -L₁-R³;
L₁ is an unsubstituted or substituted C₁₋₃ alkylene chain;
V₁ is —C(O)—, —C(S)—, —C(O)—N(R^{4a})—, —C(O)—O—, —N(R^{4a})—, —N(R^{4a})—C(O)—, —N(R^{4a})—SO₂—, —O—, —N(R^{4a})—C(O)—N(R^{4a})—, —N(R^{4a})—C(O)—O—, —O—C(O)—N(R^{4a})—, or —N(R^{4a})—SO₂—N(R^{4a})—;
R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; and
each occurrence of R^{4a} is independently hydrogen, or unsubstituted or substituted C₁₋₄ aliphatic; or when V₁ is —N(R^{4a})—C(O)—, —N(R^{4a})—SO₂—, or —N(R^{4a})—C(O)—N(R^{4a})—, one occurrence of R^{4a} can be taken together with an R^{1a} attached to a ring carbon atom that is not adjacent to the ring carbon atom to which G is attached to form a substituted or unsubstituted 5-7 membered bridged heterocyclyl;
provided that the compound is other than 8-(2-amino-8-bromo-1,6-dihydro-6-oxo-9H-purin-9-yl)-5,6,7,8-tetrahydro-N-hydroxy-2-naphthalenecarboxamide.

2. The compound of claim 1, wherein G is —R³, -V₁-R³, -V₁-L₁-R³, -L₁-V₁-R³, or -L₁-R³.

3. The compound of claim 2, wherein:
G is -V₁-R³, -L₁-R³, or —R³;
L₁ is CH₂— or CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

4. The compound of claim 2, wherein:
each occurrence of R^{1a} is independently hydrogen, fluoro, or methyl;
each occurrence of R^{1b} is independently hydrogen, fluoro, or methyl; and
each occurrence of R¹ is independently hydrogen, chloro, fluoro, cyano, hydroxy, methoxy, ethoxy, trifluoromethoxy, trifluoromethyl, methyl, or ethyl.

5. The compound of claim 2, wherein:
each substitutable carbon chain atom in R³ is unsubstituted or substituted with 1-2 occurrences of —R^{5dd};
each substitutable saturated ring carbon atom in R³ is unsubstituted or substituted with ═O, ═C(R⁵)₂, or R^{5aa};
each substitutable unsaturated ring carbon atom in R³ is unsubstituted or is substituted with —R^{5a};
each substitutable ring nitrogen atom in R³ is unsubstituted or substituted with —R^{9b};
each R^{5a} is independently halogen, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —NR⁴CO₂R⁶, —C(O)N(R⁴)₂, —C(O)R⁶, —C(O)N(R⁴)₂, —N(R⁴)SO₂R⁶, —N(R⁴)SO₂N(R⁴)₂, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two adjacent R^{5a}, taken together with the intervening ring atoms, form an unsubstituted or substituted fused 5-10 membered aromatic ring or an unsubstituted or substituted 4-10 membered non-aromatic ring having 0-3 heteroatoms independently selected from nitrogen, oxygen, and sulfur;
each occurrence of R^{5aa} is independently chloro, fluoro, hydroxy, unsubstituted or substituted C₁₋₆ aliphatic, —O(C₁₋₆ alkyl), —C₁₋₆ fluoroalkyl, —O—C₁₋₆ fluoroalkyl, cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, or —NHS(O)₂C₁₋₆ alkyl;
each occurrence of R^{5dd} is independently fluoro, hydroxy, —O(C₁₋₆ alkyl), cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, or —NHS(O)₂C₁₋₆ alkyl;
each R⁴ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two R⁴ on the same nitrogen atom, taken together with the nitrogen atom, form an unsubstituted or substituted 5- to 6-membered heteroaryl or an unsubstituted or substituted 4- to 8-membered heterocyclyl having, in addition to the nitrogen atom, 0-2 ring heteroatoms selected from nitrogen, oxygen, and sulfur;
each R⁵ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur;
each R⁶ is independently unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur;
each R^{9b} is independently —C(O)R⁶, —C(O)N(R⁴)₂, —CO₂R⁶, —SO₂R⁶, —SO₂N(R⁴)₂, unsubstituted C₃₋₁₀ cycloaliphatic, C₃₋₁₀ cycloaliphatic substituted with 1-2 independent occurrences of R⁷ or R⁸, unsubstituted C₁₋₆ aliphatic, or C₁₋₆ aliphatic substituted with 1-2 independent occurrences of R⁷ or R⁸;
each R⁷ is independently unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; and
each R⁸ is independently chloro, fluoro, —OH, —O(C₁₋₆ alkyl), —CN, —N(R⁴)₂, —C(O)(C₁₋₆ alkyl), —CO₂H, —CO₂(C₁₋₆ alkyl), —C(O)NH₂, —C(O)NH(C₁₋₆ alkyl), or —C(O)N(C₁₋₆ alkyl)₂.

6. The compound of claim 5, wherein:
each substitutable saturated ring carbon atom in R³ is unsubstituted or substituted with —R^{5aa};
the total number of R^{5a} and R^{5aa} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted O—C₁₋₆ alkyl, —O—C₁₋₆ alkyl substituted with 1-2 independent occurrences of R⁷ or R⁸, C₁₋₆ fluoroalkyl, fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆ alkyl)₂, 3-10-membered cycloaliphatic substituted with 0-2 occurrences of R^{7a}, 4-10-membered heterocyclyl having 1-4 heteroatoms independently selected from nitrogen, oxygen, and sulfur substituted with 0-2 occurrences of R^{7a}, 6-10-membered aryl substituted with 0-2 occurrences of R^{7a}, or 5-10-membered heteroaryl having 1-5 heteroatoms independently, selected from nitrogen, oxygen, and sulfur substituted with 0-2 occurrences of R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆ alkyl)₂, —NHC(O)NHC₁₋₆ alkyl, —NHC(O)N(C₁₋₆ alkyl)₂, or —NHS(O)₂C₁₋₆ alkyl.

7. The compound of claim 5, represented by formula (I-b):

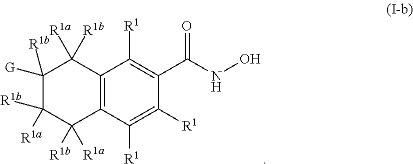

8. The compound of claim 7, wherein:
each occurrence of R^{1a} is independently hydrogen, fluoro, or methyl;
each occurrence of R^{1b} is independently hydrogen, fluoro, or methyl; and
each occurrence of R¹ is independently hydrogen, chloro, fluoro, cyano, hydroxy, methoxy, ethoxy, trifluoromethoxy, trifluoromethyl, methyl, or ethyl.

9. The compound of claim 7, wherein:
G is -V₁-R³, -L₁-R³, or —R³;
L₁ is CH₂— or CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

10. The compound of claim 7, wherein:
each occurrence of R^{1a} is hydrogen;
each occurrence of R^{1b} is hydrogen; and
each occurrence of R¹ is hydrogen.

11. The compound of claim 7, wherein:
each substitutable saturated ring carbon atom in R³ is unsubstituted or substituted with —R^{5aa};
the total number of R^{5a} and R^{5aa} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆alkyl)₂, 3-10-membered cycloaliphatic substituted with 0-2 occurrences of R^{7a}, 4-10-membered heterocyclyl having 1-4 heteroatoms independently selected from nitrogen, oxygen, and sulfur substituted with 0-2 occurrences of R^{7a}, 6-10-membered aryl substituted with 0-2 occurrences of R^{7a}, or 5-10-membered heteroaryl having 1-5 heteroatoms independently selected from nitrogen, oxygen, and sulfur substituted with 0-2 occurrences of R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆ alkyl)₂, —NHC(O)NHC₁₋₆ alkyl, —NHC(O)N(C₁₋₆alkyl)₂, or —NHS(O)₂C₁₋₆ alkyl.

12. The compound of claim 5, represented by formula (I-c):

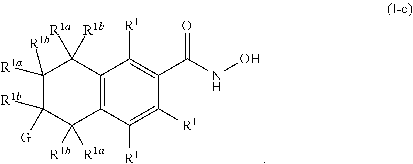

13. The compound of claim 12, wherein:
each occurrence of R^{1a} is independently hydrogen, fluoro, or methyl;
each occurrence of R^{1b} is independently hydrogen, fluoro, or methyl; and
each occurrence of R¹ is independently hydrogen, chloro, fluoro, cyano, hydroxy, methoxy, ethoxy, trifluoromethoxy, trifluoromethyl, methyl, or ethyl.

14. The compound of claim 12, wherein:
G is -V₁-R³, -L₁-R³, or —R³;
L₁ is CH₂— or CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

15. The compound of claim 12, wherein:
each occurrence of R^{1a} is hydrogen;
each occurrence of R^{1b} is hydrogen; and
each occurrence of R¹ is hydrogen.

16. The compound of claim 12, wherein:
each substitutable saturated ring carbon atom in R³ is unsubstituted or substituted with —R^{5aa};
the total number of R^{5a} and R^{5aa} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted O—C₁₋₆ alkyl, —O—C₁₋₆ alkyl substituted with 1-2 independent occurrences of R⁷ or R⁸, C₁₋₆ fluoroalkyl, fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆alkyl)₂, 3-10-membered cycloaliphatic substituted with 0-2 occurrences of R^{7a}, 4-10-membered heterocyclyl having 1-4 heteroatoms independently selected from nitrogen, oxygen, and sulfur substituted with 0-2 occurrences of R^{7a}, 6-10-membered aryl substituted with 0-2 occurrences of R^{ea}, or 5-10-membered heteroaryl having 1-5 heteroatoms independently selected from nitrogen, oxygen, and sulfur substituted with 0-2 occurrences of R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆alkyl)₂, —C(O)NHC₁₋₆ alkyl, —C(O)N(C₁₋₆ alkyl)₂, —NHC(O)NHC₁₋₆ alkyl, —NHC(O)N(C₁₋₆alkyl)₂, or —NHS(O)₂C₁₋₆ alkyl.

17. The compound of claim 7, represented by formula (I-d):

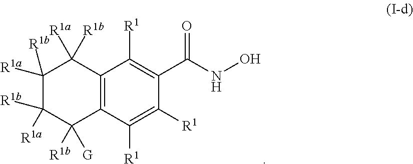

18. The compound of claim 17, wherein:
each occurrence of R^{1a} is independently hydrogen, fluoro, or methyl;
each occurrence of R^{1b} is independently hydrogen, fluoro, or methyl; and
each occurrence of R¹ is independently hydrogen, chloro, fluoro, cyano, hydroxy, methoxy, ethoxy, trifluoromethoxy, trifluoromethyl, methyl, or ethyl.

19. The compound of claim 17, wherein:
G is -V₁-R³, -L₁-R³, or —R³;
L₁ is CH₂— or CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

20. The compound of claim 17, wherein:
each occurrence of R^{1a} is hydrogen;
each occurrence of R^{1b} is hydrogen; and
each occurrence of R¹ is hydrogen.

21. The compound of claim 17, wherein:
each substitutable saturated ring carbon atom in R³ is unsubstituted or substituted with —R^{5aa};
the total number of R^{5a} and R^{5aa} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆alkyl)₂, 3-10-membered cycloaliphatic substituted with 0-2 occurrences of R^{7a}, 4-10-membered heterocyclyl having 1-4 heteroatoms independently selected from nitrogen, oxygen, and sulfur substituted with 0-2 occurrences of R^{6a}, 6-10-membered aryl substituted with 0-2 occurrences of R^{7a}, or 5-10-membered heteroaryl having 1-5 heteroatoms independently selected from nitrogen, oxygen, and sulfur substituted with 0-2 occurrences of R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆ alkyl)₂, —NHC(O)NHC₁₋₆ alkyl, —NHC(O)N(C₁₋₆ alkyl)₂, or —NHS(O)₂C₁₋₆ alkyl.

22. A pharmaceutical composition comprising a compound of claim 1 and a pharmaceutically acceptable carrier.

23. A method of treating a proliferative disorder in a patient comprising administering to said patient a therapeutically effective amount of a compound of claim 1.

24. The method of claim 23, wherein the proliferative disorder is breast cancer, lung cancer, ovarian cancer, multiple myeloma, acute myelogenous leukemia, or acute lymphoblastic leukemia.

25. A method for inhibiting HDAC6 activity in a patient comprising administering a pharmaceutical composition comprising an amount of a compound of claim 1 effective to inhibit HDAC6 activity in the patient.
